SEQUENCE LISTING (2 sequences)

SEQ ID NO: 1 - protein, 262 aa, Heliothis virescens
VQELSIERLLEMESLVADPSEEFQFLRVGPDSNVPPKFRAPVSSLCQIGNKQIAALVVWARDIPHFSQLEMEDQILLIKGSWNELLLFAIAWRSMEFLTEERDGVDGTGNRTTSPPQLMCLMPGMTLHRNSALQAGVGQIFDRVLSELSLKMRTLRVDQAEYVALKAIILLNPDVKGLKNRQEVEVLREKMFLCLDEYCRRSRSSEEGRFAALLLRLPALRSISLKSFEHLFFFHLVADTSIAGYIRDALRNHAPPIDTNMM

SEQ ID NO: 2 - protein, 466 aa, Heliothis virescens
MSVAKKDKPTMSVTALINWARPLPPGQQQQPMTPTSPGNMLQPMATPSNLPTVDCSLDIQWLNLEGGFMSPMSPPEMKPDTAMLDGLRDDSTPPPAFKNYPPNHPLSGSKHLCSICGDRASGKHYGVYSCEGCKGFFKRTVRKDLTYACREERNCIIDKRQRNRCQYCRYQKCLACGMKREAVQEERQRAARGTEDAHPSSSVQVQELSIERLLEMESLVADPSEEFQFLRVGPDSNVPPKFRAPVSSLCQIGNKQIAALVVWARDIPHFSQLEMEDQILLIKGSWNELLLFAIAWRSMEFLTEERDGVDGTGNRTTSPPQLMCLMPGMTLHRNSALQAGVGQIFDRVLSELSLKMRTLRVDQAEYVALKAIILLNPDVKGLKNRQEVEVLREKMFLCLDEYCRRSRSSEEGRFAALLLRLPALRSISLKSFEHLFFFHLVADTSIAGYIRDALRNHAPPIDTNMM

(19) United States
(12) Patent Application Publication
(10) Pub. No.: US 2003/0027984 A1
(43) Pub. Date: Feb. 6, 2003

(54) Ligand-binding domain of the ultraspiracle (USP) protein

(76) Inventors: Eva-Maria Franken, Leichlingen (DE); Martina Janssen, Konigswinter (DE); Michael Schindler, Bergisch Gladbach (DE); Klaus Tietjen, Landgenfeld (DE); Dino Moras, Lampertingen (FR); Jean-Marie Wurtz, Drusenheim (FR); Natacha Rochel-Guiberteau, Strasbourg (FR); Isabelle Billas-Massobrio, Strasbourg (FR)

Correspondence Address: Patent Department, Bayer Corporation, 100 Bayer Road, Pittsburgh, PA, 15205-9741, US

(21) Appl. No.: 09/909,556

(22) Filed: Jul. 20, 2001

(30) Foreign Application Priority Data
Jul. 25, 2000 (DE) 10036461.6

(51) Int. Cl. C07K 14/435; G06F 19/00; G01N 33/48; G01N 33/50

(52) U.S. Cl. 530/350; 702/19

(57) ABSTRACT

The invention relates to the spatial structure of the ligand-binding domain of the ultraspiracle protein, to the use of this structure for generating protein models of this protein in various conformations and of related proteins, and to methods of finding ligands of the ultraspriracle protein and of related proteins.

The invention relates to the spatial structure of the ligand-binding domain of the ultraspiracle protein, to the use of this structure for generating protein models of this protein in various conformations and of related proteins, and to methods of finding ligands of the ultraspriracle protein and of related proteins.

The ultraspiracle protein (termed USP hereinbelow) is the insect orthologue of the vertebrate retinoid X receptor (RXR). Like RXR, it belongs to the family of the nuclear receptors (NR). These nuclear receptors are located in the interior of the cell. They bind to responsive elements on the DNA as homo- or heterodimers and regulate the expression of genes. In order to be active they must bind specific small, frequently hydrophobic, ligands (for example steroids, retinoids, vitamin D). Nuclear receptors have a modular structure with functional domains for transactivation, DNA-binding and ligand-binding. While the DNA-binding domain of the nuclear receptors is highly conserved, the ligand-binding domains only show moderate homologies among each other. The spatial structures of various ligand-binding domains have already been determined (summary in 2) and allow an insight into the mechanism on which the activation is based, which comprises pronounced changes in the conformation of the ligand-binding domains. The binding of agonists leads to activation owing to the displacement of bound corepressors and the binding of coactivators, while the binding of antagonists prevents the interaction with the coactivator.

No spatial structures are available yet of insect nuclear receptors. In insects, for example the development from the larva to the adult insect is governed by nuclear receptors and involves the steroid hormone ecdysone and the isoprenoid juvenile hormone (3, 4, 5, 6). The ecdysone receptor, a nuclear receptor composed of two different subunits, EcR and USP, plays a key role in this process (7, 8, 9). It has been known for a long time that the hormone ecdysone (in its active form 20-hydroxy-ecdysone) acts as ligand for the EcR subunit.

The ecdysone receptor constitutes an important insecticide target. If it is activated outside the windows in the period provided for insect development, this leads to severe damage or even to the death of the insects. The insecticidal action of ecdysone agonists is based on this mechanism (10, 11). Non-steroidal ligands of the EcR subunit which act specifically on Lepidoptera are already being used commercially as insecticides (12).

USP is an orphan receptor for which no ligand is known as yet. While various authors have assumed that USP constitutes a receptor for juvenile hormones, this has never been proven by actual experiments (9). Indeed, it has been assumed that USP has no ligand at all, as this is described for some other nuclear receptors known from animals.

It was therefore an object of the present invention to provide the spatial structure of the ligand-binding domain (termed LBD hereinbelow) of the USP and to describe the possible ligand-binding pocket.

The object was achieved by providing a USP-LBD in crystalline form and by successfully carrying out the X-ray structure analysis of the crystals thus obtained.

The crystalline LBD according to the invention is preferably an LBD of the Heliothis virescens USP. The LBD according to the invention especially preferably has an amino acid sequence shown in SEQ ID NO: 1.

The subject-matter of the present invention is also a crystalline complex of a USP-LBD with a ligand.

The LBD according to the invention preferably has the structure coordinates defined in Table 1. The three-dimensional structure was solved and fully refined with the aid of protein crystals which are accessible to X-ray structure analysis at high resolution by means of molecular replacement. Subject-matter of the present invention is thus also the three-dimensional structure of the USP-LBD which can be determined with the aid of these structure coordinates.

A ligand-binding pocket, into which—like in the case of the other known structures of nuclear receptors—the ligands bind, has been identified in the three-dimensional structure according to the invention of the USP-LBD described herein. This is the first actual confirmation for the fact that USP has a functional ligand-binding pocket.

Subject-matter of the present invention is furthermore a USP-LBD comprising a ligand-binding pocket which is defined by the amino acids LEU230, VAL238, PRO239, PHE242, LEU249, LEU291, ILE294, MET323, LEU331, GLN338, ALA339, VAL341, PHE345, SER431, HIS434, LEU435, PHE438 and LEU440 as shown in SEQ ID NO: 2 and Table 1.

Subject-matter of the present invention is furthermore a USP-LBD comprising a ligand-binding pocket defined by the amino acids LEU230, VAL238, PRO239, PHE242, PRO245, VAL246, LEU249, CYS250, GLY253, ASN287, LEU290, LEU291, ILE294, MET323, LEU325, LEU331, SER335, ALA336, GLN338, ALA339, VAL341, ILE344, PHE345, VAL348, SER431, HIS434, LEU435, PHE438 and LEU440 as shown in SEQ ID NO: 2 and Table 1.

Subject-matter of the present invention is furthermore a USP-LBD comprising a ligand-binding pocket which is defined by the above-described amino acids and in which one or more of these amino acids are mutated. These are preferably conservative mutations, where an amino acid is exchanged for an amino acid with similar physical properties.

Such conservative substitutions encompass variations in which an amino acid is replaced by another amino acid from amongst the following group:

1. Small aliphatic residues, nonpolar residues or residues of little polarity: Ala, Ser, Thr, Pro and Gly;

2. Polar, negatively charged residues and their amides: Asp, Asn, Glu and Gln;

3. Polar, positively charged residues: His, Arg and Lys;

4. Large aliphatic nonpolar residues: Met, Leu, Ile, Val and Cys; and

5. Aromatic residues: Phe, Tyr and Trp.

Preferred conservative substitutions can be seen from the following list:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, His |
| Met | Leu, Tyr, Ile |
| Phe | Met, Leu, Tyr |
| Ser | Thr |
| Thr | Ser |
| Trp | Tyr, Phe |
| Tyr | Trp, Phe |
| Val | Ile, Leu |

The three-dimensional structure described herein of a USP-LBD is of great importance for the search for ligands with practical application. Such ligands can be used, for example, as insecticides with a novel mechanism of action. The ecdysone/juvenile hormone-governed development is only found in invertebrates and not in vertebrates; thus, it constitutes an insecticidal mechanism which is safe for the user and the environment.

Using the three-dimensional structure according to the invention of the USP-LBD, databases which contain the structures of a large number of compounds can be screened with the aid of established, automated computer protocols (virtual screening). Algorithms such as FLEXX (13) or GOLD (14) are examples which can be used for virtual screening. With this procedure, compounds can be identified whose three-dimensional structure makes it possible to enter the binding pocket and to bind there, for example by forming hydrogen bonds, by hydrophobic interaction, by electrostatic interactions, by van-der-Waals interactions or by dipole interactions. The compounds identified thus can be synthesized and then used as, for example, insecticides or as effectors in expression systems (gene switch) based on the USP.

Another application of the three-dimensional structure according to the invention of the USP-LBD is the generation of new ligands. To this end, structural formulae for new ligands are generated on the computer using this structure and with the aid of established de-novo design programs, and these new ligands can enter the binding pocket, where they can bind, for example by forming hydrogen bonds, by hydrophobic interaction, by electrostatic interactions, by van-der-Waals interactions or by dipole interactions. Examples of de-novo design programs which are possible are LUDI (15), LEGEND (16) or GROW (17). Compounds generated thus can be synthesized and then also be used as, for example, insecticides or as effectors in expression systems (gene switch) based on the USP.

The three-dimensional structure according to the invention of the USP-LBD also makes it possible to predict the three-dimensional structure of a USP-LBD from other organisms by means of modelling methods. Such protein models can be used in the same manner as the three-dimensional structure solved herein. Comparison of the differences in the amino acid sequences makes it possible to predict differences in the ligand-binding pockets of various organisms. This is of use when specific ligands are searched for for specific organisms, or, conversely, when it is precisely unspecific ligands that are searched for. In addition, the three-dimensional structure according to the invention can be used for establishing protein models of other nuclear receptors with related sequences.

The present invention encompasses in particular the following subject matters and methods:

A computer-readable data storage medium comprising a data storage material on which the structure coordinates of an LBD according to the present invention are stored.

A computer-readable data storage medium in a form which makes it possible to generate a three-dimensional image of an LBD according to the present invention on a computer screen.

A method of generating protein models of USP-LBDs, characterized by the computer-aided generation of a three-dimensional image of an LBD according to the present invention.

A method of generating protein models of USP-LBDs in an agonistic conformation, characterized by the computer-aided generation of a three-dimensional image of an LBD according to the present invention in an agonistic conformation.

A method of generating protein models of nuclear receptors which have homologies with USP-LBDs, characterized by the computer-aided generation of a three-dimensional image of an LBD according to the present invention with a mutated amino acid sequence.

A method for generating protein models of nuclear receptors which have homologies with USP-LBDs in an agonistic conformation, characterized by the computer-aided generation of a three-dimensional image of an LBD according to the present invention with a mutated amino acid sequence in an agonistic conformation.

A method of finding USP ligands, characterized by the following steps:

(a) the computer-aided generation of a three-dimensional image of an LBD according to the present invention, and

(b) the computer-aided (virtual) screening of databases which contain structural data of chemical compounds for those structures which are capable of undergoing specific interactions with an LBD according to the present invention.

A method of finding USP ligands, characterized by the following steps:

(a) the computer-aided generation of a three-dimensional image of an LBD according to the present invention, and

(b) the computer-aided modelling of chemical compounds with structures which are capable of undergoing specific interactions with an LBD according to the present invention.

A method of finding USP-LBD ligands in an agonistic conformation, characterized by the following steps:

(a) the computer-aided generation of a three-dimensional image of an LBD according to the present invention in an agonistic conformation, and

(b) the computer-aided (virtual) screening of databases which contain structural data of chemical compounds for those structures which are capable of undergoing specific interactions with an LBD in an agonistic conformation.

A method of finding USP-LBD ligands in an agonistic conformation, characterized by the following steps:

(a) the computer-aided generation of a three-dimensional image of an LBD according to the present invention in an agonistic conformation, and

(b) the computer-aided modelling of chemical compounds with structures which are capable of undergoing specific interactions with an LBD in an agonistic conformation.

A method of finding active compounds for crop protection, in particular chemical compounds which, owing to binding to an LBD according to the present invention, bring about the activation or inhibition of USP, with the following steps:

(a) carrying out one of the abovementioned methods for finding USP ligands,

(b) synthesizing the compound(s) identified as ligands, and

(c) detecting the bioactivity of the compound synthesized in step (b) by transactivation assays, displacement assays or bioassays.

A method of finding active compounds for crop protection, in particular chemical compounds which, owing to binding to an LBD according to the present invention in an agonistic conformation, bring about the activation or inhibition of USP, with the following steps:

(a) carrying out one of the abovementioned methods for finding USP-LBD ligands in an agonistic conformation,

(b) synthesizing the compound(s) identified as ligands, and

(c) detecting the bioactivity of the compound synthesized in step (b) by transactivation assays, displacement assays or bioassays.

A method of finding effectors for systems for the inducible expression of target genes by means of USP, with the following steps:

(a) carrying out one of the abovementioned methods for finding USP ligands,

(b) synthesizing the compound(s) identified as ligands,

(c) applying a compound synthesized in step (b) to host cells or host organisms which contain a USP-based expression system, and

(d) detecting an induction or inhibition of the expression system.

The use of an LBD according to the present invention or of a computer-readable data storage medium according to the present invention for finding active compounds for crop protection or effectors for the controlled expression of target genes in host cells or intact host organisms.

The present invention is described in greater detail with reference to the examples which follow.

### EXAMPLES

### Example 1

Protein Expression and Purification

The Heliothis virescens USP-LBD (AS Val-205 to Met 466) was cloned in a pET-15b expression vector as N-terminal fusion protein with a His-tag and overexpressed in the E. coli strain BL21(DE3). The cells were cultured in 2× LB medium at 37° C. and induced for 2 hours with 0.8 mM isopropyl-β-D-thiogalactopyranoside at 24° C. The protein extract was purified over a cobalt chelate column with subsequent gel filtration over a Superdex 200 16/60 column. The His-tag was then removed by digestion with thrombin and the protein was removed by gel filtration. A homogeneous monomeric protein species was present in the solution and was confirmed by means of SDS and native polyacrylamide gel electrophoresis and by denaturing and native electrospray ionization mass spectrometry.

Crystallization

Crystallization was effected by gas diffusion on hanging drops. The protein concentration employed was 3-9 mg/ml. Crystals 200×200×400 mm in size formed within 10 days from a solution containing 10% of polyethylene glycol (PEG) 4000, 50 mM Tris (pH 7.5), 100 mM NaCl and 5 mM dithiothreitol and which was equilibrated in the reservoir against a solution of 20% polyethylene glycol (PEG) 4000 and 100 mM Tris (pH 7.5). The crystals belong to the tetragonal P4₃22 spatial group with one monomer per asymmetric unit. The parameters of the standard cell are a=58.21 Å, b=58.21 Å, c=144.69 Å and α=β=γ=90°. The solvent content is 32%, and the B-factor estimated in the Wilson Plot is 27 Å².

Data Gathering, Structure Determination and Refining

Crystals were immersed briefly in a 10% glycerol solution and shock-frozen in liquid nitrogen. The native data set was produced with a single crystal at measuring station ID14-EH2 at the ESRF (Grenoble, France). The data were processed with the aid of HKL programs (18). The crystal structure was solved by the molecular replacement method (19) by means of a partial hRXRα structure (20) as search model. A poor solution was achieved with a correlation of 24.8% and R_{fee}=54.5% after refining as a rigid body. The phasing power of the model was low and required a number of manual generateing cycles with O (21). The wARP method (22) was used to verify the correctness of the partially-built structures. Refining was performed with CNS (23) using a maximum likelihood target function and solvent correction. Cycles of manual modelling and least-square minimization with subsequent simulated annealing and individual anisotropic B-factor refining gave rise to the final model. Solvent molecules were contoured in an F₀−F_{c} map at a surface of 3σ. The final model, refined to a resolution of 1.65 Å, comprises 246 amino acid residues, 259 water molecules and one ligand molecule. A large portion of the connecting loop between Helix H5 and the beginning of the β-pleated sheet (amino acid residues 306-315) and the C-terminal extension of H12 (amino acid residues 459-466) could not be shown, owing to the poor electron density in these regions. The quality of the final model was checked with Procheck (24).

Characterization of the USP-LBD crystals by electrospray time-of-flight mass spectrometry (ESI TOF-MS) under natural conditions shows a heterogeneous mass distribution around 740±50 Da in addition to the peak of the pure protein (30.2 kDa). This suggested that a ligand is present which is bound in LBD. The presence of a ligand was confirmed by the electron density. Various complementary techniques were used to characterize the ligand. The ligand, which is located in the binding pocket of the USP-LBD, was characterized as a phospholipid molecule. A phosphatidylglycerol or a phosphatidylethanolamine or a phosphatidylcholine would match the crystallographic data and are consistent with the results from mass spectroscopy and chemical analysis. These amphiphilic molecules have a head group consisting of a phosphorylglycerol or a phosphorylethanolamine group and a tail of two different fatty acids which are bonded to the glycerol-3-phosphate by ester bonds. A detailed description of the ligand and its interactions with the USP-LBD residues are given in the following text.

### Example 2

Architecture of the Heliothis Virescens USP-LBD

In general, the architecture of the USP-LBD exhibits canonic NR folding with 11 α-helices (H1, H3-H12) and two short β-strands (s1-s2). This structure was compared with two other crystal structures which have essential properties of NRs and which are closely related to the Heliothis virescens USP: the binding pocket of agonist-bound RXRα (hRXRα/9-cis RA) and antagonist-bound mice RXRα (msRXRα/oleic acid). Superposing the USP-LBD with the structure of the holo-RXRα-LBD was carried out with the aid of a least-square fit [LSQ]. In total, the secondary structural elements of the USP-LBD are capable of reasonably good superposition by those of the holo-RXRα-LBD. The root mean square deviation (r.m.s.d.) is 1.22 Å for 183 out of 246 superposed Cα atoms. Seven helices are accessible to reasonably good super-position (r.m.s.d. 1.13, 0.88, 0.57, 1.18, 0.67, 0.69, 0.75 Å for H4, H5, H7-H11). The C-terminus of H1 is curved by approximately 2 Å relative to helix H3, and its r.m.s.d. is 1.63 A. H3, H6 and the P-pleated sheet show larger deviations. The structure of the USP-LBD demonstrates that the activation helix H12 assumes a conformation which is similar to that of the antagonist-RXRα. The antagonistic AF-2 conformation of the USP-LBD is discussed further below.

The connecting loop L1-3 of most NRs usually behaves as a highly flexible region. In the case of hRXRα, the crystal structures of both the apo and the holo conformations show substantial differences in the regions which connect helices H1 and H3. In the holo-LDB structure, L1-3 consists of an extended loop which extends beyone the β-pleated sheet and a ω-loop. The apo form contains an additional helix in this region which unfolds in the holo-form. During the transition from the apo-form to the holo-form, L1-3 undergoes substantial movement. In particular, the ω-loop becomes oriented towards the opposite side of the protein centre. As has been proposed on the basis of the comparison of the two structures, L1-3 might act as a molecular spring which accompanies the conformational changes which are linked to ligand binding. For the ligand-bound RARγ-LBD, the conformation of L1-3 resembles that of the holo-RXRα. Interestingly, L1-3 for ER-LBDs follows a path other than in the case of holo-RXRα. It runs between helix H3 and the P-pleated sheet, packed tightly to the protein centre.

In the case of the USP-LBD, L1-3 assumes none of the conformations which are otherwise found in the other NRs. Its course (Val-220 to Pro-239) was derived unambiguously from the electron density maps. Only few residues at the beginning of the loop, namely Asp-222, Pro-223 and Ser-224, were treated as alanins owing to the poor electron density of the side chains. The temperature factors of these residues are therefore higher (60-64 Å²) than those of the other amino acids of L1-3 (on average 36 Å² over L1-3). The first residues of L1-3 form a path which crosses helix H3 in the region Gln-256 to Val-262. The next residues (Glu-226 to Pro-234) form an extended loop which runs along H3, and, finally, the last five residues of L1-3 (Asp-235 to Pro-239) form a loop which has substantial similarity with the ω-loop observed in the LBDs of RXRα and RARγ. L1-3 assumes quite a tight conformation which makes it possible to establish direct contacts with the residues of helices H3, H 11 and H12 and to stabilize their actual positions. This is important in as far as these helices are those structural elements which are subject to the greatest conformational changes owing to ligand binding.

The particular conformation of L1-3 is not based on crystal packing effects. In the region of the loop L1-3 of RXRα-LBD, the USP-LBD interacts with its symmetry-equivalent molecule via the β-pleated sheets. It is extremely likely that this interaction takes place since L1-3 does not already occupy this region when the protein is in solution. If, owing to packing effects, L1-3 would be forced to swing and to move away from a conformation which is similar to the actual conformation of RXRα, several elements of the secondary structure would have to move drastically from this hypothetical conformation to their final position. It is therefore highly unlikely that this drastic reorganization of all of the LBD takes place, in particular because L1-3 lies in a region of the LBD in which L1-3 establishes very specific interactions with adjacent elements of the secondary structure.

Directly linked to loop L1 -3, helix H3 differs from its counterparts in RXRα both regard to length and with regard to the position of the N- and C-terminal portion. In Heliothis virescens USP, H3 starts at Pro-240 and is therefore one turn longer than H3 in the ligand-bound RXRα (start at RXRα-Pro-264). The residues of H3 in the middle portion of the helix assume almost identical positions compared with the positions of the corresponding residues in the apo- and holo-RXRα-LBDs. However, both N- and C-terminal regions are curved towards the exterior of the protein centre. The N-terminal region of H3 (Pro-240 to Cys-250) is shifted substantially towards H11. It is tilted by approximately 24° in comparison with the same region in the holo-RXRα (approx. 7.2 Å between USP-Pro-245 and holo-RXRα-Pro-264). This position lies between those of the N-terminal regions in the apo-RXRα and the holo-RXRα-LBD structure. The outwardly curved C terminus of H3 (by approx. 10°) has effects on the arrangement of the adjacent loops L3-4 and L8-9. Loop L3-4, which is part of the signature region of NRs, is shifted laterally by approximately 1.8 Å and curved towards L8-9, while loop L8-9 itself is shifted outwardly by approximately 1.5 Å.

### Example 3

The Ligand-Binding Pocket

The ligand-binding pocket of Heliothis virescens USP is formed by residues of loop L1-3, helices H3, H5, H6 and H7, the β-pleated sheet and loop L11-12. As described above, the N-terminal portion of helix H3 is markedly shifted outwardly compared with its opposite number in RXRα. Two other secondary structures which contribute to the binding pocket also differ from those in RXRα: 1) Helix 6 has moved inwardly by approximately 1.9 Å, and 2) the curvature of the β-pleated sheet points towards H1. The shift of the three structural elements which this entails lead to a widening of the ligand-binding pocket compared with that of the RXRα-LBD. The edge of the binding pocket is formed by the ω-loop of L1-3, the N terminus of H3 and H6, while in the case of RXRα the opening of the pocket is formed by loop L11-12 and H6. At its opening, the binding pocket is approximately 13.5 Å wide (distance between Lys-241 in H3 and Gln-338 in H6). This opening is much wider than in the case of RXRα (7,1 Å from Pro-264 in H3 to Ala-340 in H6). The topology of the ligand-binding pocket is relatively unusual with a gap between H3 and H6. In RXRα and other NRs, this region forms fixed contacts with the connecting loop L1-3. The volume of the cavity of the USP-LBD achieves that of the hRXRα-LBD by a factor of 2.5 (1256 Å³ in the case of USP compared with 489 Å³ in the case of hRXRα).

### Example 4

The Putative Ligand of USP in the Crystal Structure

Unexpectedly, the ligand-binding pocket of Heliothis virescens USP contains a molecule which was copurified and cocrystallized together with the USP-LBD. The fit of the electron density agrees well with the characterization of the molecules by mass spectroscopy and analytical chemistry. Similarly, recent crystallographic studies of the heterodimeric RARα/RXRα-LBD show an E. coli-endogenous oleic acid (C18) or a similar compound (stearic (C18) or palmitic (C16) acid) in the RXRα subunit. Even though this molecule is not the natural ligand of vertebrate NR, it induces and stabilizes an antagonistic AF-2 conformation which in all probability is very similar to the actual antagonist-bound RXRα.

In the present case, the best fit of the electron density was assumed with the assumption of a phospholipid whose first tail consists of a fatty acid with a length of 18 carbon atoms at C1 and a second chain at C2 which is 16 carbon atoms in length. The longer fatty acid of the two has a relatively twisted shape with two largish peaks, while the other fatty acid assumes a more normal form within the pocket. The tail of the phospholipid is hidden within the ligand-binding pocket. The glycerol moiety and the two fatty acids form van-der-Waals contacts with the residues in L1-3 (Leu-230, Val-238), H3 (Phe-242, Leu-249), H5 (Leu-291), L6-7 (Ala-339), H7 (Phe-345), H11 (Ser-431, His-434, Phe-438) and L11-12 (Leu-440). The head group of the phospholipid is positioned at the front at the opening of the pocket between H3 and H6. A strong hydrogen bond with Gln-338 (H6) is formed by the carbonyl group of the phosphorylglycerol in the case of phosphatidylglycerol and by the amino group of the ethanolamine in the case of phosphatidylethanolamine. In addition, an oxygen of the phosphate group is bound to a residue L1-3 (Cγ of Pro-239) by a hydrogen bridge.

It is assumed that the phospholipid found herein constitutes no natural USP ligands. However, it is shown unambiguously that USP ligands exist.

The residues which interact with the ligands are highly conserved within lepidopteran USPs, with the exception of Ser-431, which is replaced by a cysteine in msUSP. In contrast, among the 16 residues of the RXRα-LBD which interact with 9-cis RA, only 3 of the corresponding USP residues interact with the phospholipid (Leu-249, Ser-431 and His-434). The reason for this behaviour is mainly the different position of the ligands in the corresponding pockets. The 9-cis RA is very deep within the pocket, where its carboxylate group forms a salt bridge to Arg-316 of helix H5 of the hRXRα. In contrast, the phospholipid does not penetrate far into the inside of the pocket. For example the tail of the longer fatty acid lies approximately at atom C9 of 9-cis RA in hRXRα-LBD, while the tail of the other fatty acid extends almost to the β-ionone ring of 9-cis RA. As a consequence, Arg-297 does not participate in the anchorage of the ligand, as is observed in the case of the agonistic RXRα-, RARγ- and other NR-LBDs. Nevertheless, it assumes almost the same position as Arg-316 of the holo-RXRα and not the position of the apo-RXRα conformation, which is exposed to the solvent. Instead of interacting with the ligand, Arg-297 forms hydrogen bonds with the backbone carbonyl group of Leu-325 (P-pleated sheet) and participates in a hydrogen bond network with Leu-290 (H5) and the side chain of Gln-256 (H3), with participation of water. In particular, two water molecules which are positioned spatially approximately at the two oxygen atoms of the carboxylate group of 9-cis RA participate in these interactions.

### Example 5

The Antagonistic Conformation of the USP-LBD

The AF-2 domain in the structure of the USP-LBD exhibits an antagonistic conformation generated by the ligands in the ligand-binding pocket. H12 assumes the same conformation which has been found in the case of other antagonist-bound nuclear receptors such as RXRα/oleic acid, RARα/BMS614 and ER. In all these cases, it is observed that the groove in which H12 is positioned corresponds to the binding site for the helical nuclear receptor box of nuclear receptor coactivators. This helical nuclear receptor box is distinguished by the consensus sequence LXXLL, as has been shown for the ligand-binding domain of PPARγ, TRβ and ERα. In the case of the Heliothis virescens USP, Ile-450, Ala-453 and Leu-454 of H12 are approximately in the same position as the first, second and third leucin residue of the LXXLL binding motif (IXXAL instead of LXXLL). As in other antagonistic conformations of nuclear receptors, H12 is packed into a groove of residues of H3 and H4 and of L3-4 (Val-261, Arg-265, Met-275, Glu-276, Ile-279, Ile-282, Lys-283). However, in the case of the USP-LBD, L1-3 is also involved in the groove entopology and, with the residues Phe-227, Gln-228 and Phe-229, has van-der-Waals contacts with H12.

The length of H12 in the USP-LBD is identical to that of H12 in the antagonist-bound form of the RXRα-LBD. However, the structural principle which has been observed in another case of an antagonistic conformation of a nuclear receptor ligand-binding pocket is not found in its entirety in the case of the USP-LBD. Indeed, it has been found there that H11 coils up and thus permits H12 to bind to the binding groove of the nuclear receptor coactivator binding motif LXXLL. H11 is located in the extension of H10 and superposes very readily with H 11 in the holo-RXRα-LBD structure, with the exception that the H 11 of the USP-LBD is shorter by two residues. This is followed by a region 6 residues in length which connects H11 and H12 (His-439 to Thr-444). These amino acids of loop L11-12 span, in an extended conformation, a strand 12 Å in length. The C terminus of H11 contains three phenylalanins which are also found in RXRα. In apo-RXRα, the first two phenylalanins point towards the hydrophobic ligand-binding pocket while the third phenylalanin faces the solvent. In the agonist-bound form, the phenylalanins swap roles. In the USP-LBD, the situation is similar to the agonist-bound form of the RXRα-LBD: Phe-436 and Phe-437 face the solvent, while Phe-438 contributes to the ligand-binding pocket. In comparison with its counterpart in RXRα, the side chain of Phe-438 is rotated slightly and touches the ligand at the level of its shorter fatty acid. In the antagonist-bound form of RXRα, the first residue of the phenylalanin triplet corresponds to the end of H11. This residue is in approximately the position of the C-α atom of Phe-437. In the ligand-binding pocket, the two other phenylalanin residues, which are already part of L11-12, are orientated inwardly towards the inside of the protein. In a superposition of the Heliothis virescens USP and the antagonist-bound RXRα-LBD, these two residues collide with the phospholipid ligands.

### Example 6

The Connecting Region L1-3 interacts with H3 and L11-12 and Prevents an Agonistic Conformation

The binding of the phospholipid in the ligand-binding pocket of the USP-LBD probably generates important structural rearrangements in the USP-LBD. The comparison with apo- and holo-RXRα-LBD structures allows the assumption that in the USP-LBD, too, the molecular mechanisms which bring about the ligand-bound LBD conformation comprise the displacement of H3 and H11. However, in contrast to all other nuclear receptor LBDs known to date, the structural element L1-3 plays an essential role in the Heliothis virescens USP.

Loop L1-3 interacts with H3, H11, L11-12 and H12. These structural elements are most effective by the ligand binding. L1-3 stabilizes the N-terminus of H3 via a hydrogen bridge network with Arg-243 and Asn-254 of H3. The guanidinium moiety of the Arg-243 is anchored to the backbone carbonyls of Gly-233, Ser-236 and Val-238 by strong hydrogen bridges (distances 2.61, 2.97 and 2.78 A, respectively) and shows a van-der-Waals contact with the side chain of Val-232. In addition, the backbone amide group of the Arg-243 is bound to the carbonyl group of Pro-239 (3.20 Å) by a hydrogen bond. The side chain of Asn-254 forms hydrogen bonds with the carbonyl group of Leu-230 (2.83 A), to the amide group of Phe-229 (3.10 Å) and, via a water molecule, to the side chain of Gln-228. Moreover, it is in van-der-Waals contact with the carbonyl group of Phe-227. The backbone carbonyl group of Asn-254 forms a strong hydrogen bridge to the side chain of Glu-226 (2.74 Å).

L1-3 (Gln-228 to Arg-231, Asp-235 and Ser-236) is also in contact with N-terminal region of H11 and with L11-12. The backbone carbonyl group of Gln-228 forms a hydrogen bond with Ala-442 (3.20 Å), and the backbone carbonyl group of Phe-229 forms a strong hydrogen bridge with the amide group of Ala-442 (2.88 Å). In addition, Arg-231 stabilizes the loop L11-12 by means of strong interactions: the backbone amide group forms a strong hydrogen bond with the carbonyl group of Leu-440 (2.90 Å), while the side chain forms a strong hydrogen bond with the carbonyl group of His-439 (3.00 Å) and shows van-der-Waals contacts with Val-441 and Ala-442. Other interactions concern the backbone carbonyl of Asp-235 with the side chain of His-439 and a water-mediated interaction with Val-441. The hydroxyl group of Ser-236 forms a van-der-Waals contact with the side chain of Leu-440.

It is important to state that a high degree of sequence conservation exists in all residues which participate in the interaction of L1-3 with H3 and with L11-12. The main interaction partners of H3, Arg-243 and Asn-254, are conserved strictly in all lepidopteran USPs. Likewise all interaction partners in L1-3 (Glu-226, Phe-227, Gln-228, Phe-229, Leu-230, Val-232, Gly-233, Ser-236, Val-238, Pro-239) are conserved strictly in all lepidopteran USPs, with the exception of Phe-227 and Phe-229, which are replaced by leucine and isoleucine in the Bombyx miori USP. In the case of the interactions of L1-3 with L11-12, too, the residues involved (L1-3: Gln-228 to Arg-231, Asp-235 and Ser-236; L11-12: His-439 to Ala-442) are conserved strictly in all lepidopteran USPs, with the exception of Phe-229 and Asp-235. This strongly suggests that interaction patterns of L1-3 with H3 and of L1-3 with L11-12 are similar in all lepidopteran USPs.

In the superposition of Heliothis virescens USP with the holo-RXRα-LBDs, it can be observed that some residues from L1-3 are approximately in the same position in the Heliothis virescens USP (Asn-237, Ser-236 and Phe-229), such as residues from L11-12 of holo-RXRα (Asp-444, Thr-445 and Phe-450). This comparison permits the informative conclusion that L1-3 in its actual conformation excludes the existence of an agonistic conformation since this would be hindered at loop L11-12. In any case, this is no crystallization artefact and reflects the particular role of this structural element in the lepidopteran USPs.

The sterical hindering of the agonistic position of H12 here is a constitutive component of the receptor structure and not the consequence of the bulky shape of the ligand, as is the case in other nuclear receptor LBDs which are occupied by fully antagonistic ligands.

It can be predicted that ligand binding of agonists generates a change in the conformation of the USP-LBD, which makes L1-3, H12 and the other LBD residues which have been described jump into an antagonistic position.

In any case, this is not a crystallization artefact and constitutes the particular role of this structural element in the lepidopteran USPs.

### Example 7

Agonistic Conformation of the Heliothis Virescens USP-LBD by Homology Modelling, Based on the RXRα/9-cis-RA Complex

In order to generate a 3D model of the Heliothis virescens USP-LBD, the lacking residues of the loop between helices H1 and H3 (L1-3) were complemented from the hRXRα crystal structure in such a way that a continuous backbone is formed. The resulting structure is the experimental hRXRα reference model.

Two hRXRα monomers were observed in the hRXRα standard cell, and the L1-3 region was poorly resolved in each of these monomers. Superposition of the two structures, which had been refined independently of one another, led to a suggestion as to where this loop should be modelled. A complete 3D model of hRXRα based on crystal structure and in which the residues in L1-3 are completed was built. The hydrogen atoms were completed with the aid of the Hgenerate option of the Charmm program.

The L1-3 region was relaxed by Powell minimization of the Charmm program (1000 optimization steps, dielectric constant: 4, gradient tolerance: 10⁻⁶, step width 0.02, cutoff for non-binding interactions: 15 Å).

This optimized structure was used as template for the homology model of the Heliothis virescens USP-LBD.

The amino acid sequences of the Heliothis virescens USP-LBD and of hRXRα were assigned in accordance with Table 2.

With the aid of the software package Modeller and its standard settings, a 3D model was built with the aid of the assignment. The USP-LBD sequence shows a few insertions in loop 1-3 and in the loop before the first β-pleated sheet. In order to establish meaningful conformations for these two regions, the option lego-loop of the software package O was used. The USP model structure was subsequently subjected to Powell minimization (2000 optimization steps, dielectric constant: 4, gradient tolerance: 10⁻⁶, step width 0.02, cutoff for non-binding interactions: 15 Å). The quality of the structure thus obtained is analyzed with the program PROCHECK. Accordingly, 97% of the residues are in permitted regions and less than 2% of the residues are in prohibited regions. The latter are in the above-described modified regions.

### Example 8

Comparison Between the Agonistic USP-LBD Structure Obtained from the hRXRα/9-cis RA Complex and the USP-LBD Crystal Structure

The largest differences between these two structures are in the position of the activation helix (H12) and the path of the loop between the helices H1 and H3. The activation helix H12 is located in the experimental structure in the antagonistic position, while in the model structure it assumes an agonistic conformation which closes the ligand-binding niche. In the experimental structure, the loop L1-3 lies above the helix H3 and stabilizes the antagonistic position of H12 by hydrophobic contact. In contrast, this loop lies at a considerable distance from the central AFS-AD helix in the agonistic homology model. Loop L1-3 is separated from helix H3 by the β-pleated sheet.

Moreover, the size of the ligand-binding niche differs substantially between the two structures. The presence of the large fatty acid residue in the USP-LBD crystal structure causes a great cavity by shifting helices H3, H6 and H11. In the USP agonistic conformation, these helices are packed densely and produce a smaller ligand-binding niche.

The regions of the C-terminal ends of H3, H4, H5, H8 and H9 are rigid and capable of very good superposition in the two structures. In contrast, loop L1-3 and the C-termini of H3, H6 and H11 in the two structures are displaced relative to each other. These segments form the most mobile region of the ligand-binding domain of nuclear receptors. This movement is probably specific for each receptor and the ligand-generated displacement.

Information on the Sequence Listing

SEQ ID NO: 1 shows the amino acid sequence of the Heliothis virescens USP-LBD.

SEQ ID NO: 2 shows the amino acid sequence of the Heliothis virescens USP.

Information on the Tables

Table 1 shows the structure coordinates of the LBD of the Heliothis virescens USP.

Table 2 shows the amino acid sequence assignment for hRXRα and USP of Heliothis virescens and of further nuclear receptor LBDs for generateing a homology model of the agonistic USP conformation.

TABLE 1
REMARK coordinates from restrained individual B-factor refinement
REMARK refinement resolution: 20.0 - 1.65 A
REMARK starting r= 0.2151 free_r= 0.2506
REMARK final r= 0.2112 free_r= 0.2459
REMARK B rmsd for bonded mainchain atoms= 1.437 target= 1.5
REMARK B rmsd for bonded sidechain atoms= 2.272 target= 2.0
REMARK B rmsd for angle mainchain atoms= 2.299 target= 2.0
REMARK B rmsd for angle sidechain atoms= 3.310 target= 2.5
REMARK rweight= 0.1000 (with wa= 1.12122)
REMARK target= mlf steps= 30
REMARK sg= P4(3)22 a= 58.211 b= 58.211 c= 144.687
alpha= 90 beta= 90 gamma= 90
REMARK parameter file 1 : CNS_TOPPAR:protein_rep.param
REMARK parameter file 2 : CNS_TOPPAR:water_rep.param
REMARK parameter file 3 : eph.par
REMARK molecular structure file: alternate.mtf
REMARK input coordinates: anneal_2.pdb
REMARK reflection file=
/home/billas/USP/SCALE0400/merge1A65/usp_20a1a65.10.cv
REMARK ncs= none
REMARK B-correction resolution: 6.0 - 1.65
REMARK initial B-factor correction applied to fobs:
REMARK B11= −1.985 B22= −1.985 B33= 3.970
REMARK B12= 0.000 B13= 0.000 B23= 0.000
REMARK B-factor correction applied to coordinate array B: −0.193
REMARK bulk solvent: density level= 0.33501 e/A\hat{ }3,
B-factor= 48.7849 A\hat{ }2
REMARK reflections with |Fobs|/sigma_F < 0.0 rejected
REMARK reflections with |Fobs| > 10000 * rms(Fobs) rejected
REMARK theoretical total number of refl. in resol. range:
30842 (100.0 %)
REMARK number of unobserved reflections (no entry or |F|=0):
1417 ( 4.6 %)

| | |
|---|---|
| REMARK number of reflections rejected: | 0 ( 0.0 %) |

| | |
|---|---|
| REMARK total number of reflections used: | 29425( 95.4%) |

| | |
|---|---|
| REMARK number of reflections in working set: | 26453 ( 85.8 %) |

| | |
|---|---|
| REMARK number of reflections in test set: | 2972 ( 9.6 %) |

CRYST1 58.211 58.211 144.687 90.00 90.00 90.00 P 43 2 2
REMARK FILENAME=“/home/billas/LUC/13cns/bind_2.pdb”
REMARK DATE: 4-Jun-00 14:33:10 created by user: billas
REMARK VERSION:1.0

| | | | |
|---|---|---|---|
| ATOM | 1 CB ALA 203 | 16.114 28.799 41.997 1.00 66.21 | |
| ATOM | 2 C ALA 203 | 15.029 28.899 39.746 1.00 66.49 | |
| ATOM | 3 O ALA 203 | 14.609 30.031 39.487 1.00 66.58 | |
| ATOM | 4 N ALA 203 | 17.364 29.707 40.068 1.00 66.33 | |
| ATOM | 5 CA ALA 203 | 16.347 28.703 40.490 1.00 66.40 | |
| ATOM | 6 N ALA 204 | 14.387 27.790 39.393 1.00 66.05 | |
| ATOM | 7 CA ALA 204 | 13.106 27.833 38.698 1.00 65.56 | |
| ATOM | 8 CB ALA 204 | 12.933 26.584 37.843 1.00 65.11 | |
| ATOM | 9 C ALA 204 | 12.028 27.888 39.776 1.00 64.97 | |
| ATOM | 10 O ALA 204 | 12.259 27.413 40.890 1.00 65.54 | |
| ATOM | 11 N ALA 205 | 10.872 28.478 39.463 1.00 63.97 | |
| ATOM | 12 CA ALA 205 | 9.773 28.563 40.431 1.00 62.46 | |
| ATOM | 13 CB ALA 205 | 8.437 28.736 39.705 1.00 62.71 | |
| ATOM | 14 C ALA 205 | 9.798 27.243 41.190 1.00 61.33 | |
| ATOM | 15 O ALA 205 | 9.426 26.199 40.647 1.00 61.43 | |
| ATOM | 16 N GLN 206 | 10.251 27.285 42.439 1.00 59.43 | |
| ATOM | 17 CA GLN 206 | 10.372 26.060 43.211 1.00 57.60 | |
| ATOM | 18 CB GLN 206 | 11.198 26.298 44.472 1.00 58.55 | |
| ATOM | 19 CG GLN 206 | 11.976 25.062 44.863 1.00 60.08 | |
| ATOM | 20 CD GLN 206 | 12.831 24.542 43.712 1.00 61.69 | |
| ATOM | 21 OE1 GLN 206 | 13.892 25.094 43.411 1.00 62.30 | |
| ATOM | 22 NE2 GLN 206 | 12.360 23.486 43.053 1.00 61.64 | |
| ATOM | 23 C GLN 206 | 9.072 25.355 43.567 1.00 55.19 | |
| ATOM | 24 O GLN 206 | 8.089 25.972 43.983 1.00 55.16 | |
| ATOM | 25 N GLU 207 | 9.099 24.040 43.382 1.00 52.39 | |
| ATOM | 26 CA GLU 207 | 7.970 23.165 43.644 1.00 49.47 | |
| ATOM | 27 CB GLU 207 | 7.755 22.243 42.447 1.00 52.06 | |
| ATOM | 28 CG GLU 207 | 6.603 21.264 42.591 1.00 55.45 | |
| ATOM | 29 CD GLU 207 | 5.266 21.897 42.275 1.00 57.54 | |
| ATOM | 30 OE1 GLU 207 | 4.249 21.167 42.254 1.00 58.63 | |
| ATOM | 31 OE2 GLU 207 | 5.235 23.125 42.043 1.00 58.53 | |
| ATOM | 32 C GLU 207 | 8.273 22.311 44.861 1.00 45.64 | |
| ATOM | 33 O GLU 207 | 9.419 21.945 45.089 1.00 44.54 | |
| ATOM | 34 N LEU 208 | 7.244 21.996 45.637 1.00 41.48 | |
| ATOM | 35 CA LEU 208 | 7.408 21.142 46.810 1.00 38.35 | |
| ATOM | 36 CB LEU 208 | 6.204 21.323 47.752 1.00 36.20 | |
| ATOM | 37 CG LEU 208 | 6.211 20.671 49.134 1.00 34.07 | |
| ATOM | 38 CD1 LEU 208 | 7.495 21.026 49.867 1.00 32.61 | |
| ATOM | 39 CD2 LEU 208 | 5.003 21.158 49.926 1.00 33.18 | |
| ATOM | 40 C LEU 208 | 7.472 19.709 46.267 1.00 38.07 | |
| ATOM | 41 O LEU 208 | 6.443 19.122 45.919 1.00 38.80 | |
| ATOM | 42 N SER 209 | 8.682 19.155 46.174 1.00 34.84 | |
| ATOM | 43 CA SER 209 | 8.882 17.803 45.647 1.00 33.19 | |
| ATOM | 44 CB SER 209 | 9.257 17.883 44.165 1.00 32.93 | |
| ATOM | 45 OG SER 209 | 10.582 18.382 44.024 1.00 33.12 | |
| ATOM | 46 C SER 209 | 10.005 17.062 46.393 1.00 32.84 | |
| ATOM | 47 O SER 209 | 10.824 17.685 47.057 1.00 32.20 | |
| ATOM | 48 N ILE 210 | 10.048 15.736 46.261 1.00 32.33 | |
| ATOM | 49 CA ILE 210 | 11.092 14.945 46.917 1.00 34.10 | |
| ATOM | 50 CB ILE 210 | 10.961 13.438 46.613 1.00 35.86 | |
| ATOM | 51 CG2 ILE 210 | 12.017 12.667 47.387 1.00 37.65 | |
| ATOM | 52 CG1 ILE 210 | 9.565 12.929 46.980 1.00 36.30 | |
| ATOM | 53 CD1 ILE 210 | 9.239 13.004 48.447 1.00 35.19 | |
| ATOM | 54 C ILE 210 | 12.478 15.370 46.436 1.00 33.53 | |
| ATOM | 55 O ILE 210 | 13.420 15.467 47.225 1.00 30.63 | |
| ATOM | 56 N GLU 211 | 12.607 15.609 45.136 1.00 33.07 | |
| ATOM | 57 CA GLU 211 | 13.898 16.012 44.587 1.00 33.86 | |
| ATOM | 58 CB GLU 211 | 13.797 16.199 43.066 1.00 36.35 | |
| ATOM | 59 CG GLU 211 | 15.042 16.838 42.436 1.00 40.50 | |
| ATOM | 60 CD GLU 211 | 14.880 17.104 40.941 1.00 43.91 | |
| ATOM | 61 OE1 GLU 211 | 15.777 17.748 40.348 1.00 45.56 | |
| ATOM | 62 OE2 GLU 211 | 13.857 16.667 40.365 1.00 44.68 | |
| ATOM | 63 C GLU 211 | 14.396 17.299 45.246 1.00 32.93 | |
| ATOM | 64 O GLU 211 | 15.552 17.395 45.637 1.00 32.96 | |
| ATOM | 65 N ARG 212 | 13.524 18.292 45.365 1.00 31.58 | |
| ATOM | 66 CA ARG 212 | 13.914 19.545 45.994 1.00 30.93 | |
| ATOM | 67 CB ARG 212 | 12.799 20.579 45.850 1.00 31.30 | |
| ATOM | 68 CG ARG 212 | 13.111 21.897 46.547 1.00 34.79 | |
| ATOM | 69 CD ARG 212 | 14.482 22.417 46.130 1.00 36.82 | |
| ATOM | 70 NE ARG 212 | 14.880 23.599 46.886 1.00 40.98 | |
| ATOM | 71 CZ ARG 212 | 16.109 24.103 46.876 1.00 41.42 | |
| ATOM | 72 NH1 ARG 212 | 17.055 23.527 46.148 1.00 43.03 | |
| ATOM | 73 NH2 ARG 212 | 16.395 25.178 47.596 1.00 43.71 | |
| ATOM | 74 C ARG 212 | 14.277 19.375 47.478 1.00 29.92 | |
| ATOM | 75 O ARG 212 | 15.218 20.000 47.970 1.00 30.04 | |
| ATOM | 76 N LEU 213 | 13.529 18.541 48.195 1.00 28.52 | |
| ATOM | 77 CA LEU 213 | 13.819 18.322 49.612 1.00 27.44 | |
| ATOM | 78 CB LEU 213 | 12.711 17.471 50.272 1.00 25.96 | |
| ATOM | 79 CG LEU 213 | 11.361 18.184 50.410 1.00 27.73 | |
| ATOM | 80 CD1 LEU 213 | 10.306 17.200 50.892 1.00 24.70 | |
| ATOM | 81 CD2 LEU 213 | 11.492 19.344 51.358 1.00 27.22 | |
| ATOM | 82 C LEU 213 | 15.172 17.625 49.757 1.00 27.41 | |
| ATOM | 83 O LEU 213 | 15.895 17.861 50.722 1.00 27.85 | |
| ATOM | 84 N LEU 214 | 15.510 16.761 48.802 1.00 28.81 | |
| ATOM | 85 CA LEU 214 | 16.792 16.068 48.874 1.00 30.82 | |
| ATOM | 86 CB LEU 214 | 16.863 14.926 47.851 1.00 29.94 | |
| ATOM | 87 CG LEU 214 | 16.112 13.655 48.272 1.00 30.12 | |
| ATOM | 88 CD1 LEU 214 | 16.047 12.663 47.117 1.00 30.68 | |
| ATOM | 89 CD2 LEU 214 | 16.820 13.015 49.467 1.00 31.09 | |
| ATOM | 90 C LEU 214 | 17.914 17.067 48.646 1.00 33.09 | |
| ATOM | 91 O LEU 214 | 18.954 16.985 49.297 1.00 32.71 | |
| ATOM | 92 N GLU 215 | 17.700 18.018 47.736 1.00 35.01 | |
| ATOM | 93 CA GLU 215 | 18.702 19.046 47.471 1.00 36.86 | |
| ATOM | 94 CB GLU 215 | 18.274 19.977 46.319 1.00 38.74 | |
| ATOM | 95 CG GLU 215 | 18.045 19.310 44.962 1.00 42.50 | |
| ATOM | 96 CD GLU 215 | 17.655 20.307 43.862 1.00 44.43 | |
| ATOM | 97 OE1 GLU 215 | 17.034 21.350 44.174 1.00 44.44 | |
| ATOM | 98 OE2 GLU 215 | 17.957 20.039 42.674 1.00 46.05 | |
| ATOM | 99 C GLU 215 | 18.881 19.886 48.735 1.00 36.72 | |
| ATOM | 100 O GLU 215 | 19.996 20.278 49.082 1.00 37.76 | |
| ATOM | 101 N MET 216 | 17.779 20.159 49.430 1.00 35.08 | |
| ATOM | 102 CA MET 216 | 17.814 20.970 50.644 1.00 34.20 | |
| ATOM | 103 CB MET 216 | 16.393 21.402 51.018 1.00 32.09 | |
| ATOM | 104 CG MET 216 | 15.820 22.514 50.188 1.00 31.14 | |
| ATOM | 105 SD MET 216 | 14.067 22.656 50.591 1.00 29.63 | |
| ATOM | 106 CE MET 216 | 14.187 23.383 52.223 1.00 30.33 | |
| ATOM | 107 C MET 216 | 18.447 20.309 51.868 1.00 33.78 | |
| ATOM | 108 O MET 216 | 18.955 20.998 52.755 1.00 32.43 | |
| ATOM | 109 N GLU 217 | 18.381 18.982 51.923 1.00 34.02 | |
| ATOM | 110 CA GLU 217 | 18.908 18.212 53.046 1.00 35.35 | |
| ATOM | 111 CB GLU 217 | 18.470 16.745 52.919 1.00 33.44 | |
| ATOM | 112 CG GLU 217 | 18.729 15.871 54.148 1.00 32.91 | |
| ATOM | 113 CD GLU 217 | 17.999 16.372 55.389 1.00 32.03 | |
| ATOM | 114 OE1 GLU 217 | 17.001 17.104 55.242 1.00 33.02 | |
| ATOM | 115 OE2 GLU 217 | 18.419 16.032 56.511 1.00 34.58 | |
| ATOM | 116 C GLU 217 | 20.421 18.293 53.105 1.00 37.01 | |
| ATOM | 117 O GLU 217 | 21.006 18.276 54.190 1.00 37.08 | |
| ATOM | 118 N SER 218 | 21.036 18.406 51.928 1.00 39.66 | |
| ATOM | 119 CA SER 218 | 22.491 18.476 51.795 1.00 42.68 | |
| ATOM | 120 CB SER 218 | 22.913 18.001 50.408 1.00 43.25 | |
| ATOM | 121 OG SER 218 | 22.571 16.640 50.214 1.00 46.19 | |
| ATOM | 122 C SER 218 | 23.083 19.856 52.027 1.00 44.17 | |
| ATOM | 123 O SER 218 | 24.250 19.978 52.395 1.00 43.66 | |
| ATOM | 124 N LEU 219 | 22.291 20.895 51.797 1.00 46.04 | |
| ATOM | 125 CA LEU 219 | 22.773 22.254 51.982 1.00 48.34 | |
| ATOM | 126 CB LEU 219 | 21.706 23.265 51.556 1.00 49.36 | |
| ATOM | 127 CG LEU 219 | 21.670 23.631 50.072 1.00 50.71 | |
| ATOM | 128 CD1 LEU 219 | 21.879 22.395 49.219 1.00 51.07 | |
| ATOM | 129 CD2 LEU 219 | 20.341 24.307 49.760 1.00 51.61 | |
| ATOM | 130 C LEU 219 | 23.183 22.530 53.416 1.00 49.65 | |
| ATOM | 131 O LEU 219 | 22.501 22.140 54.363 1.00 49.02 | |
| ATOM | 132 N VAL 220 | 24.318 23.205 53.552 1.00 51.55 | |
| ATOM | 133 CA VAL 220 | 24.868 23.592 54.843 1.00 53.29 | |
| ATOM | 134 CB VAL 220 | 26.250 22.945 55.085 1.00 52.35 | |
| ATOM | 135 CG1 VAL 220 | 26.774 23.341 56.456 1.00 51.60 | |
| ATOM | 136 CG2 VAL 220 | 26.142 21.426 54.965 1.00 50.84 | |
| ATOM | 137 C VAL 220 | 25.030 25.106 54.770 1.00 55.56 | |
| ATOM | 138 O VAL 220 | 25.929 25.613 54.097 1.00 56.16 | |
| ATOM | 139 N ALA 221 | 24.147 25.823 55.450 1.00 57.83 | |
| ATOM | 140 CA ALA 221 | 24.190 27.278 55.428 1.00 60.51 | |
| ATOM | 141 CB ALA 221 | 22.782 27.845 55.601 1.00 60.73 | |
| ATOM | 142 C ALA 221 | 25.108 27.838 56.499 1.00 61.87 | |
| ATOM | 143 O ALA 221 | 25.475 27.143 57.452 1.00 62.13 | |
| ATOM | 144 N ALA 222 | 25.490 29.100 56.326 1.00 62.85 | |
| ATOM | 145 CA ALA 222 | 26.345 29.756 57.295 1.00 63.37 | |
| ATOM | 146 CB ALA 222 | 26.612 31.196 56.871 1.00 63.59 | |
| ATOM | 147 C ALA 222 | 25.564 29.719 58.601 1.00 63.51 | |
| ATOM | 148 O ALA 222 | 24.422 30.176 58.659 1.00 63.95 | |
| ATOM | 149 N ALA 223 | 26.173 29.150 59.636 1.00 63.63 | |
| ATOM | 150 CA ALA 223 | 25.532 29.044 60.939 1.00 63.10 | |
| ATOM | 151 CB ALA 223 | 26.558 28.627 61.984 1.00 63.64 | |
| ATOM | 152 C ALA 223 | 24.874 30.365 61.339 1.00 62.58 | |
| ATOM | 153 O ALA 223 | 25.557 31.333 61.678 1.00 63.33 | |
| ATOM | 154 N ALA 224 | 23.544 30.399 61.290 1.00 61.31 | |
| ATOM | 155 CA ALA 224 | 22.789 31.599 61.644 1.00 59.25 | |
| ATOM | 156 CB ALA 224 | 21.323 31.440 61.220 1.00 58.51 | |
| ATOM | 157 C ALA 224 | 22.878 31.880 63.143 1.00 58.07 | |
| ATOM | 158 O ALA 224 | 22.988 30.943 63.939 1.00 58.18 | |
| ATOM | 159 N GLU 225 | 22.844 33.159 63.528 1.00 56.07 | |
| ATOM | 160 CA GLU 225 | 22.909 33.507 64.950 1.00 54.05 | |
| ATOM | 161 CB GLU 225 | 22.498 34.969 65.221 1.00 54.53 | |
| ATOM | 162 CG GLU 225 | 22.700 35.401 66.703 1.00 55.83 | |
| ATOM | 163 CD GLU 225 | 21.439 35.872 67.407 1.00 56.62 | |
| ATOM | 164 OE1 GLU 225 | 21.407 35.822 68.663 1.00 57.35 | |
| ATOM | 165 OE2 GLU 225 | 20.464 36.314 66.743 1.00 57.42 | |
| ATOM | 166 C GLU 225 | 21.901 32.594 65.611 1.00 52.71 | |
| ATOM | 167 O GLU 225 | 20.737 32.557 65.201 1.00 52.66 | |
| ATOM | 168 N GLU 226 | 22.334 31.840 66.612 1.00 50.26 | |
| ATOM | 169 CA GLU 226 | 21.391 30.960 67.256 1.00 47.92 | |
| ATOM | 170 CB GLU 226 | 21.858 29.513 67.196 1.00 49.61 | |
| ATOM | 171 CG GLU 226 | 20.778 28.525 67.641 1.00 49.78 | |
| ATOM | 172 CD GLU 226 | 19.333 28.959 67.359 1.00 51.91 | |
| ATOM | 173 OE1 GLU 226 | 18.452 28.312 67.956 1.00 50.08 | |
| ATOM | 174 OE2 GLU 226 | 19.039 29.909 66.543 1.00 51.77 | |
| ATOM | 175 C GLU 226 | 21.015 31.344 68.670 1.00 46.28 | |
| ATOM | 176 O GLU 226 | 21.839 31.794 69.476 1.00 47.17 | |
| ATOM | 177 N PHE 227 | 19.733 31.155 68.942 1.00 41.52 | |
| ATOM | 178 CA PHE 227 | 19.138 31.503 70.206 1.00 38.73 | |
| ATOM | 179 CB PHE 227 | 17.723 32.022 69.963 1.00 40.03 | |
| ATOM | 180 CG PHE 227 | 17.640 33.061 68.876 1.00 40.91 | |
| ATOM | 181 CD1 PHE 227 | 17.775 32.703 67.542 1.00 40.72 | |
| ATOM | 182 CD2 PHE 227 | 17.464 34.402 69.192 1.00 42.62 | |
| ATOM | 183 CE1 PHE 227 | 17.739 33.661 66.535 1.00 42.04 | |
| ATOM | 184 CE2 PHE 227 | 17.426 35.374 68.188 1.00 42.27 | |
| ATOM | 185 CZ PHE 227 | 17.564 34.996 66.857 1.00 42.02 | |
| ATOM | 186 C PHE 227 | 19.119 30.339 71.174 1.00 34.83 | |
| ATOM | 187 O PHE 227 | 19.218 29.178 70.778 1.00 34.52 | |
| ATOM | 188 N GLN 228 | 18.982 30.687 72.445 1.00 33.69 | |
| ATOM | 189 CA GLN 228 | 18.979 29.735 73.540 1.00 32.01 | |
| ATOM | 190 CB GLN 228 | 19.290 30.468 74.847 1.00 34.80 | |
| ATOM | 191 CG GLN 228 | 20.680 31.080 74.935 1.00 39.39 | |
| ATOM | 192 CD GLN 228 | 21.768 30.029 74.916 1.00 40.63 | |
| ATOM | 193 OE1 GLN 228 | 22.117 29.504 73.860 1.00 44.27 | |
| ATOM | 194 NE2 GLN 228 | 22.301 29.706 76.093 1.00 42.65 | |
| ATOM | 195 C GLN 228 | 17.678 28.974 73.736 1.00 28.92 | |
| ATOM | 196 O GLN 228 | 17.035 29.174 74.750 1.00 28.29 | |
| ATOM | 197 N PHE 229 | 17.283 28.120 72.794 1.00 28.04 | |
| ATOM | 198 CA PHE 229 | 16.056 27.340 72.996 1.00 25.30 | |
| ATOM | 199 CB PHE 229 | 15.767 26.411 71.821 1.00 27.02 | |
| ATOM | 200 CG PHE 229 | 15.066 27.055 70.680 1.00 28.71 | |
| ATOM | 201 CD1 PHE 229 | 15.599 28.170 70.052 1.00 28.96 | |
| ATOM | 202 CD2 PHE 229 | 13.903 26.480 70.173 1.00 30.61 | |
| ATOM | 203 CE1 PHE 229 | 14.991 28.708 68.918 1.00 29.32 | |
| ATOM | 204 CE2 PHE 229 | 13.284 26.998 69.046 1.00 31.44 | |
| ATOM | 205 CZ PHE 229 | 13.829 28.117 68.412 1.00 31.43 | |
| ATOM | 206 C PHE 229 | 16.276 26.434 74.195 1.00 23.81 | |
| ATOM | 207 O PHE 229 | 15.385 26.202 75.014 1.00 22.15 | |
| ATOM | 208 N LEU 230 | 17.487 25.899 74.263 1.00 20.44 | |
| ATOM | 209 CA LEU 230 | 17.848 24.964 75.302 1.00 21.42 | |
| ATOM | 210 CB LEU 230 | 18.255 23.631 74.650 1.00 20.34 | |
| ATOM | 211 CG LEU 230 | 17.191 22.855 73.834 1.00 22.70 | |
| ATOM | 212 CD1 LEU 230 | 17.860 21.762 73.013 1.00 23.14 | |
| ATOM | 213 CD2 LEU 230 | 16.130 22.252 74.773 1.00 21.55 | |
| ATOM | 214 C LEU 230 | 19.017 25.540 76.094 1.00 20.71 | |
| ATOM | 215 O LEU 230 | 19.977 26.038 75.524 1.00 20.61 | |
| ATOM | 216 N ARG 231 | 18.931 25.467 77.411 1.00 20.48 | |
| ATOM | 217 CA ARG 231 | 20.018 25.997 78.211 1.00 20.62 | |
| ATOM | 218 CB ARG 231 | 20.023 27.525 78.176 1.00 20.50 | |
| ATOM | 219 CG ARG 231 | 18.907 28.184 79.017 1.00 24.52 | |
| ATOM | 220 CD ARG 231 | 17.560 28.025 78.342 1.00 27.62 | |
| ATOM | 221 NE ARG 231 | 16.465 28.674 79.060 1.00 28.14 | |
| ATOM | 222 CZ ARG 231 | 15.846 28.165 80.118 1.00 28.35 | |
| ATOM | 223 NH1 ARG 231 | 16.208 26.986 80.598 1.00 27.27 | |
| ATOM | 224 NH2 ARG 231 | 14.856 28.835 80.692 1.00 28.94 | |
| ATOM | 225 C ARG 231 | 19.957 25.585 79.663 1.00 20.62 | |
| ATOM | 226 O ARG 231 | 18.884 25.350 80.219 1.00 20.08 | |
| ATOM | 227 N VAL 232 | 21.138 25.549 80.265 1.00 20.70 | |
| ATOM | 228 CA VAL 232 | 21.276 25.244 81.684 1.00 23.23 | |
| ATOM | 229 CB VAL 232 | 22.596 24.499 81.961 1.00 24.57 | |
| ATOM | 230 CG1 VAL 232 | 22.800 24.335 83.460 1.00 24.88 | |
| ATOM | 231 CG2 VAL 232 | 22.565 23.135 81.289 1.00 23.34 | |
| ATOM | 232 C VAL 232 | 21.306 26.619 82.359 1.00 25.86 | |
| ATOM | 233 O VAL 232 | 22.284 27.349 82.225 1.00 29.87 | |
| ATOM | 234 N GLY 233 | 20.232 26.960 83.062 1.00 24.64 | |
| ATOM | 235 CA GLY 233 | 20.137 28.249 83.730 1.00 26.40 | |
| ATOM | 236 C GLY 233 | 20.170 28.129 85.240 1.00 27.72 | |
| ATOM | 237 O GLY 233 | 20.449 27.041 85.755 1.00 26.07 | |
| ATOM | 238 N PRO 234 | 19.863 29.206 85.980 1.00 28.00 | |
| ATOM | 239 CD PRO 234 | 19.340 30.507 85.515 1.00 30.45 | |
| ATOM | 240 CA PRO 234 | 19.886 29.158 87.447 1.00 28.48 | |
| ATOM | 241 CB PRO 234 | 19.772 30.627 87.833 1.00 30.42 | |
| ATOM | 242 CG PRO 234 | 18.804 31.123 86.802 1.00 32.04 | |
| ATOM | 243 C PRO 234 | 18.752 28.323 88.037 1.00 27.91 | |
| ATOM | 244 O PRO 234 | 18.781 27.975 89.229 1.00 28.03 | |
| ATOM | 245 N ASP 235 | 17.757 27.996 87.210 1.00 26.13 | |
| ATOM | 246 CA ASP 235 | 16.636 27.186 87.680 1.00 23.93 | |
| ATOM | 247 CB ASP 235 | 15.333 27.686 87.064 1.00 28.77 | |
| ATOM | 248 CG ASP 235 | 15.405 27.820 85.544 1.00 31.24 | |
| ATOM | 249 OD1 ASP 235 | 16.519 27.836 84.964 1.00 32.56 | |
| ATOM | 250 OD2 ASP 235 | 14.327 27.922 84.919 1.00 34.70 | |
| ATOM | 251 C ASP 235 | 16.828 25.700 87.362 1.00 20.99 | |
| ATOM | 252 O ASP 235 | 16.019 24.881 87.751 1.00 19.94 | |
| ATOM | 253 N SER 236 | 17.914 25.369 86.666 0.50 20.63 | AC1 |
| ATOM | 254 CA SER 236 | 18.208 23.979 86.280 0.50 20.15 | AC1 |
| ATOM | 255 CB SER 236 | 19.196 23.969 85.111 0.50 19.44 | AC1 |
| ATOM | 256 OG SER 236 | 18.708 24.734 84.013 0.50 19.16 | AC1 |
| ATOM | 257 C SER 236 | 18.787 23.137 87.425 0.50 21.81 | AC1 |
| ATOM | 258 O SER 236 | 19.849 23.457 87.946 0.50 20.58 | AC1 |
| ATOM | 259 N ASN 237 | 18.125 22.037 87.782 1.00 21.94 | |
| ATOM | 260 CA ASN 237 | 18.621 21.202 88.886 1.00 25.34 | |
| ATOM | 261 CB ASN 237 | 17.498 20.331 89.450 1.00 30.84 | |
| ATOM | 262 CG ASN 237 | 17.020 19.301 88.472 1.00 34.04 | |
| ATOM | 263 OD1 ASN 237 | 17.786 18.838 87.623 1.00 39.37 | |
| ATOM | 264 ND2 ASN 237 | 15.748 18.913 88.588 1.00 39.05 | |
| ATOM | 265 C ASN 237 | 19.843 20.325 88.581 1.00 26.93 | |
| ATOM | 266 O ASN 237 | 20.180 19.405 89.339 1.00 32.05 | |
| ATOM | 267 N VAL 238 | 20.537 20.621 87.503 1.00 23.03 | |
| ATOM | 268 CA VAL 238 | 21.713 19.862 87.114 1.00 20.81 | |
| ATOM | 269 CB VAL 238 | 22.096 20.241 85.663 1.00 20.25 | |
| ATOM | 270 CG1 VAL 238 | 23.336 19.495 85.207 1.00 19.20 | |
| ATOM | 271 CG2 VAL 238 | 20.919 19.922 84.741 1.00 20.14 | |
| ATOM | 272 C VAL 238 | 22.844 20.185 88.106 1.00 20.62 | |
| ATOM | 273 O VAL 238 | 23.147 21.343 88.363 1.00 18.94 | |
| ATOM | 274 N PRO 239 | 23.428 19.153 88.719 1.00 20.32 | |
| ATOM | 275 CD PRO 239 | 23.144 17.726 88.541 1.00 20.40 | |
| ATOM | 276 CA PRO 239 | 24.515 19.328 89.688 1.00 21.49 | |
| ATOM | 277 CB PRO 239 | 24.911 17.892 90.023 1.00 22.13 | |
| ATOM | 278 CG PRO 239 | 23.696 17.174 89.847 1.00 20.51 | |
| ATOM | 279 C PRO 239 | 25.663 20.039 89.023 1.00 24.59 | |
| ATOM | 280 O PRO 239 | 25.924 19.804 87.847 1.00 23.65 | |
| ATOM | 281 N PRO 240 | 26.388 20.879 89.777 1.00 24.92 | |
| ATOM | 282 CD PRO 240 | 26.142 21.311 91.167 1.00 26.02 | |
| ATOM | 283 CA PRO 240 | 27.524 21.603 89.202 1.00 26.88 | |
| ATOM | 284 CB PRO 240 | 28.222 22.161 90.438 1.00 26.73 | |
| ATOM | 285 CG PRO 240 | 27.039 22.543 91.299 1.00 29.18 | |
| ATOM | 286 C PRO 240 | 28.434 20.733 88.363 1.00 26.35 | |
| ATOM | 287 O PRO 240 | 28.847 21.125 87.259 1.00 27.72 | |
| ATOM | 288 N LYS 241 | 28.745 19.533 88.838 1.00 28.50 | |
| ATOM | 289 CA LYS 241 | 29.642 18.682 88.071 1.00 28.19 | |
| ATOM | 290 CB LYS 241 | 30.073 17.457 88.878 1.00 32.48 | |
| ATOM | 291 CG LYS 241 | 28.970 16.508 89.292 1.00 34.64 | |
| ATOM | 292 CD LYS 241 | 29.627 15.241 89.832 1.00 37.47 | |
| ATOM | 293 CE LYS 241 | 28.627 14.162 90.157 1.00 39.69 | |
| ATOM | 294 NZ LYS 241 | 29.359 12.932 90.578 1.00 42.50 | |
| ATOM | 295 C LYS 241 | 29.144 18.233 86.700 1.00 27.86 | |
| ATOM | 296 O LYS 241 | 29.935 17.790 85.868 1.00 27.57 | |
| ATOM | 297 N PHE 242 | 27.840 18.335 86.459 1.00 25.10 | |
| ATOM | 298 CA PHE 242 | 27.309 17.953 85.154 1.00 22.57 | |
| ATOM | 299 CB PHE 242 | 26.161 16.954 85.319 1.00 24.91 | |
| ATOM | 300 CG PHE 242 | 26.600 15.619 85.856 1.00 28.41 | |
| ATOM | 301 CD1 PHE 242 | 27.411 14.790 85.102 1.00 30.20 | |
| ATOM | 302 CD2 PHE 242 | 26.194 15.191 87.108 1.00 28.96 | |
| ATOM | 303 CE1 PHE 242 | 27.813 13.544 85.583 1.00 31.58 | |
| ATOM | 304 CE2 PHE 242 | 26.594 13.943 87.596 1.00 30.20 | |
| ATOM | 305 CZ PHE 242 | 27.400 13.127 86.832 1.00 32.03 | |
| ATOM | 306 C PHE 242 | 26.837 19.151 84.330 1.00 19.63 | |
| ATOM | 307 O PHE 242 | 26.386 18.991 83.190 1.00 20.79 | |
| ATOM | 308 N ARG 243 | 26.971 20.350 84.873 1.00 19.69 | |
| ATOM | 309 CA ARG 243 | 26.513 21.529 84.153 1.00 20.41 | |
| ATOM | 310 CB ARG 243 | 26.538 22.749 85.066 1.00 20.87 | |
| ATOM | 311 CG ARG 243 | 25.523 22.602 86.208 1.00 23.08 | |
| ATOM | 312 CD ARG 243 | 25.562 23.759 87.130 1.00 26.38 | |
| ATOM | 313 NE ARG 243 | 24.725 24.856 86.687 1.00 27.91 | |
| ATOM | 314 CZ ARG 243 | 23.394 24.873 86.728 1.00 28.35 | |
| ATOM | 315 NH1 ARG 243 | 22.697 23.825 87.192 1.00 26.91 | |
| ATOM | 316 NH2 ARG 243 | 22.764 25.975 86.343 1.00 28.11 | |
| ATOM | 317 C ARG 243 | 27.238 21.813 82.846 1.00 20.13 | |
| ATOM | 318 O ARG 243 | 26.596 22.114 81.836 1.00 19.71 | |
| ATOM | 319 N ALA 244 | 28.564 21.734 82.838 1.00 20.90 | |
| ATOM | 320 CA ALA 244 | 29.273 21.974 81.577 1.00 22.06 | |
| ATOM | 321 CB ALA 244 | 30.773 21.896 81.796 1.00 21.20 | |
| ATOM | 322 C ALA 244 | 28.842 20.984 80.492 1.00 21.05 | |
| ATOM | 323 O ALA 244 | 28.527 21.365 79.366 1.00 21.57 | |
| ATOM | 324 N PRO 245 | 28.783 19.696 80.822 1.00 21.63 | |
| ATOM | 325 CD PRO 245 | 29.335 19.087 82.049 1.00 25.65 | |
| ATOM | 326 CA PRO 245 | 28.377 18.672 79.861 1.00 22.28 | |
| ATOM | 327 CB PRO 245 | 28.516 17.380 80.666 1.00 24.91 | |
| ATOM | 328 CG PRO 245 | 29.614 17.694 81.605 1.00 25.26 | |
| ATOM | 329 C PRO 245 | 26.943 18.859 79.329 1.00 19.49 | |
| ATOM | 330 O PRO 245 | 26.691 18.778 78.118 1.00 21.31 | |
| ATOM | 331 N VAL 246 | 26.013 19.123 80.230 1.00 20.18 | |
| ATOM | 332 CA VAL 246 | 24.624 19.294 79.805 1.00 18.44 | |
| ATOM | 333 CB VAL 246 | 23.693 19.269 81.028 1.00 18.35 | |
| ATOM | 334 CG1 VAL 246 | 22.221 19.536 80.620 1.00 18.11 | |
| ATOM | 335 CG2 VAL 246 | 23.766 17.845 81.678 1.00 17.69 | |
| ATOM | 336 C VAL 246 | 24.501 20.568 78.968 1.00 18.23 | |
| ATOM | 337 O VAL 246 | 23.773 20.603 77.977 1.00 18.23 | |
| ATOM | 338 N SER 247 | 25.234 21.609 79.351 0.50 20.08 | AC1 |
| ATOM | 339 CA SER 247 | 25.198 22.858 78.599 0.50 21.19 | AC1 |
| ATOM | 340 CB SER 247 | 26.058 23.910 79.289 0.50 21.37 | AC1 |
| ATOM | 341 OG SER 247 | 25.484 24.292 80.517 0.50 23.67 | AC1 |
| ATOM | 342 C SER 247 | 25.712 22.621 77.189 0.50 21.32 | AC1 |
| ATOM | 343 O SER 247 | 25.180 23.161 76.217 0.50 21.62 | AC1 |
| ATOM | 344 N SER 248 | 26.756 21.811 77.076 1.00 20.43 | |
| ATOM | 345 CA SER 248 | 27.322 21.502 75.766 1.00 21.62 | |
| ATOM | 346 CB SER 248 | 28.541 20.594 75.916 1.00 24.31 | |
| ATOM | 347 OG SER 248 | 29.660 21.350 76.354 1.00 24.89 | |
| ATOM | 348 C SER 248 | 26.271 20.800 74.903 1.00 22.36 | |
| ATOM | 349 O SER 248 | 26.158 21.061 73.697 1.00 22.32 | |
| ATOM | 350 N LEU 249 | 25.513 19.895 75.519 1.00 22.35 | |
| ATOM | 351 CA LEU 249 | 24.462 19.185 74.791 1.00 22.28 | |
| ATOM | 352 CB LEU 249 | 23.904 18.046 75.668 1.00 22.23 | |
| ATOM | 353 CG LEU 249 | 24.970 16.972 75.954 1.00 25.52 | |
| ATOM | 354 CD1 LEU 249 | 24.441 15.919 76.922 1.00 26.39 | |
| ATOM | 355 CD2 LEU 249 | 25.398 16.341 74.643 1.00 27.01 | |
| ATOM | 356 C LEU 249 | 23.358 20.152 74.352 1.00 21.63 | |
| ATOM | 357 O LEU 249 | 22.822 20.039 73.231 1.00 19.99 | |
| ATOM | 358 N CYS 250 | 23.012 21.102 75.217 1.00 21.10 | |
| ATOM | 359 CA CYS 250 | 21.995 22.098 74.856 1.00 21.22 | |
| ATOM | 360 CB CYS 250 | 21.701 23.044 76.019 1.00 20.65 | |
| ATOM | 361 SG CYS 250 | 20.853 22.249 77.396 1.00 22.08 | |
| ATOM | 362 C CYS 250 | 22.471 22.932 73.684 1.00 22.49 | |
| ATOM | 363 O CYS 250 | 21.687 23.302 72.828 1.00 23.08 | |
| ATOM | 364 N GLN 251 | 23.765 23.239 73.656 1.00 22.61 | |
| ATOM | 365 CA GLN 251 | 24.313 24.064 72.577 1.00 24.50 | |
| ATOM | 366 CB GLN 251 | 25.738 24.482 72.925 1.00 26.13 | |
| ATOM | 367 CG GLN 251 | 25.827 25.428 74.123 1.00 31.89 | |
| ATOM | 368 CD GLN 251 | 27.270 25.602 74.609 1.00 36.32 | |
| ATOM | 369 OE1 GLN 251 | 28.146 26.020 73.846 1.00 38.70 | |
| ATOM | 370 NE2 GLN 251 | 27.520 25.275 75.883 1.00 36.33 | |
| ATOM | 371 C GLN 251 | 24.290 23.309 71.255 1.00 23.96 | |
| ATOM | 372 O GLN 251 | 24.080 23.899 70.200 1.00 25.04 | |
| ATOM | 373 N ILE 252 | 24.506 21.998 71.323 1.00 23.74 | |
| ATOM | 374 CA ILE 252 | 24.475 21.143 70.140 1.00 24.05 | |
| ATOM | 375 CB ILE 252 | 24.877 19.690 70.493 1.00 25.71 | |
| ATOM | 376 CG2 ILE 252 | 24.385 18.727 69.419 1.00 25.12 | |
| ATOM | 377 CG1 ILE 252 | 26.405 19.596 70.658 1.00 26.17 | |
| ATOM | 378 CD1 ILE 252 | 26.874 18.335 71.359 1.00 27.45 | |
| ATOM | 379 C ILE 252 | 23.032 21.183 69.642 1.00 25.30 | |
| ATOM | 380 O ILE 252 | 22.760 21.312 68.448 1.00 25.88 | |
| ATOM | 381 N GLY 253 | 22.101 21.105 70.580 1.00 25.76 | |
| ATOM | 382 CA GLY 253 | 20.698 21.167 70.213 1.00 24.72 | |
| ATOM | 383 C GLY 253 | 20.327 22.497 69.573 1.00 24.98 | |
| ATOM | 384 O GLY 253 | 19.611 22.527 68.561 1.00 24.56 | |
| ATOM | 385 N ASN 254 | 20.779 23.600 70.165 1.00 23.66 | |
| ATOM | 386 CA ASN 254 | 20.483 24.929 69.642 1.00 24.18 | |
| ATOM | 387 CB ASN 254 | 21.063 25.999 70.566 1.00 23.04 | |
| ATOM | 388 CG ASN 254 | 20.336 26.061 71.884 1.00 24.90 | |
| ATOM | 389 OD1 ASN 254 | 19.147 25.729 71.954 1.00 22.87 | |
| ATOM | 390 ND2 ASN 254 | 21.020 26.511 72.934 1.00 25.39 | |
| ATOM | 391 C ASN 254 | 20.990 25.142 68.215 1.00 25.59 | |
| ATOM | 392 O ASN 254 | 20.349 25.832 67.418 1.00 25.49 | |
| ATOM | 393 N LYS 255 | 22.154 24.575 67.915 1.00 24.87 | |
| ATOM | 394 CA LYS 255 | 22.718 24.669 66.572 1.00 26.12 | |
| ATOM | 395 CB LYS 255 | 24.121 24.061 66.518 1.00 29.40 | |
| ATOM | 396 CG LYS 255 | 25.162 24.983 67.112 1.00 33.68 | |
| ATOM | 397 CD LYS 255 | 26.481 24.293 67.392 1.00 34.86 | |
| ATOM | 398 CE LYS 255 | 27.280 24.027 66.150 1.00 35.57 | |
| ATOM | 399 NZ LYS 255 | 28.719 23.853 66.527 1.00 33.75 | |
| ATOM | 400 C LYS 255 | 21.816 23.924 65.608 1.00 26.04 | |
| ATOM | 401 O LYS 255 | 21.539 24.415 64.507 1.00 25.57 | |
| ATOM | 402 N GLN 256 | 21.358 22.741 66.009 1.00 24.02 | |
| ATOM | 403 CA GLN 256 | 20.498 21.967 65.121 1.00 23.89 | |
| ATOM | 404 CB GLN 256 | 20.325 20.546 65.658 1.00 25.47 | |
| ATOM | 405 CG GLN 256 | 21.676 19.880 65.887 1.00 28.63 | |
| ATOM | 406 CD GLN 256 | 21.565 18.517 66.534 1.00 30.93 | |
| ATOM | 407 OE1 GLN 256 | 20.710 18.301 67.387 1.00 32.63 | |
| ATOM | 408 NE2 GLN 256 | 22.439 17.599 66.149 1.00 30.18 | |
| ATOM | 409 C GLN 256 | 19.156 22.658 64.915 1.00 24.45 | |
| ATOM | 410 O GLN 256 | 18.596 22.598 63.828 1.00 24.20 | |
| ATOM | 411 N ILE 257 | 18.662 23.338 65.942 1.00 23.21 | |
| ATOM | 412 CA ILE 257 | 17.390 24.059 65.816 1.00 22.41 | |
| ATOM | 413 CB ILE 257 | 16.895 24.535 67.180 1.00 20.26 | |
| ATOM | 414 CG2 ILE 257 | 15.636 25.401 67.021 1.00 21.15 | |
| ATOM | 415 CG1 ILE 257 | 16.607 23.298 68.039 1.00 22.12 | |
| ATOM | 416 CD1 ILE 257 | 16.309 23.620 69.517 1.00 21.36 | |
| ATOM | 417 C ILE 257 | 17.558 25.253 64.877 1.00 23.91 | |
| ATOM | 418 O ILE 257 | 16.677 25.544 64.060 1.00 21.49 | |
| ATOM | 419 N ALA 258 | 18.684 25.949 64.994 1.00 23.86 | |
| ATOM | 420 CA ALA 258 | 18.939 27.081 64.103 1.00 25.37 | |
| ATOM | 421 CB ALA 258 | 20.313 27.705 64.416 1.00 26.26 | |
| ATOM | 422 C ALA 258 | 18.906 26.588 62.656 1.00 25.01 | |
| ATOM | 423 O ALA 258 | 18.306 27.238 61.783 1.00 25.91 | |
| ATOM | 424 N ALA 259 | 19.555 25.450 62.403 1.00 23.72 | |
| ATOM | 425 CA ALA 259 | 19.602 24.865 61.063 1.00 24.51 | |
| ATOM | 426 CB ALA 259 | 20.442 23.613 61.058 1.00 24.62 | |
| ATOM | 427 C ALA 259 | 18.187 24.525 60.623 1.00 24.36 | |
| ATOM | 428 O ALA 259 | 17.846 24.693 59.464 1.00 23.27 | |
| ATOM | 429 N LEU 260 | 17.374 24.015 61.544 1.00 23.02 | |
| ATOM | 430 CA LEU 260 | 15.986 23.685 61.188 1.00 24.75 | |
| ATOM | 431 CB LEU 260 | 15.237 23.070 62.366 1.00 26.22 | |
| ATOM | 432 CG LEU 260 | 15.550 21.633 62.742 1.00 31.27 | |
| ATOM | 433 CD1 LEU 260 | 14.906 21.342 64.082 1.00 32.20 | |
| ATOM | 434 CD2 LEU 260 | 15.054 20.679 61.658 1.00 33.32 | |
| ATOM | 435 C LEU 260 | 15.214 24.902 60.750 1.00 25.40 | |
| ATOM | 436 O LEU 260 | 14.391 24.821 59.834 1.00 23.40 | |
| ATOM | 437 N VAL 261 | 15.439 26.031 61.419 1.00 25.21 | |
| ATOM | 438 CA VAL 261 | 14.735 27.247 61.055 1.00 26.95 | |
| ATOM | 439 CB VAL 261 | 15.050 28.411 62.036 1.00 25.46 | |
| ATOM | 440 CG1 VAL 261 | 14.386 29.700 61.544 1.00 27.40 | |
| ATOM | 441 CG2 VAL 261 | 14.520 28.075 63.434 1.00 27.42 | |
| ATOM | 442 C VAL 261 | 15.104 27.671 59.640 1.00 26.53 | |
| ATOM | 443 O VAL 261 | 14.232 28.035 58.850 1.00 25.34 | |
| ATOM | 444 N VAL 262 | 16.396 27.611 59.320 1.00 27.29 | |
| ATOM | 445 CA VAL 262 | 16.874 27.995 57.993 1.00 28.11 | |
| ATOM | 446 CB VAL 262 | 18.430 27.910 57.905 1.00 30.08 | |
| ATOM | 447 CG1 VAL 262 | 18.883 27.872 56.441 1.00 33.75 | |
| ATOM | 448 CG2 VAL 262 | 19.051 29.104 58.606 1.00 32.97 | |
| ATOM | 449 C VAL 262 | 16.267 27.075 56.939 1.00 26.73 | |
| ATOM | 450 O VAL 262 | 15.909 27.511 55.840 1.00 26.28 | |
| ATOM | 451 N TRP 263 | 16.177 25.794 57.286 1.00 24.38 | |
| ATOM | 452 CA TRP 263 | 15.623 24.775 56.402 1.00 25.26 | |
| ATOM | 453 CB TRP 263 | 15.831 23.406 57.052 1.00 23.10 | |
| ATOM | 454 CG TRP 263 | 15.102 22.286 56.409 1.00 24.27 | |
| ATOM | 455 CD2 TRP 263 | 13.881 21.697 56.873 1.00 24.86 | |
| ATOM | 456 CE2 TRP 263 | 13.536 20.681 55.962 1.00 25.93 | |
| ATOM | 457 CE3 TRP 263 | 13.051 21.936 57.974 1.00 24.53 | |
| ATOM | 458 CD1 TRP 263 | 15.441 21.624 55.267 1.00 24.80 | |
| ATOM | 459 NE1 TRP 263 | 14.501 20.655 54.990 1.00 27.16 | |
| ATOM | 460 CZ2 TRP 263 | 12.391 19.895 56.115 1.00 26.26 | |
| ATOM | 461 CZ3 TRP 263 | 11.911 21.151 58.132 1.00 23.32 | |
| ATOM | 462 CH2 TRP 263 | 11.598 20.144 57.204 1.00 23.73 | |
| ATOM | 463 C TRP 263 | 14.125 25.047 56.175 1.00 24.66 | |
| ATOM | 464 O TRP 263 | 13.645 25.081 55.037 1.00 25.81 | |
| ATOM | 465 N ALA 264 | 13.391 25.278 57.252 1.00 24.56 | |
| ATOM | 466 CA ALA 264 | 11.949 25.506 57.103 1.00 25.04 | |
| ATOM | 467 CB ALA 264 | 11.304 25.733 58.464 1.00 26.00 | |
| ATOM | 468 C ALA 264 | 11.675 26.701 56.205 1.00 27.22 | |
| ATOM | 469 O ALA 264 | 10.838 26.640 55.293 1.00 24.71 | |
| ATOM | 470 N ARG 265 | 12.372 27.794 56.489 1.00 28.16 | |
| ATOM | 471 CA ARG 265 | 12.227 29.024 55.724 1.00 32.11 | |
| ATOM | 472 CB ARG 265 | 13.250 30.066 56.218 1.00 33.47 | |
| ATOM | 473 CG ARG 265 | 13.155 31.408 55.520 1.00 36.25 | |
| ATOM | 474 CD ARG 265 | 14.169 32.397 56.078 1.00 39.06 | |
| ATOM | 475 NE ARG 265 | 15.545 31.972 55.823 1.00 42.06 | |
| ATOM | 476 CZ ARG 265 | 16.609 32.534 56.391 1.00 43.94 | |
| ATOM | 477 NH1 ARG 265 | 16.447 33.541 57.244 1.00 44.49 | |
| ATOM | 478 NH2 ARG 265 | 17.827 32.092 56.110 1.00 43.52 | |
| ATOM | 479 C ARG 265 | 12.424 28.767 54.225 1.00 32.76 | |
| ATOM | 480 O ARG 265 | 11.843 29.460 53.392 1.00 35.61 | |
| ATOM | 481 N ASP 266 | 13.227 27.771 53.872 1.00 33.71 | |
| ATOM | 482 CA ASP 266 | 13.465 27.492 52.466 1.00 34.17 | |
| ATOM | 483 CB ASP 266 | 14.931 27.106 52.236 1.00 37.32 | |
| ATOM | 484 CG ASP 266 | 15.879 28.293 52.374 1.00 40.62 | |
| ATOM | 485 OD1 ASP 266 | 15.556 29.392 51.866 1.00 40.66 | |
| ATOM | 486 OD2 ASP 266 | 16.959 28.128 52.985 1.00 43.94 | |
| ATOM | 487 C ASP 266 | 12.544 26.447 51.833 1.00 33.93 | |
| ATOM | 488 O ASP 266 | 12.664 26.156 50.646 1.00 32.38 | |
| ATOM | 489 N ILE 267 | 11.640 25.869 52.619 1.00 32.18 | |
| ATOM | 490 CA ILE 267 | 10.694 24.897 52.077 1.00 30.21 | |
| ATOM | 491 CB ILE 267 | 9.820 24.279 53.210 1.00 29.16 | |
| ATOM | 492 CG2 ILE 267 | 8.586 23.588 52.620 1.00 31.21 | |
| ATOM | 493 CG1 ILE 267 | 10.643 23.291 54.038 1.00 29.01 | |
| ATOM | 494 CD1 ILE 267 | 11.069 22.051 53.278 1.00 28.89 | |
| ATOM | 495 C ILE 267 | 9.800 25.670 51.093 1.00 30.06 | |
| ATOM | 496 O ILE 267 | 9.256 26.715 51.421 1.00 29.41 | |
| ATOM | 497 N PRO 268 | 9.653 25.164 49.862 1.00 31.41 | |
| ATOM | 498 CD PRO 268 | 10.216 23.921 49.300 1.00 31.06 | |
| ATOM | 499 CA PRO 268 | 8.813 25.857 48.879 1.00 31.62 | |
| ATOM | 500 CB PRO 268 | 8.686 24.830 47.755 1.00 31.63 | |
| ATOM | 501 CG PRO 268 | 10.012 24.126 47.811 1.00 34.16 | |
| ATOM | 502 C PRO 268 | 7.459 26.286 49.444 1.00 31.83 | |
| ATOM | 503 O PRO 268 | 6.762 25.483 50.051 1.00 31.61 | |
| ATOM | 504 N HIS 269 | 7.128 27.566 49.267 1.00 31.08 | |
| ATOM | 505 CA HIS 269 | 5.867 28.164 49.715 1.00 31.47 | |
| ATOM | 506 CB HIS 269 | 4.677 27.277 49.300 1.00 33.82 | |
| ATOM | 507 CG HIS 269 | 4.710 26.845 47.865 1.00 36.44 | |
| ATOM | 508 CD2 HIS 269 | 4.694 25.611 47.305 1.00 37.13 | |
| ATOM | 509 ND1 HIS 269 | 4.734 27.740 46.816 1.00 39.48 | |
| ATOM | 510 CE1 HIS 269 | 4.731 27.078 45.672 1.00 37.91 | |
| ATOM | 511 NE2 HIS 269 | 4.706 25.785 45.941 1.00 39.31 | |
| ATOM | 512 C HIS 269 | 5.745 28.465 51.210 1.00 30.01 | |
| ATOM | 513 O HIS 269 | 4.796 29.133 51.638 1.00 28.74 | |
| ATOM | 514 N PHE 270 | 6.693 27.994 52.012 1.00 28.26 | |
| ATOM | 515 CA PHE 270 | 6.607 28.222 53.454 1.00 27.87 | |
| ATOM | 516 CB PHE 270 | 7.728 27.468 54.178 1.00 26.66 | |
| ATOM | 517 CG PHE 270 | 7.661 27.563 55.684 1.00 24.18 | |
| ATOM | 518 CD1 PHE 270 | 6.867 26.683 56.415 1.00 24.15 | |
| ATOM | 519 CD2 PHE 270 | 8.385 28.536 56.372 1.00 26.34 | |
| ATOM | 520 CE1 PHE 270 | 6.792 26.775 57.822 1.00 22.45 | |
| ATOM | 521 CE2 PHE 270 | 8.316 28.637 57.778 1.00 24.45 | |
| ATOM | 522 CZ PHE 270 | 7.516 27.754 58.494 1.00 23.03 | |
| ATOM | 523 C PHE 270 | 6.650 29.707 53.811 1.00 28.65 | |
| ATOM | 524 O PHE 270 | 5.866 30.175 54.634 1.00 29.19 | |
| ATOM | 525 N SER 271 | 7.558 30.448 53.184 0.50 29.83 | AC1 |
| ATOM | 526 CA SER 271 | 7.676 31.876 53.463 0.50 31.34 | AC1 |
| ATOM | 527 CB SER 271 | 8.959 32.432 52.839 0.50 31.74 | AC1 |
| ATOM | 528 OG SER 271 | 10.104 31.869 53.460 0.50 31.64 | AC1 |
| ATOM | 529 C SER 271 | 6.458 32.663 52.974 0.50 32.85 | AC1 |
| ATOM | 530 O SER 271 | 6.301 33.839 53.296 0.50 33.99 | AC1 |
| ATOM | 531 N GLN 272 | 5.599 32.009 52.197 1.00 33.79 | |
| ATOM | 532 CA GLN 272 | 4.378 32.642 51.696 1.00 35.22 | |
| ATOM | 533 CB GLN 272 | 3.910 31.928 50.423 1.00 39.02 | |
| ATOM | 534 CG GLN 272 | 4.777 32.210 49.191 1.00 43.59 | |
| ATOM | 535 CD GLN 272 | 4.608 31.169 48.086 1.00 45.89 | |
| ATOM | 536 OE1 GLN 272 | 3.488 30.794 47.727 1.00 47.68 | |
| ATOM | 537 NE2 GLN 272 | 5.729 30.703 47.534 1.00 48.15 | |
| ATOM | 538 C GLN 272 | 3.255 32.633 52.742 1.00 35.20 | |
| ATOM | 539 O GLN 272 | 2.288 33.397 52.648 1.00 33.55 | |
| ATOM | 540 N LEU 273 | 3.383 31.756 53.736 1.00 33.18 | |
| ATOM | 541 CA LEU 273 | 2.402 31.651 54.811 1.00 31.26 | |
| ATOM | 542 CB LEU 273 | 2.695 30.398 55.653 1.00 30.14 | |
| ATOM | 543 CG LEU 273 | 2.671 29.015 54.988 1.00 29.04 | |
| ATOM | 544 CD1 LEU 273 | 3.273 27.989 55.930 1.00 28.99 | |
| ATOM | 545 CD2 LEU 273 | 1.231 28.638 54.645 1.00 29.21 | |
| ATOM | 546 C LEU 273 | 2.500 32.880 55.722 1.00 30.95 | |
| ATOM | 547 O LEU 273 | 3.556 33.513 55.793 1.00 30.58 | |
| ATOM | 548 N GLU 274 | 1.416 33.208 56.420 1.00 31.28 | |
| ATOM | 549 CA GLU 274 | 1.435 34.332 57.355 1.00 32.85 | |
| ATOM | 550 CB GLU 274 | 0.154 34.378 58.195 1.00 35.60 | |
| ATOM | 551 CG GLU 274 | −1.039 35.022 57.511 1.00 40.50 | |
| ATOM | 552 CD GLU 274 | −0.954 36.543 57.494 1.00 43.35 | |
| ATOM | 553 OE1 GLU 274 | −1.788 37.171 56.807 1.00 44.88 | |
| ATOM | 554 OE2 GLU 274 | −0.062 37.109 58.169 1.00 45.29 | |
| ATOM | 555 C GLU 274 | 2.615 34.079 58.287 1.00 32.91 | |
| ATOM | 556 O GLU 274 | 2.867 32.936 58.693 1.00 31.56 | |
| ATOM | 557 N MET 275 | 3.331 35.136 58.632 1.00 31.54 | |
| ATOM | 558 CA MET 275 | 4.483 35.010 59.507 1.00 31.78 | |
| ATOM | 559 CB MET 275 | 5.094 36.392 59.748 1.00 34.73 | |
| ATOM | 560 CG MET 275 | 6.288 36.403 60.673 1.00 37.61 | |
| ATOM | 561 SD MET 275 | 7.574 35.262 60.158 1.00 39.49 | |
| ATOM | 562 CE MET 275 | 7.940 35.869 58.496 1.00 39.54 | |
| ATOM | 563 C MET 275 | 4.149 34.351 60.838 1.00 31.91 | |
| ATOM | 564 O MET 275 | 4.885 33.474 61.305 1.00 31.90 | |
| ATOM | 565 N GLU 276 | 3.052 34.764 61.458 1.00 31.07 | |
| ATOM | 566 CA GLU 276 | 2.684 34.184 62.736 1.00 30.79 | |
| ATOM | 567 CB GLU 276 | 1.499 34.938 63.341 1.00 35.06 | |
| ATOM | 568 CG GLU 276 | 1.866 36.382 63.755 1.00 37.66 | |
| ATOM | 569 CD GLU 276 | 3.043 36.434 64.731 1.00 40.34 | |
| ATOM | 570 OE1 GLU 276 | 2.978 35.751 65.774 1.00 41.45 | |
| ATOM | 571 OE2 GLU 276 | 4.034 37.157 64.464 1.00 42.59 | |
| ATOM | 572 C GLU 276 | 2.388 32.693 62.596 1.00 29.05 | |
| ATOM | 573 O GLU 276 | 2.529 31.946 63.559 1.00 28.93 | |
| ATOM | 574 N ASP 277 | 1.973 32.250 61.411 1.00 26.93 | |
| ATOM | 575 CA ASP 277 | 1.716 30.817 61.223 1.00 24.95 | |
| ATOM | 576 CB ASP 277 | 0.817 30.565 60.005 1.00 26.30 | |
| ATOM | 577 CG ASP 277 | −0.656 30.768 60.320 1.00 26.04 | |
| ATOM | 578 OD1 ASP 277 | −0.984 31.102 61.476 1.00 29.19 | |
| ATOM | 579 OD2 ASP 277 | −1.492 30.596 59.410 1.00 26.42 | |
| ATOM | 580 C ASP 277 | 3.056 30.089 61.069 1.00 25.34 | |
| ATOM | 581 O ASP 277 | 3.226 28.967 61.579 1.00 25.43 | |
| ATOM | 582 N GLN 278 | 4.007 30.721 60.373 1.00 23.47 | |
| ATOM | 583 CA GLN 278 | 5.338 30.132 60.203 1.00 23.93 | |
| ATOM | 584 CB GLN 278 | 6.275 31.094 59.467 1.00 23.08 | |
| ATOM | 585 CG GLN 278 | 5.931 31.265 57.999 1.00 26.36 | |
| ATOM | 586 CD GLN 278 | 6.858 32.238 57.324 1.00 26.00 | |
| ATOM | 587 OE1 GLN 278 | 8.075 32.122 57.439 1.00 26.10 | |
| ATOM | 588 NE2 GLN 278 | 6.293 33.210 56.609 1.00 28.94 | |
| ATOM | 589 C GLN 278 | 5.917 29.850 61.588 1.00 22.78 | |
| ATOM | 590 O GLN 278 | 6.445 28.765 61.857 1.00 20.87 | |
| ATOM | 591 N ILE 279 | 5.821 30.849 62.461 1.00 23.32 | |
| ATOM | 592 CA ILE 279 | 6.322 30.719 63.825 1.00 22.80 | |
| ATOM | 593 CB ILE 279 | 6.125 32.046 64.591 1.00 24.62 | |
| ATOM | 594 CG2 ILE 279 | 6.449 31.868 66.076 1.00 24.08 | |
| ATOM | 595 CG1 ILE 279 | 6.997 33.125 63.943 1.00 27.52 | |
| ATOM | 596 CD1 ILE 279 | 6.728 34.543 64.464 1.00 27.69 | |
| ATOM | 597 C ILE 279 | 5.638 29.560 64.573 1.00 22.81 | |
| ATOM | 598 O ILE 279 | 6.294 28.758 65.215 1.00 23.50 | |
| ATOM | 599 N LEU 280 | 4.318 29.463 64.498 1.00 20.16 | |
| ATOM | 600 CA LEU 280 | 3.653 28.382 65.186 1.00 18.30 | |
| ATOM | 601 CB LEU 280 | 2.125 28.553 65.113 1.00 19.64 | |
| ATOM | 602 CG LEU 280 | 1.589 29.739 65.931 1.00 22.60 | |
| ATOM | 603 CD1 LEU 280 | 0.093 29.931 65.623 1.00 24.59 | |
| ATOM | 604 CD2 LEU 280 | 1.759 29.496 67.399 1.00 25.05 | |
| ATOM | 605 C LEU 280 | 4.045 27.028 64.653 1.00 17.14 | |
| ATOM | 606 O LEU 280 | 4.160 26.084 65.436 1.00 18.84 | |
| ATOM | 607 N LEU 281 | 4.225 26.905 63.335 1.00 17.52 | |
| ATOM | 608 CA LEU 281 | 4.586 25.591 62.773 1.00 19.05 | |
| ATOM | 609 CB LEU 281 | 4.550 25.618 61.241 1.00 19.60 | |
| ATOM | 610 CG LEU 281 | 3.167 25.782 60.595 1.00 21.40 | |
| ATOM | 611 CD1 LEU 281 | 3.324 25.870 59.073 1.00 23.62 | |
| ATOM | 612 CD2 LEU 281 | 2.266 24.621 60.975 1.00 21.20 | |
| ATOM | 613 C LEU 281 | 5.968 25.143 63.255 1.00 19.84 | |
| ATOM | 614 O LEU 281 | 6.164 23.977 63.620 1.00 19.38 | |
| ATOM | 615 N ILE 282 | 6.923 26.067 63.269 1.00 20.43 | |
| ATOM | 616 CA ILE 282 | 8.270 25.740 63.731 1.00 19.64 | |
| ATOM | 617 CB ILE 282 | 9.274 26.881 63.361 1.00 20.44 | |
| ATOM | 618 CG2 ILE 282 | 10.619 26.706 64.102 1.00 20.04 | |
| ATOM | 619 CG1 ILE 282 | 9.486 26.873 61.839 1.00 20.45 | |
| ATOM | 620 CD1 ILE 282 | 10.278 28.076 61.323 1.00 22.03 | |
| ATOM | 621 C ILE 282 | 8.255 25.496 65.239 1.00 19.41 | |
| ATOM | 622 O ILE 282 | 8.894 24.574 65.717 1.00 19.21 | |
| ATOM | 623 N LYS 283 | 7.533 26.322 65.992 1.00 17.59 | |
| ATOM | 624 CA LYS 283 | 7.480 26.148 67.446 1.00 19.01 | |
| ATOM | 625 CB LYS 283 | 6.624 27.246 68.103 1.00 19.89 | |
| ATOM | 626 CG LYS 283 | 6.596 27.154 69.613 1.00 23.82 | |
| ATOM | 627 CD LYS 283 | 5.948 28.388 70.243 1.00 26.99 | |
| ATOM | 628 CE LYS 283 | 5.645 28.183 71.731 1.00 30.98 | |
| ATOM | 629 NZ LYS 283 | 6.837 27.917 72.599 1.00 34.38 | |
| ATOM | 630 C LYS 283 | 6.873 24.791 67.778 1.00 19.44 | |
| ATOM | 631 O LYS 283 | 7.274 24.120 68.729 1.00 19.31 | |
| ATOM | 632 N GLY 284 | 5.882 24.390 66.983 1.00 19.18 | |
| ATOM | 633 CA GLY 284 | 5.240 23.124 67.254 1.00 18.56 | |
| ATOM | 634 C GLY 284 | 5.981 21.885 66.806 1.00 18.31 | |
| ATOM | 635 O GLY 284 | 5.731 20.805 67.328 1.00 19.56 | |
| ATOM | 636 N SER 285 | 6.941 22.024 65.895 1.00 18.65 | |
| ATOM | 637 CA SER 285 | 7.633 20.844 65.384 1.00 17.51 | |
| ATOM | 638 CB SER 285 | 7.453 20.755 63.870 1.00 19.70 | |
| ATOM | 639 OG SER 285 | 8.063 21.874 63.212 1.00 18.48 | |
| ATOM | 640 C SER 285 | 9.136 20.728 65.663 1.00 15.25 | |
| ATOM | 641 O SER 285 | 9.705 19.673 65.406 1.00 15.43 | |
| ATOM | 642 N TRP 286 | 9.770 21.759 66.203 1.00 15.99 | |
| ATOM | 643 CA TRP 286 | 11.221 21.642 66.395 1.00 16.28 | |
| ATOM | 644 CB TRP 286 | 11.812 22.920 67.007 1.00 17.10 | |
| ATOM | 645 CG TRP 286 | 11.458 23.226 68.414 1.00 17.32 | |
| ATOM | 646 CD2 TRP 286 | 12.117 22.736 69.592 1.00 18.06 | |
| ATOM | 647 CE2 TRP 286 | 11.440 23.291 70.709 1.00 20.12 | |
| ATOM | 648 CE3 TRP 286 | 13.208 21.882 69.815 1.00 17.92 | |
| ATOM | 649 CD1 TRP 286 | 10.445 24.033 68.850 1.00 18.61 | |
| ATOM | 650 NE1 TRP 286 | 10.427 24.076 70.227 1.00 18.32 | |
| ATOM | 651 CZ2 TRP 286 | 11.816 23.020 72.027 1.00 17.23 | |
| ATOM | 652 CZ3 TRP 286 | 13.580 21.608 71.136 1.00 18.42 | |
| ATOM | 653 CH2 TRP 286 | 12.889 22.172 72.219 1.00 19.75 | |
| ATOM | 654 C TRP 286 | 11.668 20.443 67.219 1.00 16.59 | |
| ATOM | 655 O TRP 286 | 12.655 19.766 66.886 1.00 17.31 | |
| ATOM | 656 N ASN 287 | 10.938 20.167 68.282 1.00 15.26 | |
| ATOM | 657 CA ASN 287 | 11.301 19.059 69.157 1.00 14.41 | |
| ATOM | 658 CB ASN 287 | 10.508 19.180 70.456 1.00 17.68 | |
| ATOM | 659 CG ASN 287 | 10.894 18.145 71.474 1.00 19.61 | |
| ATOM | 660 OD1 ASN 287 | 11.757 18.374 72.348 1.00 23.92 | |
| ATOM | 661 ND2 ASN 287 | 10.251 16.991 71.384 1.00 18.83 | |
| ATOM | 662 C ASN 287 | 11.096 17.718 68.448 1.00 14.74 | |
| ATOM | 663 O ASN 287 | 11.954 16.846 68.508 1.00 16.16 | |
| ATOM | 664 N GLU 288 | 9.966 17.555 67.752 1.00 15.22 | |
| ATOM | 665 CA GLU 288 | 9.745 16.319 66.986 1.00 16.77 | |
| ATOM | 666 CB GLU 288 | 8.367 16.358 66.288 1.00 17.55 | |
| ATOM | 667 CG GLU 288 | 7.213 16.044 67.224 1.00 18.68 | |
| ATOM | 668 CD GLU 288 | 5.868 16.134 66.548 1.00 23.02 | |
| ATOM | 669 OE1 GLU 288 | 5.786 15.849 65.342 1.00 28.18 | |
| ATOM | 670 OE2 GLU 288 | 4.887 16.461 67.243 1.00 29.07 | |
| ATOM | 671 C GLU 288 | 10.838 16.122 65.921 1.00 17.49 | |
| ATOM | 672 O GLU 288 | 11.338 15.014 65.743 1.00 16.93 | |
| ATOM | 673 N LEU 289 | 11.193 17.204 65.220 1.00 14.56 | |
| ATOM | 674 CA LEU 289 | 12.196 17.130 64.173 1.00 15.76 | |
| ATOM | 675 CB LEU 289 | 12.248 18.451 63.390 1.00 14.77 | |
| ATOM | 676 CG LEU 289 | 11.006 18.639 62.483 1.00 16.67 | |
| ATOM | 677 CD1 LEU 289 | 10.932 20.093 62.002 1.00 18.25 | |
| ATOM | 678 CD2 LEU 289 | 11.083 17.667 61.287 1.00 15.71 | |
| ATOM | 679 C LEU 289 | 13.555 16.784 64.752 1.00 16.05 | |
| ATOM | 680 O LEU 289 | 14.301 16.022 64.138 1.00 16.56 | |
| ATOM | 681 N LEU 290 | 13.884 17.312 65.929 1.00 16.37 | |
| ATOM | 682 CA LEU 290 | 15.190 16.951 66.529 1.00 16.68 | |
| ATOM | 683 CB LEU 290 | 15.477 17.734 67.809 1.00 17.02 | |
| ATOM | 684 CG LEU 290 | 15.786 19.219 67.684 1.00 22.29 | |
| ATOM | 685 CD1 LEU 290 | 16.294 19.726 69.046 1.00 23.74 | |
| ATOM | 686 CD2 LEU 290 | 16.865 19.454 66.604 1.00 25.08 | |
| ATOM | 687 C LEU 290 | 15.229 15.471 66.868 1.00 16.18 | |
| ATOM | 688 O LEU 290 | 16.214 14.803 66.605 1.00 17.45 | |
| ATOM | 689 N LEU 291 | 14.142 14.958 67.465 1.00 16.16 | |
| ATOM | 690 CA LEU 291 | 14.088 13.553 67.826 1.00 17.64 | |
| ATOM | 691 CB LEU 291 | 12.828 13.289 68.666 1.00 17.32 | |
| ATOM | 692 CG LEU 291 | 12.824 13.961 70.046 1.00 20.40 | |
| ATOM | 693 CD1 LEU 291 | 11.405 13.964 70.627 1.00 22.18 | |
| ATOM | 694 CD2 LEU 291 | 13.789 13.222 70.971 1.00 23.46 | |
| ATOM | 695 C LEU 291 | 14.087 12.662 66.595 1.00 15.65 | |
| ATOM | 696 O LEU 291 | 14.645 11.569 66.611 1.00 17.39 | |
| ATOM | 697 N PHE 292 | 13.434 13.116 65.520 1.00 14.81 | |
| ATOM | 698 CA PHE 292 | 13.360 12.330 64.311 1.00 15.62 | |
| ATOM | 699 CB PHE 292 | 12.392 12.993 63.314 1.00 16.43 | |
| ATOM | 700 CG PHE 292 | 12.003 12.113 62.167 1.00 16.59 | |
| ATOM | 701 CD1 PHE 292 | 11.588 10.812 62.389 1.00 18.96 | |
| ATOM | 702 CD2 PHE 292 | 12.002 12.607 60.875 1.00 19.01 | |
| ATOM | 703 CE1 PHE 292 | 11.170 10.011 61.332 1.00 20.62 | |
| ATOM | 704 CE2 PHE 292 | 11.581 11.805 59.805 1.00 18.46 | |
| ATOM | 705 CZ PHE 292 | 11.166 10.499 60.060 1.00 18.06 | |
| ATOM | 706 C PHE 292 | 14.761 12.191 63.713 1.00 15.70 | |
| ATOM | 707 O PHE 292 | 15.132 11.119 63.236 1.00 15.72 | |
| ATOM | 708 N ALA 293 | 15.526 13.278 63.760 1.00 16.34 | |
| ATOM | 709 CA ALA 293 | 16.906 13.267 63.232 1.00 16.73 | |
| ATOM | 710 CB ALA 293 | 17.464 14.702 63.207 1.00 17.74 | |
| ATOM | 711 C ALA 293 | 17.802 12.368 64.090 1.00 17.09 | |
| ATOM | 712 O ALA 293 | 18.668 11.654 63.570 1.00 17.27 | |
| ATOM | 713 N ILE 294 | 17.623 12.423 65.407 1.00 16.09 | |
| ATOM | 714 CA ILE 294 | 18.389 11.569 66.312 1.00 16.22 | |
| ATOM | 715 CB ILE 294 | 18.032 11.857 67.768 1.00 15.67 | |
| ATOM | 716 CG2 ILE 294 | 18.584 10.765 68.679 1.00 16.66 | |
| ATOM | 717 CG1 ILE 294 | 18.615 13.218 68.160 1.00 17.00 | |
| ATOM | 718 CD1 ILE 294 | 18.206 13.635 69.535 1.00 20.77 | |
| ATOM | 719 C ILE 294 | 18.070 10.111 65.971 1.00 16.74 | |
| ATOM | 720 O ILE 294 | 18.954 9.296 65.833 1.00 16.38 | |
| ATOM | 721 N ALA 295 | 16.791 9.791 65.774 1.00 16.15 | |
| ATOM | 722 CA ALA 295 | 16.452 8.420 65.424 1.00 16.99 | |
| ATOM | 723 CB ALA 295 | 14.937 8.273 65.332 1.00 16.54 | |
| ATOM | 724 C ALA 295 | 17.083 7.993 64.088 1.00 17.97 | |
| ATOM | 725 O ALA 295 | 17.563 6.870 63.954 1.00 18.01 | |
| ATOM | 726 N TRP 296 | 17.071 8.881 63.102 1.00 18.59 | |
| ATOM | 727 CA TRP 296 | 17.625 8.570 61.782 1.00 18.81 | |
| ATOM | 728 CB TRP 296 | 17.321 9.740 60.835 1.00 20.21 | |
| ATOM | 729 CG TRP 296 | 17.849 9.622 59.451 1.00 22.61 | |
| ATOM | 730 CD2 TRP 296 | 17.398 8.716 58.433 1.00 25.62 | |
| ATOM | 731 CE2 TRP 296 | 18.125 9.007 57.265 1.00 26.25 | |
| ATOM | 732 CE3 TRP 296 | 16.448 7.683 58.402 1.00 25.95 | |
| ATOM | 733 CD1 TRP 296 | 18.807 10.403 58.871 1.00 26.23 | |
| ATOM | 734 NE1 TRP 296 | 18.975 10.043 57.556 1.00 26.50 | |
| ATOM | 735 CZ2 TRP 296 | 17.934 8.297 56.063 1.00 27.77 | |
| ATOM | 736 CZ3 TRP 296 | 16.257 6.977 57.206 1.00 27.65 | |
| ATOM | 737 CH2 TRP 296 | 16.996 7.289 56.061 1.00 25.35 | |
| ATOM | 738 C TRP 296 | 19.133 8.299 61.878 1.00 19.53 | |
| ATOM | 739 O TRP 296 | 19.647 7.296 61.345 1.00 21.43 | |
| ATOM | 740 N ARG 297 | 19.833 9.147 62.623 1.00 18.58 | |
| ATOM | 741 CA ARG 297 | 21.280 8.974 62.748 1.00 18.22 | |
| ATOM | 742 CB ARG 297 | 21.923 10.208 63.376 1.00 18.92 | |
| ATOM | 743 CG ARG 297 | 21.886 11.465 62.525 1.00 20.63 | |
| ATOM | 744 CD ARG 297 | 22.792 12.530 63.137 1.00 26.15 | |
| ATOM | 745 NE ARG 297 | 22.219 13.002 64.381 1.00 29.32 | |
| ATOM | 746 CZ ARG 297 | 21.393 14.040 64.462 1.00 28.97 | |
| ATOM | 747 NH1 ARG 297 | 21.066 14.720 63.373 1.00 29.81 | |
| ATOM | 748 NH2 ARG 297 | 20.872 14.372 65.628 1.00 31.03 | |
| ATOM | 749 C ARG 297 | 21.643 7.776 63.605 1.00 18.87 | |
| ATOM | 750 O ARG 297 | 22.738 7.225 63.473 1.00 19.01 | |
| ATOM | 751 N SER 298 | 20.749 7.404 64.511 1.00 17.52 | |
| ATOM | 752 CA SER 298 | 21.023 6.293 65.414 1.00 18.09 | |
| ATOM | 753 CB SER 298 | 20.134 6.407 66.655 1.00 18.94 | |
| ATOM | 754 OG SER 298 | 20.412 7.606 67.381 1.00 18.16 | |
| ATOM | 755 C SER 298 | 20.865 4.929 64.770 1.00 20.63 | |
| ATOM | 756 O SER 298 | 21.310 3.925 65.336 1.00 24.22 | |
| ATOM | 757 N MET 299 | 20.270 4.868 63.582 1.00 20.01 | |
| ATOM | 758 CA MET 299 | 20.106 3.558 62.953 1.00 22.16 | |
| ATOM | 759 CB MET 299 | 19.367 3.682 61.610 1.00 24.42 | |
| ATOM | 760 CG MET 299 | 17.940 4.159 61.735 1.00 24.29 | |
| ATOM | 761 SD MET 299 | 17.091 4.052 60.130 1.00 31.30 | |
| ATOM | 762 CE MET 299 | 18.221 4.950 59.082 1.00 31.06 | |
| ATOM | 763 C MET 299 | 21.431 2.825 62.725 1.00 25.30 | |
| ATOM | 764 O MET 299 | 21.493 1.600 62.859 1.00 26.84 | |
| ATOM | 765 N GLU 300 | 22.485 3.565 62.414 1.00 28.43 | |
| ATOM | 766 CA GLU 300 | 23.775 2.944 62.117 1.00 32.11 | |
| ATOM | 767 CB GLU 300 | 24.736 3.949 61.472 1.00 34.60 | |
| ATOM | 768 CG GLU 300 | 25.355 4.941 62.420 1.00 40.06 | |
| ATOM | 769 CD GLU 300 | 26.689 5.477 61.910 1.00 43.56 | |
| ATOM | 770 OE1 GLU 300 | 27.622 4.666 61.729 1.00 46.83 | |
| ATOM | 771 OE2 GLU 300 | 26.812 6.703 61.688 1.00 45.34 | |
| ATOM | 772 C GLU 300 | 24.467 2.304 63.300 1.00 32.94 | |
| ATOM | 773 O GLU 300 | 25.389 1.517 63.123 1.00 32.67 | |
| ATOM | 774 N PHE 301 | 24.033 2.649 64.507 1.00 31.51 | |
| ATOM | 775 CA PHE 301 | 24.632 2.094 65.710 1.00 32.60 | |
| ATOM | 776 CB PHE 301 | 24.822 3.214 66.736 1.00 32.49 | |
| ATOM | 777 CG PHE 301 | 25.879 4.203 66.349 1.00 34.07 | |
| ATOM | 778 CD1 PHE 301 | 27.223 3.838 66.358 1.00 33.32 | |
| ATOM | 779 CD2 PHE 301 | 25.537 5.483 65.929 1.00 33.10 | |
| ATOM | 780 CE1 PHE 301 | 28.207 4.737 65.949 1.00 34.17 | |
| ATOM | 781 CE2 PHE 301 | 26.506 6.385 65.519 1.00 34.69 | |
| ATOM | 782 CZ PHE 301 | 27.854 6.008 65.530 1.00 33.21 | |
| ATOM | 783 C PHE 301 | 23.820 0.940 66.301 1.00 33.77 | |
| ATOM | 784 O PHE 301 | 24.127 0.441 67.377 1.00 34.55 | |
| ATOM | 785 N LEU 302 | 22.780 0.516 65.589 1.00 34.80 | |
| ATOM | 786 CA LEU 302 | 21.966 −0.593 66.045 1.00 36.81 | |
| ATOM | 787 CB LEU 302 | 20.641 −0.645 65.287 1.00 34.98 | |
| ATOM | 788 CG LEU 302 | 19.779 0.596 65.488 1.00 31.61 | |
| ATOM | 789 CD1 LEU 302 | 18.551 0.528 64.577 1.00 31.62 | |
| ATOM | 790 CD2 LEU 302 | 19.386 0.717 66.955 1.00 31.48 | |
| ATOM | 791 C LEU 302 | 22.744 −1.869 65.775 1.00 40.52 | |
| ATOM | 792 O LEU 302 | 23.333 −2.038 64.701 1.00 40.16 | |
| ATOM | 793 N THR 303 | 22.733 −2.764 66.753 1.00 44.10 | |
| ATOM | 794 CA THR 303 | 23.422 −4.037 66.634 1.00 48.80 | |
| ATOM | 795 CB THR 303 | 23.126 −4.933 67.850 1.00 49.83 | |
| ATOM | 796 OG1 THR 303 | 23.211 −4.151 69.050 1.00 51.18 | |
| ATOM | 797 CG2 THR 303 | 24.132 −6.076 67.924 1.00 51.25 | |
| ATOM | 798 C THR 303 | 22.932 −4.731 65.368 1.00 50.84 | |
| ATOM | 799 O THR 303 | 21.739 −4.703 65.052 1.00 50.20 | |
| ATOM | 800 N ALA 304 | 23.864 −5.341 64.644 1.00 53.24 | |
| ATOM | 801 CA ALA 304 | 23.538 −6.040 63.410 1.00 55.88 | |
| ATOM | 802 CB ALA 304 | 24.739 −6.867 62.946 1.00 56.05 | |
| ATOM | 803 C ALA 304 | 22.317 −6.942 63.575 1.00 57.61 | |
| ATOM | 804 O ALA 304 | 22.133 −7.583 64.617 1.00 58.45 | |
| ATOM | 805 N ALA 305 | 21.481 −6.972 62.540 1.00 59.51 | |
| ATOM | 806 CA ALA 305 | 20.282 −7.797 62.535 1.00 60.68 | |
| ATOM | 807 CB ALA 305 | 19.548 −7.651 61.205 1.00 60.98 | |
| ATOM | 808 C ALA 305 | 20.714 −9.245 62.744 1.00 61.71 | |
| ATOM | 809 O ALA 305 | 19.925 −10.029 63.319 1.00 62.15 | |
| ATOM | 810 OXT ALA 305 | 21.848 −9.572 62.316 1.00 61.88 | |
| ATOM | 811 CB ALA 316 | 17.484 −11.402 70.435 1.00 59.02 | |
| ATOM | 812 C ALA 316 | 15.833 −9.781 71.403 1.00 57.95 | |
| ATOM | 813 O ALA 316 | 15.469 −9.327 70.316 1.00 58.69 | |
| ATOM | 814 N ALA 316 | 15.341 −12.224 71.375 1.00 58.18 | |
| ATOM | 815 CA ALA 316 | 16.413 −11.191 71.510 1.00 58.61 | |
| ATOM | 816 N ALA 317 | 15.747 −9.096 72.539 1.00 56.79 | |
| ATOM | 817 CA ALA 317 | 15.218 −7.739 72.587 1.00 54.70 | |
| ATOM | 818 CB ALA 317 | 13.813 −7.745 73.174 1.00 54.73 | |
| ATOM | 819 C ALA 317 | 16.142 −6.871 73.437 1.00 52.91 | |
| ATOM | 820 O ALA 317 | 15.697 −5.935 74.111 1.00 53.06 | |
| ATOM | 821 N SER 318 | 17.430 −7.207 73.404 1.00 49.42 | |
| ATOM | 822 CA SER 318 | 18.449 −6.478 74.151 1.00 46.14 | |
| ATOM | 823 CB SER 318 | 19.830 −7.056 73.835 1.00 46.72 | |
| ATOM | 824 OG SER 318 | 20.834 −6.462 74.639 1.00 47.98 | |
| ATOM | 825 C SER 318 | 18.378 −5.009 73.723 1.00 43.02 | |
| ATOM | 826 O SER 318 | 18.543 −4.686 72.541 1.00 44.27 | |
| ATOM | 827 N PRO 319 | 18.122 −4.100 74.676 0.50 41.03 | AC1 |
| ATOM | 828 CD PRO 319 | 17.740 −4.342 76.079 0.50 40.29 | AC1 |
| ATOM | 829 CA PRO 319 | 18.033 −2.672 74.351 0.50 38.31 | AC1 |
| ATOM | 830 CB PRO 319 | 17.690 −2.032 75.694 0.50 38.96 | AC1 |
| ATOM | 831 CG PRO 319 | 16.927 −3.116 76.402 0.50 39.34 | AC1 |
| ATOM | 832 C PRO 319 | 19.310 −2.080 73.756 0.50 35.85 | AC1 |
| ATOM | 833 O PRO 319 | 20.394 −2.240 74.319 0.50 35.73 | AC1 |
| ATOM | 834 N PRO 320 | 19.197 −1.403 72.599 1.00 33.69 | |
| ATOM | 835 CD PRO 320 | 18.041 −1.404 71.692 1.00 32.09 | |
| ATOM | 836 CA PRO 320 | 20.362 −0.784 71.952 1.00 30.83 | |
| ATOM | 837 CB PRO 320 | 19.769 −0.083 70.722 1.00 30.11 | |
| ATOM | 838 CG PRO 320 | 18.279 −0.174 70.892 1.00 32.74 | |
| ATOM | 839 C PRO 320 | 21.008 0.185 72.949 1.00 27.19 | |
| ATOM | 840 O PRO 320 | 20.317 0.824 73.756 1.00 27.36 | |
| ATOM | 841 N GLN 321 | 22.328 0.297 72.890 1.00 25.76 | |
| ATOM | 842 CA GLN 321 | 23.060 1.121 73.847 1.00 25.85 | |
| ATOM | 843 CB GLN 321 | 24.262 0.329 74.357 1.00 29.20 | |
| ATOM | 844 CG GLN 321 | 23.925 −1.090 74.757 1.00 34.12 | |
| ATOM | 845 CD GLN 321 | 23.408 −1.197 76.163 1.00 38.11 | |
| ATOM | 846 OE1 GLN 321 | 24.127 −0.916 77.123 1.00 40.85 | |
| ATOM | 847 NE2 GLN 321 | 22.150 −1.617 76.302 1.00 40.57 | |
| ATOM | 848 C GLN 321 | 23.546 2.495 73.423 1.00 23.90 | |
| ATOM | 849 O GLN 321 | 23.914 3.297 74.275 1.00 24.18 | |
| ATOM | 850 N LEU 322 | 23.536 2.790 72.128 1.00 22.59 | |
| ATOM | 851 CA LEU 322 | 24.050 4.080 71.666 1.00 21.41 | |
| ATOM | 852 CB LEU 322 | 25.291 3.879 70.775 1.00 22.86 | |
| ATOM | 853 CG LEU 322 | 26.560 3.362 71.432 1.00 25.45 | |
| ATOM | 854 CD1 LEU 322 | 27.625 3.130 70.345 1.00 28.06 | |
| ATOM | 855 CD2 LEU 322 | 27.036 4.384 72.497 1.00 24.21 | |
| ATOM | 856 C LEU 322 | 23.079 4.942 70.891 1.00 22.08 | |
| ATOM | 857 O LEU 322 | 22.435 4.478 69.961 1.00 23.11 | |
| ATOM | 858 N MET 323 | 23.003 6.205 71.270 1.00 20.81 | |
| ATOM | 859 CA MET 323 | 22.145 7.157 70.590 1.00 21.91 | |
| ATOM | 860 CB MET 323 | 21.213 7.835 71.598 1.00 23.20 | |
| ATOM | 861 CG MET 323 | 20.325 8.922 70.978 1.00 22.36 | |
| ATOM | 862 SD MET 323 | 19.196 9.636 72.187 1.00 22.04 | |
| ATOM | 863 CE MET 323 | 18.026 8.314 72.302 1.00 22.12 | |
| ATOM | 864 C MET 323 | 23.054 8.187 69.914 1.00 25.54 | |
| ATOM | 865 O MET 323 | 24.002 8.691 70.530 1.00 25.31 | |
| ATOM | 866 N CYS 324 | 22.781 8.506 68.650 1.00 22.41 | |
| ATOM | 867 CA CYS 324 | 23.619 9.483 67.941 1.00 24.83 | |
| ATOM | 868 CB CYS 324 | 23.854 9.029 66.512 1.00 23.91 | |
| ATOM | 869 SG CYS 324 | 24.921 10.185 65.588 1.00 27.74 | |
| ATOM | 870 C CYS 324 | 22.995 10.873 67.950 1.00 24.34 | |
| ATOM | 871 O CYS 324 | 22.010 11.148 67.249 1.00 24.90 | |
| ATOM | 872 N LEU 325 | 23.560 11.758 68.749 1.00 24.49 | |
| ATOM | 873 CA LEU 325 | 23.038 13.114 68.843 1.00 25.66 | |
| ATOM | 874 CB LEU 325 | 23.421 13.746 70.183 1.00 26.79 | |
| ATOM | 875 CG LEU 325 | 22.935 12.964 71.421 1.00 27.19 | |
| ATOM | 876 CD1 LEU 325 | 23.256 13.744 72.704 1.00 29.28 | |
| ATOM | 877 CD2 LEU 325 | 21.423 12.732 71.320 1.00 27.92 | |
| ATOM | 878 C LEU 325 | 23.513 13.990 67.682 1.00 28.10 | |
| ATOM | 879 O LEU 325 | 22.860 14.978 67.344 1.00 31.20 | |
| ATOM | 880 N MET 326 | 24.650 13.618 67.094 1.00 28.83 | |
| ATOM | 881 CA MET 326 | 25.253 14.311 65.947 1.00 30.95 | |
| ATOM | 882 CB MET 326 | 25.726 15.721 66.350 1.00 31.86 | |
| ATOM | 883 CG MET 326 | 26.894 15.710 67.335 1.00 31.84 | |
| ATOM | 884 SD MET 326 | 27.648 17.333 67.693 1.00 34.98 | |
| ATOM | 885 CE MET 326 | 29.085 16.800 68.706 1.00 30.77 | |
| ATOM | 886 C MET 326 | 26.462 13.453 65.513 1.00 33.06 | |
| ATOM | 887 O MET 326 | 26.882 12.565 66.242 1.00 32.71 | |
| ATOM | 888 N PRO 327 | 27.013 13.682 64.307 1.00 35.54 | |
| ATOM | 889 CD PRO 327 | 26.511 14.545 63.227 1.00 35.87 | |
| ATOM | 890 CA PRO 327 | 28.180 12.896 63.857 1.00 36.24 | |
| ATOM | 891 CB PRO 327 | 28.508 13.519 62.503 1.00 37.87 | |
| ATOM | 892 CG PRO 327 | 27.159 13.932 62.001 1.00 35.11 | |
| ATOM | 893 C PRO 327 | 29.353 13.035 64.836 1.00 38.75 | |
| ATOM | 894 O PRO 327 | 29.754 14.154 65.155 1.00 38.99 | |
| ATOM | 895 N GLY 328 | 29.890 11.908 65.314 1.00 38.52 | |
| ATOM | 896 CA GLY 328 | 31.001 11.933 66.257 1.00 38.65 | |
| ATOM | 897 C GLY 328 | 30.606 12.217 67.700 1.00 38.39 | |
| ATOM | 898 O GLY 328 | 31.431 12.604 68.545 1.00 39.05 | |
| ATOM | 899 N MET 329 | 29.326 12.020 67.994 1.00 36.32 | |
| ATOM | 900 CA MET 329 | 28.830 12.251 69.326 1.00 34.24 | |
| ATOM | 901 CB MET 329 | 28.343 13.693 69.451 1.00 38.91 | |
| ATOM | 902 CG MET 329 | 27.462 13.991 70.649 1.00 44.24 | |
| ATOM | 903 SD MET 329 | 28.233 13.696 72.235 1.00 50.95 | |
| ATOM | 904 CE MET 329 | 26.854 12.835 73.089 1.00 48.58 | |
| ATOM | 905 C MET 329 | 27.700 11.271 69.603 1.00 30.52 | |
| ATOM | 906 O MET 329 | 26.647 11.331 68.975 1.00 28.92 | |
| ATOM | 907 N THR 330 | 27.960 10.324 70.490 1.00 26.42 | |
| ATOM | 908 CA THR 330 | 26.933 9.370 70.865 1.00 23.65 | |
| ATOM | 909 CB THR 330 | 27.338 7.895 70.542 1.00 24.12 | |
| ATOM | 910 OG1 THR 330 | 28.672 7.654 71.005 1.00 27.18 | |
| ATOM | 911 CG2 THR 330 | 27.269 7.622 69.034 1.00 26.34 | |
| ATOM | 912 C THR 330 | 26.685 9.475 72.360 1.00 23.23 | |
| ATOM | 913 O THR 330 | 27.572 9.802 73.146 1.00 23.91 | |
| ATOM | 914 N LEU 331 | 25.450 9.211 72.748 1.00 20.37 | |
| ATOM | 915 CA LEU 331 | 25.082 9.213 74.142 1.00 21.33 | |
| ATOM | 916 CB LEU 331 | 23.773 9.986 74.340 1.00 24.38 | |
| ATOM | 917 CG LEU 331 | 23.113 9.731 75.696 1.00 25.70 | |
| ATOM | 918 CD1 LEU 331 | 23.979 10.317 76.825 1.00 30.48 | |
| ATOM | 919 CD2 LEU 331 | 21.723 10.348 75.694 1.00 29.64 | |
| ATOM | 920 C LEU 331 | 24.892 7.737 74.489 1.00 21.13 | |
| ATOM | 921 O LEU 331 | 24.158 7.026 73.819 1.00 19.21 | |
| ATOM | 922 N HIS 332 | 25.582 7.262 75.525 1.00 18.80 | |
| ATOM | 923 CA HIS 332 | 25.472 5.864 75.901 1.00 18.64 | |
| ATOM | 924 CB HIS 332 | 26.740 5.417 76.658 1.00 18.77 | |
| ATOM | 925 CG HIS 332 | 26.826 3.938 76.862 1.00 21.68 | |
| ATOM | 926 CD2 HIS 332 | 27.533 2.991 76.205 1.00 22.97 | |
| ATOM | 927 ND1 HIS 332 | 26.092 3.273 77.824 1.00 22.38 | |
| ATOM | 928 CE1 HIS 332 | 26.340 1.978 77.745 1.00 23.74 | |
| ATOM | 929 NE2 HIS 332 | 27.213 1.778 76.769 1.00 25.26 | |
| ATOM | 930 C HIS 332 | 24.262 5.721 76.820 1.00 18.95 | |
| ATOM | 931 O HIS 332 | 23.981 6.620 77.616 1.00 19.91 | |
| ATOM | 932 N ARG 333 | 23.594 4.581 76.715 1.00 19.39 | |
| ATOM | 933 CA ARG 333 | 22.394 4.282 77.509 1.00 19.71 | |
| ATOM | 934 CB ARG 333 | 21.930 2.850 77.235 1.00 20.65 | |
| ATOM | 935 CG ARG 333 | 20.582 2.512 77.896 1.00 21.54 | |
| ATOM | 936 CD ARG 333 | 20.018 1.212 77.353 1.00 21.69 | |
| ATOM | 937 NE ARG 333 | 18.703 0.862 77.899 1.00 20.98 | |
| ATOM | 938 CZ ARG 333 | 18.501 0.034 78.921 1.00 22.46 | |
| ATOM | 939 NH1 ARG 333 | 19.535 −0.547 79.531 1.00 24.12 | |
| ATOM | 940 NH2 ARG 333 | 17.255 −0.241 79.309 1.00 20.87 | |
| ATOM | 941 C ARG 333 | 22.643 4.463 79.018 1.00 21.14 | |
| ATOM | 942 O ARG 333 | 21.782 4.926 79.755 1.00 18.39 | |
| ATOM | 943 N ASN 334 | 23.832 4.107 79.484 1.00 21.27 | |
| ATOM | 944 CA ASN 334 | 24.114 4.259 80.910 1.00 23.47 | |
| ATOM | 945 CB ASN 334 | 25.486 3.653 81.244 1.00 24.93 | |
| ATOM | 946 CG ASN 334 | 25.504 2.119 81.158 1.00 25.59 | |
| ATOM | 947 OD1 ASN 334 | 24.467 1.461 81.054 1.00 26.24 | |
| ATOM | 948 ND2 ASN 334 | 26.707 1.544 81.204 1.00 29.08 | |
| ATOM | 949 C ASN 334 | 24.048 5.718 81.380 1.00 22.91 | |
| ATOM | 950 O ASN 334 | 23.649 6.000 82.530 1.00 22.99 | |
| ATOM | 951 N SER 335 | 24.441 6.657 80.524 1.00 22.89 | |
| ATOM | 952 CA SER 335 | 24.378 8.059 80.904 1.00 22.41 | |
| ATOM | 953 CB SER 335 | 25.178 8.915 79.930 1.00 27.35 | |
| ATOM | 954 OG SER 335 | 26.517 8.437 79.856 1.00 31.39 | |
| ATOM | 955 C SER 335 | 22.908 8.484 80.939 1.00 23.03 | |
| ATOM | 956 O SER 335 | 22.496 9.260 81.809 1.00 22.21 | |
| ATOM | 957 N ALA 336 | 22.126 7.970 79.989 1.00 21.81 | |
| ATOM | 958 CA ALA 336 | 20.703 8.255 79.952 1.00 21.09 | |
| ATOM | 959 CB ALA 336 | 20.060 7.595 78.718 1.00 21.81 | |
| ATOM | 960 C ALA 336 | 20.044 7.744 81.232 1.00 21.37 | |
| ATOM | 961 O ALA 336 | 19.209 8.425 81.830 1.00 19.40 | |
| ATOM | 962 N LEU 337 | 20.423 6.540 81.660 1.00 19.77 | |
| ATOM | 963 CA LEU 337 | 19.860 5.960 82.865 1.00 19.59 | |
| ATOM | 964 CB LEU 337 | 20.374 4.517 83.039 1.00 19.20 | |
| ATOM | 965 CG LEU 337 | 19.835 3.502 82.031 1.00 20.97 | |
| ATOM | 966 CD1 LEU 337 | 20.702 2.280 82.055 1.00 23.49 | |
| ATOM | 967 CD2 LEU 337 | 18.391 3.164 82.329 1.00 21.07 | |
| ATOM | 968 C LEU 337 | 20.206 6.777 84.089 1.00 20.54 | |
| ATOM | 969 O LEU 337 | 19.364 6.997 84.985 1.00 19.57 | |
| ATOM | 970 N GLN 338 | 21.454 7.226 84.131 1.00 21.36 | |
| ATOM | 971 CA GLN 338 | 21.920 7.994 85.264 1.00 22.39 | |
| ATOM | 972 CB GLN 338 | 23.439 8.206 85.179 1.00 22.44 | |
| ATOM | 973 CG GLN 338 | 23.973 8.755 86.488 1.00 27.14 | |
| ATOM | 974 CD GLN 338 | 25.467 9.026 86.481 1.00 29.23 | |
| ATOM | 975 OE1 GLN 338 | 25.955 9.827 87.278 1.00 30.73 | |
| ATOM | 976 NE2 GLN 338 | 26.196 8.360 85.596 1.00 30.79 | |
| ATOM | 977 C GLN 338 | 21.200 9.331 85.381 1.00 23.40 | |
| ATOM | 978 O GLN 338 | 20.894 9.790 86.490 1.00 21.75 | |
| ATOM | 979 N ALA 339 | 20.899 9.935 84.227 1.00 22.63 | |
| ATOM | 980 CA ALA 339 | 20.215 11.217 84.191 1.00 24.41 | |
| ATOM | 981 CB ALA 339 | 20.430 11.903 82.832 1.00 23.46 | |
| ATOM | 982 C ALA 339 | 18.727 11.082 84.463 1.00 24.73 | |
| ATOM | 983 O ALA 339 | 18.059 12.084 84.641 1.00 27.16 | |
| ATOM | 984 N GLY 340 | 18.218 9.850 84.499 1.00 22.58 | |
| ATOM | 985 CA GLY 340 | 16.803 9.622 84.754 1.00 23.89 | |
| ATOM | 986 C GLY 340 | 15.898 9.580 83.531 1.00 25.09 | |
| ATOM | 987 O GLY 340 | 14.666 9.607 83.666 1.00 25.54 | |
| ATOM | 988 N VAL 341 | 16.492 9.479 82.346 1.00 23.51 | |
| ATOM | 989 CA VAL 341 | 15.719 9.473 81.095 1.00 20.92 | |
| ATOM | 990 CB VAL 341 | 16.134 10.677 80.211 1.00 21.83 | |
| ATOM | 991 CG1 VAL 341 | 15.838 11.981 80.942 1.00 21.94 | |
| ATOM | 992 CG2 VAL 341 | 17.621 10.577 79.858 1.00 21.91 | |
| ATOM | 993 C VAL 341 | 15.891 8.185 80.300 1.00 20.24 | |
| ATOM | 994 O VAL 341 | 15.803 8.182 79.083 1.00 19.53 | |
| ATOM | 995 N GLY 342 | 16.132 7.079 80.999 1.00 19.65 | |
| ATOM | 996 CA GLY 342 | 16.324 5.827 80.301 1.00 18.76 | |
| ATOM | 997 C GLY 342 | 15.104 5.352 79.541 1.00 19.27 | |
| ATOM | 998 O GLY 342 | 15.230 4.771 78.470 1.00 18.82 | |
| ATOM | 999 N GLN 343 | 13.922 5.598 80.088 0.50 18.31 | AC1 |
| ATOM | 1000 CA GLN 343 | 12.706 5.163 79.422 0.50 19.11 | AC1 |
| ATOM | 1001 CB GLN 343 | 11.507 5.485 80.305 0.50 19.94 | AC1 |
| ATOM | 1002 CG GLN 343 | 10.200 4.866 79.867 0.50 22.91 | AC1 |
| ATOM | 1003 CD GLN 343 | 9.082 5.177 80.852 0.50 24.13 | AC1 |
| ATOM | 1004 OE1 GLN 343 | 8.669 6.326 80.995 0.50 26.85 | AC1 |
| ATOM | 1005 NE2 GLN 343 | 8.606 4.154 81.555 0.50 25.40 | AC1 |
| ATOM | 1006 C GLN 343 | 12.542 5.813 78.043 0.50 19.14 | AC1 |
| ATOM | 1007 O GLN 343 | 12.318 5.127 77.050 0.50 18.83 | AC1 |
| ATOM | 1008 N ILE 344 | 12.664 7.136 77.977 1.00 17.95 | |
| ATOM | 1009 CA ILE 344 | 12.506 7.812 76.693 1.00 17.74 | |
| ATOM | 1010 CB ILE 344 | 12.348 9.372 76.875 1.00 18.47 | |
| ATOM | 1011 CG2 ILE 344 | 13.700 10.010 77.231 1.00 20.66 | |
| ATOM | 1012 CG1 ILE 344 | 11.754 9.984 75.596 1.00 19.04 | |
| ATOM | 1013 CD1 ILE 344 | 11.445 11.499 75.662 1.00 21.07 | |
| ATOM | 1014 C ILE 344 | 13.641 7.448 75.718 1.00 17.34 | |
| ATOM | 1015 O ILE 344 | 13.440 7.389 74.499 1.00 16.12 | |
| ATOM | 1016 N PHE 345 | 14.844 7.210 76.248 1.00 16.60 | |
| ATOM | 1017 CA PHE 345 | 15.976 6.826 75.417 1.00 17.63 | |
| ATOM | 1018 CB PHE 345 | 17.208 6.636 76.322 1.00 17.86 | |
| ATOM | 1019 CG PHE 345 | 18.455 6.189 75.613 1.00 18.24 | |
| ATOM | 1020 CD1 PHE 345 | 18.657 4.848 75.273 1.00 18.20 | |
| ATOM | 1021 CD2 PHE 345 | 19.470 7.090 75.363 1.00 20.10 | |
| ATOM | 1022 CE1 PHE 345 | 19.854 4.412 74.706 1.00 19.01 | |
| ATOM | 1023 CE2 PHE 345 | 20.663 6.673 74.798 1.00 17.96 | |
| ATOM | 1024 CZ PHE 345 | 20.861 5.328 74.469 1.00 18.58 | |
| ATOM | 1025 C PHE 345 | 15.603 5.522 74.710 1.00 18.98 | |
| ATOM | 1026 O PHE 345 | 15.756 5.389 73.491 1.00 18.15 | |
| ATOM | 1027 N ASP 346 | 15.072 4.568 75.480 1.00 18.04 | |
| ATOM | 1028 CA ASP 346 | 14.695 3.300 74.894 1.00 18.83 | |
| ATOM | 1029 CB ASP 346 | 14.285 2.302 75.988 1.00 19.96 | |
| ATOM | 1030 CG ASP 346 | 15.467 1.768 76.786 1.00 24.04 | |
| ATOM | 1031 OD1 ASP 346 | 16.630 2.038 76.430 1.00 24.95 | |
| ATOM | 1032 OD2 ASP 346 | 15.232 1.043 77.784 1.00 24.86 | |
| ATOM | 1033 C ASP 346 | 13.550 3.462 73.872 1.00 18.04 | |
| ATOM | 1034 O ASP 346 | 13.549 2.782 72.841 1.00 19.39 | |
| ATOM | 1035 N ARG 347 | 12.603 4.359 74.138 1.00 19.77 | |
| ATOM | 1036 CA ARG 347 | 11.493 4.579 73.188 1.00 20.42 | |
| ATOM | 1037 CB ARG 347 | 10.443 5.558 73.708 1.00 24.20 | |
| ATOM | 1038 CG ARG 347 | 9.527 5.024 74.786 1.00 26.30 | |
| ATOM | 1039 CD ARG 347 | 8.310 5.929 74.937 1.00 28.76 | |
| ATOM | 1040 NE ARG 347 | 7.383 5.807 73.813 1.00 29.58 | |
| ATOM | 1041 CZ ARG 347 | 6.312 6.577 73.629 1.00 31.91 | |
| ATOM | 1042 NH1 ARG 347 | 6.028 7.536 74.492 1.00 32.14 | |
| ATOM | 1043 NH2 ARG 347 | 5.522 6.378 72.579 1.00 32.47 | |
| ATOM | 1044 C ARG 347 | 12.012 5.120 71.875 1.00 20.09 | |
| ATOM | 1045 O ARG 347 | 11.586 4.703 70.806 1.00 18.14 | |
| ATOM | 1046 N VAL 348 | 12.939 6.073 71.945 1.00 18.48 | |
| ATOM | 1047 CA VAL 348 | 13.480 6.621 70.708 1.00 18.94 | |
| ATOM | 1048 CB VAL 348 | 14.532 7.713 70.991 1.00 18.19 | |
| ATOM | 1049 CG1 VAL 348 | 15.286 8.052 69.706 1.00 18.18 | |
| ATOM | 1050 CG2 VAL 348 | 13.862 8.930 71.600 1.00 19.11 | |
| ATOM | 1051 C VAL 348 | 14.143 5.518 69.882 1.00 18.63 | |
| ATOM | 1052 O VAL 348 | 13.885 5.380 68.691 1.00 17.99 | |
| ATOM | 1053 N LEU 349 | 14.981 4.709 70.514 1.00 18.35 | |
| ATOM | 1054 CA LEU 349 | 15.679 3.686 69.749 1.00 19.38 | |
| ATOM | 1055 CB LEU 349 | 16.906 3.175 70.526 1.00 20.34 | |
| ATOM | 1056 CG LEU 349 | 17.920 4.286 70.807 1.00 20.49 | |
| ATOM | 1057 CD1 LEU 349 | 19.173 3.626 71.408 1.00 23.32 | |
| ATOM | 1058 CD2 LEU 349 | 18.273 5.078 69.500 1.00 18.92 | |
| ATOM | 1059 C LEU 349 | 14.822 2.509 69.294 1.00 20.39 | |
| ATOM | 1060 O LEU 349 | 15.112 1.901 68.263 1.00 19.62 | |
| ATOM | 1061 N SER 350 | 13.746 2.208 70.025 1.00 21.15 | |
| ATOM | 1062 CA SER 350 | 12.907 1.074 69.623 1.00 23.27 | |
| ATOM | 1063 CB SER 350 | 12.443 0.294 70.855 1.00 25.65 | |
| ATOM | 1064 OG SER 350 | 11.622 1.081 71.687 1.00 28.64 | |
| ATOM | 1065 C SER 350 | 11.698 1.451 68.766 1.00 21.84 | |
| ATOM | 1066 O SER 350 | 11.424 0.790 67.762 1.00 25.35 | |
| ATOM | 1067 N GLU 351 | 10.986 2.516 69.131 1.00 22.62 | |
| ATOM | 1068 CA GLU 351 | 9.801 2.906 68.368 1.00 22.04 | |
| ATOM | 1069 CB GLU 351 | 8.834 3.680 69.252 1.00 22.43 | |
| ATOM | 1070 CG GLU 351 | 8.454 2.916 70.486 1.00 26.86 | |
| ATOM | 1071 CD GLU 351 | 7.434 3.650 71.303 1.00 28.97 | |
| ATOM | 1072 OE1 GLU 351 | 7.473 3.544 72.548 1.00 31.85 | |
| ATOM | 1073 OE2 GLU 351 | 6.581 4.330 70.691 1.00 32.68 | |
| ATOM | 1074 C GLU 351 | 10.133 3.708 67.132 1.00 22.11 | |
| ATOM | 1075 O GLU 351 | 9.348 3.766 66.183 1.00 22.30 | |
| ATOM | 1076 N LEU 352 | 11.309 4.328 67.113 1.00 19.99 | |
| ATOM | 1077 CA LEU 352 | 11.696 5.083 65.934 1.00 19.01 | |
| ATOM | 1078 CB LEU 352 | 12.043 6.555 66.293 1.00 18.64 | |
| ATOM | 1079 CG LEU 352 | 10.911 7.365 66.950 1.00 19.12 | |
| ATOM | 1080 CD1 LEU 352 | 11.445 8.742 67.339 1.00 19.51 | |
| ATOM | 1081 CD2 LEU 352 | 9.703 7.505 66.018 1.00 19.25 | |
| ATOM | 1082 C LEU 352 | 12.859 4.467 65.163 1.00 19.51 | |
| ATOM | 1083 O LEU 352 | 12.704 3.977 64.038 1.00 21.10 | |
| ATOM | 1084 N SER 353 | 14.052 4.464 65.754 0.50 18.72 | AC1 |
| ATOM | 1085 CA SER 353 | 15.203 3.933 65.034 0.50 18.55 | AC1 |
| ATOM | 1086 CB SER 353 | 16.473 4.032 65.883 0.50 16.66 | AC1 |
| ATOM | 1087 OG SER 353 | 16.782 5.378 66.160 0.50 11.47 | AC1 |
| ATOM | 1088 C SER 353 | 15.045 2.500 64.546 0.50 19.60 | AC1 |
| ATOM | 1089 O SER 353 | 15.207 2.236 63.355 0.50 20.26 | AC1 |
| ATOM | 1090 N LEU 354 | 14.746 1.579 65.455 1.00 21.67 | |
| ATOM | 1091 CA LEU 354 | 14.602 0.181 65.051 1.00 23.55 | |
| ATOM | 1092 CB LEU 354 | 14.360 −0.715 66.278 1.00 24.35 | |
| ATOM | 1093 CG LEU 354 | 14.284 −2.222 66.012 1.00 30.18 | |
| ATOM | 1094 CD1 LEU 354 | 15.388 −2.664 65.055 1.00 31.88 | |
| ATOM | 1095 CD2 LEU 354 | 14.392 −2.944 67.345 1.00 31.36 | |
| ATOM | 1096 C LEU 354 | 13.489 −0.011 64.009 1.00 24.82 | |
| ATOM | 1097 O LEU 354 | 13.696 −0.684 63.004 1.00 25.66 | |
| ATOM | 1098 N LYS 355 | 12.324 0.587 64.234 1.00 26.97 | |
| ATOM | 1099 CA LYS 355 | 11.236 0.454 63.272 1.00 26.95 | |
| ATOM | 1100 CB LYS 355 | 10.003 1.226 63.735 1.00 29.30 | |
| ATOM | 1101 CG LYS 355 | 9.228 0.572 64.843 1.00 34.34 | |
| ATOM | 1102 CD LYS 355 | 8.440 −0.616 64.321 1.00 38.57 | |
| ATOM | 1103 CE LYS 355 | 7.365 −1.007 65.319 1.00 39.54 | |
| ATOM | 1104 NZ LYS 355 | 7.883 −0.958 66.713 1.00 40.20 | |
| ATOM | 1105 C LYS 355 | 11.637 0.964 61.899 1.00 27.30 | |
| ATOM | 1106 O LYS 355 | 11.281 0.369 60.883 1.00 28.58 | |
| ATOM | 1107 N MET 356 | 12.362 2.080 61.860 1.00 25.41 | |
| ATOM | 1108 CA MET 356 | 12.787 2.641 60.594 1.00 25.37 | |
| ATOM | 1109 CB MET 356 | 13.279 4.071 60.778 1.00 27.04 | |
| ATOM | 1110 CG MET 356 | 12.126 5.017 60.918 1.00 26.57 | |
| ATOM | 1111 SD MET 356 | 12.669 6.671 60.768 1.00 36.72 | |
| ATOM | 1112 CE MET 356 | 13.015 6.835 62.357 1.00 16.80 | |
| ATOM | 1113 C MET 356 | 13.836 1.808 59.896 1.00 26.93 | |
| ATOM | 1114 O MET 356 | 13.907 1.807 58.676 1.00 25.41 | |
| ATOM | 1115 N ARG 357 | 14.672 1.114 60.661 1.00 27.88 | |
| ATOM | 1116 CA ARG 357 | 15.656 0.281 60.003 1.00 29.75 | |
| ATOM | 1117 CB ARG 357 | 16.733 −0.214 60.961 1.00 31.09 | |
| ATOM | 1118 CG ARG 357 | 17.748 −1.056 60.213 1.00 35.17 | |
| ATOM | 1119 CD ARG 357 | 18.840 −1.607 61.112 1.00 38.46 | |
| ATOM | 1120 NE ARG 357 | 18.313 −2.543 62.097 1.00 41.53 | |
| ATOM | 1121 CZ ARG 357 | 19.066 −3.164 63.003 1.00 43.38 | |
| ATOM | 1122 NH1 ARG 357 | 20.376 −2.942 63.037 1.00 42.92 | |
| ATOM | 1123 NH2 ARG 357 | 18.512 −3.995 63.879 1.00 43.48 | |
| ATOM | 1124 C ARG 357 | 14.926 −0.925 59.422 1.00 29.63 | |
| ATOM | 1125 O ARG 357 | 15.218 −1.353 58.310 1.00 29.83 | |
| ATOM | 1126 N THR 358 | 13.966 −1.452 60.171 1.00 29.51 | |
| ATOM | 1127 CA THR 358 | 13.204 −2.609 59.704 1.00 30.65 | |
| ATOM | 1128 CB THR 358 | 12.273 −3.117 60.812 1.00 31.00 | |
| ATOM | 1129 OG1 THR 358 | 13.071 −3.609 61.895 1.00 32.46 | |
| ATOM | 1130 CG2 THR 358 | 11.374 −4.260 60.306 1.00 34.28 | |
| ATOM | 1131 C THR 358 | 12.408 −2.281 58.438 1.00 30.73 | |
| ATOM | 1132 O THR 358 | 12.286 −3.114 57.533 1.00 32.33 | |
| ATOM | 1133 N LEU 359 | 11.895 −1.056 58.359 1.00 29.40 | |
| ATOM | 1134 CA LEU 359 | 11.124 −0.610 57.201 1.00 29.68 | |
| ATOM | 1135 CB LEU 359 | 10.242 0.583 57.605 1.00 30.32 | |
| ATOM | 1136 CG LEU 359 | 8.732 0.609 57.321 1.00 34.26 | |
| ATOM | 1137 CD1 LEU 359 | 8.197 2.038 57.529 1.00 33.77 | |
| ATOM | 1138 CD2 LEU 359 | 8.452 0.142 55.900 1.00 34.08 | |
| ATOM | 1139 C LEU 359 | 12.046 −0.168 56.066 1.00 28.77 | |
| ATOM | 1140 O LEU 359 | 11.585 0.164 54.974 1.00 27.98 | |
| ATOM | 1141 N ARG 360 | 13.349 −0.130 56.327 1.00 30.53 | |
| ATOM | 1142 CA ARG 360 | 14.315 0.310 55.327 1.00 30.15 | |
| ATOM | 1143 CB ARG 360 | 14.381 −0.695 54.178 1.00 33.80 | |
| ATOM | 1144 CG ARG 360 | 14.798 −2.076 54.625 1.00 36.43 | |
| ATOM | 1145 CD ARG 360 | 14.018 −3.165 53.900 1.00 39.56 | |
| ATOM | 1146 NE ARG 360 | 12.588 −3.168 54.226 1.00 41.14 | |
| ATOM | 1147 CZ ARG 360 | 11.618 −2.942 53.340 1.00 42.18 | |
| ATOM | 1148 NH1 ARG 360 | 11.918 −2.688 52.071 1.00 41.02 | |
| ATOM | 1149 NH2 ARG 360 | 10.345 −2.983 53.717 1.00 42.86 | |
| ATOM | 1150 C ARG 360 | 13.975 1.699 54.787 1.00 29.92 | |
| ATOM | 1151 O ARG 360 | 14.073 1.958 53.592 1.00 28.84 | |
| ATOM | 1152 N VAL 361 | 13.569 2.600 55.677 1.00 27.64 | |
| ATOM | 1153 CA VAL 361 | 13.261 3.956 55.267 1.00 25.17 | |
| ATOM | 1154 CB VAL 361 | 12.909 4.819 56.480 1.00 24.00 | |
| ATOM | 1155 CG1 VAL 361 | 12.684 6.254 56.066 1.00 24.37 | |
| ATOM | 1156 CG2 VAL 361 | 11.688 4.249 57.166 1.00 23.19 | |
| ATOM | 1157 C VAL 361 | 14.504 4.525 54.594 1.00 25.34 | |
| ATOM | 1158 O VAL 361 | 15.613 4.407 55.133 1.00 25.79 | |
| ATOM | 1159 N ASP 362 | 14.344 5.109 53.404 1.00 24.62 | |
| ATOM | 1160 CA ASP 362 | 15.481 5.712 52.718 1.00 24.80 | |
| ATOM | 1161 CB ASP 362 | 15.540 5.316 51.223 1.00 27.01 | |
| ATOM | 1162 CG ASP 362 | 14.307 5.720 50.433 1.00 29.25 | |
| ATOM | 1163 OD1 ASP 362 | 13.697 6.764 50.731 1.00 27.58 | |
| ATOM | 1164 OD2 ASP 362 | 13.963 4.987 49.479 1.00 32.41 | |
| ATOM | 1165 C ASP 362 | 15.520 7.226 52.863 1.00 25.99 | |
| ATOM | 1166 O ASP 362 | 14.631 7.823 53.486 1.00 25.65 | |
| ATOM | 1167 N GLN 363 | 16.561 7.845 52.312 1.00 24.73 | |
| ATOM | 1168 CA GLN 363 | 16.742 9.286 52.405 1.00 26.50 | |
| ATOM | 1169 CB GLN 363 | 18.044 9.682 51.690 1.00 31.84 | |
| ATOM | 1170 CG GLN 363 | 18.339 11.179 51.662 1.00 36.89 | |
| ATOM | 1171 CD GLN 363 | 19.008 11.689 52.921 1.00 40.13 | |
| ATOM | 1172 OE1 GLN 363 | 18.615 11.346 54.047 1.00 42.87 | |
| ATOM | 1173 NE2 GLN 363 | 20.013 12.536 52.743 1.00 40.44 | |
| ATOM | 1174 C GLN 363 | 15.540 10.070 51.855 1.00 25.17 | |
| ATOM | 1175 O GLN 363 | 15.077 11.020 52.480 1.00 22.69 | |
| ATOM | 1176 N ALA 364 | 15.023 9.659 50.701 1.00 24.53 | |
| ATOM | 1177 CA ALA 364 | 13.863 10.340 50.099 1.00 22.80 | |
| ATOM | 1178 CB ALA 364 | 13.524 9.691 48.753 1.00 22.91 | |
| ATOM | 1179 C ALA 364 | 12.631 10.326 51.022 1.00 21.81 | |
| ATOM | 1180 O ALA 364 | 11.946 11.336 51.178 1.00 21.88 | |
| ATOM | 1181 N GLU 365 | 12.362 9.188 51.636 1.00 22.72 | |
| ATOM | 1182 CA GLU 365 | 11.218 9.062 52.532 1.00 21.63 | |
| ATOM | 1183 CB GLU 365 | 10.990 7.585 52.856 1.00 22.68 | |
| ATOM | 1184 CG GLU 365 | 10.724 6.806 51.583 1.00 23.15 | |
| ATOM | 1185 CD GLU 365 | 10.797 5.303 51.723 1.00 28.04 | |
| ATOM | 1186 OE1 GLU 365 | 11.632 4.813 52.501 1.00 26.39 | |
| ATOM | 1187 OE2 GLU 365 | 10.034 4.607 51.010 1.00 27.63 | |
| ATOM | 1188 C GLU 365 | 11.454 9.881 53.798 1.00 22.43 | |
| ATOM | 1189 O GLU 365 | 10.557 10.570 54.298 1.00 22.18 | |
| ATOM | 1190 N TYR 366 | 12.681 9.822 54.304 1.00 22.10 | |
| ATOM | 1191 CA TYR 366 | 13.026 10.580 55.506 1.00 22.15 | |
| ATOM | 1192 CB TYR 366 | 14.494 10.316 55.871 1.00 22.04 | |
| ATOM | 1193 CG TYR 366 | 15.010 11.209 56.986 1.00 22.63 | |
| ATOM | 1194 CD1 TYR 366 | 14.580 11.030 58.307 1.00 21.87 | |
| ATOM | 1195 CE1 TYR 366 | 15.023 11.864 59.328 1.00 22.24 | |
| ATOM | 1196 CD2 TYR 366 | 15.903 12.246 56.715 1.00 20.88 | |
| ATOM | 1197 CE2 TYR 366 | 16.366 13.092 57.731 1.00 22.98 | |
| ATOM | 1198 CZ TYR 366 | 15.913 12.899 59.041 1.00 22.25 | |
| ATOM | 1199 OH TYR 366 | 16.315 13.752 60.054 1.00 23.04 | |
| ATOM | 1200 C TYR 366 | 12.797 12.095 55.332 1.00 19.98 | |
| ATOM | 1201 O TYR 366 | 12.209 12.731 56.199 1.00 19.55 | |
| ATOM | 1202 N VAL 367 | 13.260 12.676 54.225 1.00 20.09 | |
| ATOM | 1203 CA VAL 367 | 13.080 14.106 54.034 1.00 19.12 | |
| ATOM | 1204 CB VAL 367 | 13.955 14.666 52.891 1.00 20.38 | |
| ATOM | 1205 CG1 VAL 367 | 15.446 14.473 53.249 1.00 21.37 | |
| ATOM | 1206 CG2 VAL 367 | 13.609 14.009 51.554 1.00 19.90 | |
| ATOM | 1207 C VAL 367 | 11.607 14.458 53.784 1.00 18.65 | |
| ATOM | 1208 O VAL 367 | 11.165 15.538 54.178 1.00 18.48 | |
| ATOM | 1209 N ALA 368 | 10.868 13.552 53.147 1.00 20.47 | |
| ATOM | 1210 CA ALA 368 | 9.447 13.787 52.893 1.00 19.05 | |
| ATOM | 1211 CB ALA 368 | 8.854 12.668 51.988 1.00 18.93 | |
| ATOM | 1212 C ALA 368 | 8.714 13.813 54.240 1.00 19.17 | |
| ATOM | 1213 O ALA 368 | 7.863 14.682 54.487 1.00 18.81 | |
| ATOM | 1214 N LEU 369 | 9.048 12.871 55.112 1.00 19.43 | |
| ATOM | 1215 CA LEU 369 | 8.417 12.819 56.428 1.00 17.85 | |
| ATOM | 1216 CB LEU 369 | 8.807 11.526 57.147 1.00 20.14 | |
| ATOM | 1217 CG LEU 369 | 8.153 10.250 56.565 1.00 21.25 | |
| ATOM | 1218 CD1 LEU 369 | 8.810 9.041 57.154 1.00 22.02 | |
| ATOM | 1219 CD2 LEU 369 | 6.626 10.219 56.856 1.00 21.64 | |
| ATOM | 1220 C LEU 369 | 8.772 14.076 57.235 1.00 19.07 | |
| ATOM | 1221 O LEU 369 | 7.945 14.606 57.965 1.00 17.17 | |
| ATOM | 1222 N LYS 370 | 10.007 14.567 57.120 1.00 18.01 | |
| ATOM | 1223 CA LYS 370 | 10.363 15.799 57.826 1.00 19.26 | |
| ATOM | 1224 CB LYS 370 | 11.819 16.199 57.522 1.00 18.97 | |
| ATOM | 1225 CG LYS 370 | 12.888 15.349 58.203 1.00 21.31 | |
| ATOM | 1226 CD LYS 370 | 14.304 15.860 57.845 1.00 25.49 | |
| ATOM | 1227 CE LYS 370 | 14.445 17.367 58.009 1.00 25.79 | |
| ATOM | 1228 NZ LYS 370 | 15.878 17.841 57.879 1.00 29.87 | |
| ATOM | 1229 C LYS 370 | 9.440 16.949 57.388 1.00 17.68 | |
| ATOM | 1230 O LYS 370 | 8.950 17.717 58.203 1.00 17.05 | |
| ATOM | 1231 N ALA 371 | 9.209 17.075 56.081 1.00 17.98 | |
| ATOM | 1232 CA ALA 371 | 8.368 18.154 55.596 1.00 17.26 | |
| ATOM | 1233 CB ALA 371 | 8.344 18.154 54.065 1.00 19.00 | |
| ATOM | 1234 C ALA 371 | 6.957 17.957 56.140 1.00 15.65 | |
| ATOM | 1235 O ALA 371 | 6.299 18.909 56.533 1.00 17.13 | |
| ATOM | 1236 N ILE 372 | 6.492 16.713 56.151 1.00 14.92 | |
| ATOM | 1237 CA ILE 372 | 5.146 16.450 56.673 1.00 16.38 | |
| ATOM | 1238 CB ILE 372 | 4.743 14.956 56.430 1.00 15.28 | |
| ATOM | 1239 CG2 ILE 372 | 3.477 14.602 57.245 1.00 17.98 | |
| ATOM | 1240 CG1 ILE 372 | 4.566 14.730 54.915 1.00 18.14 | |
| ATOM | 1241 CD1 ILE 372 | 4.413 13.271 54.530 1.00 18.13 | |
| ATOM | 1242 C ILE 372 | 5.038 16.795 58.163 1.00 16.98 | |
| ATOM | 1243 O ILE 372 | 4.028 17.317 58.606 1.00 16.87 | |
| ATOM | 1244 N ILE 373 | 6.089 16.522 58.942 1.00 14.47 | |
| ATOM | 1245 CA ILE 373 | 6.080 16.829 60.369 1.00 15.13 | |
| ATOM | 1246 CB ILE 373 | 7.392 16.251 61.042 1.00 13.81 | |
| ATOM | 1247 CG2 ILE 373 | 7.530 16.783 62.463 1.00 15.08 | |
| ATOM | 1248 CG1 ILE 373 | 7.340 14.717 61.052 1.00 14.66 | |
| ATOM | 1249 CD1 ILE 373 | 8.709 14.031 61.219 1.00 17.60 | |
| ATOM | 1250 C ILE 373 | 5.962 18.350 60.604 1.00 15.01 | |
| ATOM | 1251 O ILE 373 | 5.212 18.817 61.477 1.00 15.67 | |
| ATOM | 1252 N LEU 374 | 6.702 19.109 59.796 1.00 16.23 | |
| ATOM | 1253 CA LEU 374 | 6.688 20.557 59.880 1.00 15.91 | |
| ATOM | 1254 CB LEU 374 | 7.738 21.160 58.942 1.00 18.27 | |
| ATOM | 1255 CG LEU 374 | 7.702 22.700 58.759 1.00 19.49 | |
| ATOM | 1256 CD1 LEU 374 | 8.184 23.403 60.044 1.00 18.70 | |
| ATOM | 1257 CD2 LEU 374 | 8.596 23.092 57.597 1.00 19.61 | |
| ATOM | 1258 C LEU 374 | 5.316 21.128 59.490 1.00 17.14 | |
| ATOM | 1259 O LEU 374 | 4.765 21.980 60.167 1.00 16.72 | |
| ATOM | 1260 N LEU 375 | 4.803 20.662 58.367 1.00 17.78 | |
| ATOM | 1261 CA LEU 375 | 3.561 21.202 57.831 1.00 17.01 | |
| ATOM | 1262 CB LEU 375 | 3.573 21.071 56.301 1.00 19.06 | |
| ATOM | 1263 CG LEU 375 | 4.788 21.715 55.581 1.00 18.84 | |
| ATOM | 1264 CD1 LEU 375 | 4.903 21.197 54.151 1.00 18.22 | |
| ATOM | 1265 CD2 LEU 375 | 4.644 23.234 55.610 1.00 19.41 | |
| ATOM | 1266 C LEU 375 | 2.349 20.514 58.422 1.00 19.26 | |
| ATOM | 1267 O LEU 375 | 1.603 19.823 57.705 1.00 19.50 | |
| ATOM | 1268 N ASN 376 | 2.178 20.727 59.723 1.00 16.95 | |
| ATOM | 1269 CA ASN 376 | 1.087 20.136 60.510 1.00 19.78 | |
| ATOM | 1270 CB ASN 376 | 1.605 19.772 61.912 1.00 18.97 | |
| ATOM | 1271 CG ASN 376 | 0.537 19.124 62.780 1.00 23.13 | |
| ATOM | 1272 OD1 ASN 376 | −0.527 18.732 62.289 1.00 19.72 | |
| ATOM | 1273 ND2 ASN 376 | 0.827 18.989 64.081 1.00 21.10 | |
| ATOM | 1274 C ASN 376 | −0.088 21.080 60.641 1.00 17.80 | |
| ATOM | 1275 O ASN 376 | −0.043 22.028 61.412 1.00 18.77 | |
| ATOM | 1276 N PRO 377 | −1.188 20.820 59.909 1.00 20.03 | |
| ATOM | 1277 CD PRO 377 | −1.463 19.684 59.010 1.00 19.34 | |
| ATOM | 1278 CA PRO 377 | −2.342 21.728 60.011 1.00 20.40 | |
| ATOM | 1279 CB PRO 377 | −3.239 21.293 58.858 1.00 18.78 | |
| ATOM | 1280 CG PRO 377 | −2.979 19.796 58.774 1.00 19.22 | |
| ATOM | 1281 C PRO 377 | −3.086 21.705 61.336 1.00 21.89 | |
| ATOM | 1282 O PRO 377 | −3.922 22.583 61.594 1.00 22.57 | |
| ATOM | 1283 N ASP 378 | −2.771 20.723 62.177 1.00 20.09 | |
| ATOM | 1284 CA ASP 378 | −3.444 20.609 63.463 1.00 22.47 | |
| ATOM | 1285 CB ASP 378 | −3.608 19.131 63.809 1.00 23.71 | |
| ATOM | 1286 CG ASP 378 | −4.555 18.426 62.847 1.00 25.43 | |
| ATOM | 1287 OD1 ASP 378 | −5.579 19.028 62.487 1.00 28.35 | |
| ATOM | 1288 OD2 ASP 378 | −4.289 17.283 62.443 1.00 30.58 | |
| ATOM | 1289 C ASP 378 | −2.823 21.403 64.603 1.00 22.30 | |
| ATOM | 1290 O ASP 378 | −3.265 21.309 65.760 1.00 22.98 | |
| ATOM | 1291 N VAL 379 | −1.793 22.181 64.279 1.00 22.51 | |
| ATOM | 1292 CA VAL 379 | −1.167 23.053 65.266 1.00 21.52 | |
| ATOM | 1293 CB VAL 379 | 0.044 23.807 64.662 1.00 20.60 | |
| ATOM | 1294 CG1 VAL 379 | 0.424 25.004 65.532 1.00 22.27 | |
| ATOM | 1295 CG2 VAL 379 | 1.226 22.848 64.516 1.00 22.31 | |
| ATOM | 1296 C VAL 379 | −2.255 24.048 65.658 1.00 23.83 | |
| ATOM | 1297 O VAL 379 | −2.918 24.655 64.793 1.00 25.06 | |
| ATOM | 1298 N LYS 380 | −2.460 24.212 66.958 1.00 23.33 | |
| ATOM | 1299 CA LYS 380 | −3.490 25.124 67.430 1.00 24.49 | |
| ATOM | 1300 CB LYS 380 | −3.797 24.829 68.904 1.00 25.21 | |
| ATOM | 1301 CG LYS 380 | −4.182 23.365 69.131 1.00 28.03 | |
| ATOM | 1302 CD LYS 380 | −4.460 23.035 70.594 1.00 30.82 | |
| ATOM | 1303 CE LYS 380 | −4.728 21.536 70.758 1.00 31.68 | |
| ATOM | 1304 NZ LYS 380 | −4.904 21.079 72.177 1.00 33.50 | |
| ATOM | 1305 C LYS 380 | −3.055 26.573 67.250 1.00 24.85 | |
| ATOM | 1306 O LYS 380 | −1.874 26.891 67.408 1.00 25.55 | |
| ATOM | 1307 N GLY 381 | −3.988 27.445 66.861 1.00 24.69 | |
| ATOM | 1308 CA GLY 381 | −3.647 28.852 66.704 1.00 26.29 | |
| ATOM | 1309 C GLY 381 | −3.384 29.378 65.295 1.00 25.30 | |
| ATOM | 1310 O GLY 381 | −3.246 30.598 65.093 1.00 27.29 | |
| ATOM | 1311 N LEU 382 | −3.313 28.490 64.311 1.00 26.35 | |
| ATOM | 1312 CA LEU 382 | −3.054 28.914 62.941 1.00 26.95 | |
| ATOM | 1313 CB LEU 382 | −2.997 27.706 61.995 1.00 25.01 | |
| ATOM | 1314 CG LEU 382 | −1.865 26.700 62.265 1.00 24.87 | |
| ATOM | 1315 CD1 LEU 382 | −2.011 25.507 61.314 1.00 24.53 | |
| ATOM | 1316 CD2 LEU 382 | −0.518 27.384 62.064 1.00 24.40 | |
| ATOM | 1317 C LEU 382 | −4.120 29.885 62.428 1.00 28.77 | |
| ATOM | 1318 O LEU 382 | −5.310 29.719 62.695 1.00 28.39 | |
| ATOM | 1319 N LYS 383 | −3.660 30.881 61.681 1.00 30.83 | |
| ATOM | 1320 CA LYS 383 | −4.518 31.894 61.078 1.00 32.60 | |
| ATOM | 1321 CB LYS 383 | −3.670 33.104 60.699 1.00 35.65 | |
| ATOM | 1322 CG LYS 383 | −4.352 34.121 59.788 1.00 40.31 | |
| ATOM | 1323 CD LYS 383 | −5.044 35.203 60.589 1.00 42.92 | |
| ATOM | 1324 CE LYS 383 | −5.436 36.376 59.702 1.00 43.80 | |
| ATOM | 1325 NZ LYS 383 | −6.040 37.476 60.505 1.00 44.52 | |
| ATOM | 1326 C LYS 383 | −5.159 31.313 59.819 1.00 32.46 | |
| ATOM | 1327 O LYS 383 | −6.374 31.411 59.620 1.00 33.42 | |
| ATOM | 1328 N ASN 384 | −4.332 30.700 58.977 1.00 30.19 | |
| ATOM | 1329 CA ASN 384 | −4.794 30.118 57.720 1.00 29.86 | |
| ATOM | 1330 CB ASN 384 | −4.154 30.854 56.549 1.00 32.74 | |
| ATOM | 1331 CG ASN 384 | −4.460 32.336 56.570 1.00 36.46 | |
| ATOM | 1332 OD1 ASN 384 | −5.625 32.730 56.585 1.00 39.59 | |
| ATOM | 1333 ND2 ASN 384 | −3.418 33.166 56.583 1.00 37.88 | |
| ATOM | 1334 C ASN 384 | −4.466 28.636 57.636 1.00 26.29 | |
| ATOM | 1335 O ASN 384 | −3.558 28.229 56.912 1.00 26.38 | |
| ATOM | 1336 N ARG 385 | −5.218 27.845 58.387 1.00 24.62 | |
| ATOM | 1337 CA ARG 385 | −5.005 26.406 58.431 1.00 24.85 | |
| ATOM | 1338 CB ARG 385 | −5.994 25.779 59.413 1.00 24.62 | |
| ATOM | 1339 CG ARG 385 | −5.910 24.275 59.561 1.00 27.77 | |
| ATOM | 1340 CD ARG 385 | −6.705 23.839 60.785 1.00 30.37 | |
| ATOM | 1341 NE ARG 385 | −5.976 24.188 62.004 1.00 31.78 | |
| ATOM | 1342 CZ ARG 385 | −6.329 25.148 62.849 1.00 31.80 | |
| ATOM | 1343 NH1 ARG 385 | −7.432 25.867 62.629 1.00 31.71 | |
| ATOM | 1344 NH2 ARG 385 | −5.542 25.426 63.886 1.00 28.60 | |
| ATOM | 1345 C ARG 385 | −5.079 25.727 57.070 1.00 24.38 | |
| ATOM | 1346 O ARG 385 | −4.289 24.824 56.783 1.00 24.23 | |
| ATOM | 1347 N GLN 386 | −6.003 26.165 56.214 1.00 24.39 | |
| ATOM | 1348 CA GLN 386 | −6.128 25.542 54.906 1.00 25.99 | |
| ATOM | 1349 CB GLN 386 | −7.320 26.124 54.128 1.00 29.68 | |
| ATOM | 1350 CG GLN 386 | −7.256 27.619 53.863 1.00 34.93 | |
| ATOM | 1351 CD GLN 386 | −7.246 28.442 55.136 1.00 37.35 | |
| ATOM | 1352 OE1 GLN 386 | −8.016 28.183 56.072 1.00 39.44 | |
| ATOM | 1353 NE2 GLN 386 | −6.373 29.443 55.183 1.00 39.95 | |
| ATOM | 1354 C GLN 386 | −4.867 25.646 54.067 1.00 24.30 | |
| ATOM | 1355 O GLN 386 | −4.561 24.727 53.318 1.00 25.10 | |
| ATOM | 1356 N GLU 387 | −4.140 26.752 54.182 1.00 25.50 | |
| ATOM | 1357 CA GLU 387 | −2.907 26.905 53.413 1.00 25.38 | |
| ATOM | 1358 CB GLU 387 | −2.358 28.328 53.594 1.00 29.21 | |
| ATOM | 1359 CG GLU 387 | −3.234 29.420 52.915 1.00 33.12 | |
| ATOM | 1360 CD GLU 387 | −2.845 30.838 53.284 1.00 37.28 | |
| ATOM | 1361 OE1 GLU 387 | −1.634 31.125 53.407 1.00 40.75 | |
| ATOM | 1362 OE2 GLU 387 | −3.750 31.691 53.436 1.00 38.95 | |
| ATOM | 1363 C GLU 387 | −1.877 25.841 53.852 1.00 25.54 | |
| ATOM | 1364 O GLU 387 | −1.097 25.329 53.030 1.00 24.67 | |
| ATOM | 1365 N VAL 388 | −1.897 25.476 55.132 1.00 24.28 | |
| ATOM | 1366 CA VAL 388 | −0.960 24.447 55.617 1.00 21.40 | |
| ATOM | 1367 CB VAL 388 | −0.845 24.439 57.168 1.00 19.68 | |
| ATOM | 1368 CG1 VAL 388 | 0.182 23.410 57.597 1.00 22.22 | |
| ATOM | 1369 CG2 VAL 388 | −0.477 25.824 57.691 1.00 20.28 | |
| ATOM | 1370 C VAL 388 | −1.444 23.076 55.178 1.00 22.10 | |
| ATOM | 1371 O VAL 388 | −0.666 22.211 54.782 1.00 21.10 | |
| ATOM | 1372 N GLU 389 | −2.750 22.859 55.243 1.00 22.00 | |
| ATOM | 1373 CA GLU 389 | −3.284 21.577 54.825 1.00 21.41 | |
| ATOM | 1374 CB GLU 389 | −4.801 21.538 54.987 1.00 22.76 | |
| ATOM | 1375 CG GLU 389 | −5.358 20.146 54.988 1.00 31.47 | |
| ATOM | 1376 CD GLU 389 | −6.793 20.093 55.471 1.00 36.14 | |
| ATOM | 1377 OE1 GLU 389 | −7.160 20.927 56.334 1.00 39.74 | |
| ATOM | 1378 OE2 GLU 389 | −7.540 19.204 55.006 1.00 39.63 | |
| ATOM | 1379 C GLU 389 | −2.936 21.294 53.382 1.00 19.32 | |
| ATOM | 1380 O GLU 389 | −2.653 20.155 53.025 1.00 21.32 | |
| ATOM | 1381 N VAL 390 | −3.001 22.313 52.534 1.00 19.72 | |
| ATOM | 1382 CA VAL 390 | −2.685 22.148 51.125 1.00 19.82 | |
| ATOM | 1383 CB VAL 390 | −2.950 23.447 50.316 1.00 19.77 | |
| ATOM | 1384 CG1 VAL 390 | −2.307 23.344 48.919 1.00 22.22 | |
| ATOM | 1385 CG2 VAL 390 | −4.442 23.652 50.169 1.00 23.95 | |
| ATOM | 1386 C VAL 390 | −1.231 21.744 50.924 1.00 20.27 | |
| ATOM | 1387 O VAL 390 | −0.924 20.894 50.093 1.00 20.79 | |
| ATOM | 1388 N LEU 391 | −0.334 22.345 51.699 1.00 21.06 | |
| ATOM | 1389 CA LEU 391 | 1.078 22.004 51.552 1.00 19.84 | |
| ATOM | 1390 CB LEU 391 | 1.951 22.982 52.328 1.00 20.90 | |
| ATOM | 1391 CG LEU 391 | 1.895 24.438 51.836 1.00 21.65 | |
| ATOM | 1392 CD1 LEU 391 | 2.846 25.283 52.673 1.00 23.70 | |
| ATOM | 1393 CD2 LEU 391 | 2.260 24.515 50.351 1.00 25.56 | |
| ATOM | 1394 C LEU 391 | 1.327 20.583 52.031 1.00 19.59 | |
| ATOM | 1395 O LEU 391 | 2.090 19.837 51.421 1.00 19.63 | |
| ATOM | 1396 N ARG 392 | 0.680 20.192 53.130 0.50 19.30 | AC1 |
| ATOM | 1397 CA ARG 392 | 0.862 18.823 53.628 0.50 19.65 | AC1 |
| ATOM | 1398 CB ARG 392 | 0.212 18.620 55.016 0.50 19.22 | AC1 |
| ATOM | 1399 CG ARG 392 | 0.060 17.123 55.382 0.50 18.74 | AC1 |
| ATOM | 1400 CD ARG 392 | −0.380 16.857 56.840 0.50 19.04 | AC1 |
| ATOM | 1401 NE ARG 392 | 0.710 17.001 57.811 0.50 18.30 | AC1 |
| ATOM | 1402 CZ ARG 392 | 0.739 16.427 59.020 0.50 18.32 | AC1 |
| ATOM | 1403 NH1 ARG 392 | −0.262 15.660 59.432 0.50 18.09 | AC1 |
| ATOM | 1404 NH2 ARG 392 | 1.783 16.606 59.826 0.50 13.08 | AC1 |
| ATOM | 1405 C ARG 392 | 0.259 17.833 52.624 0.50 19.79 | AC1 |
| ATOM | 1406 O ARG 392 | 0.768 16.730 52.453 0.50 19.90 | AC1 |
| ATOM | 1407 N GLU 393 | −0.839 18.220 51.976 1.00 19.51 | |
| ATOM | 1408 CA GLU 393 | −1.484 17.355 50.979 1.00 21.32 | |
| ATOM | 1409 CB GLU 393 | −2.858 17.922 50.575 1.00 23.83 | |
| ATOM | 1410 CG GLU 393 | −3.631 17.066 49.588 1.00 29.44 | |
| ATOM | 1411 CD GLU 393 | −4.209 15.767 50.173 1.00 34.23 | |
| ATOM | 1412 OE1 GLU 393 | −3.940 15.434 51.348 1.00 34.71 | |
| ATOM | 1413 OE2 GLU 393 | −4.952 15.071 49.433 1.00 37.12 | |
| ATOM | 1414 C GLU 393 | −0.556 17.210 49.763 1.00 21.12 | |
| ATOM | 1415 O GLU 393 | −0.486 16.147 49.157 1.00 20.35 | |
| ATOM | 1416 N LYS 394 | 0.143 18.287 49.395 1.00 19.98 | |
| ATOM | 1417 CA LYS 394 | 1.102 18.186 48.302 1.00 21.24 | |
| ATOM | 1418 CB LYS 394 | 1.787 19.519 48.048 1.00 22.05 | |
| ATOM | 1419 CG LYS 394 | 0.903 20.468 47.258 1.00 24.02 | |
| ATOM | 1420 CD LYS 394 | 1.608 21.778 46.964 1.00 25.62 | |
| ATOM | 1421 CE LYS 394 | 0.690 22.711 46.199 1.00 28.47 | |
| ATOM | 1422 NZ LYS 394 | 1.369 24.012 45.959 1.00 30.41 | |
| ATOM | 1423 C LYS 394 | 2.153 17.137 48.667 1.00 22.08 | |
| ATOM | 1424 O LYS 394 | 2.555 16.341 47.825 1.00 20.82 | |
| ATOM | 1425 N MET 395 | 2.590 17.136 49.932 1.00 21.43 | |
| ATOM | 1426 CA MET 395 | 3.607 16.166 50.369 1.00 21.13 | |
| ATOM | 1427 CB MET 395 | 4.177 16.520 51.746 1.00 24.05 | |
| ATOM | 1428 CG MET 395 | 5.100 17.680 51.738 1.00 24.66 | |
| ATOM | 1429 SD MET 395 | 6.457 17.503 50.571 1.00 27.36 | |
| ATOM | 1430 CE MET 395 | 6.985 15.797 50.738 1.00 23.91 | |
| ATOM | 1431 C MET 395 | 3.121 14.744 50.417 1.00 20.46 | |
| ATOM | 1432 O MET 395 | 3.872 13.832 50.095 1.00 22.42 | |
| ATOM | 1433 N PHE 396 | 1.862 14.542 50.827 1.00 20.87 | |
| ATOM | 1434 CA PHE 396 | 1.285 13.202 50.858 1.00 21.65 | |
| ATOM | 1435 CB PHE 396 | −0.176 13.203 51.374 1.00 20.90 | |
| ATOM | 1436 CG PHE 396 | −0.320 13.286 52.875 1.00 22.60 | |
| ATOM | 1437 CD1 PHE 396 | 0.733 12.996 53.727 1.00 22.79 | |
| ATOM | 1438 CD2 PHE 396 | −1.544 13.640 53.436 1.00 23.58 | |
| ATOM | 1439 CE1 PHE 396 | 0.570 13.063 55.116 1.00 20.29 | |
| ATOM | 1440 CE2 PHE 396 | −1.712 13.702 54.826 1.00 23.72 | |
| ATOM | 1441 CZ PHE 396 | −0.655 13.416 55.669 1.00 22.24 | |
| ATOM | 1442 C PHE 396 | 1.281 12.652 49.429 1.00 22.43 | |
| ATOM | 1443 O PHE 396 | 1.635 11.494 49.215 1.00 21.80 | |
| ATOM | 1444 N LEU 397 | 0.844 13.462 48.458 1.00 22.96 | |
| ATOM | 1445 CA LEU 397 | 0.813 13.022 47.061 1.00 21.97 | |
| ATOM | 1446 CB LEU 397 | 0.139 14.099 46.176 1.00 22.88 | |
| ATOM | 1447 CG LEU 397 | −1.385 14.161 46.354 1.00 23.93 | |
| ATOM | 1448 CD1 LEU 397 | −1.981 15.078 45.315 1.00 25.27 | |
| ATOM | 1449 CD2 LEU 397 | −1.972 12.759 46.190 1.00 25.99 | |
| ATOM | 1450 C LEU 397 | 2.233 12.705 46.560 1.00 24.94 | |
| ATOM | 1451 O LEU 397 | 2.450 11.692 45.873 1.00 25.61 | |
| ATOM | 1452 N CYS 398 | 3.195 13.548 46.924 1.00 23.80 | |
| ATOM | 1453 CA CYS 398 | 4.593 13.333 46.519 1.00 26.51 | |
| ATOM | 1454 CB CYS 398 | 5.509 14.454 47.041 1.00 28.87 | |
| ATOM | 1455 SG CYS 398 | 5.551 15.910 46.032 1.00 39.48 | |
| ATOM | 1456 C CYS 398 | 5.111 12.027 47.090 1.00 25.35 | |
| ATOM | 1457 O CYS 398 | 5.738 11.221 46.399 1.00 25.03 | |
| ATOM | 1458 N LEU 399 | 4.861 11.821 48.371 1.00 24.81 | |
| ATOM | 1459 CA LEU 399 | 5.366 10.620 49.009 1.00 24.04 | |
| ATOM | 1460 CB LEU 399 | 5.232 10.739 50.530 1.00 24.26 | |
| ATOM | 1461 CG LEU 399 | 5.806 9.580 51.344 1.00 24.01 | |
| ATOM | 1462 CD1 LEU 399 | 7.281 9.343 50.985 1.00 22.08 | |
| ATOM | 1463 CD2 LEU 399 | 5.672 9.931 52.821 1.00 22.85 | |
| ATOM | 1464 C LEU 399 | 4.715 9.352 48.518 1.00 26.35 | |
| ATOM | 1465 O LEU 399 | 5.398 8.363 48.267 1.00 25.02 | |
| ATOM | 1466 N ASP 400 | 3.391 9.377 48.361 1.00 27.30 | |
| ATOM | 1467 CA ASP 400 | 2.680 8.200 47.874 1.00 29.09 | |
| ATOM | 1468 CB ASP 400 | 1.179 8.490 47.772 1.00 31.25 | |
| ATOM | 1469 CG ASP 400 | 0.379 7.258 47.400 1.00 34.75 | |
| ATOM | 1470 OD1 ASP 400 | 0.236 6.356 48.252 1.00 35.16 | |
| ATOM | 1471 OD2 ASP 400 | −0.091 7.188 46.250 1.00 38.49 | |
| ATOM | 1472 C ASP 400 | 3.217 7.816 46.497 1.00 28.91 | |
| ATOM | 1473 O ASP 400 | 3.436 6.642 46.212 1.00 29.66 | |
| ATOM | 1474 N GLU 401 | 3.420 8.813 45.644 1.00 28.16 | |
| ATOM | 1475 CA GLU 401 | 3.936 8.577 44.299 1.00 29.87 | |
| ATOM | 1476 CB GLU 401 | 4.052 9.895 43.533 1.00 32.50 | |
| ATOM | 1477 CG GLU 401 | 4.735 9.751 42.180 1.00 38.83 | |
| ATOM | 1478 CD GLU 401 | 4.784 11.048 41.406 1.00 42.93 | |
| ATOM | 1479 OE1 GLU 401 | 5.449 12.003 41.865 1.00 45.46 | |
| ATOM | 1480 OE2 GLU 401 | 4.147 11.114 40.334 1.00 45.87 | |
| ATOM | 1481 C GLU 401 | 5.308 7.911 44.360 1.00 31.19 | |
| ATOM | 1482 O GLU 401 | 5.574 6.926 43.650 1.00 31.37 | |
| ATOM | 1483 N TYR 402 | 6.171 8.444 45.224 1.00 28.55 | |
| ATOM | 1484 CA TYR 402 | 7.516 7.918 45.365 1.00 29.01 | |
| ATOM | 1485 CB TYR 402 | 8.314 8.737 46.384 1.00 27.40 | |
| ATOM | 1486 CG TYR 402 | 9.667 8.134 46.659 1.00 27.45 | |
| ATOM | 1487 CD1 TYR 402 | 9.849 7.238 47.709 1.00 27.18 | |
| ATOM | 1488 CE1 TYR 402 | 11.087 6.635 47.933 1.00 28.15 | |
| ATOM | 1489 CD2 TYR 402 | 10.747 8.419 45.834 1.00 27.22 | |
| ATOM | 1490 CE2 TYR 402 | 11.996 7.820 46.050 1.00 30.03 | |
| ATOM | 1491 CZ TYR 402 | 12.147 6.936 47.097 1.00 30.78 | |
| ATOM | 1492 OH TYR 402 | 13.374 6.358 47.316 1.00 31.66 | |
| ATOM | 1493 C TYR 402 | 7.503 6.453 45.769 1.00 30.16 | |
| ATOM | 1494 O TYR 402 | 8.206 5.641 45.187 1.00 29.67 | |
| ATOM | 1495 N CYS 403 | 6.700 6.113 46.763 1.00 30.42 | |
| ATOM | 1496 CA CYS 403 | 6.616 4.739 47.215 1.00 34.32 | |
| ATOM | 1497 CB CYS 403 | 5.691 4.650 48.430 1.00 33.37 | |
| ATOM | 1498 SG CYS 403 | 6.357 5.485 49.920 1.00 31.63 | |
| ATOM | 1499 C CYS 403 | 6.116 3.820 46.104 1.00 36.81 | |
| ATOM | 1500 O CYS 403 | 6.612 2.703 45.936 1.00 36.95 | |
| ATOM | 1501 N ARG 404 | 5.140 4.289 45.335 1.00 39.50 | |
| ATOM | 1502 CA ARG 404 | 4.597 3.453 44.271 1.00 42.67 | |
| ATOM | 1503 CB ARG 404 | 3.280 4.045 43.746 1.00 43.44 | |
| ATOM | 1504 CG ARG 404 | 2.135 3.802 44.718 1.00 46.08 | |
| ATOM | 1505 CD ARG 404 | 0.746 4.164 44.188 1.00 46.89 | |
| ATOM | 1506 NE ARG 404 | 0.490 5.602 44.159 1.00 46.69 | |
| ATOM | 1507 CZ ARG 404 | 0.733 6.386 43.116 1.00 47.48 | |
| ATOM | 1508 NH1 ARG 404 | 1.241 5.874 42.002 1.00 48.94 | |
| ATOM | 1509 NH2 ARG 404 | 0.462 7.684 43.181 1.00 48.22 | |
| ATOM | 1510 C ARG 404 | 5.578 3.206 43.137 1.00 43.67 | |
| ATOM | 1511 O ARG 404 | 5.640 2.102 42.598 1.00 44.97 | |
| ATOM | 1512 N ARG 405 | 6.356 4.220 42.785 1.00 45.45 | |
| ATOM | 1513 CA ARG 405 | 7.330 4.098 41.710 1.00 47.72 | |
| ATOM | 1514 CB ARG 405 | 7.700 5.481 41.167 1.00 49.80 | |
| ATOM | 1515 CG ARG 405 | 6.535 6.298 40.624 1.00 53.09 | |
| ATOM | 1516 CD ARG 405 | 7.005 7.695 40.233 1.00 56.62 | |
| ATOM | 1517 NE ARG 405 | 5.931 8.506 39.661 1.00 59.35 | |
| ATOM | 1518 CZ ARG 405 | 5.356 8.263 38.488 1.00 60.20 | |
| ATOM | 1519 NH1 ARG 405 | 5.753 7.231 37.756 1.00 61.80 | |
| ATOM | 1520 NH2 ARG 405 | 4.382 9.049 38.046 1.00 60.75 | |
| ATOM | 1521 C ARG 405 | 8.609 3.410 42.166 1.00 48.23 | |
| ATOM | 1522 O ARG 405 | 9.078 2.464 41.528 1.00 48.90 | |
| ATOM | 1523 N SER 406 | 9.163 3.891 43.278 1.00 47.66 | |
| ATOM | 1524 CA SER 406 | 10.421 3.382 43.819 1.00 48.66 | |
| ATOM | 1525 CB SER 406 | 11.099 4.477 44.638 1.00 49.49 | |
| ATOM | 1526 OG SER 406 | 11.118 5.698 43.919 1.00 51.64 | |
| ATOM | 1527 C SER 406 | 10.336 2.112 44.656 1.00 48.77 | |
| ATOM | 1528 O SER 406 | 11.355 1.507 44.968 1.00 48.91 | |
| ATOM | 1529 N ARC 407 | 9.129 1.711 45.026 1.00 49.27 | |
| ATOM | 1530 CA ARG 407 | 8.948 0.500 45.813 1.00 50.47 | |
| ATOM | 1531 CB ARG 407 | 9.106 0.811 47.302 1.00 52.25 | |
| ATOM | 1532 CG ARG 407 | 10.476 0.434 47.849 1.00 54.88 | |
| ATOM | 1533 CD ARG 407 | 10.942 1.401 48.918 1.00 55.39 | |
| ATOM | 1534 NE ARG 407 | 12.157 0.927 49.573 1.00 56.11 | |
| ATOM | 1535 CZ ARG 407 | 12.885 1.653 50.416 1.00 56.40 | |
| ATOM | 1536 NH1 ARG 407 | 12.524 2.895 50.707 1.00 56.21 | |
| ATOM | 1537 NH2 ARG 407 | 13.973 1.133 50.972 1.00 55.90 | |
| ATOM | 1538 C ARG 407 | 7.586 −0.110 45.535 1.00 50.00 | |
| ATOM | 1539 O ARG 407 | 6.847 −0.468 46.451 1.00 50.06 | |
| ATOM | 1540 N SER 408 | 7.279 −0.233 44.248 1.00 49.76 | |
| ATOM | 1541 CA SER 408 | 6.010 −0.777 43.782 1.00 49.98 | |
| ATOM | 1542 CB SER 408 | 6.060 −0.957 42.262 1.00 50.23 | |
| ATOM | 1543 OG SER 408 | 7.129 −1.806 41.880 1.00 50.48 | |
| ATOM | 1544 C SER 408 | 5.622 −2.100 44.439 1.00 49.86 | |
| ATOM | 1545 O SER 408 | 4.444 −2.354 44.698 1.00 50.10 | |
| ATOM | 1546 N SER 409 | 6.620 −2.933 44.706 1.00 50.01 | |
| ATOM | 1547 CA SER 409 | 6.412 −4.245 45.307 1.00 49.71 | |
| ATOM | 1548 CB SER 409 | 7.704 −5.055 45.217 1.00 50.85 | |
| ATOM | 1549 OG SER 409 | 8.799 −4.306 45.730 1.00 52.55 | |
| ATOM | 1550 C SER 409 | 5.954 −4.195 46.758 1.00 49.15 | |
| ATOM | 1551 O SER 409 | 5.476 −5.195 47.300 1.00 48.48 | |
| ATOM | 1552 N GLU 410 | 6.095 −3.035 47.392 1.00 47.66 | |
| ATOM | 1553 CA GLU 410 | 5.701 −2.912 48.785 1.00 45.50 | |
| ATOM | 1554 CB GLU 410 | 6.787 −2.197 49.577 1.00 46.29 | |
| ATOM | 1555 CG GLU 410 | 8.079 −2.980 49.712 1.00 47.76 | |
| ATOM | 1556 CD GLU 410 | 9.074 −2.261 50.592 1.00 48.13 | |
| ATOM | 1557 OE1 GLU 410 | 10.155 −1.882 50.092 1.00 49.66 | |
| ATOM | 1558 OE2 GLU 410 | 8.761 −2.063 51.784 1.00 48.57 | |
| ATOM | 1559 C GLU 410 | 4.373 −2.197 48.973 1.00 44.10 | |
| ATOM | 1560 O GLU 410 | 4.311 −0.974 49.114 1.00 41.70 | |
| ATOM | 1561 N GLU 411 | 3.306 −2.985 48.986 1.00 42.76 | |
| ATOM | 1562 CA GLU 411 | 1.973 −2.451 49.154 1.00 41.57 | |
| ATOM | 1563 CB GLU 411 | 0.945 −3.529 48.814 1.00 44.42 | |
| ATOM | 1564 CG GLU 411 | 1.166 −4.132 47.439 1.00 48.49 | |
| ATOM | 1565 CD GLU 411 | 0.151 −5.191 47.100 1.00 49.60 | |
| ATOM | 1566 OE1 GLU 411 | 0.111 −6.226 47.806 1.00 49.80 | |
| ATOM | 1567 OE2 GLU 411 | −0.605 −4.979 46.129 1.00 50.58 | |
| ATOM | 1568 C GLU 411 | 1.805 −2.000 50.594 1.00 38.78 | |
| ATOM | 1569 O GLU 411 | 2.254 −2.682 51.526 1.00 38.53 | |
| ATOM | 1570 N GLY 412 | 1.170 −0.845 50.768 1.00 34.78 | |
| ATOM | 1571 CA GLY 412 | 0.953 −0.313 52.103 1.00 32.19 | |
| ATOM | 1572 C GLY 412 | 2.163 0.402 52.695 1.00 29.28 | |
| ATOM | 1573 O GLY 412 | 2.133 0.770 53.865 1.00 28.68 | |
| ATOM | 1574 N ARG 413 | 3.207 0.625 51.901 1.00 28.70 | |
| ATOM | 1575 CA ARG 413 | 4.413 1.286 52.407 1.00 27.65 | |
| ATOM | 1576 CB ARG 413 | 5.513 1.319 51.348 1.00 29.50 | |
| ATOM | 1577 CG ARG 413 | 6.850 1.839 51.905 1.00 29.56 | |
| ATOM | 1578 CD ARG 413 | 8.004 1.721 50.903 1.00 32.68 | |
| ATOM | 1579 NE ARG 413 | 9.242 2.244 51.484 1.00 34.48 | |
| ATOM | 1580 CZ ARG 413 | 9.951 1.632 52.433 1.00 35.49 | |
| ATOM | 1581 NH1 ARG 413 | 9.569 0.454 52.914 1.00 34.87 | |
| ATOM | 1582 NH2 ARG 413 | 11.036 2.221 52.929 1.00 35.73 | |
| ATOM | 1583 C ARG 413 | 4.153 2.715 52.874 1.00 27.61 | |
| ATOM | 1584 O ARG 413 | 4.656 3.142 53.911 1.00 26.64 | |
| ATOM | 1585 N PHE 414 | 3.377 3.447 52.091 1.00 27.23 | |
| ATOM | 1586 CA PHE 414 | 3.025 4.829 52.406 1.00 25.72 | |
| ATOM | 1587 CB PHE 414 | 2.109 5.368 51.290 1.00 27.69 | |
| ATOM | 1588 CG PHE 414 | 1.553 6.756 51.552 1.00 26.95 | |
| ATOM | 1589 CD1 PHE 414 | 2.359 7.882 51.454 1.00 28.43 | |
| ATOM | 1590 CD2 PHE 414 | 0.217 6.921 51.891 1.00 29.19 | |
| ATOM | 1591 CE1 PHE 414 | 1.842 9.165 51.692 1.00 28.49 | |
| ATOM | 1592 CE2 PHE 414 | −0.315 8.199 52.134 1.00 29.46 | |
| ATOM | 1593 CZ PHE 414 | 0.503 9.325 52.033 1.00 28.75 | |
| ATOM | 1594 C PHE 414 | 2.336 4.891 53.777 1.00 26.40 | |
| ATOM | 1595 O PHE 414 | 2.691 5.702 54.637 1.00 26.65 | |
| ATOM | 1596 N ALA 415 | 1.355 4.027 53.997 1.00 25.07 | |
| ATOM | 1597 CA ALA 415 | 0.652 4.002 55.271 1.00 24.63 | |
| ATOM | 1598 CB ALA 415 | −0.531 3.028 55.193 1.00 23.72 | |
| ATOM | 1599 C ALA 415 | 1.572 3.622 56.436 1.00 24.02 | |
| ATOM | 1600 O ALA 415 | 1.433 4.142 57.547 1.00 23.15 | |
| ATOM | 1601 N ALA 416 | 2.518 2.722 56.174 1.00 24.35 | |
| ATOM | 1602 CA ALA 416 | 3.448 2.270 57.205 1.00 24.54 | |
| ATOM | 1603 CB ALA 416 | 4.313 1.143 56.666 1.00 24.42 | |
| ATOM | 1604 C ALA 416 | 4.319 3.436 57.639 1.00 23.97 | |
| ATOM | 1605 O ALA 416 | 4.544 3.646 58.832 1.00 25.89 | |
| ATOM | 1606 N LEU 417 | 4.799 4.192 56.664 1.00 23.99 | |
| ATOM | 1607 CA LEU 417 | 5.638 5.348 56.952 1.00 23.65 | |
| ATOM | 1608 CB LEU 417 | 6.056 6.013 55.651 1.00 23.88 | |
| ATOM | 1609 CG LEU 417 | 6.940 5.132 54.768 1.00 22.81 | |
| ATOM | 1610 CD1 LEU 417 | 7.104 5.745 53.381 1.00 25.97 | |
| ATOM | 1611 CD2 LEU 417 | 8.313 4.978 55.464 1.00 24.86 | |
| ATOM | 1612 C LEU 417 | 4.894 6.346 57.843 1.00 23.64 | |
| ATOM | 1613 O LEU 417 | 5.434 6.825 58.851 1.00 24.26 | |
| ATOM | 1614 N LEU 418 | 3.635 6.632 57.517 1.00 22.96 | |
| ATOM | 1615 CA LEU 418 | 2.896 7.617 58.305 1.00 22.78 | |
| ATOM | 1616 CB LEU 418 | 1.555 7.961 57.622 1.00 22.19 | |
| ATOM | 1617 CG LEU 418 | 1.632 8.536 56.193 1.00 22.93 | |
| ATOM | 1618 CD1 LEU 418 | 0.232 8.983 55.715 1.00 23.88 | |
| ATOM | 1619 CD2 LEU 418 | 2.554 9.749 56.179 1.00 23.65 | |
| ATOM | 1620 C LEU 418 | 2.646 7.201 59.762 1.00 24.12 | |
| ATOM | 1621 O LEU 418 | 2.372 8.046 60.620 1.00 22.61 | |
| ATOM | 1622 N LEU 419 | 2.706 5.901 60.049 1.00 25.06 | |
| ATOM | 1623 CA LEU 419 | 2.496 5.440 61.404 1.00 27.92 | |
| ATOM | 1624 CB LEU 419 | 2.406 3.909 61.451 1.00 31.65 | |
| ATOM | 1625 CG LEU 419 | 1.114 3.343 60.884 1.00 34.11 | |
| ATOM | 1626 CD1 LEU 419 | 1.026 1.848 61.218 1.00 36.06 | |
| ATOM | 1627 CD2 LEU 419 | −0.068 4.081 61.480 1.00 34.83 | |
| ATOM | 1628 C LEU 419 | 3.594 5.891 62.360 1.00 29.85 | |
| ATOM | 1629 O LEU 419 | 3.400 5.885 63.574 1.00 31.55 | |
| ATOM | 1630 N ARG 420 | 4.736 6.296 61.828 1.00 31.33 | |
| ATOM | 1631 CA ARG 420 | 5.801 6.725 62.718 1.00 31.87 | |
| ATOM | 1632 CB ARG 420 | 7.145 6.755 61.985 1.00 32.74 | |
| ATOM | 1633 CG ARG 420 | 7.648 5.387 61.490 1.00 33.67 | |
| ATOM | 1634 CD ARG 420 | 7.856 4.364 62.622 1.00 36.93 | |
| ATOM | 1635 NE ARG 420 | 6.709 3.481 62.820 1.00 35.35 | |
| ATOM | 1636 CZ ARG 420 | 6.179 3.194 64.003 1.00 37.82 | |
| ATOM | 1637 NH1 ARG 420 | 6.692 3.714 65.116 1.00 39.26 | |
| ATOM | 1638 NH2 ARG 420 | 5.112 2.408 64.079 1.00 39.04 | |
| ATOM | 1639 C ARG 420 | 5.477 8.092 63.303 1.00 32.15 | |
| ATOM | 1640 O ARG 420 | 5.995 8.456 64.362 1.00 31.60 | |
| ATOM | 1641 N LEU 421 | 4.591 8.845 62.655 1.00 30.38 | |
| ATOM | 1642 CA LEU 421 | 4.278 10.175 63.164 1.00 29.53 | |
| ATOM | 1643 CB LEU 421 | 3.519 10.976 62.121 1.00 30.72 | |
| ATOM | 1644 CG LEU 421 | 4.322 11.027 60.808 1.00 31.31 | |
| ATOM | 1645 CD1 LEU 421 | 3.645 12.019 59.885 1.00 32.38 | |
| ATOM | 1646 CD2 LEU 421 | 5.800 11.411 61.053 1.00 31.74 | |
| ATOM | 1647 C LEU 421 | 3.582 10.256 64.521 1.00 28.39 | |
| ATOM | 1648 O LEU 421 | 3.977 11.061 65.363 1.00 26.36 | |
| ATOM | 1649 N PRO 422 | 2.533 9.446 64.762 1.00 27.69 | |
| ATOM | 1650 CD PRO 422 | 1.678 8.672 63.839 1.00 27.47 | |
| ATOM | 1651 CA PRO 422 | 1.915 9.559 66.090 1.00 26.04 | |
| ATOM | 1652 CB PRO 422 | 0.717 8.603 66.005 1.00 28.29 | |
| ATOM | 1653 CG PRO 422 | 0.350 8.664 64.557 1.00 28.91 | |
| ATOM | 1654 C PRO 422 | 2.906 9.143 67.198 1.00 25.36 | |
| ATOM | 1655 O PRO 422 | 2.832 9.637 68.322 1.00 24.06 | |
| ATOM | 1656 N ALA 423 | 3.821 8.228 66.872 1.00 24.34 | |
| ATOM | 1657 CA ALA 423 | 4.817 7.779 67.840 1.00 22.88 | |
| ATOM | 1658 CB ALA 423 | 5.589 6.599 67.273 1.00 26.29 | |
| ATOM | 1659 C ALA 423 | 5.765 8.942 68.158 1.00 24.20 | |
| ATOM | 1660 O ALA 423 | 6.094 9.197 69.315 1.00 22.14 | |
| ATOM | 1661 N LEU 424 | 6.212 9.640 67.119 1.00 22.92 | |
| ATOM | 1662 CA LEU 424 | 7.103 10.773 67.309 1.00 22.78 | |
| ATOM | 1663 CB LEU 424 | 7.512 11.306 65.936 1.00 23.40 | |
| ATOM | 1664 CG LEU 424 | 8.405 12.531 65.875 1.00 24.54 | |
| ATOM | 1665 CD1 LEU 424 | 9.777 12.226 66.517 1.00 22.52 | |
| ATOM | 1666 CD2 LEU 424 | 8.589 12.892 64.405 1.00 22.78 | |
| ATOM | 1667 C LEU 424 | 6.422 11.861 68.153 1.00 22.46 | |
| ATOM | 1668 O LEU 424 | 7.036 12.468 69.038 1.00 21.68 | |
| ATOM | 1669 N ARG 425 | 5.136 12.101 67.900 1.00 22.38 | |
| ATOM | 1670 CA ARG 425 | 4.386 13.095 68.663 1.00 23.67 | |
| ATOM | 1671 CB ARG 425 | 2.969 13.240 68.087 1.00 26.56 | |
| ATOM | 1672 CG ARG 425 | 2.066 14.140 68.903 1.00 30.90 | |
| ATOM | 1673 CD ARG 425 | 0.977 14.732 68.031 1.00 36.25 | |
| ATOM | 1674 NE ARG 425 | 0.469 13.774 67.044 1.00 40.86 | |
| ATOM | 1675 CZ ARG 425 | −0.070 12.592 67.339 1.00 42.76 | |
| ATOM | 1676 NH1 ARG 425 | −0.183 12.196 68.604 1.00 46.63 | |
| ATOM | 1677 NH2 ARG 425 | −0.498 11.800 66.367 1.00 44.97 | |
| ATOM | 1678 C ARG 425 | 4.309 12.737 70.150 1.00 21.12 | |
| ATOM | 1679 O ARG 425 | 4.418 13.604 71.021 1.00 21.30 | |
| ATOM | 1680 N SER 426 | 4.124 11.452 70.436 1.00 19.60 | |
| ATOM | 1681 CA SER 426 | 4.021 10.989 71.820 1.00 19.49 | |
| ATOM | 1682 CB SER 426 | 3.534 9.539 71.855 1.00 20.63 | |
| ATOM | 1683 OG SER 426 | 3.491 9.086 73.198 1.00 23.17 | |
| ATOM | 1684 C SER 426 | 5.374 11.096 72.535 1.00 17.38 | |
| ATOM | 1685 O SER 426 | 5.458 11.496 73.698 1.00 18.97 | |
| ATOM | 1686 N ILE 427 | 6.419 10.742 71.812 1.00 18.11 | |
| ATOM | 1687 CA ILE 427 | 7.770 10.812 72.368 1.00 15.88 | |
| ATOM | 1688 CB ILE 427 | 8.763 10.095 71.449 1.00 15.75 | |
| ATOM | 1689 CG2 ILE 427 | 10.213 10.351 71.913 1.00 17.78 | |
| ATOM | 1690 CG1 ILE 427 | 8.479 8.591 71.526 1.00 18.23 | |
| ATOM | 1691 CD1 ILE 427 | 9.082 7.789 70.440 1.00 20.33 | |
| ATOM | 1692 C ILE 427 | 8.146 12.272 72.599 1.00 16.69 | |
| ATOM | 1693 O ILE 427 | 8.779 12.589 73.606 1.00 16.41 | |
| ATOM | 1694 N SER 428 | 7.767 13.159 71.685 1.00 17.02 | |
| ATOM | 1695 CA SER 428 | 8.059 14.590 71.884 1.00 17.37 | |
| ATOM | 1696 CB SER 428 | 7.636 15.393 70.633 1.00 18.11 | |
| ATOM | 1697 OG SER 428 | 7.745 16.794 70.876 1.00 19.14 | |
| ATOM | 1698 C SER 428 | 7.377 15.138 73.154 1.00 18.08 | |
| ATOM | 1699 O SER 428 | 7.968 15.916 73.928 1.00 16.98 | |
| ATOM | 1700 N LEU 429 | 6.134 14.741 73.425 1.00 18.84 | |
| ATOM | 1701 CA LEU 429 | 5.518 15.249 74.633 1.00 19.23 | |
| ATOM | 1702 CB LEU 429 | 4.054 14.807 74.723 1.00 22.17 | |
| ATOM | 1703 CG LEU 429 | 3.187 15.432 73.625 1.00 25.96 | |
| ATOM | 1704 CD1 LEU 429 | 1.800 14.809 73.624 1.00 28.92 | |
| ATOM | 1705 CD2 LEU 429 | 3.087 16.943 73.855 1.00 29.57 | |
| ATOM | 1706 C LEU 429 | 6.296 14.782 75.860 1.00 17.99 | |
| ATOM | 1707 O LEU 429 | 6.465 15.527 76.816 1.00 18.52 | |
| ATOM | 1708 N LYS 430 | 6.778 13.544 75.830 1.00 18.39 | |
| ATOM | 1709 CA LYS 430 | 7.540 13.047 76.957 1.00 17.66 | |
| ATOM | 1710 CB LYS 430 | 7.780 11.544 76.826 1.00 19.73 | |
| ATOM | 1711 CG LYS 430 | 8.539 10.975 78.020 1.00 23.74 | |
| ATOM | 1712 CD LYS 430 | 7.625 11.004 79.260 1.00 27.95 | |
| ATOM | 1713 CE LYS 430 | 8.192 10.194 80.414 1.00 34.36 | |
| ATOM | 1714 NZ LYS 430 | 7.167 10.122 81.490 1.00 36.64 | |
| ATOM | 1715 C LYS 430 | 8.865 13.795 77.077 1.00 18.37 | |
| ATOM | 1716 O LYS 430 | 9.346 14.034 78.191 1.00 18.47 | |
| ATOM | 1717 N SER 431 | 9.435 14.174 75.936 0.50 17.09 | AC1 |
| ATOM | 1718 CA SER 431 | 10.693 14.931 75.919 0.50 18.08 | AC1 |
| ATOM | 1719 CB SER 431 | 11.102 15.238 74.471 0.50 17.50 | AC1 |
| ATOM | 1720 OG SER 431 | 12.180 16.159 74.413 0.50 19.63 | AC1 |
| ATOM | 1721 C SER 431 | 10.472 16.244 76.663 0.50 18.02 | AC1 |
| ATOM | 1722 O SER 431 | 11.297 16.668 77.464 0.50 17.75 | AC1 |
| ATOM | 1723 N PHE 432 | 9.326 16.875 76.415 1.00 17.68 | |
| ATOM | 1724 CA PHE 432 | 9.020 18.162 77.057 1.00 17.81 | |
| ATOM | 1725 CB PHE 432 | 7.778 18.817 76.425 1.00 18.74 | |
| ATOM | 1726 CG PHE 432 | 8.099 19.713 75.249 1.00 17.54 | |
| ATOM | 1727 CD1 PHE 432 | 8.649 20.976 75.448 1.00 18.72 | |
| ATOM | 1728 CD2 PHE 432 | 7.851 19.290 73.958 1.00 17.78 | |
| ATOM | 1729 CE1 PHE 432 | 8.941 21.800 74.351 1.00 18.20 | |
| ATOM | 1730 CE2 PHE 432 | 8.135 20.093 72.863 1.00 18.04 | |
| ATOM | 1731 CZ PHE 432 | 8.684 21.358 73.060 1.00 19.15 | |
| ATOM | 1732 C PHE 432 | 8.817 17.989 78.550 1.00 18.32 | |
| ATOM | 1733 O PHE 432 | 9.170 18.872 79.330 1.00 20.66 | |
| ATOM | 1734 N GLU 433 | 8.249 16.852 78.964 1.00 17.15 | |
| ATOM | 1735 CA GLU 433 | 8.073 16.592 80.382 1.00 20.50 | |
| ATOM | 1736 CB GLU 433 | 7.520 15.192 80.613 1.00 19.84 | |
| ATOM | 1737 CG GLU 433 | 6.045 15.053 80.320 1.00 27.71 | |
| ATOM | 1738 CD GLU 433 | 5.533 13.688 80.744 1.00 31.07 | |
| ATOM | 1739 OE1 GLU 433 | 5.964 13.224 81.823 1.00 34.98 | |
| ATOM | 1740 OE2 GLU 433 | 4.714 13.094 80.006 1.00 34.76 | |
| ATOM | 1741 C GLU 433 | 9.437 16.691 81.052 1.00 19.32 | |
| ATOM | 1742 O GLU 433 | 9.574 17.322 82.106 1.00 20.27 | |
| ATOM | 1743 N HIS 434 | 10.438 16.059 80.435 1.00 18.45 | |
| ATOM | 1744 CA HIS 434 | 11.810 16.086 80.972 1.00 19.10 | |
| ATOM | 1745 CB HIS 434 | 12.708 15.091 80.247 1.00 20.67 | |
| ATOM | 1746 CG HIS 434 | 12.346 13.662 80.495 1.00 21.88 | |
| ATOM | 1747 CD2 HIS 434 | 12.196 12.621 79.642 1.00 22.64 | |
| ATOM | 1748 ND1 HIS 434 | 12.153 13.151 81.763 1.00 24.64 | |
| ATOM | 1749 CE1 HIS 434 | 11.901 11.857 81.678 1.00 26.38 | |
| ATOM | 1750 NE2 HIS 434 | 11.924 11.509 80.403 1.00 25.49 | |
| ATOM | 1751 C HIS 434 | 12.448 17.471 80.900 1.00 19.47 | |
| ATOM | 1752 O HIS 434 | 13.029 17.930 81.868 1.00 20.51 | |
| ATOM | 1753 N LEU 435 | 12.360 18.122 79.749 1.00 17.30 | |
| ATOM | 1754 CA LEU 435 | 12.926 19.464 79.614 1.00 17.70 | |
| ATOM | 1755 CB LEU 435 | 12.660 20.017 78.219 1.00 17.54 | |
| ATOM | 1756 CG LEU 435 | 13.350 19.239 77.100 1.00 17.06 | |
| ATOM | 1757 CD1 LEU 435 | 12.933 19.793 75.764 1.00 19.34 | |
| ATOM | 1758 CD2 LEU 435 | 14.874 19.343 77.242 1.00 19.09 | |
| ATOM | 1759 C LEU 435 | 12.334 20.411 80.653 1.00 17.96 | |
| ATOM | 1760 O LEU 435 | 13.034 21.260 81.193 1.00 18.73 | |
| ATOM | 1761 N PHE 436 | 11.035 20.302 80.921 1.00 17.35 | |
| ATOM | 1762 CA PHE 436 | 10.460 21.192 81.936 1.00 18.85 | |
| ATOM | 1763 CB PHE 436 | 8.920 21.188 81.897 1.00 19.91 | |
| ATOM | 1764 CG PHE 436 | 8.340 21.916 80.720 1.00 22.43 | |
| ATOM | 1765 CD1 PHE 436 | 8.885 23.113 80.288 1.00 23.77 | |
| ATOM | 1766 CD2 PHE 436 | 7.243 21.399 80.040 1.00 23.20 | |
| ATOM | 1767 CE1 PHE 436 | 8.353 23.783 79.202 1.00 25.60 | |
| ATOM | 1768 CE2 PHE 436 | 6.703 22.063 78.942 1.00 22.41 | |
| ATOM | 1769 CZ PHE 436 | 7.250 23.249 78.521 1.00 26.06 | |
| ATOM | 1770 C PHE 436 | 10.916 20.802 83.332 1.00 20.10 | |
| ATOM | 1771 O PHE 436 | 11.195 21.670 84.161 1.00 22.89 | |
| ATOM | 1772 N PHE 437 | 11.001 19.500 83.605 1.00 19.98 | |
| ATOM | 1773 CA PHE 437 | 11.412 19.023 84.922 1.00 21.11 | |
| ATOM | 1774 CB PHE 437 | 11.364 17.484 84.974 1.00 21.57 | |
| ATOM | 1775 CG PHE 437 | 11.628 16.913 86.339 1.00 25.91 | |
| ATOM | 1776 CD1 PHE 437 | 10.633 16.924 87.313 1.00 27.17 | |
| ATOM | 1777 CD2 PHE 437 | 12.881 16.419 86.665 1.00 27.22 | |
| ATOM | 1778 CE1 PHE 437 | 10.891 16.447 88.599 1.00 30.66 | |
| ATOM | 1779 CE2 PHE 437 | 13.153 15.942 87.944 1.00 31.40 | |
| ATOM | 1780 CZ PHE 437 | 12.158 15.957 88.910 1.00 29.78 | |
| ATOM | 1781 C PHE 437 | 12.807 19.496 85.305 1.00 22.67 | |
| ATOM | 1782 O PHE 437 | 13.046 19.895 86.464 1.00 24.17 | |
| ATOM | 1783 N PHE 438 | 13.724 19.453 84.346 1.00 19.58 | |
| ATOM | 1784 CA PHE 438 | 15.103 19.888 84.567 1.00 22.15 | |
| ATOM | 1785 CB PHE 438 | 16.038 19.027 83.718 1.00 22.90 | |
| ATOM | 1786 CG PHE 438 | 16.093 17.595 84.171 1.00 23.82 | |
| ATOM | 1787 CD1 PHE 438 | 16.725 17.262 85.361 1.00 24.00 | |
| ATOM | 1788 CD2 PHE 438 | 15.509 16.584 83.419 1.00 27.39 | |
| ATOM | 1789 CE1 PHE 438 | 16.773 15.942 85.795 1.00 25.11 | |
| ATOM | 1790 CE2 PHE 438 | 15.557 15.253 83.847 1.00 27.37 | |
| ATOM | 1791 CZ PHE 438 | 16.188 14.935 85.033 1.00 27.90 | |
| ATOM | 1792 C PHE 438 | 15.334 21.383 84.256 1.00 21.04 | |
| ATOM | 1793 O PHE 438 | 16.454 21.875 84.352 1.00 22.98 | |
| ATOM | 1794 N HIS 439 | 14.267 22.077 83.867 1.00 19.84 | |
| ATOM | 1795 CA HIS 439 | 14.311 23.503 83.508 1.00 19.79 | |
| ATOM | 1796 CB HIS 439 | 14.550 24.380 84.742 1.00 21.68 | |
| ATOM | 1797 CG HIS 439 | 13.463 24.294 85.763 1.00 24.96 | |
| ATOM | 1798 CD2 HIS 439 | 12.345 25.037 85.939 1.00 27.66 | |
| ATOM | 1799 ND1 HIS 439 | 13.440 23.331 86.747 1.00 26.44 | |
| ATOM | 1800 CE1 HIS 439 | 12.353 23.481 87.481 1.00 27.37 | |
| ATOM | 1801 NE2 HIS 439 | 11.672 24.511 87.012 1.00 28.08 | |
| ATOM | 1802 C HIS 439 | 15.375 23.803 82.469 1.00 20.93 | |
| ATOM | 1803 O HIS 439 | 16.185 24.726 82.626 1.00 23.42 | |
| ATOM | 1804 N LEU 440 | 15.318 23.068 81.366 1.00 16.51 | |
| ATOM | 1805 CA LEU 440 | 16.289 23.200 80.301 1.00 18.97 | |
| ATOM | 1806 CB LEU 440 | 16.820 21.813 79.917 1.00 19.31 | |
| ATOM | 1807 CG LEU 440 | 17.585 21.101 81.017 1.00 21.82 | |
| ATOM | 1808 CD1 LEU 440 | 17.920 19.672 80.567 1.00 24.43 | |
| ATOM | 1809 CD2 LEU 440 | 18.839 21.874 81.341 1.00 21.97 | |
| ATOM | 1810 C LEU 440 | 15.751 23.859 79.042 1.00 20.05 | |
| ATOM | 1811 O LEU 440 | 16.497 24.030 78.099 1.00 21.82 | |
| ATOM | 1812 N VAL 441 | 14.478 24.229 79.038 1.00 19.87 | |
| ATOM | 1813 CA VAL 441 | 13.879 24.814 77.833 1.00 21.84 | |
| ATOM | 1814 CB VAL 441 | 12.795 23.830 77.247 1.00 22.63 | |
| ATOM | 1815 CG1 VAL 441 | 11.601 23.713 78.192 1.00 22.79 | |
| ATOM | 1816 CG2 VAL 441 | 12.365 24.276 75.855 1.00 24.87 | |
| ATOM | 1817 C VAL 441 | 13.306 26.224 78.045 1.00 22.01 | |
| ATOM | 1818 O VAL 441 | 12.587 26.478 79.009 1.00 20.37 | |
| ATOM | 1819 N ALA 442 | 13.646 27.122 77.120 1.00 23.96 | |
| ATOM | 1820 CA ALA 442 | 13.233 28.529 77.172 1.00 27.08 | |
| ATOM | 1821 CB ALA 442 | 14.246 29.393 76.392 1.00 28.21 | |
| ATOM | 1822 C ALA 442 | 11.846 28.718 76.591 1.00 28.96 | |
| ATOM | 1823 O ALA 442 | 11.655 29.494 75.658 1.00 30.12 | |
| ATOM | 1824 N ASP 443 | 10.895 28.011 77.178 1.00 30.46 | |
| ATOM | 1825 CA ASP 443 | 9.493 27.994 76.764 1.00 31.83 | |
| ATOM | 1826 CB ASP 443 | 8.678 27.367 77.906 1.00 33.17 | |
| ATOM | 1827 CG ASP 443 | 7.208 27.218 77.581 1.00 34.98 | |
| ATOM | 1828 OD1 ASP 443 | 6.856 26.945 76.404 1.00 37.47 | |
| ATOM | 1829 OD2 ASP 443 | 6.404 27.346 78.524 1.00 31.58 | |
| ATOM | 1830 C ASP 443 | 8.873 29.320 76.318 1.00 31.97 | |
| ATOM | 1831 O ASP 443 | 8.426 29.453 75.180 1.00 30.41 | |
| ATOM | 1832 N THR 444 | 8.854 30.304 77.205 1.00 32.53 | |
| ATOM | 1833 CA THR 444 | 8.236 31.586 76.891 1.00 32.98 | |
| ATOM | 1834 CB THR 444 | 7.965 32.371 78.198 1.00 34.53 | |
| ATOM | 1835 OG1 THR 444 | 9.196 32.581 78.900 1.00 37.04 | |
| ATOM | 1836 CG2 THR 444 | 7.020 31.577 79.102 1.00 35.95 | |
| ATOM | 1837 C THR 444 | 8.981 32.486 75.901 1.00 32.99 | |
| ATOM | 1838 O THR 444 | 8.399 33.436 75.370 1.00 32.71 | |
| ATOM | 1839 N SER 445 | 10.248 32.179 75.639 1.00 30.56 | |
| ATOM | 1840 CA SER 445 | 11.071 32.977 74.727 1.00 29.90 | |
| ATOM | 1841 CB SER 445 | 12.481 33.132 75.313 1.00 29.52 | |
| ATOM | 1842 OG SER 445 | 12.418 33.766 76.576 1.00 31.76 | |
| ATOM | 1843 C SER 445 | 11.199 32.429 73.308 1.00 29.39 | |
| ATOM | 1844 O SER 445 | 11.580 33.153 72.387 1.00 28.30 | |
| ATOM | 1845 N ILE 446 | 10.875 31.151 73.133 1.00 26.98 | |
| ATOM | 1846 CA ILE 446 | 11.010 30.511 71.840 1.00 25.95 | |
| ATOM | 1847 CB ILE 446 | 10.656 29.013 71.961 1.00 26.36 | |
| ATOM | 1848 CG2 ILE 446 | 10.295 28.431 70.595 1.00 24.39 | |
| ATOM | 1849 CG1 ILE 446 | 11.864 28.300 72.575 1.00 27.57 | |
| ATOM | 1850 CD1 ILE 446 | 11.637 26.869 72.971 1.00 27.13 | |
| ATOM | 1851 C ILE 446 | 10.312 31.144 70.640 1.00 25.28 | |
| ATOM | 1852 O ILE 446 | 10.917 31.247 69.571 1.00 23.47 | |
| ATOM | 1853 N ALA 447 | 9.058 31.549 70.809 1.00 26.37 | |
| ATOM | 1854 CA ALA 447 | 8.316 32.183 69.725 1.00 28.80 | |
| ATOM | 1855 CB ALA 447 | 6.932 32.606 70.219 1.00 30.52 | |
| ATOM | 1856 C ALA 447 | 9.114 33.394 69.218 1.00 28.51 | |
| ATOM | 1857 O ALA 447 | 9.229 33.608 68.005 1.00 29.41 | |
| ATOM | 1858 N GLY 448 | 9.675 34.164 70.155 1.00 28.53 | |
| ATOM | 1859 CA GLY 448 | 10.474 35.337 69.811 1.00 27.89 | |
| ATOM | 1860 C GLY 448 | 11.762 34.993 69.095 1.00 28.77 | |
| ATOM | 1861 O GLY 448 | 12.167 35.692 68.162 1.00 28.23 | |
| ATOM | 1862 N TYR 449 | 12.435 33.927 69.536 1.00 27.26 | |
| ATOM | 1863 CA TYR 449 | 13.666 33.502 68.872 1.00 28.14 | |
| ATOM | 1864 CB TYR 449 | 14.262 32.267 69.553 1.00 26.16 | |
| ATOM | 1865 CG TYR 449 | 14.683 32.492 70.990 1.00 28.82 | |
| ATOM | 1866 CD1 TYR 449 | 14.913 33.782 71.482 1.00 29.84 | |
| ATOM | 1867 CE1 TYR 449 | 15.336 33.988 72.802 1.00 32.66 | |
| ATOM | 1868 CD2 TYR 449 | 14.881 31.412 71.853 1.00 29.90 | |
| ATOM | 1869 CE2 TYR 449 | 15.306 31.604 73.173 1.00 30.71 | |
| ATOM | 1870 CZ TYR 449 | 15.532 32.887 73.641 1.00 32.74 | |
| ATOM | 1871 OH TYR 449 | 15.979 33.070 74.939 1.00 36.98 | |
| ATOM | 1872 C TYR 449 | 13.361 33.150 67.420 1.00 27.51 | |
| ATOM | 1873 O TYR 449 | 14.116 33.491 66.513 1.00 27.99 | |
| ATOM | 1874 N ILE 450 | 12.254 32.442 67.207 1.00 27.41 | |
| ATOM | 1875 CA ILE 450 | 11.876 32.053 65.861 1.00 27.70 | |
| ATOM | 1876 CB ILE 450 | 10.662 31.102 65.863 1.00 26.64 | |
| ATOM | 1877 CG2 ILE 450 | 10.292 30.744 64.413 1.00 26.88 | |
| ATOM | 1878 CG1 ILE 450 | 11.003 29.846 66.690 1.00 27.46 | |
| ATOM | 1879 CD1 ILE 450 | 9.811 28.956 67.032 1.00 24.45 | |
| ATOM | 1880 C ILE 450 | 11.534 33.295 65.041 1.00 29.34 | |
| ATOM | 1881 O ILE 450 | 11.994 33.440 63.911 1.00 30.32 | |
| ATOM | 1882 N ARG 451 | 10.735 34.187 65.617 1.00 30.43 | |
| ATOM | 1883 CA ARG 451 | 10.351 35.416 64.923 1.00 33.00 | |
| ATOM | 1884 CB ARG 451 | 9.514 36.306 65.851 1.00 32.56 | |
| ATOM | 1885 CG ARG 451 | 8.874 37.519 65.161 1.00 34.91 | |
| ATOM | 1886 CD ARG 451 | 7.955 38.328 66.076 1.00 36.14 | |
| ATOM | 1887 NE ARG 451 | 6.768 37.599 66.518 1.00 37.46 | |
| ATOM | 1888 CZ ARG 451 | 6.669 36.943 67.672 1.00 40.02 | |
| ATOM | 1889 NH1 ARG 451 | 7.690 36.921 68.521 1.00 41.16 | |
| ATOM | 1890 NH2 ARG 451 | 5.547 36.299 67.976 1.00 40.59 | |
| ATOM | 1891 C ARG 451 | 11.629 36.138 64.472 1.00 34.57 | |
| ATOM | 1892 O ARG 451 | 11.761 36.516 63.298 1.00 34.59 | |
| ATOM | 1893 N ASP 452 | 12.578 36.304 65.392 1.00 36.05 | |
| ATOM | 1894 CA ASP 452 | 13.837 36.975 65.070 1.00 39.88 | |
| ATOM | 1895 CB ASP 452 | 14.690 37.184 66.331 1.00 42.41 | |
| ATOM | 1896 CG ASP 452 | 14.004 38.065 67.364 1.00 46.01 | |
| ATOM | 1897 OD1 ASP 452 | 13.162 38.897 66.961 1.00 46.57 | |
| ATOM | 1898 OD2 ASP 452 | 14.315 37.934 68.576 1.00 48.55 | |
| ATOM | 1899 C ASP 452 | 14.657 36.212 64.039 1.00 40.80 | |
| ATOM | 1900 O ASP 452 | 15.219 36.805 63.112 1.00 42.52 | |
| ATOM | 1901 N ALA 453 | 14.730 34.896 64.197 1.00 41.16 | |
| ATOM | 1902 CA ALA 453 | 15.493 34.071 63.272 1.00 42.41 | |
| ATOM | 1903 CB ALA 453 | 15.585 32.643 63.796 1.00 41.71 | |
| ATOM | 1904 C ALA 453 | 14.904 34.070 61.863 1.00 44.06 | |
| ATOM | 1905 O ALA 453 | 15.635 33.910 60.887 1.00 44.96 | |
| ATOM | 1906 N LEU 454 | 13.590 34.259 61.759 1.00 45.34 | |
| ATOM | 1907 CA LEU 454 | 12.921 34.261 60.460 1.00 47.12 | |
| ATOM | 1908 CB LEU 454 | 11.419 33.989 60.631 1.00 42.61 | |
| ATOM | 1909 CG LEU 454 | 11.078 32.514 60.884 1.00 38.89 | |
| ATOM | 1910 CD1 LEU 454 | 9.576 32.329 60.950 1.00 36.29 | |
| ATOM | 1911 CD2 LEU 454 | 11.660 31.657 59.764 1.00 36.23 | |
| ATOM | 1912 C LEU 454 | 13.149 35.544 59.669 1.00 50.37 | |
| ATOM | 1913 O LEU 454 | 13.255 35.506 58.443 1.00 51.61 | |
| ATOM | 1914 N ARG 455 | 13.218 36.679 60.360 1.00 54.48 | |
| ATOM | 1915 CA ARG 455 | 13.486 37.951 59.688 1.00 58.31 | |
| ATOM | 1916 CB ARG 455 | 13.128 39.147 60.582 1.00 59.22 | |
| ATOM | 1917 CG ARG 455 | 11.635 39.352 60.806 1.00 60.97 | |
| ATOM | 1918 CD ARG 455 | 11.318 40.787 61.228 1.00 62.50 | |
| ATOM | 1919 NE ARG 455 | 10.998 40.919 62.648 1.00 64.04 | |
| ATOM | 1920 CZ ARG 455 | 11.873 40.784 63.640 1.00 64.71 | |
| ATOM | 1921 NH1 ARG 455 | 13.146 40.511 63.378 1.00 64.92 | |
| ATOM | 1922 NH2 ARG 455 | 11.470 40.923 64.897 1.00 64.48 | |
| ATOM | 1923 C ARG 455 | 14.988 37.938 59.452 1.00 60.72 | |
| ATOM | 1924 O ARG 455 | 15.597 38.964 59.147 1.00 61.55 | |
| ATOM | 1925 N ASN 456 | 15.557 36.743 59.597 1.00 63.01 | |
| ATOM | 1926 CA ASN 456 | 16.983 36.482 59.463 1.00 64.82 | |
| ATOM | 1927 CB ASN 456 | 17.434 36.512 57.987 1.00 66.21 | |
| ATOM | 1928 CG ASN 456 | 17.254 37.871 57.327 1.00 67.51 | |
| ATOM | 1929 OD1 ASN 456 | 17.901 38.850 57.702 1.00 68.69 | |
| ATOM | 1930 ND2 ASN 456 | 16.377 37.930 56.326 1.00 68.01 | |
| ATOM | 1931 C ASN 456 | 17.795 37.442 60.317 1.00 65.37 | |
| ATOM | 1932 O ASN 456 | 17.456 37.680 61.480 1.00 65.63 | |
| ATOM | 1933 N GLY 457 | 18.858 37.997 59.749 1.00 65.99 | |
| ATOM | 1934 CA GLY 457 | 19.704 38.896 60.510 1.00 66.46 | |
| ATOM | 1935 C GLY 457 | 20.739 38.015 61.176 1.00 66.78 | |
| ATOM | 1936 O GLY 457 | 21.568 38.471 61.968 1.00 67.21 | |
| ATOM | 1937 N GLY 458 | 20.669 36.728 60.844 1.00 66.86 | |
| ATOM | 1938 CA GLY 458 | 21.594 35.753 61.384 1.00 66.84 | |
| ATOM | 1939 C GLY 458 | 22.018 34.761 60.315 1.00 66.86 | |
| ATOM | 1940 O GLY 458 | 21.450 34.801 59.199 1.00 66.65 | |
| ATOM | 1941 OXT GLY 458 | 22.922 33.943 60.593 1.00 65.49 | |
| ATOM | 1942 OH2 TIP 1003 | 30.252 23.128 74.386 1.00 27.69 | |
| ATOM | 1943 OH2 TIP 1005 | 14.203 25.558 89.644 1.00 25.22 | |
| ATOM | 1944 OH2 TIP 1006 | 8.388 25.042 72.262 1.00 22.81 | |
| ATOM | 1945 OH2 TIP 1008 | 8.367 21.538 69.460 1.00 19.23 | |
| ATOM | 1946 OH2 TIP 1009 | −7.350 22.030 52.884 1.00 80.11 | |
| ATOM | 1947 OH2 TIP 1010 | −4.017 19.644 67.897 1.00 33.26 | |
| ATOM | 1948 OH2 TIP 1011 | 8.365 3.022 77.974 1.00 47.93 | |
| ATOM | 1949 OH2 TIP 1012 | 30.690 8.779 67.839 1.00 26.30 | |
| ATOM | 1950 OH2 TIP 1013 | 12.264 8.843 80.249 1.00 26.01 | |
| ATOM | 1951 OH2 TIP 1014 | −1.764 16.382 62.652 1.00 44.82 | |
| ATOM | 1952 OH2 TIP 1015 | 20.301 34.946 75.498 1.00 51.92 | |
| ATOM | 1953 OH2 TIP 1016 | 14.443 15.693 61.296 1.00 22.04 | |
| ATOM | 1954 OH2 TIP 1017 | 12.487 31.635 78.951 1.00 36.76 | |
| ATOM | 1955 OH2 TIP 1018 | 16.579 6.557 83.739 1.00 27.86 | |
| ATOM | 1956 OH2 TIP 1019 | −0.626 26.615 50.499 1.00 30.82 | |
| ATOM | 1957 OH2 TIP 1021 | 3.543 20.127 64.859 1.00 23.80 | |
| ATOM | 1958 OH2 TIP 1022 | 4.772 0.996 47.855 1.00 40.67 | |
| ATOM | 1959 OH2 TIP 1023 | 9.799 29.451 51.621 1.00 30.93 | |
| ATOM | 1960 OH2 TIP 1024 | 7.476 19.030 68.589 1.00 22.30 | |
| ATOM | 1961 OH2 TIP 1025 | 20.355 7.131 58.551 1.00 52.44 | |
| ATOM | 1962 OH2 TIP 1026 | −0.829 29.526 57.153 1.00 31.90 | |
| ATOM | 1963 OH2 TIP 1027 | 11.560 −6.342 53.442 1.00 52.29 | |
| ATOM | 1964 OH2 TIP 1028 | 15.278 0.625 72.808 1.00 27.12 | |
| ATOM | 1965 OH2 TIP 1029 | 22.593 26.832 76.012 1.00 35.56 | |
| ATOM | 1966 OH2 TIP 1031 | 3.001 25.878 68.078 1.00 22.76 | |
| ATOM | 1967 OH2 TIP 1032 | 13.489 25.800 47.958 1.00 47.50 | |
| ATOM | 1968 OH2 TIP 1033 | −7.554 18.088 60.905 1.00 30.53 | |
| ATOM | 1969 OH2 TIP 1034 | 24.742 18.595 64.446 1.00 44.88 | |
| ATOM | 1970 OH2 TIP 1035 | 13.751 37.059 78.800 1.00 60.77 | |
| ATOM | 1971 OH2 TIP 1036 | −0.515 10.167 75.163 1.00 36.51 | |
| ATOM | 1972 OH2 TIP 1037 | 12.373 35.911 72.901 1.00 32.65 | |
| ATOM | 1973 OH2 TIP 1039 | 23.543 26.270 78.523 1.00 24.40 | |
| ATOM | 1974 OH2 TIP 1040 | 17.896 20.961 59.259 1.00 39.57 | |
| ATOM | 1975 OH2 TIP 1041 | 8.248 15.187 89.930 1.00 59.85 | |
| ATOM | 1976 OH2 TIP 1042 | 7.418 31.128 73.133 1.00 34.33 | |
| ATOM | 1977 OH2 TIP 1043 | 21.123 8.890 53.894 1.00 67.39 | |
| ATOM | 1978 OH2 TIP 1045 | 15.162 18.243 53.355 1.00 28.26 | |
| ATOM | 1979 OH2 TIP 1050 | 4.216 23.224 44.827 1.00 46.56 | |
| ATOM | 1980 OH2 TIP 1051 | 17.523 1.262 73.909 1.00 23.12 | |
| ATOM | 1981 OH2 TIP 1052 | −0.169 20.149 67.166 1.00 67.45 | |
| ATOM | 1982 OH2 TIP 1053 | 20.135 12.837 55.866 1.00 51.70 | |
| ATOM | 1983 OH2 TIP 1054 | 10.612 35.387 77.215 1.00 57.20 | |
| ATOM | 1984 OH2 TIP 1055 | 14.587 38.805 73.912 1.00 56.14 | |
| ATOM | 1985 OH2 TIP 1056 | 22.658 15.094 55.769 1.00 63.46 | |
| ATOM | 1986 OH2 TIP 1057 | 8.196 1.415 39.058 1.00 55.65 | |
| ATOM | 1987 OH2 TIP 1058 | 10.807 2.725 77.173 1.00 22.15 | |
| ATOM | 1988 OH2 TIP 1059 | 19.013 20.604 62.130 1.00 32.43 | |
| ATOM | 1989 OH2 TIP 1061 | 2.388 16.861 45.084 1.00 25.70 | |
| ATOM | 1990 OH2 TIP 1063 | 5.229 6.816 86.424 1.00 59.99 | |
| ATOM | 1991 OH2 TIP 1501 | 18.919 15.965 66.146 1.00 24.36 | |
| ATOM | 1992 OH2 TIP 1502 | 2.744 33.258 66.246 1.00 30.10 | |
| ATOM | 1993 OH2 TIP 1503 | 4.527 17.244 77.877 1.00 23.98 | |
| ATOM | 1994 OH2 TIP 1504 | −0.815 22.723 68.903 1.00 24.06 | |
| ATOM | 1995 OH2 TIP 1506 | 22.697 1.204 69.760 1.00 28.71 | |
| ATOM | 1996 OH2 TIP 1507 | 12.438 25.185 81.547 1.00 28.20 | |
| ATOM | 1997 OH2 TIP 1508 | 17.107 31.275 76.636 1.00 33.34 | |
| ATOM | 1998 OH2 TIP 1509 | 17.900 15.686 59.270 1.00 37.88 | |
| ATOM | 1999 OH2 TIP 1510 | 7.197 12.183 44.002 1.00 29.62 | |
| ATOM | 2000 OH2 TIP 1511 | −4.834 15.832 60.463 1.00 33.76 | |
| ATOM | 2001 OH2 TIP 1512 | 11.093 1.186 74.736 1.00 29.08 | |
| ATOM | 2002 OH2 TIP 1513 | −0.145 2.568 51.845 1.00 30.78 | |
| ATOM | 2003 OH2 TIP 1514 | −6.100 23.488 73.541 1.00 27.96 | |
| ATOM | 2004 OH2 TIP 1515 | 8.298 14.512 44.198 1.00 34.89 | |
| ATOM | 2005 OH2 TIP 1516 | 0.418 26.098 68.989 1.00 28.71 | |
| ATOM | 2006 OH2 TIP 1517 | −7.177 16.116 59.030 1.00 32.04 | |
| ATOM | 2007 OH2 TIP 1519 | 18.000 18.387 62.314 1.00 32.49 | |
| ATOM | 2008 OH2 TIP 1520 | 21.777 20.403 61.898 1.00 38.66 | |
| ATOM | 2009 OH2 TIP 1521 | −1.379 32.714 63.883 1.00 40.86 | |
| ATOM | 2010 OH2 TIP 1522 | 1.931 22.610 68.721 1.00 31.49 | |
| ATOM | 2011 OH2 TIP 1523 | −3.158 9.157 64.790 1.00 46.08 | |
| ATOM | 2012 OH2 TIP 1524 | 2.081 4.709 65.432 1.00 38.87 | |
| ATOM | 2013 OH2 TIP 1525 | 3.829 11.325 75.940 1.00 34.36 | |
| ATOM | 2014 OH2 TIP 1527 | 21.845 33.747 71.839 1.00 51.86 | |
| ATOM | 2015 OH2 TIP 1528 | 12.196 0.941 78.760 1.00 46.53 | |
| ATOM | 2016 OH2 TIP 1529 | 30.316 21.478 85.009 1.00 28.49 | |
| ATOM | 2017 OH2 TIP 1530 | 9.786 2.798 91.182 1.00 58.36 | |
| ATOM | 2018 OH2 TIP 1531 | 16.571 8.007 48.772 1.00 38.32 | |
| ATOM | 2019 OH2 TIP 1532 | 3.764 24.595 70.409 1.00 31.40 | |
| ATOM | 2020 OH2 TIP 1533 | −0.952 5.111 57.996 1.00 39.42 | |
| ATOM | 2021 OH2 TIP 1534 | 8.395 29.793 48.733 1.00 45.92 | |
| ATOM | 2022 OH2 TIP 1535 | 18.190 −0.943 54.382 1.00 55.57 | |
| ATOM | 2023 OH2 TIP 1536 | 4.583 13.859 64.203 1.00 30.44 | |
| ATOM | 2024 OH2 TIP 1538 | 12.012 14.232 84.365 1.00 33.97 | |
| ATOM | 2025 OH2 TIP 1539 | −1.284 36.017 69.736 1.00 57.91 | |
| ATOM | 2026 OH2 TIP 1540 | 2.454 15.898 79.022 1.00 40.22 | |
| ATOM | 2027 OH2 TIP 1544 | 2.719 2.670 49.088 1.00 32.00 | |
| ATOM | 2028 OH2 TIP 1545 | 13.537 37.410 71.136 1.00 41.29 | |
| ATOM | 2029 OH2 TIP 1546 | 22.697 0.071 79.248 1.00 32.01 | |
| ATOM | 2030 OH2 TIP 1548 | −0.239 7.542 39.851 1.00 52.37 | |
| ATOM | 2031 OH2 TIP 1549 | 0.076 10.603 44.453 1.00 41.67 | |
| ATOM | 2032 OH2 TIP 1550 | 31.157 3.039 59.611 1.00 43.66 | |
| ATOM | 2033 OH2 TIP 1551 | 4.226 34.045 72.549 1.00 53.58 | |
| ATOM | 2034 OH2 TIP 1554 | 10.022 33.359 56.088 1.00 41.48 | |
| ATOM | 2035 OH2 TIP 1555 | −1.058 37.708 61.917 1.00 54.81 | |
| ATOM | 2036 OH2 TIP 1556 | −4.583 15.870 53.480 1.00 37.02 | |
| ATOM | 2037 OH2 TIP 1557 | 23.851 8.517 92.595 1.00 36.91 | |
| ATOM | 2038 OH2 TIP 1558 | −7.204 28.744 59.955 1.00 35.72 | |
| ATOM | 2039 OH2 TIP 1560 | 19.483 16.334 88.331 1.00 34.61 | |
| ATOM | 2040 OH2 TIP 1561 | 1.968 8.086 38.648 1.00 57.90 | |
| ATOM | 2041 OH2 TIP 1562 | 32.430 −4.459 71.625 1.00 63.22 | |
| ATOM | 2042 OH2 TIP 1563 | 7.819 12.682 83.368 1.00 47.71 | |
| ATOM | 2043 OH2 TIP 1564 | −5.435 18.376 72.810 1.00 41.90 | |
| ATOM | 2044 OH2 TIP 1565 | 19.550 17.394 63.917 1.00 31.79 | |
| ATOM | 2045 OH2 TIP 1566 | 24.069 28.502 85.703 1.00 50.24 | |
| ATOM | 2046 OH2 TIP 1568 | 26.854 12.830 56.392 1.00 51.68 | |
| ATOM | 2047 OH2 TIP 1570 | 3.595 32.325 68.760 1.00 45.07 | |
| ATOM | 2048 OH2 TIP 1571 | 24.805 8.300 62.036 1.00 28.27 | |
| ATOM | 2049 OH2 TIP 1572 | 4.194 17.554 63.640 1.00 26.21 | |
| ATOM | 2050 OH2 TIP 1573 | 2.589 20.195 67.352 1.00 34.52 | |
| ATOM | 2051 OH2 TIP 1574 | 15.713 17.937 61.017 1.00 52.03 | |
| ATOM | 2052 OH2 TIP 1575 | −9.321 14.210 59.772 1.00 33.92 | |
| ATOM | 2053 OH2 TIP 1576 | 13.215 7.332 82.542 1.00 31.45 | |
| ATOM | 2054 OH2 TIP 1577 | 10.470 24.539 83.194 1.00 35.29 | |
| ATOM | 2055 OH2 TIP 1578 | 25.712 17.999 53.496 1.00 41.46 | |
| ATOM | 2056 OH2 TIP 1579 | 9.445 −0.239 41.882 1.00 41.51 | |
| ATOM | 2057 OH2 TIP 1580 | 6.603 16.005 42.611 1.00 32.35 | |
| ATOM | 2058 OH2 TIP 1581 | −1.523 7.654 59.739 1.00 50.11 | |
| ATOM | 2059 OH2 TIP 1582 | 8.397 34.515 72.891 1.00 33.81 | |
| ATOM | 2060 OH2 TIP 1583 | 2.742 39.191 60.949 1.00 39.77 | |
| ATOM | 2061 OH2 TIP 1584 | 18.933 6.009 52.002 1.00 45.27 | |
| ATOM | 2062 OH2 TIP 1585 | −1.653 20.171 69.665 1.00 37.15 | |
| ATOM | 2063 OH2 TIP 1586 | −2.633 4.655 52.475 1.00 49.05 | |
| ATOM | 2064 OH2 TIP 1587 | 36.297 28.180 83.444 1.00 41.56 | |
| ATOM | 2065 OH2 TIP 1588 | −0.851 31.806 55.808 1.00 34.41 | |
| ATOM | 2066 OH2 TIP 1589 | 4.002 34.625 70.007 1.00 46.13 | |
| ATOM | 2067 OH2 TIP 1590 | 32.711 20.152 84.581 1.00 53.56 | |
| ATOM | 2068 OH2 TIP 1591 | 19.998 6.099 87.630 1.00 31.12 | |
| ATOM | 2069 OH2 TIP 1593 | −0.189 3.637 35.682 1.00 54.58 | |
| ATOM | 2070 OH2 TIP 1594 | 12.455 12.705 39.358 1.00 55.12 | |
| ATOM | 2071 OH2 TIP 1596 | −2.554 −6.074 47.925 1.00 55.01 | |
| ATOM | 2072 OH2 TIP 1597 | 5.017 28.176 75.017 1.00 42.02 | |
| ATOM | 2073 OH2 TIP 1598 | 28.617 32.433 80.891 1.00 65.40 | |
| ATOM | 2074 OH2 TIP 1599 | 8.680 7.258 78.481 1.00 52.56 | |
| ATOM | 2075 OH2 TIP 1600 | 18.188 12.950 87.437 1.00 47.03 | |
| ATOM | 2076 OH2 TIP 1601 | −11.532 19.931 55.756 1.00 48.92 | |
| ATOM | 2077 OH2 TIP 1602 | 22.073 14.215 52.571 1.00 49.32 | |
| ATOM | 2078 OH2 TIP 1603 | −3.860 34.262 53.170 1.00 48.97 | |
| ATOM | 2079 OH2 TIP 1604 | 1.118 10.847 82.180 1.00 44.11 | |
| ATOM | 2080 OH2 TIP 1605 | 19.335 32.031 77.782 1.00 48.61 | |
| ATOM | 2081 OH2 TIP 1606 | 19.174 9.955 48.654 1.00 40.42 | |
| ATOM | 2082 OH2 TIP 1607 | 23.632 −1.631 71.300 1.00 37.97 | |
| ATOM | 2083 OH2 TIP 1608 | 26.622 26.695 85.361 1.00 44.14 | |
| ATOM | 2084 OH2 TIP 1609 | 22.586 −1.769 57.526 1.00 48.15 | |
| ATOM | 2085 OH2 TIP 1610 | 21.977 5.567 60.712 1.00 37.76 | |
| ATOM | 2086 OH2 TIP 1611 | 21.634 2.725 67.903 1.00 41.42 | |
| ATOM | 2087 OH2 TIP 1612 | 4.046 4.187 75.513 1.00 55.86 | |
| ATOM | 2088 OH2 TIP 1614 | 0.807 25.979 47.960 1.00 38.30 | |
| ATOM | 2089 OH2 TIP 1615 | 17.333 37.351 72.160 1.00 55.70 | |
| ATOM | 2090 OH2 TIP 1616 | 2.475 15.902 62.566 1.00 38.40 | |
| ATOM | 2091 OH2 TIP 1618 | 0.658 14.983 64.592 1.00 60.57 | |
| ATOM | 2092 OH2 TIP 1619 | −6.509 17.844 52.643 1.00 41.17 | |
| ATOM | 2093 OH2 TIP 1621 | 27.000 −1.287 80.946 1.00 51.49 | |
| ATOM | 2094 OH2 TIP 1622 | 3.271 9.154 86.392 1.00 55.17 | |
| ATOM | 2095 OH2 TIP 1627 | 3.433 19.409 44.225 1.00 50.54 | |
| ATOM | 2096 OH2 TIP 1628 | 2.390 26.629 72.360 1.00 42.60 | |
| ATOM | 2097 OH2 TIP 1629 | 9.893 39.104 69.833 1.00 54.40 | |
| ATOM | 2098 OH2 TIP 1630 | 2.709 14.153 43.455 1.00 34.37 | |
| ATOM | 2099 OH2 TIP 1631 | 11.049 12.448 88.232 1.00 45.81 | |
| ATOM | 2100 OH2 TIP 1632 | 4.576 31.506 72.757 1.00 39.34 | |
| ATOM | 2101 OH2 TIP 1634 | 6.784 36.285 71.148 1.00 51.53 | |
| ATOM | 2102 OH2 TIP 1635 | 6.667 43.335 56.568 1.00 51.21 | |
| ATOM | 2103 OH2 TIP 1636 | −5.771 9.260 60.442 1.00 44.79 | |
| ATOM | 2104 OH2 TIP 1638 | 0.052 33.418 66.937 1.00 47.03 | |
| ATOM | 2105 OH2 TIP 1641 | 0.354 1.055 46.133 1.00 54.03 | |
| ATOM | 2106 OH2 TIP 1642 | 24.406 30.113 88.300 1.00 48.82 | |
| ATOM | 2107 OH2 TIP 1643 | 26.619 20.182 66.495 1.00 38.01 | |
| ATOM | 2108 OH2 TIP 1644 | 17.492 7.024 42.815 1.00 65.02 | |
| ATOM | 2109 OH2 TIP 1645 | 25.942 26.481 82.676 1.00 49.52 | |
| ATOM | 2110 OH2 TIP 1646 | 20.601 16.199 68.672 1.00 37.35 | |
| ATOM | 2111 OH2 TIP 1649 | 27.616 9.156 63.460 1.00 37.92 | |
| ATOM | 2112 OH2 TIP 1650 | 0.428 −3.038 44.190 1.00 54.50 | |
| ATOM | 2113 OH2 TIP 1652 | −7.028 20.462 59.299 1.00 33.58 | |
| ATOM | 2114 OH2 TIP 1653 | −2.848 32.314 67.354 1.00 49.02 | |
| ATOM | 2115 OH2 TIP 1654 | −0.686 17.762 66.362 1.00 46.03 | |
| ATOM | 2116 OH2 TIP 1655 | 19.583 17.275 60.162 1.00 41.00 | |
| ATOM | 2117 OH2 TIP 1656 | 13.719 36.618 75.139 1.00 51.89 | |
| ATOM | 2118 OH2 TIP 1657 | 9.386 −0.422 71.399 1.00 43.15 | |
| ATOM | 2119 OH2 TIP 1659 | 23.690 28.880 79.578 1.00 42.62 | |
| ATOM | 2120 OH2 TIP 1660 | 22.069 3.800 58.682 1.00 46.06 | |
| ATOM | 2121 OH2 TIP 1661 | 20.671 13.353 58.841 1.00 57.05 | |
| ATOM | 2122 OH2 TIP 1662 | 27.473 10.135 82.332 1.00 47.43 | |
| ATOM | 2123 OH2 TIP 1664 | 9.564 26.542 84.601 1.00 44.55 | |
| ATOM | 2124 OH2 TIP 1666 | 29.122 9.606 65.764 1.00 45.20 | |
| ATOM | 2125 OH2 TIP 1668 | 13.135 20.507 41.865 1.00 59.09 | |
| ATOM | 2126 OH2 TIP 1669 | 22.639 11.672 58.999 1.00 54.98 | |
| ATOM | 2127 OH2 TIP 1670 | −1.845 6.027 76.197 1.00 48.89 | |
| ATOM | 2128 OH2 TIP 1672 | 4.883 25.252 42.734 1.00 51.13 | |
| ATOM | 2129 OH2 TIP 1675 | 1.329 39.322 66.763 1.00 68.30 | |
| ATOM | 2130 OH2 TIP 1676 | 12.783 29.313 87.079 1.00 54.62 | |
| ATOM | 2131 OH2 TIP 1679 | 25.035 18.339 57.364 1.00 54.53 | |
| ATOM | 2132 OH2 TIP 1682 | 29.392 −1.856 57.721 1.00 37.30 | |
| ATOM | 2133 OH2 TIP 1683 | 28.780 9.970 58.622 1.00 54.22 | |
| ATOM | 2134 OH2 TIP 1685 | 4.741 39.274 62.499 1.00 46.58 | |
| ATOM | 2135 OH2 TIP 1686 | −3.084 6.977 49.478 1.00 57.17 | |
| ATOM | 2136 OH2 TIP 1687 | 26.519 30.868 83.197 1.00 64.53 | |
| ATOM | 2137 OH2 TIP 1688 | −2.784 37.278 67.289 1.00 59.53 | |
| ATOM | 2138 OH2 TIP 1689 | 18.691 10.604 88.296 1.00 52.44 | |
| ATOM | 2139 OH2 TIP 1690 | 27.919 6.703 82.226 1.00 44.84 | |
| ATOM | 2140 OH2 TIP 1691 | −4.338 11.103 48.033 1.00 55.91 | |
| ATOM | 2141 OH2 TIP 1692 | −7.853 9.429 46.864 1.00 63.74 | |
| ATOM | 2142 OH2 TIP 1693 | 10.901 −1.686 67.477 1.00 41.21 | |
| ATOM | 2143 OH2 TIP 1694 | −2.114 6.315 55.259 1.00 56.21 | |
| ATOM | 2144 OH2 TIP 1695 | 17.482 15.932 44.391 1.00 41.18 | |
| ATOM | 2145 OH2 TIP 1696 | −12.326 38.088 61.786 1.00 53.30 | |
| ATOM | 2146 OH2 TIP 1697 | −2.176 40.471 68.230 1.00 68.88 | |
| ATOM | 2147 OH2 TIP 1700 | 6.514 −1.974 53.366 1.00 51.67 | |
| ATOM | 2148 OH2 TIP 1701 | 21.800 10.610 55.773 1.00 60.93 | |
| ATOM | 2149 OH2 TIP 1702 | 3.975 27.046 41.446 1.00 44.88 | |
| ATOM | 2150 OH2 TIP 1703 | 26.678 −3.660 64.081 1.00 62.42 | |
| ATOM | 2151 OH2 TIP 1704 | 2.958 12.027 86.133 1.00 53.52 | |
| ATOM | 2152 OH2 TIP 1705 | 4.264 22.050 63.018 1.00 16.96 | |
| ATOM | 2153 OH2 TIP 1706 | 22.999 26.329 63.006 1.00 32.17 | |
| ATOM | 2154 OH2 TIP 1707 | 5.614 2.688 68.201 1.00 42.08 | |
| ATOM | 2155 OH2 TIP 1708 | −2.967 17.730 54.394 1.00 38.89 | |
| ATOM | 2156 OH2 TIP 1709 | 25.853 10.594 62.118 1.00 43.50 | |
| ATOM | 2157 OH2 TIP 1711 | 13.060 12.966 86.563 1.00 41.22 | |
| ATOM | 2158 OH2 TIP 1712 | 19.784 15.472 45.489 1.00 58.17 | |
| ATOM | 2159 OH2 TIP 1713 | 10.567 14.806 42.991 1.00 43.48 | |
| ATOM | 2160 OH2 TIP 1714 | 24.079 30.190 83.477 1.00 47.61 | |
| ATOM | 2161 OH2 TIP 1715 | 23.927 21.975 63.464 1.00 44.77 | |
| ATOM | 2162 OH2 TIP 1716 | 15.801 20.193 58.769 1.00 34.32 | |
| ATOM | 2163 OH2 TIP 1717 | 23.867 27.717 72.712 1.00 40.47 | |
| ATOM | 2164 OH2 TIP 1718 | 24.567 27.201 69.884 1.00 45.97 | |
| ATOM | 2165 OH2 TIP 1719 | 32.141 −1.375 73.278 1.00 62.31 | |
| ATOM | 2166 OH2 TIP 1720 | 19.799 24.122 57.454 1.00 35.07 | |
| ATOM | 2167 OH2 TIP 1721 | 18.297 23.286 53.598 1.00 43.88 | |
| ATOM | 2168 OH2 TIP 1722 | 8.617 1.105 73.470 1.00 48.55 | |
| ATOM | 2169 OH2 TIP 1723 | 28.598 25.728 64.296 1.00 46.24 | |
| ATOM | 2170 OH2 TIP 1725 | 19.225 33.547 73.276 1.00 44.07 | |
| ATOM | 2171 OH2 TIP 1726 | 1.762 4.546 47.584 1.00 50.27 | |
| ATOM | 2172 OH2 TIP 1727 | 10.895 28.774 83.657 1.00 54.87 | |
| ATOM | 2173 OH2 TIP 1728 | 9.989 36.628 73.713 1.00 46.56 | |
| ATOM | 2174 OH2 TIP 1729 | −1.331 8.332 70.133 1.00 46.76 | |
| ATOM | 2175 OH2 TIP 1730 | 24.262 12.802 55.386 1.00 59.24 | |
| ATOM | 2176 OH2 TIP 1731 | 28.623 25.788 86.798 1.00 51.87 | |
| ATOM | 2177 OH2 TIP 1732 | −0.501 4.843 68.521 1.00 47.96 | |
| ATOM | 2178 OH2 TIP 1736 | 18.422 4.635 54.793 1.00 51.00 | |
| ATOM | 2179 OH2 TIP 1737 | −5.388 27.319 50.727 1.00 46.53 | |
| ATOM | 2180 OH2 TIP 1738 | −2.286 20.842 72.915 1.00 45.95 | |
| ATOM | 2181 OH2 TIP 1739 | 0.996 4.268 39.511 1.00 52.67 | |
| ATOM | 2182 OH2 TIP 1740 | −10.886 28.616 64.116 1.00 45.22 | |
| ATOM | 2183 OH2 TIP 1741 | 20.353 −4.883 70.512 1.00 61.31 | |
| ATOM | 2184 OH2 TIP 1742 | 22.491 16.164 60.365 1.00 58.19 | |
| ATOM | 2185 OH2 TIP 3001 | 15.272 21.419 87.789 1.00 27.44 | |
| ATOM | 2186 OH2 TIP 3002 | 13.055 32.876 52.925 1.00 53.35 | |
| ATOM | 2187 OH2 TIP 3006 | 16.014 18.841 64.083 1.00 56.45 | |
| ATOM | 2188 OH2 TIP 3008 | 16.802 30.100 54.388 1.00 48.87 | |
| ATOM | 2189 OH2 TIP 3009 | 13.673 27.099 82.740 1.00 32.07 | |
| ATOM | 2190 OH2 TIP 3010 | 30.041 24.325 84.969 1.00 41.40 | |
| ATOM | 2191 OH2 TIP 3007 | −2.102 35.612 60.958 1.00 52.05 | |
| ATOM | 2192 OH2 TIP 3011 | 7.242 14.501 40.017 1.00 51.46 | |
| ATOM | 2193 OH2 TIP 3012 | 1.031 36.834 60.593 1.00 49.05 | |
| ATOM | 2194 OH2 TIP 3013 | 0.026 24.244 72.355 1.00 42.35 | |
| ATOM | 2195 OH2 TIP 3015 | −7.871 31.986 57.037 1.00 46.09 | |
| ATOM | 2196 OH2 TIP 3019 | 8.655 −3.490 62.423 1.00 44.56 | |
| ATOM | 2197 OH2 TIP 3020 | −0.191 30.553 51.677 1.00 54.31 | |
| ATOM | 2198 OH2 TIP 3023 | 3.107 37.905 57.599 1.00 48.07 | |
| ATOM | 2199 OH2 TIP 3024 | 26.217 6.182 84.277 1.00 47.75 | |
| ATOM | 2200 OH2 TIP 3025 | 2.594 16.520 65.838 1.00 40.67 | |
| ATOM | 2201 C1 EPH 4000 | 23.874 12.843 85.264 1.00 42.79 | |
| ATOM | 2202 C2 EPH 4000 | 23.099 13.772 86.129 1.00 44.39 | |
| ATOM | 2203 C4 EPH 4000 | 24.923 13.000 83.062 1.00 45.55 | |
| ATOM | 2204 O2 EPH 4000 | 24.221 13.543 84.059 1.00 44.96 | |
| ATOM | 2205 O4 EPH 4000 | 25.350 11.876 83.028 1.00 47.53 | |
| ATOM | 2206 C18 EPH 4000 | 25.126 14.045 81.931 1.00 47.05 | |
| ATOM | 2207 C19 EPH 4000 | 24.674 13.569 80.547 1.00 46.98 | |
| ATOM | 2208 C20 EPH 4000 | 23.168 13.412 80.473 1.00 49.00 | |
| ATOM | 2209 C21 EPH 4000 | 22.772 12.939 79.111 1.00 51.06 | |
| ATOM | 2210 C22 EPH 4000 | 21.365 12.327 79.073 1.00 52.88 | |
| ATOM | 2211 C23 EPH 4000 | 20.291 13.230 78.423 1.00 54.24 | |
| ATOM | 2212 C24 EPH 4000 | 20.651 13.777 77.022 1.00 55.08 | |
| ATOM | 2213 C25 EPH 4000 | 19.987 15.129 76.704 1.00 56.13 | |
| ATOM | 2214 C26 EPH 4000 | 20.807 16.375 77.147 1.00 56.19 | |
| ATOM | 2215 C27 EPH 4000 | 19.981 17.687 77.287 1.00 56.74 | |
| ATOM | 2216 C28 EPH 4000 | 19.188 18.077 76.015 1.00 56.93 | |
| ATOM | 2217 C29 EPH 4000 | 20.055 18.393 74.792 1.00 57.07 | |
| ATOM | 2218 C30 EPH 4000 | 19.294 18.387 73.442 1.00 58.24 | |
| ATOM | 2219 C31 EPH 4000 | 19.391 17.124 72.550 1.00 58.70 | |
| ATOM | 2220 C32 EPH 4000 | 18.019 16.479 72.198 1.00 59.07 | |
| ATOM | 2221 C33 EPH 4000 | 16.762 17.158 72.768 1.00 59.61 | |
| ATOM | 2222 C34 EPH 4000 | 15.463 16.541 72.231 1.00 60.17 | |
| ATOM | 2223 C37 EPH 4000 | 22.780 13.059 87.421 1.00 47.03 | |
| ATOM | 2224 O5 EPH 4000 | 22.047 13.939 88.273 1.00 53.23 | |
| ATOM | 2225 P1 EPH 4000 | 21.699 13.222 89.578 1.00 56.71 | |
| ATOM | 2226 O6 EPH 4000 | 20.350 13.536 89.939 1.00 58.07 | |
| ATOM | 2227 O7 EPH 4000 | 22.579 12.360 90.311 1.00 56.72 | |
| ATOM | 2228 O8 EPH 4000 | 22.167 14.340 90.336 1.00 55.93 | |
| ATOM | 2229 C3 EPH 4000 | 21.561 15.394 85.253 1.00 42.20 | |
| ATOM | 2230 O1 EPH 4000 | 21.886 14.117 85.454 1.00 40.69 | |
| ATOM | 2231 O3 EPH 4000 | 22.221 16.347 85.571 1.00 40.97 | |
| ATOM | 2232 C5 EPH 4000 | 20.215 15.530 84.546 1.00 40.77 | |
| ATOM | 2233 C6 EPH 4000 | 20.313 15.776 83.050 1.00 42.19 | |
| ATOM | 2234 C7 EPH 4000 | 18.924 15.916 82.453 1.00 43.05 | |
| ATOM | 2235 C8 EPH 4000 | 18.900 15.944 80.947 1.00 44.57 | |
| ATOM | 2236 C9 EPH 4000 | 17.477 16.101 80.445 1.00 45.49 | |
| ATOM | 2237 C10 EPH 4000 | 17.167 15.209 79.273 1.00 47.18 | |
| ATOM | 2238 C11 EPH 4000 | 16.561 15.987 78.117 1.00 47.85 | |
| ATOM | 2239 C12 EPH 4000 | 15.158 15.514 77.781 1.00 49.75 | |
| ATOM | 2240 C13 EPH 4000 | 15.158 14.254 76.932 1.00 49.27 | |
| ATOM | 2241 C14 EPH 4000 | 14.899 14.563 75.454 1.00 51.41 | |
| ATOM | 2242 C15 EPH 4000 | 14.958 13.341 74.490 1.00 51.93 | |
| ATOM | 2243 C16 EPH 4000 | 16.376 12.870 74.074 1.00 52.39 | |
| ATOM | 2244 C17 EPH 4000 | 16.681 11.465 74.554 1.00 52.37 | |
| ATOM | 2245 C35 EPH 4000 | 17.830 11.518 75.525 1.00 52.96 | |
| ATOM | 2246 C36 EPH 4000 | 17.968 10.168 76.193 1.00 53.00 | |
| ATOM | 2247 C38 EPH 4000 | 22.197 10.885 90.057 1.00 56.75 | |
| ATOM | 2248 C39 EPH 4000 | 23.458 10.026 89.911 1.00 56.42 | |
| ATOM | 2249 N1 EPH 4000 | 24.546 10.825 89.334 1.00 54.45 | |
| ATOM | 2250 N SER 236 | 17.914 25.370 86.674 0.50 20.48 | AC2 |
| ATOM | 2251 CA SER 236 | 18.176 23.976 86.323 0.50 19.91 | AC2 |
| ATOM | 2252 CB SER 236 | 19.157 23.889 85.166 0.50 18.72 | AC2 |
| ATOM | 2253 OG SER 236 | 19.325 22.538 84.787 0.50 17.70 | AC2 |
| ATOM | 2254 C SER 236 | 18.741 23.171 87.483 0.50 21.62 | AC2 |
| ATOM | 2255 O SER 236 | 19.744 23.549 88.075 0.50 20.94 | AC2 |
| ATOM | 2256 N SER 247 | 25.235 21.608 79.357 0.50 19.68 | AC2 |
| ATOM | 2257 CA SER 247 | 25.203 22.865 78.619 0.50 20.48 | AC2 |
| ATOM | 2258 CB SER 247 | 26.051 23.917 79.337 0.50 19.95 | AC2 |
| ATOM | 2259 OG SER 247 | 26.032 25.152 78.637 0.50 20.60 | AC2 |
| ATOM | 2260 C SER 247 | 25.725 22.638 77.203 0.50 20.84 | AC2 |
| ATOM | 2261 O SER 247 | 25.203 23.202 76.238 0.50 21.05 | AC2 |
| ATOM | 2262 N SER 271 | 7.551 30.448 53.176 0.50 29.97 | AC2 |
| ATOM | 2263 CA SER 271 | 7.680 31.880 53.442 0.50 31.61 | AC2 |
| ATOM | 2264 CB SER 271 | 8.888 32.443 52.695 0.50 32.22 | AC2 |
| ATOM | 2265 OG SER 271 | 8.666 32.395 51.295 0.50 32.71 | AC2 |
| ATOM | 2266 C SER 271 | 6.432 32.648 53.010 0.50 32.99 | AC2 |
| ATOM | 2267 O SER 271 | 6.229 33.796 53.408 0.50 34.05 | AC2 |
| ATOM | 2268 N PRO 319 | 18.143 −4.099 74.681 0.50 41.20 | AC2 |
| ATOM | 2269 CD PRO 319 | 18.070 −4.311 76.139 0.50 40.63 | AC2 |
| ATOM | 2270 CA PRO 319 | 18.053 −2.673 74.356 0.50 38.50 | AC2 |
| ATOM | 2271 CB PRO 319 | 17.702 −2.038 75.699 0.50 39.26 | AC2 |
| ATOM | 2272 CG PRO 319 | 18.406 −2.938 76.680 0.50 39.94 | AC2 |
| ATOM | 2273 C PRO 319 | 19.321 −2.077 73.756 0.50 35.96 | AC2 |
| ATOM | 2274 O PRO 319 | 20.410 −2.230 74.313 0.50 35.87 | AC2 |
| ATOM | 2275 N GLN 343 | 13.913 5.584 80.085 0.50 18.73 | AC2 |
| ATOM | 2276 CA GLN 343 | 12.714 5.137 79.387 0.50 19.83 | AC2 |
| ATOM | 2277 CB GLN 343 | 11.463 5.365 80.243 0.50 21.34 | AC2 |
| ATOM | 2278 CG GLN 343 | 10.903 4.078 80.837 0.50 26.09 | AC2 |
| ATOM | 2279 CD GLN 343 | 9.539 4.244 81.488 0.50 27.02 | AC2 |
| ATOM | 2280 OE1 GLN 343 | 9.412 4.823 82.562 0.50 29.15 | AC2 |
| ATOM | 2281 NE2 GLN 343 | 8.508 3.730 80.829 0.50 29.67 | AC2 |
| ATOM | 2282 C GLN 343 | 12.545 5.813 78.025 0.50 19.53 | AC2 |
| ATOM | 2283 O GLN 343 | 12.317 5.141 77.022 0.50 19.10 | AC2 |
| ATOM | 2284 N SER 353 | 14.027 4.461 65.783 0.50 19.97 | AC2 |
| ATOM | 2285 CA SER 353 | 15.191 3.950 65.107 0.50 20.43 | AC2 |
| ATOM | 2286 CB SER 353 | 16.391 4.058 66.033 0.50 20.57 | AC2 |
| ATOM | 2287 OG SER 353 | 17.540 4.234 65.262 0.50 19.64 | AC2 |
| ATOM | 2288 C SER 353 | 15.054 2.524 64.574 0.50 21.08 | AC2 |
| ATOM | 2289 O SER 353 | 15.234 2.291 63.378 0.50 21.51 | AC2 |
| ATOM | 2290 N ARG 392 | 0.696 20.186 53.122 0.50 19.50 | AC2 |
| ATOM | 2291 CA ARG 392 | 0.935 18.822 53.549 0.50 19.96 | AC2 |
| ATOM | 2292 CB ARG 392 | 0.325 18.551 54.925 0.50 19.99 | AC2 |
| ATOM | 2293 CG ARG 392 | 0.603 17.129 55.384 0.50 19.08 | AC2 |
| ATOM | 2294 CD ARG 392 | 0.140 16.884 56.805 0.50 19.85 | AC2 |
| ATOM | 2295 NE ARG 392 | −1.315 16.885 56.925 0.50 19.23 | AC2 |
| ATOM | 2296 CZ ARG 392 | −1.962 16.577 58.046 0.50 21.63 | AC2 |
| ATOM | 2297 NH1 ARG 392 | −1.283 16.242 59.138 0.50 19.92 | AC2 |
| ATOM | 2298 NH2 ARG 392 | −3.289 16.611 58.086 0.50 22.19 | AC2 |
| ATOM | 2299 C ARG 392 | 0.338 17.878 52.501 0.50 19.95 | AC2 |
| ATOM | 2300 O ARG 392 | 0.940 16.867 52.149 0.50 20.64 | AC2 |
| ATOM | 2301 N SER 431 | 9.466 14.172 75.955 0.50 16.96 | AC2 |
| ATOM | 2302 CA SER 431 | 10.735 14.900 76.047 0.50 17.25 | AC2 |
| ATOM | 2303 CB SER 431 | 11.346 15.109 74.659 0.50 17.09 | AC2 |
| ATOM | 2304 OG SER 431 | 10.765 16.211 73.998 0.50 16.93 | AC2 |
| ATOM | 2305 C SER 431 | 10.466 16.249 76.719 0.50 17.71 | AC2 |
| ATOM | 2306 O SER 431 | 11.267 16.716 77.521 0.50 17.14 | AC2 |
| END | | | |

TABLE 2

| | |
|---|---|
| bdrxra | ----FNEEMPVEKILDAELAV--------------EPKTEAYME---------------- |
| murxra | ----ANEDMPVEKILEAELAV--------------EPKTETYVE---------------- |
| rnrxra | ----ANEDMPVEKILEAELAV--------------EPKTETYVE---------------- |
| horxra | ----ANEDMPVEKILEAELAV--------------EPKTETYVE---------------- |
| xlrxra | ----ANEDMPVEKILEAEHAV--------------EPKTETYTE---------------- |
| surxrb | ------------------------------------------------------------ |
| aeuspa | -----VRDVTIERIHEAEQLS-----E--------QKSGDNAIPYLR------------- |
| dmusp | MTNSVSRDFSIERIIEAEQRA-----E--------TQCGDRALTFLR------------- |
| horxrb | ----APEEMPVDRTLEAELAV--------------EQKSDQGVEGP-------------- |
| murxrb | ----APEEMPVDRILEAELAV--------------EQKSDQGVEGP-------------- |
| rnrxrb | ----APEEMPVDRILEAELAV--------------EQKSDQGVEGP-------------- |
| xlrxrbb | ----INEEMPVEKILEAELAV--------------EQKSDQSLE---------------- |
| xlrxrba | ----INEEMPVEKILEAELAV--------------EQKSDQSLE---------------- |
| murxrg | ----SHEDMPVERILEAELAV--------------EPKTESYGD---------------- |
| horxrg | ----GHEDMPVERILEAELAV--------------EPKTESYGD---------------- |
| garxrg | ----GSEDMPVERILEAELAV--------------EPKTEAYSD---------------- |
| xlrxrg | ----TSEEMPVERILEAELAV--------------DPKIEAFGD---------------- |
| pmrxr | ----PNDDMPVDKILEAELIS--------------DPKVEQVVP---------------- |
| lmrxr | ----LHTDMPVERILEAEKRV--------------E------------------------ |
| smrxr | ---TDLPNLTLRCLLSAELSM--------------DPKLAVSERG--------------- |
| amusp | ----LHSDMPTERILEAEKRV--------------ECKMEQQGN---------------- |
| tmusp | ----MQAEMPLDRIIEAEKRI--------------ECTPAGGSG---------------- |
| aarxr | ---GAPPEMPLERILEAELRV--------------ESQTGTLSES--------------- |
| aarxr2 | --P-GSPDMPLERILEAEMRV--------------EQPAPSVLAQ--------------- |
| uprxr | ----AISDMPIASIREAELSV--------------DPIDEQPLDQGVRLQVPLAPPDSEK |
| cfusp | --VQVSDELSIERLTEMESLV------ADPSEE------FQFLR---------------- |
| msusp | -----VQELSIERLLETESLV------ADPPEE------FQFLR---------------- |
| bmusp | -----VQELSIERLLELEALV------ADSAEE------LQILR---------------- |
| ctusp | --NGPGRDITVERLMEADQMS------EARCGDKS----IQYLR---------V------ |
| uspx | ---AAAQELSIERLLEMESLVAAAAEE------------FQFLR---------------- |
| rxrmin | -ASSANEDMPVEKILEAELAV--------------EPKTETYVE---------------- |
| bdrxra | ---------SSM---SNSTNDPVTNICQAADKQLFTLVEWAKRIPHFSDL---PLDDQVI |
| murxra | ---------ANMGLNPSSPNDPVTNICQAADKQLFTLVEWAKRIPHFSEL---PLDDQVI |
| rnrxra | ---------ANMGLNPSSPNDPVTNICQAADKQLFTLVEWAKRIPHFSEL---PLDDQVI |
| horxra | ---------ANMGLNPSSPNDPVTNICQAADKQLFTLVEWAKRIPHFSEL---PLDDQVI |
| xlrxra | ---------ANMGLAPNSPSDPVTNICQAADKQLFTLVEWAKRIPHFSDV---PLDDQVI |
| surxrb | -------------GSGSSPNDPVTNICQAADKQLFTLVEWAKRIPHFSSL---PLDDQVI |
| aeuspa | --------VGSNSMIPPEYKGAVSHLCQMVNKQIYQLIDFARRVPHFINL---PRDDQVM |
| dmusp | --------VGPYSTVQPDYKGAVSALCQVVNKQLFQMVEYARMMPHFAQV---PLDDQVI |
| horxrb | ---------GATGGGGSSPNDPVTNICQAADKQLFTLVEWAKRIPHFSSL---PLDDQVI |
| murxrb | ---------GATGGGGSSPNDPVTNICQAADKQLFTLVEWAKRIPHFSSL---PLDDQVI |
| rnrxrb | ---------GATGGGGSSPNDPVTNICQAADKQLFTLVEWAKRIPHFSSL---PLDDQVI |
| xlrxrbb | --------------GGGSPSDPVTNICQAADKQLFTLVEWAKRIPHFSEL---ALDDQVI |
| xlrxrba | --------------GGGSPSDPVTNICQDADKQLFTLVEWAKRIPHFSELPELPLDDQVI |
| murxrg | ------------MNVENSTNDPVTNICHAADKQLFTLVEWAKRIPHFSDL---TLEDQVI |
| horxrg | ------------MNMENSTNDPVTNICHAADKQLFTLVEWAKRIPHFSDL---TLEDQVI |
| garxrg | ------------VNTESSTNDPVTNICHAADKQLFTLVEWAKRIPHFSDL---TLEDQVI |
| xlrxrg | ------------AGLPNSTNDPVTNICHAADKQLFTLVEWAKRIPYFSDL---PLEDQVI |
| pmrxr | -------------FEQVNENDPVSNICKAADRQLVTLVEWAKRIPHFSSL---PLEDQVI |
| lmrxr | --------------------------CKAENQVEYELVEWAKHIPHFTSL---PLEDQVL |
| smrxr | -----EAIYEDIPGDDDTGLHPLTIICQSIEQQLPRIVNWARQLPVFSSVY-LSFDDQFC |
| amusp | ------------------YENAVSHICNATNKQLFQLVAWAKHIPHFTSL---PLEDQVL |
| tmusp | --------------GVGEQHDGVNNICQATNKQLFQLVQWAKLIPHFTSL---PMSDQVL |
| aarxr | ----------------AQQQDPVSSTCQAADRQLHQLVQWAKHIPHFEEL---PLEDRMV |
| aarxr2 | --------------TAASGRDPVNSMCQAAP-PLHELVQWARRIPHFEEL---PIEDRTA |
| uprxr | CSFTLPFHPVSEVSCANPLQDVVSNICQAADRHLVQLVEWAKHIPHFTDL---PIEDQVV |
| cfusp | --------VGPDSNVPPRYRAPVSSLCQIGNKQIAALVVWAPDIPHFGQL---ELDDQVV |
| msusp | --------VGPESGVPAKYRAPVSSLCQIGNKQTAALVVWARDIPHFGQL---ELEDQIL |
| bmusp | --------VGPESGVPAKYRAPVSSLCQIGNKQIAALIVWARDIPHFGQL---EIDDQIL |
| ctusp | --------AASNTMIPPEYRAPVSAICAMVNKQVFQHMDFCRRLPHFTKL---PLNDQMY |
| uspx | --------VGPDSNVPPKFRAPVSSLCQIGNKQIAALVVWARDIPHFSQL---EMEDQIL |
| rxrmin | ----------ANMGLNPSSPNDPVTNICQAAKQLFTLVEWAKRIPHFSEL---PLDDQVI |
| | * : :.: :* * .: .*: |
| bdrxra | LLRAGWNELLIASFSHRSVTVKDG--------------------------ILLATGLHVH |
| murxra | LLRAGWNELLIASFSHRSIAVKDG--------------------------ILLATGLHVH |
| rnrxra | LLRAGWNELLIASFSHRSIAVKDG--------------------------ILLATGLHVH |
| horxra | LLRAGWNELLIASFSHRSIAVKDG--------------------------ILLATGLHVH |
| xlrxra | LLPAGWNELLIASFSHRSIAVKDG--------------------------ILLATGLHVH |
| surxrb | LLRAGWNELLIASFSHRSIDVRDG--------------------------ILLATGLHVH |
| aeuspa | LLRCGWNEMLIAAVAWRSMEYIETER----------SSDGSRITVRQPQLMCLGPNFTLH |
| dmusp | LLKAAWIELLIMJVAWCSIVSLDDGGGGGGGGLGHDGSFERRSPGLQPQQLFLNQSFSYH |
| horxrb | LLRAGWNELLIASFSHRSIDVRDG--------------------------ILLATGLHVH |
| murxrb | LLRAGWNELLIASFSHRSIDVRDG--------------------------ILLATGLHVH |
| rnrxrb | LLRAGWNELLIASFSHRSIDVRDG--------------------------ILLATGLHVH |
| xlrxrbb | LLRAGWNELLIASFSHRSISVKDG--------------------------ILLATGLHVH |
| xlrxrba | LLRAGWNELLIASFSHRSISEKDG--------------------------ILLATGLHVH |
| murxrg | LLRAGWNELLIASFSHRSVSVQDG--------------------------ILLATGLHVH |
| horxrg | LLRAGWNELLIASFSHRSVSVQDG--------------------------ILLATGLHVH |
| garxrg | LLRAGWNELLIASFSHRSVSVQDG--------------------------ILLATGLHVH |
| xlrxrg | LLRAGWNELLIASFSHRSVSVQDG--------------------------ILLATGLHVH |
| pmrxr | LLRAGWNELLIASFSHRSIDVKDS--------------------------ILLASGLHVH |
| lmrxr | LLRAGWNELLIAAFSHRSVDVKDG--------------------------IVLATGLTVH |
| smrxr | LIKAAWPELVLISSAYHSTVIRDG--------------------------LLLSIGRHLG |
| amusp | LLPAGWNELLTASFSHRSIDVKDG--------------------------IVLATGITVH |
| tmusp | LLRAGWNELLIAAFSHRSIQAQDA--------------------------IVLATGLTVN |
| aarxr | LLKAGWNELLIAAFSHRSVDVRDG--------------------------IVLATGLVVQ |
| aarxr2 | LLKAGWNELLIAAFSHRSVAVRDG--------------------------IVLATGLVVQ |
| uprxr | LLKAGWNELLIASFSHRSMGVEDG--------------------------IVLATGLVIH |
| cfusp | LIKASWNELLLFAIAWRSMEYLEDER----------ENGDGTRSTTQPQLMCLMPGMTLH |
| msusp | LIKNSWNELLLFAIAWRSMEYLTDER----------ENVD-SRSTAPPQLMCLMPGMTLH |
| bmusp | LIKGSWNELLLFAIAWRSMEFLNDER----------ENVD-SRNTAPPQLICLMPGMTLH |
| ctusp | LLKQSLNELLILNIAYMSIQYVEPDRRNADG---------SLERRQISQQMCLSRNYTLG |
| uspx | LIKGSWNELLLFAIAWRSMEFLTAAAAS------------------PPQLMCLMPGMTLH |
| rxrmin | LLRAGWNELLIASFSHRSIAVK--------------------------DGILLATGLHVH |
| | *:: . *::: : * : * . |
| bdrxra | RSSAHSAGVGSIFDRVLTELVSKMPDMQMDKTELGCLRAIVLFNPDAKGLSNPSEVEALR |
| murxra | RNSAHSAGVGAIFDRVLTELVSKMRDMQMDKTELGCLRAIVLFNPDSKGLSNPAEVEALR |
| rnrxra | RNSAHSAGVGAIFDRVLTELVSKMRDMQMDKTELGCLRAIVLFNPDSKGLSNPAEVEALR |
| horxra | RNSAHSAGVGAIFDRVLTELVSKMRDMQMDKTELGCLRAIVLFNPDSKGLSNPAEVEALR |
| xlrxra | PNSAESAGVGAIFDRVLTELVSKMRDMQMDKTELGCLRAIVLFNPDSKGLSNPLEVEALR |
| surxrb | RNSAHSAGVGAIFDRVLTELVSKMRDMRMDKTELGCLRAIILFNPDAKGLSNPSEVEVLR |
| aeuspa | PNSAQQAGVDTLFDRILCELGIKMKRLDVTRAELGVLKAIILFNPDIRGLKCQKEIDGMR |
| dmusp | RNSAIKAGVSAIFDRILSELSVKMKRLNLDRRELSCLKAIILYNPDIRGIKSRABIEMCR |
| horxrb | RNSAHSAGVGAIFDRVLTELVSKMRDMRMDKTELGCLRAIILFNPDAKGLSNPGEVEILR |
| murxrb | RNSAHSAGVGAIFDRVLTELVSKMRDMRMDKTELGCLRAIILFNPDAKGLSNPGEVEILR |
| rnrxrb | RNSAHSAGVGAIFDRVLTELVSKMRDMRMDKTELGCLPAIILFNPDAKGLSNPGEVEILR |
| xlrxrbb | RNSAHSAGVGAIFDRVLTELVSKMRDMRMDKTELGCLRAIILFNPDAKGLSNPGDVEVLR |
| xlrxrba | RNSAISAGVGAIFERVLTELVSKMRDMRMDKTELGCLRAIILFNPDAKGLSNPGDVEVLR |
| murxrg | RSSAHSRGVGSIFDRVLTELVSKMKDMQMDKSELGCLPAIVLFNPDAKGLSNPSEVETLR |
| horxrg | RSSAHSAGVGSIFDRVLTELVSKMKDMQMDKSELGCLRAIVLFNPDAKGLSNPSEVETLR |
| garxrg | RSSAHSAGVGSIFDRVLTELVSKMKDMQMDKSELGCLRAIVLFNPDAKGLSSPSEVESLR |
| xlrxrg | RSSAHNAGVGSIFDRVLTELVSKMKDMDMDKSELGCLRAIVLFNPDAKGLSNAAEVEALR |
| pmrxr | RHSAHQAGVGPIFDRVLTELVSKMRDMMMDKTELGCLRAIVLFNPDVKNLSDSAHIESLR |
| lmrxr | PNSAHQAGVGTIFDRVLTELVAKMREMKMDKTELGCLRSVILFNPEVRGLKSAQEVELLR |
| smrxr | REVAKSHGLGPLVDRILHELVARFRDLSLQRTELALLRAIILFNPDANGLSSRHRVEAVR |
| amusp | RNSAQQAGVGTIFDRVLSELVSKMREMKMDRTELGCLRSIILFNPEVRGLKSIQEVTLLR |
| tmusp | KTSAHAVGVGNIYDRVLSELVNKMKEMKMDKTELGCLPAITLYNPTCRGIKSVQEVEMLR |
| aarxr | RHSAHGAGVGAIFDRVLTELVAKMREMKMDRTELGCLLAVVLFNPEAKGLRTCPSGGPEG |
| aarxr2 | RHSAHGAGVGDIFDRVLAELVAKMRDMKMDKTELGCLRAVVLFNPDAKGLRNATRVEALR |
| uprxr | RSSAHQAGVGAIFDRVLSELVAKMKEMKIDKTELGCLRSIVLFNPDAKGLNCVNDVEALR |
| cfusp | RNSAQQAGVGAIFDRVLSELSLKMRTLRMDQAEYVALKAIVLLNPDVKGLKNRQEVDVLR |
| msusp | RNSALQAGVGQIFDRVLSELSLKNRTLRMDQAEYVALKAIILLNPDVKGLKNKPEVVVLR |
| bmusp | RNSALQAGVGQTFDRVLSELSLKMRSLRMDQAECVALKAIILLNPDVKGLKNKQEVDVLR |
| ctusp | RNMAVQAGVVQIFDRILSELSVKMKRLDLDATELCLLKSIVVFNPDVRTLDDRKSIDLLR |
| uspx | RNSALQAGVGQIFDRVLSELSLKMRTLRVDQAEYVALKAIILLNPDVKGLKNRQEVEVLR |
| rxrmin | RNSAHSAGVGAIFDRVLTELVSKMRDMQMDKTELGCLRAIVLFNPDSKGLSNPAEVEALR |
| | : * *: : :*:* ** ::: : : * * :::: ** . : |
| bdrxra | EKVYASLEGYTKHNYPDQPGRFAKLLLRLPALRSIGLKCLEHLFFFKLIGDTPIDTFLME |
| murxra | EKVYASLEAYCKHKYPEQPGRFAKLLLRLPALRSIGLKCLEHLFFFKLIGDTPIDTFLME |
| rnrxra | EKVYASLEAYCKHKYPEQPGRFAKLLLRLPALRSIGLKCLEHLFFFKLIGDTPIDTFLME |
| horxra | EKVYASLEAYCKHKYPEQPGRFAKLLLRLPALRSIGLKCLEHLFFFKLIGDTPIDTFLME |
| xlrxra | EKVYASLEAYCKQKYPEQPGRFAKLLLRLPALRSIGLKCLEHLFFFKLIGDTPIDTFLME |
| surxrb | EKVYASLETYCKQKYPEQQGRFAKLLLRLPALRSIGLKCLEHLFFFKILI---------- |
| aeuspa | EKIYACLDEHCKQQHPSEDGRFAQLLLRLPALRSISLKCLDHLNFIRLLSDKHLDSFIVE |
| dmusp | EKVYACLDEHCRLEHPGDDGRFAQLLLRLPALRSISLKCQDHLFLFRITSDRPLEELFLE |
| horxrb | EKVYASLETYCKQKYPEQQGRFAKLLLRLPALRSIGLKCLEHLFFFKLIGDTPIDTFLME |
| murxrb | EKVYASLETYCKQKYPEQQGRFAKLLLRLPALRSIGLKCLEHLFFFKLIGDTPIDTFLME |
| rnrxrb | EKVYASLETYCKQKYPEQQGRFAKLLLRLPALRSIGLKCLEHLFFFKLIGDTPIDTFLME |
| xlrxrbb | EKVYASLESYCKQKYPDQQGRFAKLLLRLPALRSIGLKCLEHLFFFKLIGDTPIDTFLME |
| xlrxrba | EKVYACLESYCKQKYPDQQGRFAKLLLRLPALRSIGLKCLEHLFFFKLIGDTPIDTFLME |
| murxrg | EKVYATLEAYTKQKYPEQPGRFAKLLLRLPALRSIGLKCLEHLFFFKLIGDTPIDSFLME |
| horxrg | EKVYATLEAYTKQKYPEQPGRFAKLLLRLPALRSIGLKCLEHLFFFKLIGDTPIDTFLME |
| garxrg | EKVYATLEAYTKQKYPEQPGRFAKLLLRLPALRSIGLKCLEHLFFFKLIGDTPIDTFLME |
| xlrxrg | EKVYATLESYTKQKYPDQPGRFAKLLLRLPALRSIGLKCLEHLFFFKLIGDTPIDTFLME |
| pmrxr | EKVYASLEAYCRSKYPDQPGRFAKLLLRLPALRSIGLKCLEHLFFFKLIGDTPIDKFLMN |
| lmrxr | EKVYAALEEYTRTTHPDEPGRFAKLLLRLPSLRSIGLKCLEHLFFFRLIGDVPIDTFLME |
| smrxr | EQLYSALHSYCTTNQPQDTSRFTKLLLRLPPLRSIASKCLEHLVFVKLAAEDPTSCRLIN |
| amusp | EKIYGALEGYCRVAWPDDAGRFAKLLLRLPAIRSIGLKCLEYLFFFKMIGDVPIDDFLVE |
| tmusp | EKIYGVLEEYTRTTHPNEPGRFAKLLLRLPALRSIGLKCSEHLFFFKLIGDVPIDTFLME |
| aarxr | ESV-SALEEHCRQQYPDQPGRFAKLLLRLPALRSIGLKCLEHLFFFKLIGDTPIDNFLLS |
| aarxr2 | EKVYAALEEHCRRHHPDQPGRFGKLLLRLPALRSIGLKCLEHLFFFKLIGDTPIDSFLLN |
| uprxr | EKVYAALEEYTRTTYPDEPGRFAKLLLRLPALRSIGLKCLEYLFLFKLIGDTPLDSYLMK |
| cfusp | EKMFSCLDDYCRRSRSNEEGRFASLLLRLPALRSISLKSFEHLYFFHLVAEGSISGYIRE |
| msusp | EKMFSCLDEYVRRSRCAEEGRFAALLLRLPALRSISLKCFEHLYFFHLVADTSIASYIHD |
| bmusp | EKMFLCLDEYCRRSRGGEEGRFAALLLRLPALRSISLKSFEHLYLFHLVAEGSVSSYIRD |
| ctusp | SRIYASLDEYCRQKHPNEDGRFAQLLLRLPALRSISLKCLDNLFYFQLIDDKNVENSVIE |
| uspx | EKMFLCLDEYCRRSRSSEEGRFAALLLRLPALRSISLKSFEELFFFHLVADTSTAGYIRD |
| rxrmin | EKVYASLEAYCKHKYPEQPGRFAKLLLRLPALRSIGLKCLEHLFFFKLIGDTPIDTFLME |
| | . : *. : : .** ******.:***. *. ::* .:: |
| bdrxra | MLEAPHQIT |
| murxra | MLEAPHQAT |
| rnrxra | MLEAPHQTT |
| horxra | MLEAPHQMT |
| xlrxra | MLEAPHQMT |
| surxrb | --------- |
| aeuspa | MLDMPI--- |
| dmusp | QLEAPPPPG |
| horxrb | MLEAPHQLA |
| murxrb | MLEAPHQLA |
| rnrxrb | MLEAPHQLA |
| xlrxrbb | MLEAPHQLS |
| xlrxrba | MLEAPHQLS |
| murxrg | MLETPLQIT |
| horxrg | MLETPLQIT |
| garxrg | MLETPLQVT |
| xlrxrg | MLETPHQIS |
| pmrxr | MLETTSDFP |
| lmrxr | MLESPSDS- |
| smrxr | LVEHGVWPI |
| amusp | MLESRSDP- |
| tmusp | MLESPADA- |
| aarxr | MLEAPSDP- |
| aarxr2 | MLEAPADP- |
| uprxr | MLVDNPNTS |
| cfusp | ALRNHAPPI |
| msusp | ALPNHAPSI |
| bmusp | ALCNHAPPI |
| ctusp | EFHKLN--- |
| uspx | ALRNGG--- |
| rxrmin | MLEAP---- |

### REFERENCES

1. Oro A. E. et al. (1990): Relationship between the product of the Drosophila ultraspiracle locus and the vertebrate retinoid X receptor. Nature 347, 298-301

2. Moras D. & Gronemeyer H. (1998): The nuclear receptor ligand-binding domain: structure and fiction. Current Opinion in Cell Biology 10, 384-391

3. Segraves W. A. (1994): Steroid Receptors and Other Transcription Factors in Ecdysone Response. Recent Progress in Hormone Research, 49, 167-195

4. Henrich V. C. & Brown N. E. (1995): Insect Nuclear Receptors: A Developmental and Comparative Perspective. Insect Biochem. Mol. Biol. 25 (8), 881-897

5. Thummel C. S. (1995): From Embryogenesis to Metamorphosis: The Regulation and Function of Drosophila Nuclear Receptor Superfamily Members. Cell 83, 871-877

6. Truman J. W. (1996): Ecdysis Control Sheds Another Layer. Science 271, 40-41

7. Yao T. et al. (1993): Functional ecdysone receptor is the product of EcR and Ultraspiracle genes. Nature 366, 476-479

8. Hall B. L. & Thummel C. S. (1998): The RXR homolog Ultraspiracle is an essential component of the Drosophila ecdysone receptor. Development 125, 4709-4717

9. Lezzi M. et al. (1999): The Ecdysone Receptor Puzzle. Arch. Insect Biochem. Physiol. 41, 99-106

10. Mikitani K. (1996): Ecdysteroid Receptor Binding Activity and Ecdysteroid Agonist Activity at the Level of Gene Expression are Correlated with the Activity of Dibenzoyl Hydrazines in Larvae of Bombyx mori. J. Insect Physiol. 42 (10), 937-941

11. Dhadialla T. S. et al. (1998): New Insecticides with Ecdysteroidal and Juvenile Hormone Activity. Annu. Rev. Entomol. 43, 545-569

12. Sundaram M. et al. (1998): Basis for selective action of a synthetic molting hormone agonist, RH-5992 on lepidopteran insects. Insect Biochem. Mol. Biol. 28, 693-704

13. Rarey M. et al. (1996): Predicting Receptor-Ligand Interactions by an Incremental Construction Algorithm. J. Mol. Biol. 261(3), 470-489

14. Jones G. et al. (1997): Development and Validation of a Genetic Algorithm for Flexible Docking. J. Mol. Biol. 267 (3), 727-748

15. Böhm H. J. (1992): LUDI: Rule-Based Automatic Design of New Substituents for Enzyme Inhibitor Leads. J. Comp.-Aided Mol. Design 6, 593-606

16. Nishibata Y. & Itai A. (1991): Automatic creation of drug candidate structures based on receptor structure. Starting point for artificial lead generation. Tetrahedron 47, 8985-8990

17. Moon J. B. & Howe W. J. (1991): Computer design of bioactive molecules: a method for receptor-based de novo ligand design. Proteins 11 (4), 314-328

18. Otwinowski Z. & Minor W. (1997): Processing of X-ray diffraction data collected in oscillation mode. Methods Enzymol. 276, 307-326

19. Navaza J. (1994): Amore: an automated package for molecular replacement. Acta Cryst. A 50, 157-163

20. Egea P. F. et al. (2000): Crystal structure of the human RXRαalpha ligand-binding domain bound to its natural ligand 9-cis retinoic acid. EMBO J. 19, 2592-2601

21. Jones T. A. et al. (1991): Improved methods for generateing protein models in electron density maps and the location of errors in these models. Acta Cryst. A 47, 110-119

22. Perrakis A. et al (1997): wARP: improvement and extension of crystallographic phases by weighted averaging of multiple-refined dummy atomic models. Acta Cryst. D 53, 448-455

23. Brünger A. T. et al. (1998): Crystallography & NMR System: A New Software Suite for Macromolecular Structure Determination. Acta Cryst. D 54, 905-921

24. Laskowski R. A. et al. (1993): PROCHECK:a programme to check the stereochemical quality of protein structure coordinates. J. Appl. Crystallogr. 26, 283-291

1. A ligand-binding domain (LBD) of the ultraspiracle protein (USP) in crystalline form.

2. A LBD according to claim 1, wherein the LBD is a LBD of the Heliothis virescens USP.

3. A LBD according to claim 1, wherein the LBD comprises the amino acid sequence as shown in SEQ ID NO: 1.

4. A LBD according to claim 1, wherein the LBD exists in the form of a complex with a ligand.

5. A LBD according to claim 1, wherein the LBD has the structure coordinates defined in Table 1, below:
TABLE 1
REMARK coordinates from restrained individual B-factor refinement
REMARK refinement resolution: 20.0 - 1.65 A
REMARK starting r= 0.2151 free_r= 0.2506
REMARK final r= 0.2112 free_r= 0.2459
REMARK B rmsd for bonded mainchain atoms= 1.437 target= 1.5
REMARK B rmsd for bonded sidechain atoms= 2.272 target= 2.0
REMARK B rmsd for angle mainchain atoms= 2.299 target= 2.0
REMARK B rmsd for angle sidechain atoms= 3.310 target= 2.5
REMARK rweight= 0.1000 (with wa= 1.12122)
REMARK target= mlf steps= 30
REMARK sg= P4(3)22 a= 58.211 b= 58.211 c= 144.687 alpha= 90 beta= 90 gamma= 90
REMARK parameter file 1 : CNS_TOPPAR:protein_rep.param
REMARK parameter file 2 : CNS_TOPPAR:water_rep.param
REMARK parameter file 3 : eph.par
REMARK molecular structure file: alternate.mtf
REMARK input coordinates: anneal_2.pdb
REMARK reflection file= /home/billas/USP/SCALE0400/merge1A65/usp_20a1a65.10.cv
REMARK ncs= none
REMARK B-correction resolution: 6.0 - 1.65
REMARK initial B-factor correction applied to fobs:
REMARK B11= −1.985 B22= −1.985 B33= 3.970
REMARK B12= 0.000 B13= 0.000 B23= 0.000
REMARK B-factor correction applied to coordinate array B: −0.193
REMARK bulk solvent: density level= 0.33501 e/Aˆ 3, B-factor= 48.7849 Aˆ 2
REMARK reflections with |Fobs|/sigma_F < 0.0 rejected
REMARK reflections with |Fobs| > 10000 * rms(Fobs) rejected
REMARK theoretical total number of refl. in resol. range: 30842 (100.0%)
REMARK number of unobserved reflections (no entry or |F|=0): 1417 (4.6%)
REMARK number of reflection rejected: 0(0.0%)
REMARK total number of reflections used: 29425 (95.4%)
REMARK number of reflections in working set: 26453 (85.8%)
REMARK number of reflections in test set: 2972 (9.6%)
CRYST1 58.211 58.211 144.687 90.00 90.00 90.00 P 43 2 2
REMARK FILENAME=“/home/billas/LUC/13cns/bind_2.pdb”
REMARK DATE: 4-Jun-00 14:33:10 created by user: billas
REMARK VERSION: 1.0

| | | | | | | | | | | |
|---|---|---|---|---|---|---|---|---|---|---|
| ATOM | 1 | CB | ALA | 203 | 16.114 | 28.799 | 41.997 | 1.00 | 66.21 | |
| ATOM | 2 | C | ALA | 203 | 15.029 | 28.899 | 39.746 | 1.00 | 66.49 | |
| ATOM | 3 | O | ALA | 203 | 14.609 | 30.031 | 39.487 | 1.00 | 66.58 | |
| ATOM | 4 | N | ALA | 203 | 17.364 | 29.707 | 40.068 | 1.00 | 66.33 | |
| ATOM | 5 | CA | ALA | 203 | 16.347 | 28.703 | 40.490 | 1.00 | 66.40 | |
| ATOM | 6 | N | ALA | 204 | 14.387 | 27.790 | 39.393 | 1.00 | 66.05 | |
| ATOM | 7 | CA | ALA | 204 | 13.106 | 27.833 | 38.698 | 1.00 | 65.56 | |
| ATOM | 8 | CB | ALA | 204 | 12.933 | 26.584 | 37.843 | 1.00 | 65.11 | |
| ATOM | 9 | C | ALA | 204 | 12.028 | 27.888 | 39.776 | 1.00 | 64.97 | |
| ATOM | 10 | O | ALA | 204 | 12.259 | 27.413 | 40.890 | 1.00 | 65.54 | |
| ATOM | 11 | N | ALA | 205 | 10.872 | 28.478 | 39.463 | 1.00 | 63.97 | |
| ATOM | 12 | CA | ALA | 205 | 9.773 | 28.563 | 40.431 | 1.00 | 62.46 | |
| ATOM | 13 | CB | ALA | 205 | 8.437 | 28.736 | 39.705 | 1.00 | 62.71 | |
| ATOM | 14 | C | ALA | 205 | 9.798 | 27.243 | 41.190 | 1.00 | 61.33 | |
| ATOM | 15 | O | ALA | 205 | 9.426 | 26.199 | 40.647 | 1.00 | 61.43 | |
| ATOM | 16 | N | GLN | 206 | 10.251 | 27.285 | 42.439 | 1.00 | 59.43 | |
| ATOM | 17 | CA | GLN | 206 | 10.372 | 26.060 | 43.211 | 1.00 | 57.60 | |
| ATOM | 18 | CB | GLN | 206 | 11.198 | 26.298 | 44.472 | 1.00 | 58.55 | |
| ATOM | 19 | CG | GLN | 206 | 11.976 | 25.062 | 44.863 | 1.00 | 60.08 | |
| ATOM | 20 | CD | GLN | 206 | 12.831 | 24.542 | 43.712 | 1.00 | 61.69 | |
| ATOM | 21 | OE1 | GLN | 206 | 13.892 | 25.094 | 43.411 | 1.00 | 62.30 | |
| ATOM | 22 | NE2 | GLN | 206 | 12.360 | 23.486 | 43.053 | 1.00 | 61.64 | |
| ATOM | 23 | C | GLN | 206 | 9.072 | 25.355 | 43.567 | 1.00 | 55.19 | |
| ATOM | 24 | O | GLN | 206 | 8.089 | 25.972 | 43.983 | 1.00 | 55.16 | |
| ATOM | 25 | N | GLU | 207 | 9.099 | 24.040 | 43.382 | 1.00 | 52.39 | |
| ATOM | 26 | CA | GLU | 207 | 7.970 | 23.165 | 43.644 | 1.00 | 49.47 | |
| ATOM | 27 | CB | GLU | 207 | 7.755 | 22.243 | 42.447 | 1.00 | 52.06 | |
| ATOM | 28 | CG | GLU | 207 | 6.603 | 21.264 | 42.591 | 1.00 | 55.45 | |
| ATOM | 29 | CD | GLU | 207 | 5.266 | 21.897 | 42.275 | 1.00 | 57.54 | |
| ATOM | 30 | OE1 | GLU | 207 | 4.249 | 21.167 | 42.254 | 1.00 | 58.63 | |
| ATOM | 31 | OE2 | GLU | 207 | 5.235 | 23.125 | 42.043 | 1.00 | 58.53 | |
| ATOM | 32 | C | GLU | 207 | 8.273 | 22.311 | 44.861 | 1.00 | 45.64 | |
| ATOM | 33 | O | GLU | 207 | 9.419 | 21.945 | 45.089 | 1.00 | 44.54 | |
| ATOM | 34 | N | LEU | 208 | 7.244 | 21.996 | 45.637 | 1.00 | 41.48 | |
| ATOM | 35 | CA | LEU | 208 | 7.408 | 21.142 | 46.810 | 1.00 | 38.35 | |
| ATOM | 36 | CB | LEU | 208 | 6.204 | 21.323 | 47.752 | 1.00 | 36.20 | |
| ATOM | 37 | CG | LEU | 208 | 6.211 | 20.671 | 49.134 | 1.00 | 34.07 | |
| ATOM | 38 | CD1 | LEU | 208 | 7.495 | 21.026 | 49.867 | 1.00 | 32.61 | |
| ATOM | 39 | CD2 | LEU | 208 | 5.003 | 21.158 | 49.926 | 1.00 | 33.18 | |
| ATOM | 40 | C | LEU | 208 | 7.472 | 19.709 | 46.267 | 1.00 | 38.07 | |
| ATOM | 41 | O | LEU | 208 | 6.443 | 19.122 | 45.919 | 1.00 | 38.80 | |
| ATOM | 42 | N | SER | 209 | 8.682 | 19.155 | 46.174 | 1.00 | 34.84 | |
| ATOM | 43 | CA | SER | 209 | 8.882 | 17.803 | 45.647 | 1.00 | 33.19 | |
| ATOM | 44 | CB | SER | 209 | 9.257 | 17.883 | 44.165 | 1.00 | 32.93 | |
| ATOM | 45 | OG | SER | 209 | 10.582 | 18.382 | 44.024 | 1.00 | 33.12 | |
| ATOM | 46 | C | SER | 209 | 10.005 | 17.062 | 46.393 | 1.00 | 32.84 | |
| ATOM | 47 | O | SER | 209 | 10.824 | 17.685 | 47.057 | 1.00 | 32.20 | |
| ATOM | 48 | N | ILE | 210 | 10.048 | 15.736 | 46.261 | 1.00 | 32.33 | |
| ATOM | 49 | CA | ILE | 210 | 11.092 | 14.945 | 46.917 | 1.00 | 34.10 | |
| ATOM | 50 | CB | ILE | 210 | 10.961 | 13.438 | 46.613 | 1.00 | 35.86 | |
| ATOM | 51 | CG2 | ILE | 210 | 12.017 | 12.667 | 47.387 | 1.00 | 37.65 | |
| ATOM | 52 | CG1 | ILE | 210 | 9.565 | 12.929 | 46.980 | 1.00 | 36.30 | |
| ATOM | 53 | CD1 | ILE | 210 | 9.239 | 13.004 | 48.447 | 1.00 | 35.19 | |
| ATOM | 54 | C | ILE | 210 | 12.478 | 15.370 | 46.436 | 1.00 | 33.53 | |
| ATOM | 55 | O | ILE | 210 | 13.420 | 15.467 | 47.225 | 1.00 | 30.63 | |
| ATOM | 56 | N | GLU | 211 | 12.607 | 15.609 | 45.136 | 1.00 | 33.07 | |
| ATOM | 57 | CA | GLU | 211 | 13.898 | 16.012 | 44.587 | 1.00 | 33.86 | |
| ATOM | 58 | CB | GLU | 211 | 13.797 | 16.199 | 43.066 | 1.00 | 36.35 | |
| ATOM | 59 | CG | GLU | 211 | 15.042 | 16.838 | 42.436 | 1.00 | 40.50 | |
| ATOM | 60 | CD | GLU | 211 | 14.880 | 17.104 | 40.941 | 1.00 | 43.91 | |
| ATOM | 61 | OE1 | GLU | 211 | 15.777 | 17.748 | 40.348 | 1.00 | 45.56 | |
| ATOM | 62 | OE2 | GLU | 211 | 13.857 | 16.667 | 40.365 | 1.00 | 44.68 | |
| ATOM | 63 | C | GLU | 211 | 14.396 | 17.299 | 45.246 | 1.00 | 32.93 | |
| ATOM | 64 | O | GLU | 211 | 15.552 | 17.395 | 45.637 | 1.00 | 32.96 | |
| ATOM | 65 | N | ARG | 212 | 13.524 | 18.292 | 45.365 | 1.00 | 31.58 | |
| ATOM | 66 | CA | ARG | 212 | 13.914 | 19.545 | 45.994 | 1.00 | 30.93 | |
| ATOM | 67 | CB | ARG | 212 | 12.799 | 20.579 | 45.850 | 1.00 | 31.30 | |
| ATOM | 68 | CG | ARG | 212 | 13.111 | 21.897 | 46.547 | 1.00 | 34.79 | |
| ATOM | 69 | CD | ARG | 212 | 14.482 | 22.417 | 46.130 | 1.00 | 36.82 | |
| ATOM | 70 | NE | ARG | 212 | 14.880 | 23.599 | 46.886 | 1.00 | 40.98 | |
| ATOM | 71 | CZ | ARG | 212 | 16.109 | 24.103 | 46.876 | 1.00 | 41.42 | |
| ATOM | 72 | NH1 | ARG | 212 | 17.055 | 23.527 | 46.148 | 1.00 | 43.03 | |
| ATOM | 73 | NH2 | ARG | 212 | 16.395 | 25.178 | 47.596 | 1.00 | 43.71 | |
| ATOM | 74 | C | ARG | 212 | 14.277 | 19.375 | 47.478 | 1.00 | 29.92 | |
| ATOM | 75 | O | ARG | 212 | 15.218 | 20.000 | 47.970 | 1.00 | 30.04 | |
| ATOM | 76 | N | LEU | 213 | 13.529 | 18.541 | 48.195 | 1.00 | 28.52 | |
| ATOM | 77 | CA | LEU | 213 | 13.819 | 18.322 | 49.612 | 1.00 | 27.44 | |
| ATOM | 78 | CB | LEU | 213 | 12.711 | 17.471 | 50.272 | 1.00 | 25.96 | |
| ATOM | 79 | CG | LEU | 213 | 11.361 | 18.184 | 50.410 | 1.00 | 27.73 | |
| ATOM | 80 | CD1 | LEU | 213 | 10.306 | 17.200 | 50.892 | 1.00 | 24.70 | |
| ATOM | 81 | CD2 | LEU | 213 | 11.492 | 19.344 | 51.358 | 1.00 | 27.22 | |
| ATOM | 82 | C | LEU | 213 | 15.172 | 17.625 | 49.757 | 1.00 | 27.41 | |
| ATOM | 83 | O | LEU | 213 | 15.895 | 17.861 | 50.722 | 1.00 | 27.85 | |
| ATOM | 84 | N | LEU | 214 | 15.510 | 16.761 | 48.802 | 1.00 | 28.81 | |
| ATOM | 85 | CA | LEU | 214 | 16.792 | 16.068 | 48.874 | 1.00 | 30.82 | |
| ATOM | 86 | CB | LEU | 214 | 16.863 | 14.926 | 47.851 | 1.00 | 29.94 | |
| ATOM | 87 | CG | LEU | 214 | 16.112 | 13.655 | 48.272 | 1.00 | 30.12 | |
| ATOM | 88 | CD1 | LEU | 214 | 16.047 | 12.663 | 47.117 | 1.00 | 30.68 | |
| ATOM | 89 | CD2 | LEU | 214 | 16.820 | 13.015 | 49.467 | 1.00 | 31.09 | |
| ATOM | 90 | C | LEU | 214 | 17.914 | 17.067 | 48.646 | 1.00 | 33.09 | |
| ATOM | 91 | O | LEU | 214 | 18.954 | 16.985 | 49.297 | 1.00 | 32.71 | |
| ATOM | 92 | N | GLU | 215 | 17.700 | 18.018 | 47.736 | 1.00 | 35.01 | |
| ATOM | 93 | CA | GLU | 215 | 18.702 | 19.046 | 47.471 | 1.00 | 36.86 | |
| ATOM | 94 | CB | GLU | 215 | 18.274 | 19.977 | 46.319 | 1.00 | 38.74 | |
| ATOM | 95 | CG | GLU | 215 | 18.045 | 19.310 | 44.962 | 1.00 | 42.50 | |
| ATOM | 96 | CD | GLU | 215 | 17.655 | 20.307 | 43.862 | 1.00 | 44.43 | |
| ATOM | 97 | OE1 | GLU | 215 | 17.034 | 21.350 | 44.174 | 1.00 | 44.44 | |
| ATOM | 98 | OE2 | GLU | 215 | 17.957 | 20.039 | 42.674 | 1.00 | 46.05 | |
| ATOM | 99 | C | GLU | 215 | 18.881 | 19.886 | 48.735 | 1.00 | 36.72 | |
| ATOM | 100 | O | GLU | 215 | 19.996 | 20.278 | 49.082 | 1.00 | 37.76 | |
| ATOM | 101 | N | MET | 216 | 17.779 | 20.159 | 49.430 | 1.00 | 35.08 | |
| ATOM | 102 | CA | MET | 216 | 17.814 | 20.970 | 50.644 | 1.00 | 34.20 | |
| ATOM | 103 | CB | MET | 216 | 16.393 | 21.402 | 51.018 | 1.00 | 32.09 | |
| ATOM | 104 | CG | MET | 216 | 15.820 | 22.514 | 50.188 | 1.00 | 31.14 | |
| ATOM | 105 | SD | MET | 216 | 14.067 | 22.656 | 50.591 | 1.00 | 29.63 | |
| ATOM | 106 | CE | MET | 216 | 14.187 | 23.383 | 52.223 | 1.00 | 30.33 | |
| ATOM | 107 | C | MET | 216 | 18.447 | 20.309 | 51.868 | 1.00 | 33.78 | |
| ATOM | 108 | O | MET | 216 | 18.955 | 20.998 | 52.755 | 1.00 | 32.43 | |
| ATOM | 109 | N | GLU | 217 | 18.381 | 18.982 | 51.923 | 1.00 | 34.02 | |
| ATOM | 110 | CA | GLU | 217 | 18.908 | 18.212 | 53.046 | 1.00 | 35.35 | |
| ATOM | 111 | CB | GLU | 217 | 18.470 | 16.745 | 52.919 | 1.00 | 33.44 | |
| ATOM | 112 | CG | GLU | 217 | 18.729 | 15.871 | 54.148 | 1.00 | 32.91 | |
| ATOM | 113 | CD | GLU | 217 | 17.999 | 16.372 | 55.389 | 1.00 | 32.03 | |
| ATOM | 114 | OE1 | GLU | 217 | 17.001 | 17.104 | 55.242 | 1.00 | 33.02 | |
| ATOM | 115 | OE2 | GLU | 217 | 18.419 | 16.032 | 56.511 | 1.00 | 34.58 | |
| ATOM | 116 | C | GLU | 217 | 20.421 | 18.293 | 53.105 | 1.00 | 37.01 | |
| ATOM | 117 | O | GLU | 217 | 21.006 | 18.276 | 54.190 | 1.00 | 37.08 | |
| ATOM | 118 | N | SER | 218 | 21.036 | 18.406 | 51.928 | 1.00 | 39.66 | |
| ATOM | 119 | CA | SER | 218 | 22.491 | 18.476 | 51.795 | 1.00 | 42.68 | |
| ATOM | 120 | CB | SER | 218 | 22.913 | 18.001 | 50.408 | 1.00 | 43.25 | |
| ATOM | 121 | OG | SER | 218 | 22.571 | 16.640 | 50.214 | 1.00 | 46.19 | |
| ATOM | 122 | C | SER | 218 | 23.083 | 19.856 | 52.027 | 1.00 | 44.17 | |
| ATOM | 123 | O | SER | 218 | 24.250 | 19.978 | 52.395 | 1.00 | 43.66 | |
| ATOM | 124 | N | LEU | 219 | 22.291 | 20.895 | 51.797 | 1.00 | 46.04 | |
| ATOM | 125 | CA | LEU | 219 | 22.773 | 22.254 | 51.982 | 1.00 | 48.34 | |
| ATOM | 126 | CB | LEU | 219 | 21.706 | 23.265 | 51.556 | 1.00 | 49.36 | |
| ATOM | 127 | CG | LEU | 219 | 21.670 | 23.631 | 50.072 | 1.00 | 50.71 | |
| ATOM | 128 | CD1 | LEU | 219 | 21.879 | 22.395 | 49.219 | 1.00 | 51.07 | |
| ATOM | 129 | CD2 | LEU | 219 | 20.341 | 24.307 | 49.760 | 1.00 | 51.61 | |
| ATOM | 130 | C | LEU | 219 | 23.183 | 22.530 | 53.416 | 1.00 | 49.65 | |
| ATOM | 131 | O | LEU | 219 | 22.501 | 22.140 | 54.363 | 1.00 | 49.02 | |
| ATOM | 132 | N | VAL | 220 | 24.318 | 23.205 | 53.552 | 1.00 | 51.55 | |
| ATOM | 133 | CA | VAL | 220 | 24.868 | 23.592 | 54.843 | 1.00 | 53.29 | |
| ATOM | 134 | CB | VAL | 220 | 26.250 | 22.945 | 55.085 | 1.00 | 52.35 | |
| ATOM | 135 | CG1 | VAL | 220 | 26.774 | 23.341 | 56.456 | 1.00 | 51.60 | |
| ATOM | 136 | CG2 | VAL | 220 | 26.142 | 21.426 | 54.965 | 1.00 | 50.84 | |
| ATOM | 137 | C | VAL | 220 | 25.030 | 25.106 | 54.770 | 1.00 | 55.56 | |
| ATOM | 138 | O | VAL | 220 | 25.929 | 25.613 | 54.097 | 1.00 | 56.16 | |
| ATOM | 139 | N | ALA | 221 | 24.147 | 25.823 | 55.450 | 1.00 | 57.83 | |
| ATOM | 140 | CA | ALA | 221 | 24.190 | 27.278 | 55.428 | 1.00 | 60.51 | |
| ATOM | 141 | CB | ALA | 221 | 22.782 | 27.845 | 55.601 | 1.00 | 60.73 | |
| ATOM | 142 | C | ALA | 221 | 25.108 | 27.838 | 56.499 | 1.00 | 61.87 | |
| ATOM | 143 | O | ALA | 221 | 25.475 | 27.143 | 57.452 | 1.00 | 62.13 | |
| ATOM | 144 | N | ALA | 222 | 25.490 | 29.100 | 56.326 | 1.00 | 62.85 | |
| ATOM | 145 | CA | ALA | 222 | 26.345 | 29.756 | 57.295 | 1.00 | 63.37 | |
| ATOM | 146 | CB | ALA | 222 | 26.612 | 31.196 | 56.871 | 1.00 | 63.59 | |
| ATOM | 147 | C | ALA | 222 | 25.564 | 29.719 | 58.601 | 1.00 | 63.51 | |
| ATOM | 148 | O | ALA | 222 | 24.422 | 30.176 | 58.659 | 1.00 | 63.95 | |
| ATOM | 149 | N | ALA | 223 | 26.173 | 29.150 | 59.636 | 1.00 | 63.63 | |
| ATOM | 150 | CA | ALA | 223 | 25.532 | 29.044 | 60.939 | 1.00 | 63.10 | |
| ATOM | 151 | CB | ALA | 223 | 26.558 | 28.627 | 61.984 | 1.00 | 63.64 | |
| ATOM | 152 | C | ALA | 223 | 24.874 | 30.365 | 61.339 | 1.00 | 62.58 | |
| ATOM | 153 | O | ALA | 223 | 25.557 | 31.333 | 61.678 | 1.00 | 63.33 | |
| ATOM | 154 | N | ALA | 224 | 23.544 | 30.399 | 61.290 | 1.00 | 61.31 | |
| ATOM | 155 | CA | ALA | 224 | 22.789 | 31.599 | 61.644 | 1.00 | 59.25 | |
| ATOM | 156 | CB | ALA | 224 | 21.323 | 31.440 | 61.220 | 1.00 | 58.51 | |
| ATOM | 157 | C | ALA | 224 | 22.878 | 31.880 | 63.143 | 1.00 | 58.07 | |
| ATOM | 158 | O | ALA | 224 | 22.988 | 30.943 | 63.939 | 1.00 | 58.18 | |
| ATOM | 159 | N | GLU | 225 | 22.844 | 33.159 | 63.528 | 1.00 | 56.07 | |
| ATOM | 160 | CA | GLU | 225 | 22.909 | 33.507 | 64.950 | 1.00 | 54.05 | |
| ATOM | 161 | CB | GLU | 225 | 22.498 | 34.969 | 65.221 | 1.00 | 54.53 | |
| ATOM | 162 | CG | GLU | 225 | 22.700 | 35.401 | 66.703 | 1.00 | 55.83 | |
| ATOM | 163 | CD | GLU | 225 | 21.439 | 35.872 | 67.407 | 1.00 | 56.62 | |
| ATOM | 164 | OE1 | GLU | 225 | 21.407 | 35.822 | 68.663 | 1.00 | 57.35 | |
| ATOM | 165 | OE2 | GLU | 225 | 20.464 | 36.314 | 66.743 | 1.00 | 57.42 | |
| ATOM | 166 | C | GLU | 225 | 21.901 | 32.594 | 65.611 | 1.00 | 52.71 | |
| ATOM | 167 | O | GLU | 225 | 20.737 | 32.557 | 65.201 | 1.00 | 52.66 | |
| ATOM | 168 | N | GLU | 226 | 22.334 | 31.840 | 66.612 | 1.00 | 50.26 | |
| ATOM | 169 | CA | GLU | 226 | 21.391 | 30.960 | 67.256 | 1.00 | 47.92 | |
| ATOM | 170 | CB | GLU | 226 | 21.858 | 29.513 | 67.196 | 1.00 | 49.61 | |
| ATOM | 171 | CG | GLU | 226 | 20.778 | 28.525 | 67.641 | 1.00 | 49.78 | |
| ATOM | 172 | CD | GLU | 226 | 19.333 | 28.959 | 67.359 | 1.00 | 51.91 | |
| ATOM | 173 | OE1 | GLU | 226 | 18.452 | 28.312 | 67.956 | 1.00 | 50.08 | |
| ATOM | 174 | OE2 | GLU | 226 | 19.039 | 29.909 | 66.543 | 1.00 | 51.77 | |
| ATOM | 175 | C | GLU | 226 | 21.015 | 31.344 | 68.670 | 1.00 | 46.28 | |
| ATOM | 176 | O | GLU | 226 | 21.839 | 31.794 | 69.476 | 1.00 | 47.17 | |
| ATOM | 177 | N | PHE | 227 | 19.733 | 31.155 | 68.942 | 1.00 | 41.52 | |
| ATOM | 178 | CA | PHE | 227 | 19.138 | 31.503 | 70.206 | 1.00 | 38.73 | |
| ATOM | 179 | CB | PHE | 227 | 17.723 | 32.022 | 69.963 | 1.00 | 40.03 | |
| ATOM | 180 | CG | PHE | 227 | 17.640 | 33.061 | 68.876 | 1.00 | 40.91 | |
| ATOM | 181 | CD1 | PHE | 227 | 17.775 | 32.703 | 67.542 | 1.00 | 40.72 | |
| ATOM | 182 | CD2 | PHE | 227 | 17.464 | 34.402 | 69.192 | 1.00 | 42.62 | |
| ATOM | 183 | CE1 | PHE | 227 | 17.739 | 33.661 | 66.535 | 1.00 | 42.04 | |
| ATOM | 184 | CE2 | PHE | 227 | 17.426 | 35.374 | 68.188 | 1.00 | 42.27 | |
| ATOM | 185 | CZ | PHE | 227 | 17.564 | 34.996 | 66.857 | 1.00 | 42.02 | |
| ATOM | 186 | C | PHE | 227 | 19.119 | 30.339 | 71.174 | 1.00 | 34.83 | |
| AOTM | 187 | O | PHE | 227 | 19.218 | 29.178 | 70.778 | 1.00 | 34.52 | |
| ATOM | 188 | N | GLN | 228 | 18.982 | 30.687 | 72.445 | 1.00 | 33.69 | |
| ATOM | 189 | CA | GLN | 228 | 18.979 | 29.735 | 73.540 | 1.00 | 32.01 | |
| ATOM | 190 | CB | GLN | 228 | 19.290 | 30.468 | 74.847 | 1.00 | 34.80 | |
| ATOM | 191 | CG | GLN | 228 | 20.680 | 31.080 | 74.935 | 1.00 | 39.39 | |
| ATOM | 192 | CD | GLN | 228 | 21.768 | 30.029 | 74.916 | 1.00 | 40.63 | |
| ATOM | 193 | OE1 | GLN | 228 | 22.117 | 29.504 | 73.860 | 1.00 | 44.27 | |
| ATOM | 194 | NE2 | GLN | 228 | 22.301 | 29.706 | 76.093 | 1.00 | 42.65 | |
| ATOM | 195 | C | GLN | 228 | 17.678 | 28.974 | 73.736 | 1.00 | 28.92 | |
| ATOM | 196 | O | GLN | 228 | 17.035 | 29.174 | 74.750 | 1.00 | 28.29 | |
| ATOM | 197 | N | PHE | 229 | 17.283 | 28.120 | 72.794 | 1.00 | 28.04 | |
| ATOM | 198 | CA | PHE | 229 | 16.056 | 27.340 | 72.996 | 1.00 | 25.30 | |
| ATOM | 199 | CB | PHE | 229 | 15.767 | 26.411 | 71.821 | 1.00 | 27.02 | |
| ATOM | 200 | CG | PHE | 229 | 15.066 | 27.055 | 70.680 | 1.00 | 28.71 | |
| ATOM | 201 | CD1 | PHE | 229 | 15.599 | 28.170 | 70.052 | 1.00 | 28.96 | |
| ATOM | 202 | CD2 | PHE | 229 | 13.903 | 26.480 | 70.173 | 1.00 | 30.61 | |
| ATOM | 203 | CE1 | PHE | 229 | 14.991 | 28.708 | 68.918 | 1.00 | 29.32 | |
| ATOM | 204 | CE2 | PHE | 229 | 13.284 | 25.998 | 69.046 | 1.00 | 31.44 | |
| ATOM | 205 | CZ | PHE | 229 | 13.829 | 28.117 | 68.412 | 1.00 | 31.43 | |
| ATOM | 206 | C | PHE | 229 | 16.276 | 26.434 | 74.195 | 1.00 | 23.81 | |
| ATOM | 207 | O | PHE | 229 | 15.385 | 26.202 | 75.014 | 1.00 | 22.15 | |
| ATOM | 208 | N | LEU | 230 | 17.487 | 25.899 | 74.263 | 1.00 | 20.44 | |
| ATOM | 209 | CA | LEU | 230 | 17.848 | 24.964 | 75.302 | 1.00 | 21.42 | |
| ATOM | 210 | CB | LEU | 230 | 18.255 | 23.631 | 74.650 | 1.00 | 20.34 | |
| ATOM | 211 | CG | LEU | 230 | 17.191 | 22.855 | 73.834 | 1.00 | 22.70 | |
| ATOM | 212 | CD1 | LEU | 230 | 17.860 | 21.762 | 73.013 | 1.00 | 23.14 | |
| ATOM | 213 | CD2 | LEU | 230 | 16.130 | 22.252 | 74.773 | 1.00 | 21.55 | |
| ATOM | 214 | C | LEU | 230 | 19.017 | 25.540 | 76.094 | 1.00 | 20.71 | |
| ATOM | 215 | O | LEU | 230 | 19.977 | 26.038 | 75.524 | 1.00 | 20.61 | |
| ATOM | 216 | N | ARG | 231 | 18.931 | 25.467 | 77.411 | 1.00 | 20.48 | |
| ATOM | 217 | CA | ARG | 231 | 20.018 | 25.997 | 78.211 | 1.00 | 20.62 | |
| ATOM | 218 | CB | ARG | 231 | 20.023 | 27.525 | 78.176 | 1.00 | 20.50 | |
| ATOM | 219 | CG | ARG | 231 | 18.907 | 28.184 | 79.017 | 1.00 | 24.52 | |
| ATOM | 220 | CD | ARG | 231 | 17.560 | 28.025 | 78.342 | 1.00 | 27.62 | |
| ATOM | 221 | NE | ARG | 231 | 16.465 | 28.674 | 79.060 | 1.00 | 28.14 | |
| ATOM | 222 | CZ | ARG | 231 | 15.846 | 28.165 | 80.118 | 1.00 | 28.35 | |
| ATOM | 223 | NH1 | ARG | 231 | 16.208 | 26.986 | 80.598 | 1.00 | 27.27 | |
| ATOM | 224 | NH2 | ARG | 231 | 14.856 | 28.835 | 80.692 | 1.00 | 28.94 | |
| ATOM | 225 | C | ARG | 231 | 19.957 | 25.585 | 79.663 | 1.00 | 20.62 | |
| ATOM | 226 | O | ARG | 231 | 18.884 | 25.350 | 80.219 | 1.00 | 20.08 | |
| ATOM | 227 | N | VAL | 232 | 21.138 | 25.549 | 80.265 | 1.00 | 20.70 | |
| ATOM | 228 | CA | VAL | 232 | 21.276 | 25.244 | 81.684 | 1.00 | 23.23 | |
| ATOM | 229 | CB | VAL | 232 | 22.596 | 24.499 | 81.961 | 1.00 | 24.57 | |
| ATOM | 230 | CG1 | VAL | 232 | 22.800 | 24.335 | 83.460 | 1.00 | 24.88 | |
| ATOM | 231 | CG2 | VAL | 232 | 22.565 | 23.135 | 81.289 | 1.00 | 23.34 | |
| ATOM | 232 | C | VAL | 232 | 21.306 | 26.619 | 82.359 | 1.00 | 25.86 | |
| ATOM | 233 | O | VAL | 232 | 22.284 | 27.349 | 82.225 | 1.00 | 29.87 | |
| ATOM | 234 | N | GLY | 233 | 20.232 | 26.960 | 83.062 | 1.00 | 24.64 | |
| ATOM | 235 | CA | GLY | 233 | 20.137 | 28.249 | 83.730 | 1.00 | 26.40 | |
| ATOM | 236 | C | GLY | 233 | 20.170 | 28.129 | 85.240 | 1.00 | 27.72 | |
| ATOM | 237 | O | GLY | 233 | 20.449 | 27.041 | 85.755 | 1.00 | 26.07 | |
| ATOM | 238 | N | PRO | 234 | 19.863 | 29.206 | 85.980 | 1.00 | 28.00 | |
| ATOM | 239 | CD | PRO | 234 | 19.340 | 30.507 | 85.515 | 1.00 | 30.45 | |
| ATOM | 240 | CA | PRO | 234 | 19.886 | 29.158 | 87.447 | 1.00 | 28.48 | |
| ATOM | 241 | CB | PRO | 234 | 19.772 | 30.627 | 87.833 | 1.00 | 30.42 | |
| ATOM | 242 | CG | PRO | 234 | 18.804 | 31.123 | 86.802 | 1.00 | 32.04 | |
| ATOM | 243 | C | PRO | 234 | 18.752 | 28.323 | 88.037 | 1.00 | 27.91 | |
| ATOM | 244 | O | PRO | 234 | 18.781 | 27.975 | 89.229 | 1.00 | 28.03 | |
| ATOM | 245 | N | ASP | 235 | 17.757 | 27.996 | 87.210 | 1.00 | 26.13 | |
| ATOM | 246 | CA | ASP | 235 | 16.636 | 27.186 | 87.680 | 1.00 | 23.93 | |
| ATOM | 247 | CB | ASP | 235 | 15.333 | 27.686 | 87.064 | 1.00 | 28.77 | |
| ATOM | 248 | CG | ASP | 235 | 15.405 | 27.820 | 85.544 | 1.00 | 31.24 | |
| ATOM | 249 | OD1 | ASP | 235 | 16.519 | 27.836 | 84.964 | 1.00 | 32.56 | |
| ATOM | 250 | OD2 | ASP | 235 | 14.327 | 27.922 | 84.919 | 1.00 | 34.70 | |
| ATOM | 251 | C | ASP | 235 | 16.828 | 25.700 | 87.362 | 1.00 | 20.99 | |
| ATOM | 252 | O | ASP | 235 | 16.019 | 24.881 | 87.751 | 1.00 | 19.94 | |
| ATOM | 253 | N | SER | 236 | 17.914 | 25.369 | 86.666 | 0.50 | 20.63 | AC1 |
| ATOM | 254 | CA | SER | 236 | 18.208 | 23.979 | 86.280 | 0.50 | 20.15 | AC1 |
| ATOM | 255 | CB | SER | 236 | 19.196 | 23.969 | 85.111 | 0.50 | 19.44 | AC1 |
| ATOM | 256 | OG | SER | 236 | 18.708 24.734 | 84.013 | 0.50 | 19.16 | AC1 | |
| ATOM | 257 | C | SER | 236 | 18.787 | 23.137 | 87.425 | 0.50 | 21.81 | AC1 |
| ATOM | 258 | O | SER | 236 | 19.849 | 23.3457 | 87.946 | 0.50 | 20.58 | AC1 |
| ATOM | 259 | N | ASN | 237 | 18.125 | 22.037 | 87.782 | 1.00 | 21.94 | |
| ATOM | 260 | CA | ASN | 237 | 18.621 | 21.202 | 88.886 | 1.00 | 25.34 | |
| ATOM | 261 | CB | ASN | 237 | 17.498 | 20.331 | 89.450 | 1.00 | 30.84 | |
| ATOM | 262 | CG | ASN | 237 | 17.020 | 19.301 | 88.472 | 1.00 | 34.04 | |
| ATOM | 263 | OD1 | ASN | 237 | 17.786 | 18.838 | 87.623 | 1.00 | 39.37 | |
| ATOM | 264 | ND2 | ASN | 237 | 15.748 | 18.913 | 88.588 | 1.00 | 39.05 | |
| ATOM | 265 | C | ASN | 237 | 19.843 | 20.325 | 88.581 | 1.00 | 26.93 | |
| ATOM | 266 | O | ASN | 237 | 20.180 | 19.405 | 89.339 | 1.00 | 32.05 | |
| ATOM | 267 | N | VAL | 238 | 20.537 | 20.621 | 87.503 | 1.00 | 23.03 | |
| ATOM | 268 | CA | VAL | 238 | 21.713 | 19.862 | 87.114 | 1.00 | 20.81 | |
| ATOM | 269 | CB | VAL | 238 | 22.096 | 20.241 | 85.663 | 1.00 | 20.25 | |
| ATOM | 270 | CG1 | VAL | 238 | 23.336 | 19.495 | 85.207 | 1.00 | 19.20 | |
| ATOM | 271 | CG2 | VAL | 238 | 20.919 | 19.922 | 84.741 | 1.00 | 20.14 | |
| ATOM | 272 | C | VAL | 238 | 22.844 | 20.185 | 88.106 | 1.00 | 20.62 | |
| ATOM | 273 | O | VAL | 238 | 23.147 | 21.343 | 88.363 | 1.00 | 18.94 | |
| ATOM | 274 | N | PRO | 239 | 23.428 | 19.153 | 88.719 | 1.00 | 20.32 | |
| ATOM | 275 | CD | PRO | 239 | 23.144 | 17.726 | 88.541 | 1.00 | 20.40 | |
| ATOM | 276 | CA | PRO | 239 | 24.515 | 19.328 | 89.688 | 1.00 | 21.49 | |
| ATOM | 277 | CB | PRO | 239 | 24.911 | 17.892 | 90.023 | 1.00 | 22.13 | |
| ATOM | 278 | CG | PRO | 239 | 23.696 | 17.174 | 89.847 | 1.00 | 20.51 | |
| ATOM | 279 | C | PRO | 239 | 25.663 | 20.039 | 89.023 | 1.00 | 24.59 | |
| ATOM | 280 | O | PRO | 239 | 25.924 | 19.804 | 87.847 | 1.00 | 23.65 | |
| ATOM | 281 | N | PRO | 240 | 26.388 | 20.879 | 89.777 | 1.00 | 24.92 | |
| ATOM | 282 | CD | PRO | 240 | 26.142 | 21.311 | 91.167 | 1.00 | 26.02 | |
| ATOM | 283 | CA | PRO | 240 | 27.524 | 21.603 | 89.202 | 1.00 | 26.88 | |
| ATOM | 284 | CB | PRO | 240 | 28.222 | 22.161 | 90.438 | 1.00 | 26.73 | |
| ATOM | 285 | CG | PRO | 240 | 27.039 | 22.543 | 91.299 | 1.00 | 29.18 | |
| ATOM | 286 | C | PRO | 240 | 28.434 | 20.733 | 88.363 | 1.00 | 26.35 | |
| ATOM | 287 | O | PRO | 240 | 28.847 | 21.125 | 87.259 | 1.00 | 27.72 | |
| ATOM | 288 | N | LYS | 241 | 28.745 | 19.533 | 88.838 | 1.00 | 28.50 | |
| ATOM | 289 | CA | LYS | 241 | 29.642 | 18.682 | 88.071 | 1.00 | 28.19 | |
| ATOM | 290 | CB | LYS | 241 | 30.073 | 17.457 | 88.878 | 1.00 | 32.48 | |
| ATOM | 291 | CG | LYS | 241 | 28.970 | 16.508 | 89.292 | 1.00 | 34.64 | |
| ATOM | 292 | CD | LYS | 241 | 29.627 | 15.241 | 89.832 | 1.00 | 37.47 | |
| ATOM | 293 | CE | LYS | 241 | 28.627 | 14.162 | 90.157 | 1.00 | 39.69 | |
| ATOM | 294 | NZ | LYS | 241 | 29.359 | 12.932 | 90.578 | 1.00 | 42.50 | |
| ATOM | 295 | C | LYS | 241 | 29.144 | 18.233 | 86.700 | 1.00 | 27.86 | |
| ATOM | 296 | O | LYS | 241 | 29.935 | 17.790 | 85.868 | 1.00 | 27.57 | |
| ATOM | 297 | N | PHE | 242 | 27.840 | 18.335 | 86.459 | 1.00 | 25.10 | |
| ATOM | 298 | CA | PHE | 242 | 27.309 | 17.953 | 85.154 | 1.00 | 22.57 | |
| ATOM | 299 | CB | PHE | 242 | 26.161 | 16.954 | 85.319 | 1.00 | 24.91 | |
| ATOM | 300 | CG | PHE | 242 | 26.600 | 15.619 | 85.856 | 1.00 | 28.41 | |
| ATOM | 301 | CD1 | PHE | 242 | 27.411 | 14.790 | 85.102 | 1.00 | 30.20 | |
| ATOM | 302 | CD2 | PHE | 242 | 26.194 | 15.191 | 87.108 | 1.00 | 28.96 | |
| ATOM | 303 | CE1 | PHE | 242 | 27.813 | 13.544 | 85.583 | 1.00 | 31.58 | |
| ATOM | 304 | CE2 | PHE | 242 | 26.594 | 13.943 | 87.596 | 1.00 | 30.20 | |
| ATOM | 305 | CZ | PHE | 242 | 27.400 | 13.127 | 86.832 | 1.00 | 32.03 | |
| ATOM | 306 | C | PHE | 242 | 26.837 | 19.151 | 84.330 | 1.00 | 19.63 | |
| ATOM | 307 | O | PHE | 242 | 26.386 | 18.991 | 83.190 | 1.00 | 20.79 | |
| ATOM | 308 | N | ARG | 243 | 26.971 | 20.350 | 84.873 | 1.00 | 19.69 | |
| ATOM | 309 | CA | ARG | 243 | 26.513 | 21.529 | 84.153 | 1.00 | 20.41 | |
| ATOM | 310 | CB | ARG | 243 | 26.538 | 22.749 | 85.066 | 1.00 | 20.87 | |
| ATOM | 311 | CG | ARG | 243 | 25.523 | 22.602 | 86.208 | 1.00 | 23.08 | |
| ATOM | 312 | CD | ARG | 243 | 25.562 | 23.759 | 87.130 | 1.00 | 26.38 | |
| ATOM | 313 | NE | ARG | 243 | 24.725 | 24.856 | 86.687 | 1.00 | 27.91 | |
| ATOM | 314 | CZ | ARG | 243 | 23.394 | 24.873 | 86.728 | 1.00 | 28.35 | |
| ATOM | 315 | NH1 | ARG | 243 | 22.697 | 23.825 | 87.192 | 1.00 | 26.91 | |
| ATOM | 316 | NH2 | ARG | 243 | 22.764 | 25.975 | 86.343 | 1.00 | 28.11 | |
| ATOM | 317 | C | ARG | 243 | 27.238 | 21.813 | 82.846 | 1.00 | 20.13 | |
| ATOM | 318 | O | ARG | 243 | 26.596 | 22.114 | 81.836 | 1.00 | 19.71 | |
| ATOM | 319 | N | ALA | 244 | 28.564 | 21.734 | 82.838 | 1.00 | 20.90 | |
| ATOM | 320 | CA | ALA | 244 | 29.273 | 21.974 | 81.577 | 1.00 | 22.06 | |
| ATOM | 321 | CB | ALA | 244 | 30.773 | 21.896 | 81.796 | 1.00 | 21.20 | |
| ATOM | 322 | C | ALA | 244 | 28.842 | 20.984 | 80.492 | 1.00 | 21.05 | |
| ATOM | 323 | O | ALA | 244 | 28.527 | 21.365 | 79.366 | 1.00 | 21.57 | |
| ATOM | 324 | N | PRO | 245 | 28.783 | 19.696 | 80.822 | 1.00 | 21.63 | |
| ATOM | 325 | CD | PRO | 245 | 29.335 | 19.087 | 82.049 | 1.00 | 25.65 | |
| ATOM | 326 | CA | PRO | 245 | 28.377 | 18.672 | 79.861 | 1.00 | 22.28 | |
| ATOM | 327 | CB | PRO | 245 | 28.516 | 17.380 | 80.666 | 1.00 | 24.91 | |
| ATOM | 328 | CG | PRO | 245 | 29.614 | 17.694 | 81.605 | 1.00 | 25.26 | |
| ATOM | 329 | C | PRO | 245 | 26.943 | 18.859 | 79.329 | 1.00 | 19.49 | |
| ATOM | 330 | O | PRO | 245 | 26.691 | 18.778 | 78.118 | 1.00 | 21.31 | |
| ATOM | 331 | N | VAL | 246 | 26.013 | 19.123 | 80.230 | 1.00 | 20.18 | |
| ATOM | 332 | CA | VAL | 246 | 24.624 | 19.294 | 79.805 | 1.00 | 18.44 | |
| ATOM | 333 | CB | VAL | 246 | 23.693 | 19.269 | 81.028 | 1.00 | 18.35 | |
| ATOM | 334 | CG1 | VAL | 246 | 22.221 | 19.536 | 80.620 | 1.00 | 18.11 | |
| ATOM | 335 | CG2 | VAL | 246 | 23.766 | 17.845 | 81.678 | 1.00 | 17.69 | |
| ATOM | 336 | C | VAL | 246 | 24.501 | 20.568 | 78.968 | 1.00 | 18.23 | |
| ATOM | 337 | O | VAL | 246 | 23.773 | 20.603 | 77.977 | 1.00 | 18.23 | |
| ATOM | 338 | N | SER | 247 | 25.234 | 21.609 | 79.351 | 0.50 | 20.08 | AC1 |
| ATOM | 339 | CA | SER | 247 | 25.198 | 22.858 | 78.599 | 0.50 | 21.19 | AC1 |
| ATOM | 340 | CB | SER | 247 | 26.058 | 23.910 | 79.289 | 0.50 | 21.37 | AC1 |
| ATOM | 341 | OG | SER | 247 | 25.484 | 24.292 | 80.517 | 0.50 | 23.67 | AC1 |
| ATOM | 342 | C | SER | 247 | 25.712 | 22.621 | 77.189 | 0.50 | 21.32 | |
| ATOM | 343 | O | SER | 247 | 25.180 | 23.161 | 76.217 | 0.50 | 21.62 | |
| ATOM | 344 | N | SER | 248 | 26.756 | 21.811 | 77.076 | 1.00 | 20.43 | |
| ATOM | 345 | CA | SER | 248 | 27.322 | 21.502 | 75.766 | 1.00 | 21.62 | |
| ATOM | 346 | CB | SER | 248 | 28.541 | 20.594 | 75.916 | 1.00 | 24.31 | |
| ATOM | 347 | OG | SER | 248 | 29.660 | 21.350 | 76.354 | 1.00 | 24.89 | |
| ATOM | 348 | C | SER | 248 | 26.271 | 20.800 | 74.903 | 1.00 | 22.36 | |
| ATOM | 349 | O | SER | 248 | 26.158 | 21.061 | 73.697 | 1.00 | 22.32 | |
| ATOM | 350 | N | LEU | 249 | 25.513 | 19.895 | 75.519 | 1.00 | 22.35 | |
| ATOM | 351 | CA | LEU | 249 | 24.462 | 19.185 | 74.791 | 1.00 | 22.28 | |
| ATOM | 352 | CB | LEU | 249 | 23.904 | 18.046 | 75.668 | 1.00 | 22.23 | |
| ATOM | 353 | CG | LEU | 249 | 24.970 | 16.972 | 75.954 | 1.00 | 25.52 | |
| ATOM | 354 | CD1 | LEU | 249 | 24.441 | 15.919 | 76.922 | 1.00 | 26.39 | |
| ATOM | 355 | CD2 | LEU | 249 | 25.398 | 16.341 | 74.643 | 1.00 | 27.01 | |
| ATOM | 356 | C | LEU | 249 | 23.358 | 20.152 | 74.352 | 1.00 | 21.63 | |
| ATOM | 357 | O | LEU | 249 | 22.822 | 20.039 | 73.231 | 1.00 | 19.99 | |
| ATOM | 358 | N | CYS | 250 | 23.012 | 21.102 | 75.217 | 1.00 | 21.10 | |
| ATOM | 359 | CA | CYS | 250 | 21.995 | 22.098 | 74.856 | 1.00 | 21.22 | |
| ATOM | 360 | CB | CYS | 250 | 21.701 | 23.044 | 76.019 | 1.00 | 20.65 | |
| ATOM | 361 | SG | CYS | 250 | 20.853 | 22.249 | 77.396 | 1.00 | 22.08 | |
| ATOM | 362 | C | CYS | 250 | 22.471 | 22.932 | 73.684 | 1.00 | 22.49 | |
| ATOM | 363 | O | CYS | 250 | 21.687 | 23.302 | 72.828 | 1.00 | 23.08 | |
| ATOM | 364 | N | GLN | 251 | 23.765 | 23.239 | 73.656 | 1.00 | 22.61 | |
| ATOM | 365 | CA | GLN | 251 | 24.313 | 24.064 | 72.577 | 1.00 | 24.50 | |
| ATOM | 366 | CB | GLN | 251 | 25.738 | 24.482 | 72.925 | 1.00 | 26.13 | |
| ATOM | 367 | CG | GLN | 251 | 25.827 | 25.428 | 74.123 | 1.00 | 31.89 | |
| ATOM | 368 | CD | GLN | 251 | 27.270 | 25.602 | 74.609 | 1.00 | 36.32 | |
| ATOM | 369 | OE1 | GLN | 251 | 28.146 | 26.020 | 73.846 | 1.00 | 38.70 | |
| ATOM | 370 | NE2 | GLN | 251 | 27.520 | 25.275 | 75.883 | 1.00 | 36.33 | |
| ATOM | 371 | C | GLN | 251 | 24.290 | 23.309 | 71.255 | 1.00 | 23.96 | |
| ATOM | 372 | O | GLN | 251 | 24.080 | 23.899 | 70.200 | 1.00 | 25.04 | |
| ATOM | 373 | N | ILE | 252 | 24.506 | 21.998 | 71.323 | 1.00 | 23.74 | |
| ATOM | 374 | CA | ILE | 252 | 24.475 | 21.143 | 70.140 | 1.00 | 24.05 | |
| ATOM | 375 | CB | ILE | 252 | 24.877 | 19.690 | 70.493 | 1.00 | 25.71 | |
| ATOM | 376 | CG2 | ILE | 262 | 24.385 | 18.727 | 69.419 | 1.00 | 25.12 | |
| ATOM | 377 | CG1 | ILE | 252 | 26.405 | 19.596 | 70.658 | 1.00 | 26.17 | |
| ATOM | 378 | CD1 | ILE | 252 | 26.874 | 18.335 | 71.359 | 1.00 | 27.45 | |
| ATOM | 379 | C | ILE | 252 | 34.032 | 21.183 | 69.642 | 1.00 | 25.30 | |
| ATOM | 380 | O | ILE | 252 | 22.760 | 21.312 | 68.448 | 1.00 | 25.88 | |
| ATOM | 381 | N | GLY | 253 | 22.101 | 21.105 | 70.580 | 1.00 | 25.76 | |
| ATOM | 382 | CA | GLY | 253 | 20.698 | 21.167 | 70.213 | 1.00 | 24.72 | |
| ATOM | 383 | C | GLY | 253 | 20.327 | 22.497 | 69.573 | 1.00 | 24.98 | |
| ATOM | 384 | O | GLY | 253 | 19.611 | 22.527 | 68.561 | 1.00 | 24.56 | |
| ATOM | 385 | N | ASN | 254 | 20.779 | 23.600 | 70.165 | 1.00 | 23.66 | |
| ATOM | 386 | CA | ASN | 254 | 20.483 | 24.929 | 69.642 | 1.00 | 24.18 | |
| ATOM | 387 | CB | ASN | 254 | 21.063 | 25.999 | 70.566 | 1.00 | 23.04 | |
| ATOM | 388 | CG | ASN | 254 | 20.336 | 26.061 | 71.884 | 1.00 | 24.90 | |
| ATOM | 389 | OD1 | ASN | 254 | 19.147 | 25.729 | 71.954 | 1.00 | 22.87 | |
| ATOM | 390 | ND2 | ASN | 254 | 21.020 | 26.511 | 72.934 | 1.00 | 25.39 | |
| ATOM | 391 | C | ASN | 254 | 20.990 | 25.142 | 68.215 | 1.00 | 25.59 | |
| ATOM | 392 | O | ASN | 254 | 20.349 | 25.832 | 67.418 | 1.00 | 25.49 | |
| ATOM | 393 | N | LYS | 255 | 22.154 | 24.575 | 67.915 | 1.00 | 24.87 | |
| ATOM | 394 | CA | LYS | 255 | 22.718 | 24.669 | 66.572 | 1.00 | 26.12 | |
| ATOM | 395 | CB | LYS | 255 | 24.121 | 24.061 | 66.518 | 1.00 | 29.40 | |
| ATOM | 396 | CG | LYS | 255 | 25.162 | 24.983 | 67.112 | 1.00 | 33.68 | |
| ATOM | 397 | CD | LYS | 255 | 26.481 | 24.293 | 67.392 | 1.00 | 34.86 | |
| ATOM | 398 | CE | LYS | 255 | 27.280 | 24.027 | 66.150 | 1.00 | 35.57 | |
| ATOM | 399 | NZ | LYS | 255 | 28.719 | 23.853 | 66.527 | 1.00 | 33.75 | |
| ATOM | 400 | C | LYS | 255 | 21.816 | 23.924 | 65.608 | 1.00 | 26.04 | |
| ATOM | 401 | O | LYS | 255 | 21.539 | 24.415 | 64.507 | 1.00 | 25.57 | |
| ATOM | 402 | N | GLN | 256 | 21.358 | 22.741 | 66.009 | 1.00 | 24.02 | |
| ATOM | 403 | CA | GLN | 256 | 20.498 | 21.967 | 65.121 | 1.00 | 23.89 | |
| ATOM | 404 | CB | GLN | 256 | 20.325 | 20.546 | 65.658 | 1.00 | 25.47 | |
| ATOM | 405 | CG | GLN | 256 | 21.676 | 19.880 | 65.887 | 1.00 | 28.63 | |
| ATOM | 406 | CD | GLN | 256 | 21.565 | 18.517 | 66.534 | 1.00 | 30.93 | |
| ATOM | 407 | OE1 | GLN | 256 | 20.710 | 18.301 | 67.387 | 1.00 | 32.63 | |
| ATOM | 408 | NE2 | GLN | 256 | 22.439 | 17.599 | 66.149 | 1.00 | 30.18 | |
| ATOM | 409 | C | GLN | 256 | 19.156 | 22.658 | 64.915 | 1.00 | 24.45 | |
| ATOM | 410 | O | GLN | 256 | 18.596 | 22.598 | 63.828 | 1.00 | 24.20 | |
| ATOM | 411 | N | ILE | 257 | 18.662 | 23.338 | 65.942 | 1.00 | 23.21 | |
| ATOM | 412 | CA | ILE | 257 | 17.390 | 24.059 | 65.816 | 1.00 | 22.41 | |
| ATOM | 413 | CB | ILE | 257 | 16.895 | 24.535 | 67.180 | 1.00 | 20.26 | |
| ATOM | 414 | CG2 | ILE | 257 | 15.636 | 25.401 | 67.021 | 1.00 | 21.15 | |
| ATOM | 415 | CG1 | ILE | 257 | 16.607 | 23.298 | 68.039 | 1.00 | 22.12 | |
| ATOM | 416 | CD1 | ILE | 257 | 16.309 | 23.620 | 69.517 | 1.00 | 21.36 | |
| ATOM | 417 | C | ILE | 257 | 17.558 | 25.253 | 64.877 | 1.00 | 23.91 | |
| ATOM | 418 | O | ILE | 257 | 16.677 | 25.544 | 64.060 | 1.00 | 21.49 | |
| ATOM | 419 | N | ALA | 258 | 18.684 | 25.949 | 64.994 | 1.00 | 23.86 | |
| ATOM | 420 | CA | ALA | 258 | 18.939 | 27.081 | 64.103 | 1.00 | 25.37 | |
| ATOM | 421 | CB | ALA | 258 | 20.313 | 27.705 | 64.416 | 1.00 | 26.26 | |
| ATOM | 422 | C | ALA | 258 | 18.906 | 26.588 | 62.656 | 1.00 | 25.01 | |
| ATOM | 423 | O | ALA | 258 | 18.306 | 27.238 | 61.783 | 1.00 | 25.91 | |
| ATOM | 424 | N | ALA | 259 | 19.555 | 25.450 | 62.403 | 1.00 | 23.72 | |
| ATOM | 425 | CA | ALA | 259 | 19.602 | 24.865 | 61.063 | 1.00 | 24.51 | |
| ATOM | 426 | CB | ALA | 259 | 20.442 | 23.613 | 61.058 | 1.00 | 24.62 | |
| ATOM | 427 | C | ALA | 259 | 18.187 | 24.525 | 60.623 | 1.00 | 24.36 | |
| ATOM | 428 | O | ALA | 259 | 17.846 | 24.693 | 59.464 | 1.00 | 23.27 | |
| ATOM | 429 | N | LEU | 260 | 17.374 | 24.015 | 61.544 | 1.00 | 23.02 | |
| ATOM | 430 | CA | LEU | 260 | 15.986 | 23.685 | 61.188 | 1.00 | 24.75 | |
| ATOM | 431 | CB | LEU | 260 | 15.237 | 23.070 | 62.366 | 1.00 | 26.22 | |
| ATOM | 432 | CG | LEU | 260 | 15.550 | 21.633 | 62.742 | 1.00 | 31.27 | |
| ATOM | 433 | CD1 | LEU | 260 | 14.906 | 21.342 | 64.082 | 1.00 | 32.20 | |
| ATOM | 434 | CD2 | LEU | 260 | 15.054 | 20.679 | 61.658 | 1.00 | 33.32 | |
| ATOM | 435 | C | LEU | 260 | 15.214 | 24.902 | 60.750 | 1.00 | 25.40 | |
| ATOM | 436 | O | LEU | 260 | 14.391 | 24.821 | 59.834 | 1.00 | 23.40 | |
| ATOM | 437 | N | VAL | 261 | 15.439 | 26.031 | 61.419 | 1.00 | 25.21 | |
| ATOM | 438 | CA | VAL | 261 | 14.735 | 27.247 | 61.055 | 1.00 | 26.95 | |
| ATOM | 439 | CB | VAL | 261 | 15.050 | 28.411 | 62.036 | 1.00 | 25.46 | |
| ATOM | 440 | CG1 | VAL | 261 | 14.386 | 29.700 | 61.544 | 1.00 | 27.40 | |
| ATOM | 441 | CG2 | VAL | 261 | 14.520 | 28.075 | 63.434 | 1.00 | 27.42 | |
| ATOM | 442 | C | VAL | 261 | 15.104 | 27.671 | 59.640 | 1.00 | 26.53 | |
| ATOM | 443 | O | VAL | 261 | 14.232 | 28.035 | 58.850 | 1.00 | 25.34 | |
| ATOM | 444 | N | VAL | 262 | 16.396 | 27.611 | 59.320 | 1.00 | 27.29 | |
| ATOM | 445 | CA | VAL | 262 | 16.874 | 27.995 | 57.993 | 1.00 | 28.11 | |
| ATOM | 446 | CB | VAL | 262 | 18.430 | 27.910 | 57.905 | 1.00 | 30.08 | |
| ATOM | 447 | CG1 | VAL | 262 | 18.883 | 27.872 | 56.441 | 1.00 | 33.75 | |
| ATOM | 448 | CG2 | VAL | 262 | 19.051 | 29.104 | 58.606 | 1.00 | 32.97 | |
| ATOM | 449 | C | VAL | 262 | 16.267 | 27.075 | 56.939 | 1.00 | 26.73 | |
| ATOM | 450 | O | VAL | 262 | 15.909 | 27.511 | 55.840 | 1.00 | 26.28 | |
| ATOM | 451 | N | TRP | 263 | 16.177 | 25.794 | 57.286 | 1.00 | 24.38 | |
| ATOM | 452 | CA | TRP | 263 | 15.623 | 24.775 | 56.402 | 1.00 | 25.26 | |
| ATOM | 453 | CB | TRP | 263 | 15.831 | 23.406 | 57.052 | 1.00 | 23.10 | |
| ATOM | 454 | CG | TRP | 263 | 15.102 | 22.286 | 56.409 | 1.00 | 24.27 | |
| ATOM | 455 | CD2 | TRP | 263 | 13.881 | 21.697 | 56.873 | 1.00 | 24.86 | |
| ATOM | 456 | CE2 | TRP | 263 | 13.536 | 20.681 | 55.962 | 1.00 | 25.93 | |
| ATOM | 457 | CE3 | TRP | 263 | 13.051 | 21.936 | 57.974 | 1.00 | 24.53 | |
| ATOM | 458 | CD1 | TRP | 263 | 15.441 | 21.624 | 55.267 | 1.00 | 24.80 | |
| ATOM | 459 | NE1 | TRP | 263 | 14.501 | 20.655 | 54.990 | 1.00 | 27.16 | |
| ATOM | 460 | CZ2 | TRP | 263 | 12.391 | 19.895 | 56.115 | 1.00 | 26.26 | |
| ATOM | 461 | CZ3 | TRP | 263 | 11.911 | 21.151 | 58.132 | 1.00 | 23.32 | |
| ATOM | 462 | CH2 | TRP | 263 | 11.598 | 20.144 | 57.204 | 1.00 | 23.73 | |
| ATOM | 463 | C | TRP | 263 | 14.125 | 25.047 | 56.175 | 1.00 | 24.66 | |
| ATOM | 464 | O | TRP | 263 | 13.645 | 25.081 | 55.037 | 1.00 | 25.81 | |
| ATOM | 465 | N | ALA | 264 | 13.391 | 25.278 | 57.252 | 1.00 | 24.56 | |
| ATOM | 466 | CA | ALA | 264 | 11.949 | 25.506 | 57.103 | 1.00 | 25.04 | |
| ATOM | 467 | CB | ALA | 264 | 11.304 | 25.733 | 58.464 | 1.00 | 26.00 | |
| ATOM | 468 | C | ALA | 264 | 11.675 | 26.701 | 56.205 | 1.00 | 27.22 | |
| ATOM | 469 | O | ALA | 264 | 10.838 | 26.640 | 55.293 | 1.00 | 24.71 | |
| ATOM | 470 | N | ARG | 265 | 12.372 | 27.794 | 56.489 | 1.00 | 28.16 | |
| ATOM | 471 | CA | ARG | 265 | 12.227 | 29.024 | 55.724 | 1.00 | 32.11 | |
| ATOM | 472 | CB | ARG | 265 | 13.250 | 30.066 | 56.218 | 1.00 | 33.47 | |
| ATOM | 473 | CG | ARG | 265 | 13.155 | 31.408 | 55.520 | 1.00 | 36.25 | |
| ATOM | 474 | CD | ARG | 265 | 14.169 | 32.397 | 56.078 | 1.00 | 39.06 | |
| ATOM | 475 | NE | ARG | 265 | 15.545 | 31.972 | 55.823 | 1.00 | 42.06 | |
| ATOM | 476 | CZ | ARG | 265 | 16.609 | 32.534 | 56.391 | 1.00 | 43.94 | |
| ATOM | 477 | NH1 | ARG | 265 | 16.447 | 33.541 | 57.244 | 1.00 | 44.49 | |
| ATOM | 478 | NH2 | ARG | 265 | 17.827 | 32.092 | 56.110 | 1.00 | 43.52 | |
| ATOM | 479 | C | ARG | 265 | 12.424 | 28.767 | 54.225 | 1.00 | 32.76 | |
| ATOM | 480 | O | ARG | 265 | 11.843 | 29.460 | 53.392 | 1.00 | 35.61 | |
| ATOM | 481 | N | ASP | 266 | 13.227 | 27.771 | 53.872 | 1.00 | 33.71 | |
| ATOM | 482 | CA | ASP | 266 | 13.465 | 27.492 | 52.466 | 1.00 | 34.17 | |
| ATOM | 483 | CB | ASP | 266 | 14.931 | 27.106 | 52.236 | 1.00 | 37.32 | |
| ATOM | 484 | CG | ASP | 266 | 15.879 | 28.293 | 52.374 | 1.00 | 40.62 | |
| ATOM | 485 | OD1 | ASP | 266 | 15.556 | 29.392 | 51.866 | 1.00 | 40.66 | |
| ATOM | 486 | OD2 | ASP | 266 | 16.959 | 28.128 | 52.985 | 1.00 | 43.94 | |
| ATOM | 487 | C | ASP | 266 | 12.544 | 26.447 | 51.833 | 1.00 | 33.93 | |
| ATOM | 488 | O | ASP | 266 | 12.664 | 26.156 | 50.646 | 1.00 | 32.38 | |
| ATOM | 489 | N | ILE | 267 | 11.640 | 25.869 | 52.619 | 1.00 | 32.18 | |
| ATOM | 490 | CA | ILE | 267 | 10.694 | 24.897 | 52.077 | 1.00 | 30.21 | |
| ATOM | 491 | CB | ILE | 267 | 9.820 | 24.279 | 53.210 | 1.00 | 29.16 | |
| ATOM | 492 | CG2 | ILE | 267 | 8.586 | 23.588 | 52.620 | 1.00 | 31.21 | |
| ATOM | 493 | CG1 | ILE | 267 | 10.643 | 23.291 | 54.038 | 1.00 | 29.01 | |
| ATOM | 494 | CD1 | ILE | 267 | 11.069 | 22.051 | 53.278 | 1.00 | 28.89 | |
| ATOM | 495 | C | ILE | 267 | 9.800 | 25.670 | 51.093 | 1.00 | 30.06 | |
| ATOM | 496 | O | ILE | 267 | 9.256 | 26.715 | 51.421 | 1.00 | 29.41 | |
| ATOM | 497 | N | PRO | 268 | 9.653 | 25.164 | 49.862 | 1.00 | 31.41 | |
| ATOM | 498 | CD | PRO | 268 | 10.216 | 23.921 | 49.300 | 1.00 | 31.06 | |
| ATOM | 499 | CA | PRO | 268 | 8.813 | 25.857 | 48.879 | 1.00 | 31.62 | |
| ATOM | 500 | CB | PRO | 268 | 8.686 | 24.830 | 47.755 | 1.00 | 31.63 | |
| ATOM | 501 | CG | PRO | 268 | 10.012 | 24.126 | 47.811 | 1.00 | 34.16 | |
| ATOM | 502 | C | PRO | 268 | 7.459 | 26.286 | 49.444 | 1.00 | 31.83 | |
| ATOM | 503 | O | PRO | 268 | 6.762 | 25.483 | 50.051 | 1.00 | 31.61 | |
| ATOM | 504 | N | HIS | 269 | 7.128 | 27.566 | 49.267 | 1.00 | 31.08 | |
| ATOM | 505 | CA | HIS | 269 | 5.867 | 28.164 | 49.715 | 1.00 | 31.47 | |
| ATOM | 506 | CB | HIS | 269 | 4.677 | 27.277 | 49.300 | 1.00 | 33.82 | |
| ATOM | 507 | CG | HIS | 269 | 4.710 | 26.845 | 47.865 | 1.00 | 36.44 | |
| ATOM | 508 | CD2 | HIS | 269 | 4.694 | 25.611 | 47.305 | 1.00 | 37.13 | |
| ATOM | 509 | ND1 | HIS | 269 | 4.734 | 27.740 | 46.816 | 1.00 | 39.48 | |
| ATOM | 510 | CE1 | HIS | 269 | 4.731 | 27.078 | 45.672 | 1.00 | 37.91 | |
| ATOM | 511 | NE2 | HIS | 269 | 4.706 | 25.785 | 45.941 | 1.00 | 39.31 | |
| ATOM | 512 | C | HIS | 269 | 5.745 | 28.465 | 51.210 | 1.00 | 30.01 | |
| ATOM | 513 | O | HIS | 269 | 4.796 | 29.133 | 51.638 | 1.00 | 28.74 | |
| ATOM | 514 | N | PHE | 270 | 6.693 | 27.994 | 52.012 | 1.00 | 28.26 | |
| ATOM | 515 | CA | PHE | 270 | 6.607 | 28.222 | 53.454 | 1.00 | 27.87 | |
| ATOM | 516 | CB | PHE | 270 | 7.728 | 27.468 | 54.178 | 1.00 | 26.66 | |
| ATOM | 517 | CG | PHE | 270 | 7.661 | 27.563 | 55.684 | 1.00 | 24.18 | |
| ATOM | 518 | CD1 | PHE | 270 | 6.867 | 26.683 | 56.415 | 1.00 | 24.15 | |
| ATOM | 519 | CD2 | PHE | 270 | 8.385 | 28.536 | 56.372 | 1.00 | 26.34 | |
| ATOM | 520 | CE1 | PHE | 270 | 6.792 | 26.775 | 57.822 | 1.00 | 22.45 | |
| ATOM | 521 | CE2 | PHE | 270 | 8.316 | 28.637 | 57.778 | 1.00 | 24.45 | |
| ATOM | 522 | CZ | PHE | 270 | 7.516 | 27.754 | 58.494 | 1.00 | 23.03 | |
| ATOM | 523 | C | PHE | 270 | 6.650 | 29.707 | 53.811 | 1.00 | 28.65 | |
| ATOM | 524 | O | PHE | 270 | 5.866 | 30.175 | 54.634 | 1.00 | 29.19 | |
| ATOM | 525 | N | SER | 271 | 7.558 | 30.448 | 53.184 | 0.50 | 29.83 | AC1 |
| ATOM | 526 | CA | SER | 271 | 7.676 | 31.876 | 53.463 | 0.50 | 31.34 | AC1 |
| ATOM | 527 | CB | SER | 271 | 8.959 | 32.432 | 52.839 | 0.50 | 31.74 | AC1 |
| ATOM | 528 | OG | SER | 271 | 10.104 | 31.869 | 53.460 | 0.50 | 31.64 | AC1 |
| ATOM | 529 | C | SER | 271 | 6.458 | 32.663 | 52.974 | 0.50 | 32.85 | AC1 |
| ATOM | 530 | O | SER | 271 | 6.301 | 33.839 | 53.296 | 0.50 | 33.99 | AC1 |
| ATOM | 531 | N | GLN | 272 | 5.599 | 32.009 | 52.197 | 1.00 | 33.79 | |
| ATOM | 532 | CA | GLN | 272 | 4.378 | 32.642 | 51.696 | 1.00 | 35.22 | |
| ATOM | 533 | CB | GLN | 272 | 3.910 | 31.928 | 50.423 | 1.00 | 39.02 | |
| ATOM | 534 | CG | GLN | 272 | 4.777 | 32.210 | 49.191 | 1.00 | 43.59 | |
| ATOM | 535 | CD | GLN | 272 | 4.608 | 31.169 | 48.086 | 1.00 | 45.89 | |
| ATOM | 536 | OE1 | GLN | 272 | 3.488 | 30.794 | 47.727 | 1.00 | 47.68 | |
| ATOM | 537 | NE2 | GLN | 272 | 5.729 | 30.703 | 47.534 | 1.00 | 48.15 | |
| ATOM | 538 | C | GLN | 272 | 3.255 | 32.633 | 52.742 | 1.00 | 35.20 | |
| ATOM | 539 | O | GLN | 272 | 2.288 | 33.397 | 52.648 | 1.00 | 33.55 | |
| ATOM | 540 | N | LEU | 273 | 3.383 | 31.756 | 53.736 | 1.00 | 33.18 | |
| ATOM | 541 | CA | LEU | 273 | 2.402 | 31.651 | 54.811 | 1.00 | 31.26 | |
| ATOM | 542 | CB | LEU | 273 | 2.695 | 30.398 | 55.653 | 1.00 | 30.14 | |
| ATOM | 543 | CG | LEU | 273 | 2.671 | 29.015 | 54.988 | 1.00 | 29.04 | |
| ATOM | 544 | CD1 | LEU | 273 | 3.273 | 27.989 | 55.930 | 1.00 | 28.99 | |
| ATOM | 545 | CD2 | LEU | 273 | 1.231 | 28.638 | 54.645 | 1.00 | 29.21 | |
| ATOM | 546 | C | LEU | 273 | 2.500 | 32.880 | 55.722 | 1.00 | 30.95 | |
| ATOM | 547 | O | LEU | 273 | 3.556 | 33.513 | 55.793 | 1.00 | 30.58 | |
| ATOM | 548 | N | GLU | 274 | 1.416 | 33.208 | 56.420 | 1.00 | 31.28 | |
| ATOM | 549 | CA | GLU | 274 | 1.435 | 34.332 | 57.355 | 1.00 | 32.85 | |
| ATOM | 550 | CB | GLU | 274 | 0.154 | 34.378 | 58.195 | 1.00 | 35.60 | |
| ATOM | 551 | CG | GLU | 274 | −1.039 | 35.022 | 57.511 | 1.00 | 40.50 | |
| ATOM | 552 | CD | GLU | 274 | −0.954 | 36.543 | 57.494 | 1.00 | 43.35 | |
| ATOM | 553 | OE1 | GLU | 274 | −1.788 | 37.171 | 56.807 | 1.00 | 44.88 | |
| ATOM | 554 | OE2 | GLU | 274 | −0.062 | 37.109 | 58.169 | 1.00 | 45.29 | |
| ATOM | 555 | C | GLU | 274 | 2.615 | 34.079 | 58.287 | 1.00 | 32.91 | |
| ATOM | 556 | O | GLU | 274 | 2.867 | 32.936 | 58.693 | 1.00 | 31.56 | |
| ATOM | 557 | N | MET | 275 | 3.331 | 35.136 | 58.632 | 1.00 | 31.54 | |
| ATOM | 558 | CA | MET | 275 | 4.483 | 35.010 | 59.507 | 1.00 | 31.78 | |
| ATOM | 559 | CB | MET | 275 | 5.094 | 36.392 | 59.748 | 1.00 | 34.73 | |
| ATOM | 560 | CG | MET | 275 | 6.288 | 36.403 | 60.673 | 1.00 | 37.61 | |
| ATOM | 561 | SD | MET | 275 | 7.574 | 35.262 | 60.158 | 1.00 | 39.49 | |
| ATOM | 562 | CE | MET | 275 | 7.940 | 35.869 | 58.496 | 1.00 | 39.54 | |
| ATOM | 563 | C | MET | 275 | 4.149 | 34.351 | 60.838 | 1.00 | 31.91 | |
| ATOM | 564 | O | MET | 275 | 4.885 | 33.474 | 61.305 | 1.00 | 31.90 | |
| ATOM | 565 | N | GLU | 276 | 3.052 | 34.764 | 61.458 | 1.00 | 31.07 | |
| ATOM | 566 | CA | GLU | 276 | 2.684 | 34.184 | 62.736 | 1.00 | 30.79 | |
| ATOM | 567 | CB | GLU | 276 | 1.499 | 34.938 | 63.341 | 1.00 | 35.06 | |
| ATOM | 568 | CG | GLU | 276 | 1.866 | 36.382 | 63.755 | 1.00 | 37.66 | |
| ATOM | 569 | CD | GLU | 276 | 3.043 | 36.434 | 64.731 | 1.00 | 40.34 | |
| ATOM | 570 | OE1 | GLU | 276 | 2.978 | 35.751 | 65.774 | 1.00 | 41.45 | |
| ATOM | 571 | OE2 | GLU | 276 | 4.034 | 37.157 | 64.464 | 1.00 | 42.59 | |
| ATOM | 572 | C | GLU | 276 | 2.388 | 32.693 | 62.596 | 1.00 | 29.05 | |
| ATOM | 573 | O | GLU | 276 | 2.529 | 31.946 | 63.559 | 1.00 | 28.93 | |
| ATOM | 574 | N | ASP | 277 | 1.973 | 32.250 | 61.411 | 1.00 | 26.93 | |
| ATOM | 575 | CA | ASP | 277 | 1.716 | 30.817 | 61.223 | 1.00 | 24.95 | |
| ATOM | 576 | CB | ASP | 277 | 0.817 | 30.565 | 60.005 | 1.00 | 26.30 | |
| ATOM | 577 | CG | ASP | 277 | −0.656 | 30.768 | 60.320 | 1.00 | 26.04 | |
| ATOM | 578 | OD1 | ASP | 277 | −0.984 | 31.102 | 61.476 | 1.00 | 29.19 | |
| ATOM | 579 | OD2 | ASP | 277 | −1.492 | 30.596 | 59.410 | 1.00 | 26.42 | |
| ATOM | 580 | C | ASP | 277 | 3.056 | 30.089 | 61.069 | 1.00 | 25.34 | |
| ATOM | 581 | O | ASP | 277 | 3.226 | 28.967 | 61.579 | 1.00 | 25.43 | |
| ATOM | 582 | N | GLN | 278 | 4.007 | 30.721 | 60.373 | 1.00 | 23.47 | |
| ATOM | 583 | CA | GLN | 278 | 5.338 | 30.132 | 60.203 | 1.00 | 23.93 | |
| ATOM | 584 | CB | GLN | 278 | 6.275 | 31.094 | 59.467 | 1.00 | 23.08 | |
| ATOM | 585 | CG | GLN | 278 | 5.931 | 31.265 | 57.999 | 1.00 | 26.36 | |
| ATOM | 586 | CD | GLN | 278 | 6.858 | 32.238 | 57.324 | 1.00 | 26.00 | |
| ATOM | 587 | OE1 | GLN | 278 | 8.075 | 32.122 | 57.439 | 1.00 | 26.10 | |
| ATOM | 588 | NE2 | GLN | 278 | 6.293 | 33.210 | 56.609 | 1.00 | 28.94 | |
| ATOM | 589 | C | GLN | 278 | 5.917 | 29.850 | 61.588 | 1.00 | 22.78 | |
| ATOM | 590 | O | GLN | 278 | 6.445 | 28.765 | 61.857 | 1.00 | 20.87 | |
| ATOM | 591 | N | ILE | 279 | 5.821 | 30.849 | 62.461 | 1.00 | 23.32 | |
| ATOM | 592 | CA | ILE | 279 | 6.322 | 30.719 | 63.825 | 1.00 | 22.80 | |
| ATOM | 593 | CB | ILE | 279 | 6.125 | 32.046 | 64.591 | 1.00 | 24.62 | |
| ATOM | 594 | CG2 | ILE | 279 | 6.449 | 31.868 | 66.076 | 1.00 | 24.08 | |
| ATOM | 595 | CG1 | ILE | 279 | 6.997 | 33.125 | 63.943 | 1.00 | 27.52 | |
| ATOM | 596 | CD1 | ILE | 279 | 6.728 | 34.543 | 64.464 | 1.00 | 27.69 | |
| ATOM | 597 | C | ILE | 279 | 5.638 | 29.560 | 64.573 | 1.00 | 22.81 | |
| ATOM | 598 | O | ILE | 279 | 6.294 | 28.758 | 65.215 | 1.00 | 23.50 | |
| ATOM | 599 | N | LEU | 280 | 4.318 | 29.463 | 64.498 | 1.00 | 20.16 | |
| ATOM | 600 | CA | LEU | 280 | 3.653 | 28.382 | 65.186 | 1.00 | 18.30 | |
| ATOM | 601 | CB | LEU | 280 | 2.125 | 28.553 | 65.113 | 1.00 | 19.64 | |
| ATOM | 602 | CG | LEU | 280 | 1.589 | 29.739 | 65.931 | 1.00 | 22.60 | |
| ATOM | 603 | CD1 | LEU | 280 | 0.093 | 29.931 | 65.623 | 1.00 | 24.59 | |
| ATOM | 604 | CD2 | LEU | 280 | 1.759 | 29.496 | 67.399 | 1.00 | 25.05 | |
| ATOM | 605 | C | LEU | 280 | 4.045 | 27.028 | 64.653 | 1.00 | 17.14 | |
| ATOM | 606 | O | LEU | 280 | 4.160 | 26.084 | 65.436 | 1.00 | 18.84 | |
| ATOM | 607 | N | LEU | 281 | 4.225 | 26.905 | 63.335 | 1.00 | 17.52 | |
| ATOM | 608 | CA | LEU | 281 | 4.586 | 25.591 | 62.773 | 1.00 | 19.05 | |
| ATOM | 609 | CB | LEU | 281 | 4.550 | 25.618 | 61.241 | 1.00 | 19.60 | |
| ATOM | 610 | CG | LEU | 281 | 3.167 | 25.782 | 60.595 | 1.00 | 21.40 | |
| ATOM | 611 | CD1 | LEU | 281 | 3.324 | 25.870 | 59.073 | 1.00 | 23.62 | |
| ATOM | 612 | CD2 | LEU | 281 | 2.266 | 24.621 | 60.975 | 1.00 | 21.20 | |
| ATOM | 613 | C | LEU | 281 | 5.968 | 25.143 | 63.255 | 1.00 | 19.84 | |
| ATOM | 614 | O | LEU | 281 | 6.164 | 23.977 | 63.620 | 1.00 | 19.38 | |
| ATOM | 615 | N | ILE | 282 | 6.923 | 26.067 | 63.269 | 1.00 | 20.43 | |
| ATOM | 616 | CA | ILE | 282 | 8.270 | 25.740 | 63.731 | 1.00 | 19.64 | |
| ATOM | 617 | CB | ILE | 282 | 9.274 | 26.881 | 63.361 | 1.00 | 20.44 | |
| ATOM | 618 | CG2 | ILE | 282 | 10.619 | 26.706 | 64.102 | 1.00 | 20.04 | |
| ATOM | 619 | CG1 | ILE | 282 | 9.486 | 26.873 | 61.839 | 1.00 | 20.45 | |
| ATOM | 620 | CD1 | ILE | 282 | 10.278 | 28.076 | 61.323 | 1.00 | 22.03 | |
| ATOM | 621 | C | ILE | 282 | 8.255 | 25.496 | 65.239 | 1.00 | 19.41 | |
| ATOM | 622 | O | ILE | 282 | 8.894 | 24.574 | 65.717 | 1.00 | 19.21 | |
| ATOM | 623 | N | LYS | 283 | 7.533 | 26.322 | 65.992 | 1.00 | 17.59 | |
| ATOM | 624 | CA | LYS | 283 | 7.480 | 26.148 | 67.446 | 1.00 | 19.01 | |
| ATOM | 625 | CB | LYS | 283 | 6.624 | 27.246 | 68.103 | 1.00 | 19.89 | |
| ATOM | 626 | CG | LYS | 283 | 6.596 | 27.154 | 69.613 | 1.00 | 23.82 | |
| ATOM | 627 | CD | LYS | 283 | 5.948 | 28.388 | 70.243 | 1.00 | 26.99 | |
| ATOM | 628 | CE | LYS | 283 | 5.645 | 28.183 | 71.731 | 1.00 | 30.98 | |
| ATOM | 629 | NZ | LYS | 283 | 6.837 | 27.917 | 72.599 | 1.00 | 34.38 | |
| ATOM | 630 | C | LYS | 283 | 6.873 | 24.791 | 67.778 | 1.00 | 19.44 | |
| ATOM | 631 | O | LYS | 283 | 7.274 | 24.120 | 68.729 | 1.00 | 19.31 | |
| ATOM | 632 | N | GLY | 284 | 5.882 | 24.390 | 66.983 | 1.00 | 19.18 | |
| ATOM | 633 | CA | GLY | 284 | 5.240 | 23.124 | 67.254 | 1.00 | 18.56 | |
| ATOM | 634 | C | GLY | 284 | 5.981 | 21.885 | 66.806 | 1.00 | 18.31 | |
| ATOM | 635 | O | GLY | 284 | 5.731 | 20.805 | 67.328 | 1.00 | 19.56 | |
| ATOM | 636 | N | SER | 285 | 6.941 | 22.024 | 65.895 | 1.00 | 18.65 | |
| ATOM | 637 | CA | SER | 285 | 7.633 | 20.844 | 65.384 | 1.00 | 17.51 | |
| ATOM | 638 | CB | SER | 285 | 7.453 | 20.755 | 63.870 | 1.00 | 19.70 | |
| ATOM | 639 | OG | SER | 285 | 8.063 | 21.874 | 63.212 | 1.00 | 18.48 | |
| ATOM | 640 | C | SER | 285 | 9.136 | 20.728 | 65.663 | 1.00 | 15.25 | |
| ATOM | 641 | O | SER | 285 | 9.705 | 19.673 | 65.406 | 1.00 | 15.43 | |
| ATOM | 642 | N | TRP | 286 | 9.770 | 21.759 | 66.203 | 1.00 | 15.99 | |
| ATOM | 643 | CA | TRP | 286 | 11.221 | 21.642 | 66.395 | 1.00 | 16.28 | |
| ATOM | 644 | CB | TRP | 286 | 11.812 | 22.920 | 67.007 | 1.00 | 17.10 | |
| ATOM | 645 | CG | TRP | 286 | 11.458 | 23.226 | 68.414 | 1.00 | 17.32 | |
| ATOM | 646 | CD2 | TRP | 286 | 12.117 | 22.736 | 69.592 | 1.00 | 18.06 | |
| ATOM | 647 | CE2 | TRP | 286 | 11.440 | 23.291 | 70.709 | 1.00 | 20.12 | |
| ATOM | 648 | CE3 | TRP | 286 | 13.208 | 21.882 | 69.815 | 1.00 | 17.92 | |
| ATOM | 649 | CD1 | TRP | 286 | 10.445 | 24.033 | 68.850 | 1.00 | 18.61 | |
| ATOM | 650 | NE1 | TRP | 286 | 10.427 | 24.076 | 70.227 | 1.00 | 18.32 | |
| ATOM | 651 | CZ2 | TRP | 286 | 11.816 | 23.020 | 72.027 | 1.00 | 17.23 | |
| ATOM | 652 | CZ3 | TRP | 286 | 13.580 | 21.608 | 71.136 | 1.00 | 18.42 | |
| ATOM | 653 | CH2 | TRP | 286 | 12.889 | 22.172 | 72.219 | 1.00 | 19.75 | |
| ATOM | 654 | C | TRP | 286 | 11.668 | 20.443 | 67.219 | 1.00 | 16.59 | |
| ATOM | 655 | O | TRP | 286 | 12.655 | 19.766 | 66.886 | 1.00 | 17.31 | |
| ATOM | 656 | N | ASN | 287 | 10.938 | 20.167 | 68.282 | 1.00 | 15.26 | |
| ATOM | 657 | CA | ASN | 287 | 11.301 | 19.059 | 69.157 | 1.00 | 14.41 | |
| ATOM | 658 | CB | ASN | 287 | 10.508 | 19.180 | 70.456 | 1.00 | 17.68 | |
| ATOM | 659 | CG | ASN | 287 | 10.894 | 18.145 | 71.474 | 1.00 | 19.61 | |
| ATOM | 660 | OD1 | ASN | 287 | 11.757 | 18.374 | 72.348 | 1.00 | 23.92 | |
| ATOM | 661 | ND2 | ASN | 287 | 10.251 | 16.991 | 71.384 | 1.00 | 18.83 | |
| ATOM | 662 | C | ASN | 287 | 11.096 | 17.718 | 68.448 | 1.00 | 14.74 | |
| ATOM | 663 | O | ASN | 287 | 11.954 | 16.846 | 68.508 | 1.00 | 16.16 | |
| ATOM | 664 | N | GLU | 288 | 9.966 | 17.555 | 67.752 | 1.00 | 15.22 | |
| ATOM | 665 | CA | GLU | 288 | 9.745 | 16.319 | 66.986 | 1.00 | 16.77 | |
| ATOM | 666 | CB | GLU | 288 | 8.367 | 16.358 | 66.288 | 1.00 | 17.55 | |
| ATOM | 667 | CG | GLU | 288 | 7.213 | 16.044 | 67.224 | 1.00 | 18.68 | |
| ATOM | 668 | CD | GLU | 288 | 5.868 | 16.134 | 66.548 | 1.00 | 23.02 | |
| ATOM | 669 | OE1 | GLU | 288 | 5.786 | 15.849 | 65.342 | 1.00 | 28.18 | |
| ATOM | 670 | OE2 | GLU | 288 | 4.887 | 16.461 | 67.243 | 1.00 | 29.07 | |
| ATOM | 671 | C | GLU | 288 | 10.838 | 16.122 | 65.921 | 1.00 | 17.49 | |
| ATOM | 672 | O | GLU | 288 | 11.338 | 15.014 | 65.743 | 1.00 | 16.93 | |
| ATOM | 673 | N | LEU | 289 | 11.193 | 17.204 | 65.220 | 1.00 | 14.56 | |
| ATOM | 674 | CA | LEU | 289 | 12.196 | 17.130 | 64.173 | 1.00 | 15.76 | |
| ATOM | 675 | CB | LEU | 289 | 12.248 | 18.451 | 63.390 | 1.00 | 14.77 | |
| ATOM | 676 | CG | LEU | 289 | 11.006 | 18.639 | 62.483 | 1.00 | 16.67 | |
| ATOM | 677 | CD1 | LEU | 289 | 10.932 | 20.093 | 62.002 | 1.00 | 18.25 | |
| ATOM | 678 | CD2 | LEU | 289 | 11.083 | 17.667 | 61.287 | 1.00 | 15.71 | |
| ATOM | 679 | C | LEU | 289 | 13.555 | 16.784 | 64.752 | 1.00 | 16.05 | |
| ATOM | 680 | O | LEU | 289 | 14.301 | 16.022 | 64.138 | 1.00 | 16.56 | |
| ATOM | 681 | N | LEU | 290 | 13.884 | 17.312 | 65.929 | 1.00 | 16.37 | |
| ATOM | 682 | CA | LEU | 290 | 15.190 | 16.951 | 66.529 | 1.00 | 16.68 | |
| ATOM | 683 | CB | LEU | 290 | 15.477 | 17.734 | 67.809 | 1.00 | 17.02 | |
| ATOM | 684 | CG | LEU | 290 | 15.786 | 19.219 | 67.684 | 1.00 | 22.29 | |
| ATOM | 685 | CD1 | LEU | 290 | 16.294 | 19.726 | 69.046 | 1.00 | 23.74 | |
| ATOM | 686 | CD2 | LEU | 290 | 16.865 | 19.454 | 66.604 | 1.00 | 25.08 | |
| ATOM | 687 | C | LEU | 290 | 15.229 | 15.471 | 66.868 | 1.00 | 16.18 | |
| ATOM | 688 | O | LEU | 290 | 16.214 | 14.803 | 66.605 | 1.00 | 17.45 | |
| ATOM | 689 | N | LEU | 291 | 14.142 | 14.958 | 67.465 | 1.00 | 16.16 | |
| ATOM | 690 | CA | LEU | 291 | 14.088 | 13.553 | 67.826 | 1.00 | 17.64 | |
| ATOM | 691 | CB | LEU | 291 | 12.828 | 13.289 | 68.666 | 1.00 | 17.32 | |
| ATOM | 692 | CG | LEU | 291 | 12.824 | 13.961 | 70.046 | 1.00 | 20.40 | |
| ATOM | 693 | CD1 | LEU | 291 | 11.405 | 13.964 | 70.627 | 1.00 | 22.18 | |
| ATOM | 694 | CD2 | LEU | 291 | 13.789 | 13.222 | 70.971 | 1.00 | 23.46 | |
| ATOM | 695 | C | LEU | 291 | 14.087 | 12.662 | 66.595 | 1.00 | 15.65 | |
| ATOM | 696 | O | LEU | 291 | 14.645 | 11.569 | 66.611 | 1.00 | 17.39 | |
| ATOM | 697 | N | PHE | 292 | 13.434 | 13.116 | 65.520 | 1.00 | 14.81 | |
| ATOM | 698 | CA | PHE | 292 | 13.360 | 12.330 | 64.311 | 1.00 | 15.62 | |
| ATOM | 699 | CB | PHE | 292 | 12.392 | 12.993 | 63.314 | 1.00 | 16.43 | |
| ATOM | 700 | CG | PHE | 292 | 12.003 | 12.113 | 62.167 | 1.00 | 16.59 | |
| ATOM | 701 | CD1 | PHE | 292 | 11.588 | 10.812 | 62.389 | 1.00 | 18.96 | |
| ATOM | 702 | CD2 | PHE | 292 | 12.002 | 12.607 | 60.875 | 1.00 | 19.01 | |
| ATOM | 703 | CE1 | PHE | 292 | 11.170 | 10.011 | 61.332 | 1.00 | 20.62 | |
| ATOM | 704 | CE2 | PHE | 292 | 11.581 | 11.805 | 59.805 | 1.00 | 18.46 | |
| ATOM | 705 | CZ | PHE | 292 | 11.166 | 10.499 | 60.060 | 1.00 | 18.06 | |
| ATOM | 706 | C | PHE | 292 | 14.761 | 12.191 | 63.713 | 1.00 | 15.70 | |
| ATOM | 707 | O | PHE | 292 | 15.132 | 11.119 | 63.236 | 1.00 | 15.72 | |
| ATOM | 708 | N | ALA | 293 | 15.526 | 13.278 | 63.760 | 1.00 | 16.34 | |
| ATOM | 709 | CA | ALA | 293 | 16.906 | 13.267 | 63.232 | 1.00 | 16.73 | |
| ATOM | 710 | CB | ALA | 293 | 17.464 | 14.702 | 63.207 | 1.00 | 17.74 | |
| ATOM | 711 | C | ALA | 293 | 17.802 | 12.368 | 64.090 | 1.00 | 17.09 | |
| ATOM | 712 | O | ALA | 293 | 18.668 | 11.654 | 63.570 | 1.00 | 17.27 | |
| ATOM | 713 | N | ILE | 294 | 17.623 | 12.423 | 65.407 | 1.00 | 16.09 | |
| ATOM | 714 | CA | ILE | 294 | 18.389 | 11.569 | 66.312 | 1.00 | 16.22 | |
| ATOM | 715 | CB | ILE | 294 | 18.032 | 11.857 | 67.768 | 1.00 | 15.67 | |
| ATOM | 716 | CG2 | ILE | 294 | 18.584 | 10.765 | 68.679 | 1.00 | 16.66 | |
| ATOM | 717 | CG1 | ILE | 294 | 18.615 | 13.218 | 68.160 | 1.00 | 17.00 | |
| ATOM | 718 | CD1 | ILE | 294 | 18.206 | 13.635 | 69.535 | 1.00 | 20.77 | |
| ATOM | 719 | C | ILE | 294 | 18.070 | 10.111 | 65.971 | 1.00 | 16.74 | |
| ATOM | 720 | O | ILE | 294 | 18.954 | 9.296 | 65.833 | 1.00 | 16.38 | |
| ATOM | 721 | N | ALA | 295 | 16.791 | 9.791 | 65.774 | 1.00 | 16.15 | |
| ATOM | 722 | CA | ALA | 295 | 16.452 | 8.420 | 65.424 | 1.00 | 16.99 | |
| ATOM | 723 | CB | ALA | 295 | 14.937 | 8.273 | 65.332 | 1.00 | 16.54 | |
| ATOM | 724 | C | ALA | 295 | 17.083 | 7.993 | 64.088 | 1.00 | 17.97 | |
| ATOM | 725 | O | ALA | 295 | 17.563 | 6.870 | 63.954 | 1.00 | 18.01 | |
| ATOM | 726 | N | TRP | 296 | 17.071 | 8.881 | 63.102 | 1.00 | 18.59 | |
| ATOM | 727 | CA | TRP | 296 | 17.625 | 8.570 | 61.782 | 1.00 | 18.81 | |
| ATOM | 728 | CB | TRP | 296 | 17.321 | 9.740 | 60.835 | 1.00 | 20.21 | |
| ATOM | 729 | CG | TRP | 296 | 17.849 | 9.622 | 59.451 | 1.00 | 22.61 | |
| ATOM | 730 | CD2 | TRP | 296 | 17.398 | 8.716 | 58.433 | 1.00 | 25.62 | |
| ATOM | 731 | CE2 | TRP | 296 | 18.125 | 9.007 | 57.265 | 1.00 | 26.25 | |
| ATOM | 732 | CE3 | TRP | 296 | 16.448 | 7.683 | 58.402 | 1.00 | 25.95 | |
| ATOM | 733 | CD1 | TRP | 296 | 18.807 | 10.403 | 58.871 | 1.00 | 26.23 | |
| ATOM | 734 | NE1 | TRP | 296 | 18.975 | 10.043 | 57.556 | 1.00 | 26.50 | |
| ATOM | 735 | CZ2 | TRP | 296 | 17.934 | 8.297 | 56.063 | 1.00 | 27.77 | |
| ATOM | 736 | CZ3 | TRP | 296 | 16.257 | 6.977 | 57.206 | 1.00 | 27.65 | |
| ATOM | 737 | CH2 | TRP | 296 | 16.996 | 7.289 | 56.061 | 1.00 | 25.35 | |
| ATOM | 738 | C | TRP | 296 | 19.133 | 8.299 | 61.878 | 1.00 | 19.53 | |
| ATOM | 739 | O | TRP | 296 | 19.647 | 7.296 | 61.345 | 1.00 | 21.43 | |
| ATOM | 740 | N | ARG | 297 | 19.833 | 9.147 | 62.623 | 1.00 | 18.58 | |
| ATOM | 741 | CA | ARG | 297 | 21.280 | 8.974 | 62.748 | 1.00 | 18.22 | |
| ATOM | 742 | CB | ARG | 297 | 21.923 | 10.208 | 63.376 | 1.00 | 18.92 | |
| ATOM | 743 | CG | ARG | 297 | 21.886 | 11.465 | 62.525 | 1.00 | 20.63 | |
| ATOM | 744 | CD | ARG | 297 | 22.792 | 12.530 | 63.137 | 1.00 | 26.15 | |
| ATOM | 745 | NE | ARG | 297 | 22.219 | 13.002 | 64.381 | 1.00 | 29.32 | |
| ATOM | 746 | CZ | ARG | 297 | 21.393 | 14.040 | 64.462 | 1.00 | 28.97 | |
| ATOM | 747 | NH1 | ARG | 297 | 21.066 | 14.720 | 63.373 | 1.00 | 29.81 | |
| ATOM | 748 | NH2 | ARG | 297 | 20.872 | 14.372 | 65.628 | 1.00 | 31.03 | |
| ATOM | 749 | C | ARG | 297 | 21.643 | 7.776 | 63.605 | 1.00 | 18.87 | |
| ATOM | 750 | O | ARG | 297 | 22.738 | 7.225 | 63.473 | 1.00 | 19.01 | |
| ATOM | 751 | N | SER | 298 | 20.749 | 7.404 | 64.511 | 1.00 | 17.52 | |
| ATOM | 752 | CA | SER | 298 | 21.023 | 6.293 | 65.414 | 1.00 | 18.09 | |
| ATOM | 753 | CB | SER | 298 | 20.134 | 6.407 | 66.655 | 1.00 | 18.94 | |
| ATOM | 754 | OG | SER | 298 | 20.412 | 7.606 | 67.381 | 1.00 | 18.16 | |
| ATOM | 755 | C | SER | 298 | 20.865 | 4.929 | 64.770 | 1.00 | 20.63 | |
| ATOM | 756 | O | SER | 298 | 21.310 | 3.925 | 65.336 | 1.00 | 24.22 | |
| ATOM | 757 | N | MET | 299 | 20.270 | 4.868 | 63.582 | 1.00 | 20.01 | |
| ATOM | 758 | CA | MET | 299 | 20.106 | 3.558 | 62.953 | 1.00 | 22.16 | |
| ATOM | 759 | CB | MET | 299 | 19.367 | 3.682 | 61.610 | 1.00 | 24.42 | |
| ATOM | 760 | CG | MET | 299 | 17.940 | 4.159 | 61.735 | 1.00 | 24.29 | |
| ATOM | 761 | SD | MET | 299 | 17.091 | 4.052 | 60.130 | 1.00 | 31.30 | |
| ATOM | 762 | CE | MET | 299 | 18.221 | 4.950 | 59.082 | 1.00 | 31.06 | |
| ATOM | 763 | C | MET | 299 | 21.431 | 2.825 | 62.725 | 1.00 | 25.30 | |
| ATOM | 764 | O | MET | 299 | 21.493 | 1.600 | 62.859 | 1.00 | 26.84 | |
| ATOM | 765 | N | GLU | 300 | 22.485 | 3.565 | 62.414 | 1.00 | 28.43 | |
| ATOM | 766 | CA | GLU | 300 | 23.775 | 2.944 | 62.117 | 1.00 | 32.11 | |
| ATOM | 767 | CB | GLU | 300 | 24.736 | 3.949 | 61.472 | 1.00 | 34.60 | |
| ATOM | 768 | CG | GLU | 300 | 25.355 | 4.941 | 62.420 | 1.00 | 40.06 | |
| ATOM | 769 | CD | GLU | 300 | 26.689 | 5.477 | 61.910 | 1.00 | 43.56 | |
| ATOM | 770 | OE1 | GLU | 300 | 27.622 | 4.666 | 61.729 | 1.00 | 46.83 | |
| ATOM | 771 | OE2 | GLU | 300 | 26.812 | 6.703 | 61.688 | 1.00 | 45.34 | |
| ATOM | 772 | C | GLU | 300 | 24.467 | 2.304 | 63.300 | 1.00 | 32.94 | |
| ATOM | 773 | O | GLU | 300 | 25.389 | 1.517 | 63.123 | 1.00 | 32.67 | |
| ATOM | 774 | N | PHE | 301 | 24.033 | 2.649 | 64.507 | 1.00 | 31.51 | |
| ATOM | 775 | CA | PHE | 301 | 24.632 | 2.094 | 65.710 | 1.00 | 32.60 | |
| ATOM | 776 | CB | PHE | 301 | 24.822 | 3.214 | 66.736 | 1.00 | 32.49 | |
| ATOM | 777 | CG | PHE | 301 | 25.879 | 4.203 | 66.349 | 1.00 | 34.07 | |
| ATOM | 778 | CD1 | PHE | 301 | 27.223 | 3.838 | 66.358 | 1.00 | 33.32 | |
| ATOM | 779 | CD2 | PHE | 301 | 25.537 | 5.483 | 65.929 | 1.00 | 33.10 | |
| ATOM | 780 | CE1 | PHE | 301 | 28.207 | 4.737 | 65.949 | 1.00 | 34.17 | |
| ATOM | 781 | CE2 | PHE | 301 | 26.506 | 6.385 | 65.519 | 1.00 | 34.69 | |
| ATOM | 782 | CZ | PHE | 301 | 27.854 | 6.008 | 65.530 | 1.00 | 33.21 | |
| ATOM | 783 | C | PHE | 301 | 23.820 | 0.940 | 66.301 | 1.00 | 33.77 | |
| ATOM | 784 | O | PHE | 301 | 24.127 | 0.441 | 67.377 | 1.00 | 34.55 | |
| ATOM | 785 | N | LEU | 302 | 22.780 | 0.516 | 65.589 | 1.00 | 34.80 | |
| ATOM | 786 | CA | LEU | 302 | 21.966 | −0.593 | 66.045 | 1.00 | 36.81 | |
| ATOM | 787 | CB | LEU | 302 | 20.641 | −0.645 | 65.287 | 1.00 | 34.98 | |
| ATOM | 788 | CG | LEU | 302 | 19.779 | 0.596 | 65.488 | 1.00 | 31.61 | |
| ATOM | 789 | CD1 | LEU | 302 | 18.551 | 0.528 | 64.577 | 1.00 | 31.62 | |
| ATOM | 790 | CD2 | LEU | 302 | 19.386 | 0.717 | 66.955 | 1.00 | 31.48 | |
| ATOM | 791 | C | LEU | 302 | 22.744 | −1.869 | 65.775 | 1.00 | 40.52 | |
| ATOM | 792 | O | LEU | 302 | 23.333 | −2.038 | 64.701 | 1.00 | 40.16 | |
| ATOM | 793 | N | THR | 303 | 22.733 | −2.764 | 66.753 | 1.00 | 44.10 | |
| ATOM | 794 | CA | THR | 303 | 23.422 | −4.037 | 66.634 | 1.00 | 48.80 | |
| ATOM | 795 | CB | THR | 303 | 23.126 | −4.933 | 67.850 | 1.00 | 49.83 | |
| ATOM | 796 | OG1 | THR | 303 | 23.211 | −4.151 | 69.050 | 1.00 | 51.18 | |
| ATOM | 797 | CG2 | THR | 303 | 24.132 | −6.076 | 67.924 | 1.00 | 51.25 | |
| ATOM | 798 | C | THR | 303 | 22.932 | −4.731 | 65.368 | 1.00 | 50.84 | |
| ATOM | 799 | O | THR | 303 | 21.739 | −4.703 | 65.052 | 1.00 | 50.20 | |
| ATOM | 800 | N | ALA | 304 | 23.864 | −5.341 | 64.644 | 1.00 | 53.24 | |
| ATOM | 801 | CA | ALA | 304 | 23.538 | −6.040 | 63.410 | 1.00 | 55.88 | |
| ATOM | 802 | CB | ALA | 304 | 24.739 | −6.867 | 62.946 | 1.00 | 56.05 | |
| ATOM | 803 | C | ALA | 304 | 22.317 | −6.942 | 63.575 | 1.00 | 57.61 | |
| ATOM | 804 | O | ALA | 304 | 22.133 | −7.583 | 64.617 | 1.00 | 58.45 | |
| ATOM | 805 | N | ALA | 305 | 21.481 | −6.972 | 62.540 | 1.00 | 59.51 | |
| ATOM | 806 | CA | ALA | 305 | 20.282 | −7.797 | 62.535 | 1.00 | 60.68 | |
| ATOM | 807 | CB | ALA | 305 | 19.548 | −7.651 | 61.205 | 1.00 | 60.98 | |
| ATOM | 808 | C | ALA | 305 | 20.714 | −9.245 | 62.744 | 1.00 | 61.71 | |
| ATOM | 809 | O | ALA | 305 | 19.925 | −10.029 | 63.319 | 1.00 | 62.15 | |
| ATOM | 810 | OXT | ALA | 305 | 21.848 | −9.572 | 62.316 | 1.00 | 61.88 | |
| ATOM | 811 | CB | ALA | 316 | 17.484 | −11.402 | 70.435 | 1.00 | 59.02 | |
| ATOM | 812 | C | ALA | 316 | 15.833 | −9.781 | 71.403 | 1.00 | 57.95 | |
| ATOM | 813 | O | ALA | 316 | 15.469 | −9.327 | 70.316 | 1.00 | 58.69 | |
| ATOM | 814 | N | ALA | 316 | 15.341 | −12.224 | 71.375 | 1.00 | 58.18 | |
| ATOM | 815 | CA | ALA | 316 | 16.413 | −11.191 | 71.510 | 1.00 | 58.61 | |
| ATOM | 816 | N | ALA | 317 | 15.747 | −9.096 | 72.539 | 1.00 | 56.79 | |
| ATOM | 817 | CA | ALA | 317 | 15.218 | −7.739 | 72.587 | 1.00 | 54.70 | |
| ATOM | 818 | CB | ALA | 317 | 13.813 | −7.745 | 73.174 | 1.00 | 54.73 | |
| ATOM | 819 | C | ALA | 317 | 16.142 | −6.871 | 73.437 | 1.00 | 52.91 | |
| ATOM | 820 | O | ALA | 317 | 15.697 | −5.935 | 74.111 | 1.00 | 53.06 | |
| ATOM | 821 | N | SER | 318 | 17.430 | −7.207 | 73.404 | 1.00 | 49.42 | |
| ATOM | 822 | CA | SER | 318 | 18.449 | −6.478 | 74.151 | 1.00 | 46.14 | |
| ATOM | 823 | CB | SER | 318 | 19.830 | −7.056 | 73.835 | 1.00 | 46.72 | |
| ATOM | 824 | OG | SER | 318 | 20.834 | −6.462 | 74.639 | 1.00 | 47.98 | |
| ATOM | 825 | C | SER | 318 | 18.378 | −5.009 | 73.723 | 1.00 | 43.02 | |
| ATOM | 826 | O | SER | 318 | 18.543 | −4.686 | 72.541 | 1.00 | 44.27 | |
| ATOM | 827 | N | PRO | 319 | 18.122 | −4.100 | 74.676 | 0.50 | 41.03 | AC1 |
| ATOM | 828 | CD | PRO | 319 | 17.740 | −4.342 | 76.079 | 0.50 | 40.29 | AC1 |
| ATOM | 829 | CA | PRO | 319 | 18.033 | −2.672 | 74.351 | 0.50 | 38.31 | AC1 |
| ATOM | 830 | CB | PRO | 319 | 17.690 | −2.032 | 75.694 | 0.50 | 38.96 | AC1 |
| ATOM | 831 | CG | PRO | 319 | 16.927 | −3.116 | 76.402 | 0.50 | 39.34 | AC1 |
| ATOM | 832 | C | PRO | 319 | 19.310 | −2.080 | 73.756 | 0.50 | 35.85 | AC1 |
| ATOM | 833 | O | PRO | 319 | 20.394 | −2.240 | 74.319 | 0.50 | 35.73 | AC1 |
| ATOM | 834 | N | PRO | 320 | 19.197 | −1.403 | 72.599 | 1.00 | 33.69 | |
| ATOM | 835 | CD | PRO | 320 | 18.041 | −1.404 | 71.692 | 1.00 | 32.09 | |
| ATOM | 836 | CA | PRO | 320 | 20.362 | −0.784 | 71.952 | 1.00 | 30.83 | |
| ATOM | 837 | CB | PRO | 320 | 19.769 | −0.083 | 70.722 | 1.00 | 30.11 | |
| ATOM | 838 | CG | PRO | 320 | 18.279 | −0.174 | 70.892 | 1.00 | 32.74 | |
| ATOM | 839 | C | PRO | 320 | 21.008 | 0.185 | 72.949 | 1.00 | 27.19 | |
| ATOM | 840 | O | PRO | 320 | 20.317 | 0.824 | 73.756 | 1.00 | 27.36 | |
| ATOM | 841 | N | GLN | 321 | 22.328 | 0.297 | 72.890 | 1.00 | 25.76 | |
| ATOM | 842 | CA | GLN | 321 | 23.060 | 1.121 | 73.847 | 1.00 | 25.85 | |
| ATOM | 843 | CB | GLN | 321 | 24.262 | 0.329 | 74.357 | 1.00 | 29.20 | |
| ATOM | 844 | CG | GLN | 321 | 23.925 | −1.090 | 74.757 | 1.00 | 34.12 | |
| ATOM | 845 | CD | GLN | 321 | 23.408 | −1.197 | 76.163 | 1.00 | 38.11 | |
| ATOM | 846 | OE1 | GLN | 321 | 24.127 | −0.916 | 77.123 | 1.00 | 40.85 | |
| ATOM | 847 | NE2 | GLN | 321 | 22.150 | −1.617 | 76.302 | 1.00 | 40.57 | |
| ATOM | 848 | C | GLN | 321 | 23.546 | 2.495 | 73.423 | 1.00 | 23.90 | |
| ATOM | 849 | O | GLN | 321 | 23.914 | 3.297 | 74.275 | 1.00 | 24.18 | |
| ATOM | 850 | N | LEU | 322 | 23.536 | 2.790 | 72.128 | 1.00 | 22.59 | |
| ATOM | 851 | CA | LEU | 322 | 24.050 | 4.080 | 71.666 | 1.00 | 21.41 | |
| ATOM | 852 | CB | LEU | 322 | 25.291 | 3.879 | 70.775 | 1.00 | 22.86 | |
| ATOM | 853 | CG | LEU | 322 | 26.560 | 3.362 | 71.432 | 1.00 | 25.45 | |
| ATOM | 854 | CD1 | LEU | 322 | 27.625 | 3.130 | 70.345 | 1.00 | 28.06 | |
| ATOM | 855 | CD2 | LEU | 322 | 27.036 | 4.384 | 72.497 | 1.00 | 24.21 | |
| ATOM | 856 | C | LEU | 322 | 23.079 | 4.942 | 70.891 | 1.00 | 22.08 | |
| ATOM | 857 | O | LEU | 322 | 22.435 | 4.478 | 69.961 | 1.00 | 23.11 | |
| ATOM | 858 | N | MET | 323 | 23.003 | 6.205 | 71.270 | 1.00 | 20.81 | |
| ATOM | 859 | CA | MET | 323 | 22.145 | 7.157 | 70.590 | 1.00 | 21.91 | |
| ATOM | 860 | CB | MET | 323 | 21.213 | 7.835 | 71.598 | 1.00 | 23.20 | |
| ATOM | 861 | CG | MET | 323 | 20.325 | 8.922 | 70.978 | 1.00 | 22.36 | |
| ATOM | 862 | SD | MET | 323 | 19.196 | 9.636 | 72.187 | 1.00 | 22.04 | |
| ATOM | 863 | CE | MET | 323 | 18.026 | 8.314 | 72.302 | 1.00 | 22.12 | |
| ATOM | 864 | C | MET | 323 | 23.054 | 8.187 | 69.914 | 1.00 | 25.54 | |
| ATOM | 865 | O | MET | 323 | 24.002 | 8.691 | 70.530 | 1.00 | 25.31 | |
| ATOM | 866 | N | CYS | 324 | 22.781 | 8.506 | 68.650 | 1.00 | 22.41 | |
| ATOM | 867 | CA | CYS | 324 | 23.619 | 9.483 | 67.941 | 1.00 | 24.83 | |
| ATOM | 868 | CB | CYS | 324 | 23.854 | 9.029 | 66.512 | 1.00 | 23.91 | |
| ATOM | 869 | SG | CYS | 324 | 24.921 | 10.185 | 65.588 | 1.00 | 27.74 | |
| ATOM | 870 | C | CYS | 324 | 22.995 | 10.873 | 67.950 | 1.00 | 24.34 | |
| ATOM | 871 | O | CYS | 324 | 22.010 | 11.148 | 67.249 | 1.00 | 24.90 | |
| ATOM | 872 | N | LEU | 325 | 23.560 | 11.758 | 68.749 | 1.00 | 24.49 | |
| ATOM | 873 | CA | LEU | 325 | 23.038 | 13.114 | 68.843 | 1.00 | 25.66 | |
| ATOM | 874 | CB | LEU | 325 | 23.421 | 13.746 | 70.183 | 1.00 | 26.79 | |
| ATOM | 875 | CG | LEU | 325 | 22.935 | 12.964 | 71.421 | 1.00 | 27.19 | |
| ATOM | 876 | CD1 | LEU | 325 | 23.256 | 13.744 | 72.704 | 1.00 | 29.28 | |
| ATOM | 877 | CD2 | LEU | 325 | 21.423 | 12.732 | 71.320 | 1.00 | 27.92 | |
| ATOM | 878 | C | LEU | 325 | 23.513 | 13.990 | 67.682 | 1.00 | 28.10 | |
| ATOM | 879 | O | LEU | 325 | 22.860 | 14.978 | 67.344 | 1.00 | 31.20 | |
| ATOM | 880 | N | MET | 326 | 24.650 | 13.618 | 67.094 | 1.00 | 28.83 | |
| ATOM | 881 | CA | MET | 326 | 25.253 | 14.311 | 65.947 | 1.00 | 30.95 | |
| ATOM | 882 | CB | MET | 326 | 25.726 | 15.721 | 66.350 | 1.00 | 31.86 | |
| ATOM | 883 | CG | MET | 326 | 26.894 | 15.710 | 67.335 | 1.00 | 31.84 | |
| ATOM | 884 | SD | MET | 326 | 27.648 | 17.333 | 67.693 | 1.00 | 34.98 | |
| ATOM | 885 | CE | MET | 326 | 29.085 | 16.800 | 68.706 | 1.00 | 30.77 | |
| ATOM | 886 | C | MET | 326 | 26.462 | 13.453 | 65.513 | 1.00 | 33.06 | |
| ATOM | 887 | O | MET | 326 | 26.882 | 12.565 | 66.242 | 1.00 | 32.71 | |
| ATOM | 888 | N | PRO | 327 | 27.013 | 13.682 | 64.307 | 1.00 | 35.54 | |
| ATOM | 889 | CD | PRO | 327 | 26.511 | 14.545 | 63.227 | 1.00 | 35.87 | |
| ATOM | 890 | CA | PRO | 327 | 28.180 | 12.896 | 63.857 | 1.00 | 36.24 | |
| ATOM | 891 | CB | PRO | 327 | 28.508 | 13.519 | 62.503 | 1.00 | 37.87 | |
| ATOM | 892 | CG | PRO | 327 | 27.159 | 13.932 | 62.001 | 1.00 | 35.11 | |
| ATOM | 893 | C | PRO | 327 | 29.353 | 13.035 | 64.836 | 1.00 | 38.75 | |
| ATOM | 894 | O | PRO | 327 | 29.754 | 14.154 | 65.155 | 1.00 | 38.99 | |
| ATOM | 895 | N | GLY | 328 | 29.890 | 11.908 | 65.314 | 1.00 | 38.52 | |
| ATOM | 896 | CA | GLY | 328 | 31.001 | 11.933 | 66.257 | 1.00 | 38.65 | |
| ATOM | 897 | C | GLY | 328 | 30.606 | 12.217 | 67.700 | 1.00 | 38.39 | |
| ATOM | 898 | O | GLY | 328 | 31.431 | 12.604 | 68.545 | 1.00 | 39.05 | |
| ATOM | 899 | N | MET | 329 | 29.326 | 12.020 | 67.994 | 1.00 | 36.32 | |
| ATOM | 900 | CA | MET | 329 | 28.830 | 12.251 | 69.326 | 1.00 | 34.24 | |
| ATOM | 901 | CB | MET | 329 | 28.343 | 13.693 | 69.451 | 1.00 | 38.91 | |
| ATOM | 902 | CG | MET | 329 | 27.462 | 13.991 | 70.649 | 1.00 | 44.24 | |
| ATOM | 903 | SD | MET | 329 | 28.233 | 13.696 | 72.235 | 1.00 | 50.95 | |
| ATOM | 904 | CE | MET | 329 | 26.854 | 12.835 | 73.089 | 1.00 | 48.58 | |
| ATOM | 905 | C | MET | 329 | 27.700 | 11.271 | 69.603 | 1.00 | 30.52 | |
| ATOM | 906 | O | MET | 329 | 26.647 | 11.331 | 68.975 | 1.00 | 28.92 | |
| ATOM | 907 | N | THR | 330 | 27.960 | 10.324 | 70.490 | 1.00 | 26.42 | |
| ATOM | 908 | CA | THR | 330 | 26.933 | 9.370 | 70.865 | 1.00 | 23.65 | |
| ATOM | 909 | CB | THR | 330 | 27.338 | 7.895 | 70.542 | 1.00 | 24.12 | |
| ATOM | 910 | OG1 | THR | 330 | 28.672 | 7.654 | 71.005 | 1.00 | 27.18 | |
| ATOM | 911 | CG2 | THR | 330 | 27.269 | 7.622 | 69.034 | 1.00 | 26.34 | |
| ATOM | 912 | C | THR | 330 | 26.685 | 9.475 | 72.360 | 1.00 | 23.23 | |
| ATOM | 913 | O | THR | 330 | 27.572 | 9.802 | 73.146 | 1.00 | 23.91 | |
| ATOM | 914 | N | LEU | 331 | 25.450 | 9.211 | 72.748 | 1.00 | 20.37 | |
| ATOM | 915 | CA | LEU | 331 | 25.082 | 9.213 | 74.142 | 1.00 | 21.33 | |
| ATOM | 916 | CB | LEU | 331 | 23.773 | 9.986 | 74.340 | 1.00 | 24.38 | |
| ATOM | 917 | CG | LEU | 331 | 23.113 | 9.731 | 75.696 | 1.00 | 25.70 | |
| ATOM | 918 | CD1 | LEU | 331 | 23.979 | 10.317 | 76.825 | 1.00 | 30.48 | |
| ATOM | 919 | CD2 | LEU | 331 | 21.723 | 10.348 | 75.694 | 1.00 | 29.64 | |
| ATOM | 920 | C | LEU | 331 | 24.892 | 7.737 | 74.489 | 1.00 | 21.13 | |
| ATOM | 921 | O | LEU | 331 | 24.158 | 7.026 | 73.819 | 1.00 | 19.21 | |
| ATOM | 922 | N | HIS | 332 | 25.582 | 7.262 | 75.525 | 1.00 | 18.80 | |
| ATOM | 923 | CA | HIS | 332 | 25.472 | 5.864 | 75.901 | 1.00 | 18.64 | |
| ATOM | 924 | CB | HIS | 332 | 26.740 | 5.417 | 76.658 | 1.00 | 18.77 | |
| ATOM | 925 | CG | HIS | 332 | 26.826 | 3.938 | 76.862 | 1.00 | 21.68 | |
| ATOM | 926 | CD2 | HIS | 332 | 27.533 | 2.991 | 76.205 | 1.00 | 22.97 | |
| ATOM | 927 | ND1 | HIS | 332 | 26.092 | 3.273 | 77.824 | 1.00 | 22.38 | |
| ATOM | 928 | CE1 | HIS | 332 | 26.340 | 1.978 | 77.745 | 1.00 | 23.74 | |
| ATOM | 929 | NE2 | HIS | 332 | 27.213 | 1.778 | 76.769 | 1.00 | 25.26 | |
| ATOM | 930 | C | HIS | 332 | 24.262 | 5.721 | 76.820 | 1.00 | 18.95 | |
| ATOM | 931 | O | HIS | 332 | 23.981 | 6.620 | 77.616 | 1.00 | 19.91 | |
| ATOM | 932 | N | ARG | 333 | 23.594 | 4.581 | 76.715 | 1.00 | 19.39 | |
| ATOM | 933 | CA | ARG | 333 | 22.394 | 4.282 | 77.509 | 1.00 | 19.71 | |
| ATOM | 934 | CB | ARG | 333 | 21.930 | 2.850 | 77.235 | 1.00 | 20.65 | |
| ATOM | 935 | CG | ARG | 333 | 20.582 | 2.512 | 77.896 | 1.00 | 21.54 | |
| ATOM | 936 | CD | ARG | 333 | 20.018 | 1.212 | 77.353 | 1.00 | 21.69 | |
| ATOM | 937 | NE | ARG | 333 | 18.703 | 0.862 | 77.899 | 1.00 | 20.98 | |
| ATOM | 938 | CZ | ARG | 333 | 18.501 | 0.034 | 78.921 | 1.00 | 22.46 | |
| ATOM | 939 | NH1 | ARG | 333 | 19.535 | −0.547 | 79.531 | 1.00 | 24.12 | |
| ATOM | 940 | NH2 | ARG | 333 | 17.255 | −0.241 | 79.309 | 1.00 | 20.87 | |
| ATOM | 941 | C | ARG | 333 | 22.643 | 4.463 | 79.018 | 1.00 | 21.14 | |
| ATOM | 942 | O | ARG | 333 | 21.782 | 4.926 | 79.755 | 1.00 | 18.39 | |
| ATOM | 943 | N | ASN | 334 | 23.832 | 4.107 | 79.484 | 1.00 | 21.27 | |
| ATOM | 944 | CA | ASN | 334 | 24.114 | 4.259 | 80.910 | 1.00 | 23.47 | |
| ATOM | 945 | CB | ASN | 334 | 25.486 | 3.653 | 81.244 | 1.00 | 24.93 | |
| ATOM | 946 | CG | ASN | 334 | 25.504 | 2.119 | 81.158 | 1.00 | 25.59 | |
| ATOM | 947 | OD1 | ASN | 334 | 24.467 | 1.461 | 81.054 | 1.00 | 26.24 | |
| ATOM | 948 | ND2 | ASN | 334 | 26.707 | 1.544 | 81.204 | 1.00 | 29.08 | |
| ATOM | 949 | C | ASN | 334 | 24.048 | 5.718 | 81.380 | 1.00 | 22.91 | |
| ATOM | 950 | O | ASN | 334 | 23.649 | 6.000 | 82.530 | 1.00 | 22.99 | |
| ATOM | 951 | N | SER | 335 | 24.441 | 6.657 | 80.524 | 1.00 | 22.89 | |
| ATOM | 952 | CA | SER | 335 | 24.378 | 8.059 | 80.904 | 1.00 | 22.41 | |
| ATOM | 953 | CB | SER | 335 | 25.178 | 8.915 | 79.930 | 1.00 | 27.35 | |
| ATOM | 954 | OG | SER | 335 | 26.517 | 8.437 | 79.856 | 1.00 | 31.39 | |
| ATOM | 955 | C | SER | 335 | 22.908 | 8.484 | 80.939 | 1.00 | 23.03 | |
| ATOM | 956 | O | SER | 335 | 22.496 | 9.260 | 81.809 | 1.00 | 22.21 | |
| ATOM | 957 | N | ALA | 336 | 22.126 | 7.970 | 79.989 | 1.00 | 21.81 | |
| ATOM | 958 | CA | ALA | 336 | 20.703 | 8.255 | 79.952 | 1.00 | 21.09 | |
| ATOM | 959 | CB | ALA | 336 | 20.060 | 7.595 | 78.718 | 1.00 | 21.81 | |
| ATOM | 960 | C | ALA | 336 | 20.044 | 7.744 | 81.232 | 1.00 | 21.37 | |
| ATOM | 961 | O | ALA | 336 | 19.209 | 8.425 | 81.830 | 1.00 | 19.40 | |
| ATOM | 962 | N | LEU | 337 | 20.423 | 6.540 | 81.660 | 1.00 | 19.77 | |
| ATOM | 963 | CA | LEU | 337 | 19.860 | 5.960 | 82.865 | 1.00 | 19.59 | |
| ATOM | 964 | CB | LEU | 337 | 20.374 | 4.517 | 83.039 | 1.00 | 19.20 | |
| ATOM | 965 | CG | LEU | 337 | 19.835 | 3.502 | 82.031 | 1.00 | 20.97 | |
| ATOM | 966 | CD1 | LEU | 337 | 20.702 | 2.280 | 82.055 | 1.00 | 23.49 | |
| ATOM | 967 | CD2 | LEU | 337 | 18.391 | 3.164 | 82.329 | 1.00 | 21.07 | |
| ATOM | 968 | C | LEU | 337 | 20.206 | 6.777 | 84.089 | 1.00 | 20.54 | |
| ATOM | 969 | O | LEU | 337 | 19.364 | 6.997 | 84.985 | 1.00 | 19.57 | |
| ATOM | 970 | N | GLN | 338 | 21.454 | 7.226 | 84.131 | 1.00 | 21.36 | |
| ATOM | 971 | CA | GLN | 338 | 21.920 | 7.994 | 85.264 | 1.00 | 22.39 | |
| ATOM | 972 | CB | GLN | 338 | 23.439 | 8.206 | 85.179 | 1.00 | 22.44 | |
| ATOM | 973 | CG | GLN | 338 | 23.973 | 8.755 | 86.488 | 1.00 | 27.14 | |
| ATOM | 974 | CD | GLN | 338 | 25.467 | 9.026 | 86.481 | 1.00 | 29.23 | |
| ATOM | 975 | OE1 | GLN | 338 | 25.955 | 9.827 | 87.278 | 1.00 | 30.73 | |
| ATOM | 976 | NE2 | GLN | 338 | 26.196 | 8.360 | 85.596 | 1.00 | 30.79 | |
| ATOM | 977 | C | GLN | 338 | 21.200 | 9.331 | 85.381 | 1.00 | 23.40 | |
| ATOM | 978 | O | GLN | 338 | 20.894 | 9.790 | 86.490 | 1.00 | 21.75 | |
| ATOM | 979 | N | ALA | 339 | 20.899 | 9.935 | 84.227 | 1.00 | 22.63 | |
| ATOM | 980 | CA | ALA | 339 | 20.215 | 11.217 | 84.191 | 1.00 | 24.41 | |
| ATOM | 981 | CB | ALA | 339 | 20.430 | 11.903 | 82.832 | 1.00 | 23.46 | |
| ATOM | 982 | C | ALA | 339 | 18.727 | 11.082 | 84.463 | 1.00 | 24.73 | |
| ATOM | 983 | O | ALA | 339 | 18.059 | 12.084 | 84.641 | 1.00 | 27.16 | |
| ATOM | 984 | N | GLY | 340 | 18.218 | 9.850 | 84.499 | 1.00 | 22.58 | |
| ATOM | 985 | CA | GLY | 340 | 16.803 | 9.622 | 84.754 | 1.00 | 23.89 | |
| ATOM | 986 | C | GLY | 340 | 15.898 | 9.580 | 83.531 | 1.00 | 25.09 | |
| ATOM | 987 | O | GLY | 340 | 14.666 | 9.607 | 83.666 | 1.00 | 25.54 | |
| ATOM | 988 | N | VAL | 341 | 16.492 | 9.479 | 82.346 | 1.00 | 23.51 | |
| ATOM | 989 | CA | VAL | 341 | 15.719 | 9.473 | 81.095 | 1.00 | 20.92 | |
| ATOM | 990 | CB | VAL | 341 | 16.134 | 10.677 | 80.211 | 1.00 | 21.83 | |
| ATOM | 991 | CG1 | VAL | 341 | 15.838 | 11.981 | 80.942 | 1.00 | 21.94 | |
| ATOM | 992 | CG2 | VAL | 341 | 17.621 | 10.577 | 79.858 | 1.00 | 21.91 | |
| ATOM | 993 | C | VAL | 341 | 15.891 | 8.185 | 80.300 | 1.00 | 20.24 | |
| ATOM | 994 | O | VAL | 341 | 15.803 | 8.182 | 79.083 | 1.00 | 19.53 | |
| ATOM | 995 | N | GLY | 342 | 16.132 | 7.079 | 80.999 | 1.00 | 19.65 | |
| ATOM | 996 | CA | GLY | 342 | 16.324 | 5.827 | 80.301 | 1.00 | 18.76 | |
| ATOM | 997 | C | GLY | 342 | 15.104 | 5.352 | 79.541 | 1.00 | 19.27 | |
| ATOM | 998 | O | GLY | 342 | 15.230 | 4.771 | 78.470 | 1.00 | 18.82 | |
| ATOM | 999 | N | GLN | 343 | 13.922 | 5.598 | 80.088 | 0.50 | 18.31 | AC1 |
| ATOM | 1000 | CA | GLN | 343 | 12.706 | 5.163 | 79.422 | 0.50 | 19.11 | AC1 |
| ATOM | 1001 | CB | GLN | 343 | 11.507 | 5.485 | 80.305 | 0.50 | 19.94 | AC1 |
| ATOM | 1002 | CG | GLN | 343 | 10.200 | 4.866 | 79.867 | 0.50 | 22.91 | AC1 |
| ATOM | 1003 | CD | GLN | 343 | 9.082 | 5.177 | 80.852 | 0.50 | 24.13 | AC1 |
| ATOM | 1004 | OE1 | GLN | 343 | 8.669 | 6.326 | 80.995 | 0.50 | 26.85 | AC1 |
| ATOM | 1005 | NE2 | GLN | 343 | 8.606 | 4.154 | 81.555 | 0.50 | 25.40 | AC1 |
| ATOM | 1006 | C | GLN | 343 | 12.542 | 5.813 | 78.043 | 0.50 | 19.14 | AC1 |
| ATOM | 1007 | O | GLN | 343 | 12.318 | 5.127 | 77.050 | 0.50 | 18.83 | AC1 |
| ATOM | 1008 | N | ILE | 344 | 12.664 | 7.136 | 77.977 | 1.00 | 17.95 | |
| ATOM | 1009 | CA | ILE | 344 | 12.506 | 7.812 | 76.693 | 1.00 | 17.74 | |
| ATOM | 1010 | CB | ILE | 344 | 12.348 | 9.372 | 76.875 | 1.00 | 18.47 | |
| ATOM | 1011 | CG2 | ILE | 344 | 13.700 | 10.010 | 77.231 | 1.00 | 20.66 | |
| ATOM | 1012 | CG1 | ILE | 344 | 11.754 | 9.984 | 75.596 | 1.00 | 19.04 | |
| ATOM | 1013 | CD1 | ILE | 344 | 11.445 | 11.499 | 75.662 | 1.00 | 21.07 | |
| ATOM | 1014 | C | ILE | 344 | 13.641 | 7.448 | 75.718 | 1.00 | 17.34 | |
| ATOM | 1015 | O | ILE | 344 | 13.440 | 7.389 | 74.499 | 1.00 | 16.12 | |
| ATOM | 1016 | N | PHE | 345 | 14.844 | 7.210 | 76.248 | 1.00 | 16.60 | |
| ATOM | 1017 | CA | PHE | 345 | 15.976 | 6.826 | 75.417 | 1.00 | 17.63 | |
| ATOM | 1018 | CB | PHE | 345 | 17.208 | 6.636 | 76.322 | 1.00 | 17.86 | |
| ATOM | 1019 | CG | PHE | 345 | 18.455 | 6.189 | 75.613 | 1.00 | 18.24 | |
| ATOM | 1020 | CD1 | PHE | 345 | 18.657 | 4.848 | 75.273 | 1.00 | 18.20 | |
| ATOM | 1021 | CD2 | PHE | 345 | 19.470 | 7.090 | 75.363 | 1.00 | 20.10 | |
| ATOM | 1022 | CE1 | PHE | 345 | 19.854 | 4.412 | 74.706 | 1.00 | 19.01 | |
| ATOM | 1023 | CE2 | PHE | 345 | 20.663 | 6.673 | 74.798 | 1.00 | 17.96 | |
| ATOM | 1024 | CZ | PHE | 345 | 20.861 | 5.328 | 74.469 | 1.00 | 18.58 | |
| ATOM | 1025 | C | PHE | 345 | 15.603 | 5.522 | 74.710 | 1.00 | 18.98 | |
| ATOM | 1026 | O | PHE | 345 | 15.756 | 5.389 | 73.491 | 1.00 | 18.15 | |
| ATOM | 1027 | N | ASP | 346 | 15.072 | 4.568 | 75.480 | 1.00 | 18.04 | |
| ATOM | 1028 | CA | ASP | 346 | 14.695 | 3.300 | 74.894 | 1.00 | 18.83 | |
| ATOM | 1029 | CB | ASP | 346 | 14.285 | 2.302 | 75.988 | 1.00 | 19.96 | |
| ATOM | 1030 | CG | ASP | 346 | 15.467 | 1.768 | 76.786 | 1.00 | 24.04 | |
| ATOM | 1031 | OD1 | ASP | 346 | 16.630 | 2.038 | 76.430 | 1.00 | 24.95 | |
| ATOM | 1032 | OD2 | ASP | 346 | 15.232 | 1.043 | 77.784 | 1.00 | 24.86 | |
| ATOM | 1033 | C | ASP | 346 | 13.550 | 3.462 | 73.872 | 1.00 | 18.04 | |
| ATOM | 1034 | O | ASP | 346 | 13.549 | 2.782 | 72.841 | 1.00 | 19.39 | |
| ATOM | 1035 | N | ARG | 347 | 12.603 | 4.359 | 74.138 | 1.00 | 19.77 | |
| ATOM | 1036 | CA | ARG | 347 | 11.493 | 4.579 | 73.188 | 1.00 | 20.42 | |
| ATOM | 1037 | CB | ARG | 347 | 10.443 | 5.558 | 73.708 | 1.00 | 24.20 | |
| ATOM | 1038 | CG | ARG | 347 | 9.527 | 5.024 | 74.786 | 1.00 | 26.30 | |
| ATOM | 1039 | CD | ARG | 347 | 8.310 | 5.929 | 74.937 | 1.00 | 28.76 | |
| ATOM | 1040 | NE | ARG | 347 | 7.383 | 5.807 | 73.813 | 1.00 | 29.58 | |
| ATOM | 1041 | CZ | ARG | 347 | 6.312 | 6.577 | 73.629 | 1.00 | 31.91 | |
| ATOM | 1042 | NH1 | ARG | 347 | 6.028 | 7.536 | 74.492 | 1.00 | 32.14 | |
| ATOM | 1043 | NH2 | ARG | 347 | 5.522 | 6.378 | 72.579 | 1.00 | 32.47 | |
| ATOM | 1044 | C | ARG | 347 | 12.012 | 5.120 | 71.875 | 1.00 | 20.09 | |
| ATOM | 1045 | O | ARG | 347 | 11.586 | 4.703 | 70.806 | 1.00 | 18.14 | |
| ATOM | 1046 | N | VAL | 348 | 12.939 | 6.073 | 71.945 | 1.00 | 18.48 | |
| ATOM | 1047 | CA | VAL | 348 | 13.480 | 6.621 | 70.708 | 1.00 | 18.94 | |
| ATOM | 1048 | CB | VAL | 348 | 14.532 | 7.713 | 70.991 | 1.00 | 18.19 | |
| ATOM | 1049 | CG1 | VAL | 348 | 15.286 | 8.052 | 69.706 | 1.00 | 18.18 | |
| ATOM | 1050 | CG2 | VAL | 348 | 13.862 | 8.930 | 71.600 | 1.00 | 19.11 | |
| ATOM | 1051 | C | VAL | 348 | 14.143 | 5.518 | 69.882 | 1.00 | 18.63 | |
| ATOM | 1052 | O | VAL | 348 | 13.885 | 5.380 | 68.691 | 1.00 | 17.99 | |
| ATOM | 1053 | N | LEU | 349 | 14.981 | 4.709 | 70.514 | 1.00 | 18.35 | |
| ATOM | 1054 | CA | LEU | 349 | 15.679 | 3.686 | 69.749 | 1.00 | 19.38 | |
| ATOM | 1055 | CB | LEU | 349 | 16.906 | 3.175 | 70.526 | 1.00 | 20.34 | |
| ATOM | 1056 | CG | LEU | 349 | 17.920 | 4.286 | 70.807 | 1.00 | 20.49 | |
| ATOM | 1057 | CD1 | LEU | 349 | 19.173 | 3.626 | 71.408 | 1.00 | 23.32 | |
| ATOM | 1058 | CD2 | LEU | 349 | 18.273 | 5.078 | 69.500 | 1.00 | 18.92 | |
| ATOM | 1059 | C | LEU | 349 | 14.822 | 2.509 | 69.294 | 1.00 | 20.39 | |
| ATOM | 1060 | O | LEU | 349 | 15.112 | 1.901 | 68.263 | 1.00 | 19.62 | |
| ATOM | 1061 | N | SER | 350 | 13.746 | 2.208 | 70.025 | 1.00 | 21.15 | |
| ATOM | 1062 | CA | SER | 350 | 12.907 | 1.074 | 69.623 | 1.00 | 23.27 | |
| ATOM | 1063 | CB | SER | 350 | 12.443 | 0.294 | 70.855 | 1.00 | 25.65 | |
| ATOM | 1064 | OG | SER | 350 | 11.622 | 1.081 | 71.687 | 1.00 | 28.64 | |
| ATOM | 1065 | C | SER | 350 | 11.698 | 1.451 | 68.766 | 1.00 | 21.84 | |
| ATOM | 1066 | O | SER | 350 | 11.424 | 0.790 | 67.762 | 1.00 | 25.35 | |
| ATOM | 1067 | N | GLU | 351 | 10.986 | 2.516 | 69.131 | 1.00 | 22.62 | |
| ATOM | 1068 | CA | GLU | 351 | 9.801 | 2.906 | 68.368 | 1.00 | 22.04 | |
| ATOM | 1069 | CB | GLU | 351 | 8.834 | 3.680 | 69.252 | 1.00 | 22.43 | |
| ATOM | 1070 | CG | GLU | 351 | 8.454 | 2.916 | 70.486 | 1.00 | 26.86 | |
| ATOM | 1071 | CD | GLU | 351 | 7.434 | 3.650 | 71.303 | 1.00 | 28.97 | |
| ATOM | 1072 | OE1 | GLU | 351 | 7.473 | 3.544 | 72.548 | 1.00 | 31.85 | |
| ATOM | 1073 | OE2 | GLU | 351 | 6.581 | 4.330 | 70.691 | 1.00 | 32.68 | |
| ATOM | 1074 | C | GLU | 351 | 10.133 | 3.708 | 67.132 | 1.00 | 22.11 | |
| ATOM | 1075 | O | GLU | 351 | 9.348 | 3.766 | 66.183 | 1.00 | 22.30 | |
| ATOM | 1076 | N | LEU | 352 | 11.309 | 4.328 | 67.113 | 1.00 | 19.99 | |
| ATOM | 1077 | CA | LEU | 352 | 11.696 | 5.083 | 65.934 | 1.00 | 19.01 | |
| ATOM | 1078 | CB | LEU | 352 | 12.043 | 6.555 | 66.293 | 1.00 | 18.64 | |
| ATOM | 1079 | CG | LEU | 352 | 10.911 | 7.365 | 66.950 | 1.00 | 19.12 | |
| ATOM | 1080 | CD1 | LEU | 352 | 11.445 | 8.742 | 67.339 | 1.00 | 19.51 | |
| ATOM | 1081 | CD2 | LEU | 352 | 9.703 | 7.505 | 66.018 | 1.00 | 19.25 | |
| ATOM | 1082 | C | LEU | 352 | 12.859 | 4.467 | 65.163 | 1.00 | 19.51 | |
| ATOM | 1083 | O | LEU | 352 | 12.704 | 3.977 | 64.038 | 1.00 | 21.10 | |
| ATOM | 1084 | N | SER | 353 | 14.052 | 4.464 | 65.754 | 0.50 | 18.72 | AC1 |
| ATOM | 1085 | CA | SER | 353 | 15.203 | 3.933 | 65.034 | 0.50 | 18.55 | AC1 |
| ATOM | 1086 | CB | SER | 353 | 16.473 | 4.032 | 65.883 | 0.50 | 16.66 | AC1 |
| ATOM | 1087 | OG | SER | 353 | 16.782 | 5.378 | 66.160 | 0.50 | 11.47 | AC1 |
| ATOM | 1088 | C | SER | 353 | 15.045 | 2.500 | 64.546 | 0.50 | 19.60 | AC1 |
| ATOM | 1089 | O | SER | 353 | 15.207 | 2.236 | 63.355 | 0.50 | 20.26 | AC1 |
| ATOM | 1090 | N | LEU | 354 | 14.746 | 1.579 | 65.455 | 1.00 | 21.67 | |
| ATOM | 1091 | CA | LEU | 354 | 14.602 | 0.181 | 65.051 | 1.00 | 23.55 | |
| ATOM | 1092 | CB | LEU | 354 | 14.360 | −0.715 | 66.278 | 1.00 | 24.35 | |
| ATOM | 1093 | CG | LEU | 354 | 14.284 | −2.222 | 66.012 | 1.00 | 30.18 | |
| ATOM | 1094 | CD1 | LEU | 354 | 15.388 | −2.664 | 65.055 | 1.00 | 31.88 | |
| ATOM | 1095 | CD2 | LEU | 354 | 14.392 | −2.944 | 67.345 | 1.00 | 31.36 | |
| ATOM | 1096 | C | LEU | 354 | 13.489 | −0.011 | 64.009 | 1.00 | 24.82 | |
| ATOM | 1097 | O | LEU | 354 | 13.696 | −0.684 | 63.004 | 1.00 | 25.66 | |
| ATOM | 1098 | N | LYS | 355 | 12.324 | 0.587 | 64.234 | 1.00 | 26.97 | |
| ATOM | 1099 | CA | LYS | 355 | 11.236 | 0.454 | 63.272 | 1.00 | 26.95 | |
| ATOM | 1100 | CB | LYS | 355 | 10.003 | 1.226 | 63.735 | 1.00 | 29.30 | |
| ATOM | 1101 | CG | LYS | 355 | 9.228 | 0.572 | 64.843 | 1.00 | 34.34 | |
| ATOM | 1102 | CD | LYS | 355 | 8.440 | −0.616 | 64.321 | 1.00 | 38.57 | |
| ATOM | 1103 | CE | LYS | 355 | 7.365 | −1.007 | 65.319 | 1.00 | 39.54 | |
| ATOM | 1104 | NZ | LYS | 355 | 7.883 | −0.958 | 66.713 | 1.00 | 40.20 | |
| ATOM | 1105 | C | LYS | 355 | 11.637 | 0.964 | 61.899 | 1.00 | 27.30 | |
| ATOM | 1106 | O | LYS | 355 | 11.281 | 0.369 | 60.883 | 1.00 | 28.58 | |
| ATOM | 1107 | N | MET | 356 | 12.362 | 2.080 | 61.860 | 1.00 | 25.41 | |
| ATOM | 1108 | CA | MET | 356 | 12.787 | 2.641 | 60.594 | 1.00 | 25.37 | |
| ATOM | 1109 | CB | MET | 356 | 13.279 | 4.071 | 60.778 | 1.00 | 27.04 | |
| ATOM | 1110 | CG | MET | 356 | 12.126 | 5.017 | 60.918 | 1.00 | 26.57 | |
| ATOM | 1111 | SD | MET | 356 | 12.669 | 6.671 | 60.768 | 1.00 | 36.72 | |
| ATOM | 1112 | CE | MET | 356 | 13.015 | 6.835 | 62.357 | 1.00 | 16.80 | |
| ATOM | 1113 | C | MET | 356 | 13.836 | 1.808 | 59.896 | 1.00 | 26.93 | |
| ATOM | 1114 | O | MET | 356 | 13.907 | 1.807 | 58.676 | 1.00 | 25.41 | |
| ATOM | 1115 | N | ARG | 357 | 14.672 | 1.114 | 60.661 | 1.00 | 27.88 | |
| ATOM | 1116 | CA | ARG | 357 | 15.656 | 0.281 | 60.003 | 1.00 | 29.75 | |
| ATOM | 1117 | CB | ARG | 357 | 16.733 | −0.214 | 60.961 | 1.00 | 31.09 | |
| ATOM | 1118 | CG | ARG | 357 | 17.748 | −1.056 | 60.213 | 1.00 | 35.17 | |
| ATOM | 1119 | CD | ARG | 357 | 18.840 | −1.607 | 61.112 | 1.00 | 38.46 | |
| ATOM | 1120 | NE | ARG | 357 | 18.313 | −2.543 | 62.097 | 1.00 | 41.53 | |
| ATOM | 1121 | CZ | ARG | 357 | 19.066 | −3.164 | 63.003 | 1.00 | 43.38 | |
| ATOM | 1122 | NH1 | ARG | 357 | 20.376 | −2.942 | 63.037 | 1.00 | 42.92 | |
| ATOM | 1123 | NH2 | ARG | 357 | 18.512 | −3.995 | 63.879 | 1.00 | 43.48 | |
| ATOM | 1124 | C | ARG | 357 | 14.926 | −0.925 | 59.422 | 1.00 | 29.63 | |
| ATOM | 1125 | O | ARG | 357 | 15.218 | −1.353 | 58.310 | 1.00 | 29.83 | |
| ATOM | 1126 | N | THR | 358 | 13.966 | −1.452 | 60.171 | 1.00 | 29.51 | |
| ATOM | 1127 | CA | THR | 358 | 13.204 | −2.609 | 59.704 | 1.00 | 30.65 | |
| ATOM | 1128 | CB | THR | 358 | 12.273 | −3.117 | 60.812 | 1.00 | 31.00 | |
| ATOM | 1129 | OG1 | THR | 358 | 13.071 | −3.609 | 61.895 | 1.00 | 32.46 | |
| ATOM | 1130 | CG2 | THR | 358 | 11.374 | −4.260 | 60.306 | 1.00 | 34.28 | |
| ATOM | 1131 | C | THR | 358 | 12.408 | −2.281 | 58.438 | 1.00 | 30.73 | |
| ATOM | 1132 | O | THR | 358 | 12.286 | −3.114 | 57.533 | 1.00 | 32.33 | |
| ATOM | 1133 | N | LEU | 359 | 11.895 | −1.056 | 58.359 | 1.00 | 29.40 | |
| ATOM | 1134 | CA | LEU | 359 | 11.124 | −0.610 | 57.201 | 1.00 | 29.68 | |
| ATOM | 1135 | CB | LEU | 359 | 10.242 | 0.583 | 57.605 | 1.00 | 30.32 | |
| ATOM | 1136 | CG | LEU | 359 | 8.732 | 0.609 | 57.321 | 1.00 | 34.26 | |
| ATOM | 1137 | CD1 | LEU | 359 | 8.197 | 2.038 | 57.529 | 1.00 | 33.77 | |
| ATOM | 1138 | CD2 | LEU | 359 | 8.452 | 0.142 | 55.900 | 1.00 | 34.08 | |
| ATOM | 1139 | C | LEU | 359 | 12.046 | −0.168 | 56.066 | 1.00 | 28.77 | |
| ATOM | 1140 | O | LEU | 359 | 11.585 | 0.164 | 54.974 | 1.00 | 27.98 | |
| ATOM | 1141 | N | ARG | 360 | 13.349 | −0.130 | 56.327 | 1.00 | 30.53 | |
| ATOM | 1142 | CA | ARG | 360 | 14.315 | 0.310 | 55.327 | 1.00 | 30.15 | |
| ATOM | 1143 | CB | ARG | 360 | 14.381 | −0.695 | 54.178 | 1.00 | 33.80 | |
| ATOM | 1144 | CG | ARG | 360 | 14.798 | −2.076 | 54.625 | 1.00 | 36.43 | |
| ATOM | 1145 | CD | ARG | 360 | 14.018 | −3.165 | 53.900 | 1.00 | 39.56 | |
| ATOM | 1146 | NE | ARG | 360 | 12.588 | −3.168 | 54.226 | 1.00 | 41.14 | |
| ATOM | 1147 | CZ | ARG | 360 | 11.618 | −2.942 | 53.340 | 1.00 | 42.18 | |
| ATOM | 1148 | NH1 | ARG | 360 | 11.918 | −2.688 | 52.071 | 1.00 | 41.02 | |
| ATOM | 1149 | NH2 | ARG | 360 | 10.345 | −2.983 | 53.717 | 1.00 | 42.86 | |
| ATOM | 1150 | C | ARG | 360 | 13.975 | 1.699 | 54.787 | 1.00 | 29.92 | |
| ATOM | 1151 | O | ARG | 360 | 14.073 | 1.958 | 53.592 | 1.00 | 28.84 | |
| ATOM | 1152 | N | VAL | 361 | 13.569 | 2.600 | 55.677 | 1.00 | 27.64 | |
| ATOM | 1153 | CA | VAL | 361 | 13.261 | 3.956 | 55.267 | 1.00 | 25.17 | |
| ATOM | 1154 | CB | VAL | 361 | 12.909 | 4.819 | 56.480 | 1.00 | 24.00 | |
| ATOM | 1155 | CG1 | VAL | 361 | 12.684 | 6.254 | 56.066 | 1.00 | 24.37 | |
| ATOM | 1156 | CG2 | VAL | 361 | 11.688 | 4.249 | 57.166 | 1.00 | 23.19 | |
| ATOM | 1157 | C | VAL | 361 | 14.504 | 4.525 | 54.594 | 1.00 | 25.34 | |
| ATOM | 1158 | O | VAL | 361 | 15.613 | 4.407 | 55.133 | 1.00 | 25.79 | |
| ATOM | 1159 | N | ASP | 362 | 14.344 | 5.109 | 53.404 | 1.00 | 24.62 | |
| ATOM | 1160 | CA | ASP | 362 | 15.481 | 5.712 | 52.718 | 1.00 | 24.80 | |
| ATOM | 1161 | CB | ASP | 362 | 15.540 | 5.316 | 51.223 | 1.00 | 27.01 | |
| ATOM | 1162 | CG | ASP | 362 | 14.307 | 5.720 | 50.433 | 1.00 | 29.25 | |
| ATOM | 1163 | OD1 | ASP | 362 | 13.697 | 6.764 | 50.731 | 1.00 | 27.58 | |
| ATOM | 1164 | OD2 | ASP | 362 | 13.963 | 4.987 | 49.479 | 1.00 | 32.41 | |
| ATOM | 1165 | C | ASP | 362 | 15.520 | 7.226 | 52.863 | 1.00 | 25.99 | |
| ATOM | 1166 | O | ASP | 362 | 14.631 | 7.823 | 53.486 | 1.00 | 25.65 | |
| ATOM | 1167 | N | GLN | 363 | 16.561 | 7.845 | 52.312 | 1.00 | 24.73 | |
| ATOM | 1168 | CA | GLN | 363 | 16.742 | 9.286 | 52.405 | 1.00 | 26.50 | |
| ATOM | 1169 | CB | GLN | 363 | 18.044 | 9.682 | 51.690 | 1.00 | 31.84 | |
| ATOM | 1170 | CG | GLN | 363 | 18.339 | 11.179 | 51.662 | 1.00 | 36.89 | |
| ATOM | 1171 | CD | GLN | 363 | 19.008 | 11.689 | 52.921 | 1.00 | 40.13 | |
| ATOM | 1172 | OE1 | GLN | 363 | 18.615 | 11.346 | 54.047 | 1.00 | 42.87 | |
| ATOM | 1173 | NE2 | GLN | 363 | 20.013 | 12.536 | 52.743 | 1.00 | 40.44 | |
| ATOM | 1174 | C | GLN | 363 | 15.540 | 10.070 | 51.855 | 1.00 | 25.17 | |
| ATOM | 1175 | O | GLN | 363 | 15.077 | 11.020 | 52.480 | 1.00 | 22.69 | |
| ATOM | 1176 | N | ALA | 364 | 15.023 | 9.659 | 50.701 | 1.00 | 24.53 | |
| ATOM | 1177 | CA | ALA | 364 | 13.863 | 10.340 | 50.099 | 1.00 | 22.80 | |
| ATOM | 1178 | CB | ALA | 364 | 13.524 | 9.691 | 48.753 | 1.00 | 22.91 | |
| ATOM | 1179 | C | ALA | 364 | 12.631 | 10.326 | 51.022 | 1.00 | 21.81 | |
| ATOM | 1180 | O | ALA | 364 | 11.946 | 11.336 | 51.178 | 1.00 | 21.88 | |
| ATOM | 1181 | N | GLU | 365 | 12.362 | 9.188 | 51.636 | 1.00 | 22.72 | |
| ATOM | 1182 | CA | GLU | 365 | 11.218 | 9.062 | 52.532 | 1.00 | 21.63 | |
| ATOM | 1183 | CB | GLU | 365 | 10.990 | 7.585 | 52.856 | 1.00 | 22.68 | |
| ATOM | 1184 | CG | GLU | 365 | 10.724 | 6.806 | 51.583 | 1.00 | 23.15 | |
| ATOM | 1185 | CD | GLU | 365 | 10.797 | 5.303 | 51.723 | 1.00 | 28.04 | |
| ATOM | 1186 | OE1 | GLU | 365 | 11.632 | 4.813 | 52.501 | 1.00 | 26.39 | |
| ATOM | 1187 | OE2 | GLU | 365 | 10.034 | 4.607 | 51.010 | 1.00 | 27.63 | |
| ATOM | 1188 | C | GLU | 365 | 11.454 | 9.881 | 53.798 | 1.00 | 22.43 | |
| ATOM | 1189 | O | GLU | 365 | 10.557 | 10.570 | 54.298 | 1.00 | 22.18 | |
| ATOM | 1190 | N | TYR | 366 | 12.681 | 9.822 | 54.304 | 1.00 | 22.10 | |
| ATOM | 1191 | CA | TYR | 366 | 13.026 | 10.580 | 55.506 | 1.00 | 22.15 | |
| ATOM | 1192 | CB | TYR | 366 | 14.494 | 10.316 | 55.871 | 1.00 | 22.04 | |
| ATOM | 1193 | CG | TYR | 366 | 15.010 | 11.209 | 56.986 | 1.00 | 22.63 | |
| ATOM | 1194 | CD1 | TYR | 366 | 14.580 | 11.030 | 58.307 | 1.00 | 21.87 | |
| ATOM | 1195 | CE1 | TYR | 366 | 15.023 | 11.864 | 59.328 | 1.00 | 22.24 | |
| ATOM | 1196 | CD2 | TYR | 366 | 15.903 | 12.246 | 56.715 | 1.00 | 20.88 | |
| ATOM | 1197 | CE2 | TYR | 366 | 16.366 | 13.092 | 57.731 | 1.00 | 22.98 | |
| ATOM | 1198 | CZ | TYR | 366 | 15.913 | 12.899 | 59.041 | 1.00 | 22.25 | |
| ATOM | 1199 | OH | TYR | 366 | 16.315 | 13.752 | 60.054 | 1.00 | 23.04 | |
| ATOM | 1200 | C | TYR | 366 | 12.797 | 12.095 | 55.332 | 1.00 | 19.98 | |
| ATOM | 1201 | O | TYR | 366 | 12.209 | 12.731 | 56.199 | 1.00 | 19.55 | |
| ATOM | 1202 | N | VAL | 367 | 13.260 | 12.676 | 54.225 | 1.00 | 20.09 | |
| ATOM | 1203 | CA | VAL | 367 | 13.080 | 14.106 | 54.034 | 1.00 | 19.12 | |
| ATOM | 1204 | CB | VAL | 367 | 13.955 | 14.666 | 52.891 | 1.00 | 20.38 | |
| ATOM | 1205 | CG1 | VAL | 367 | 15.446 | 14.473 | 53.249 | 1.00 | 21.37 | |
| ATOM | 1206 | CG2 | VAL | 367 | 13.609 | 14.009 | 51.554 | 1.00 | 19.90 | |
| ATOM | 1207 | C | VAL | 367 | 11.607 | 14.458 | 53.784 | 1.00 | 18.65 | |
| ATOM | 1208 | O | VAL | 367 | 11.165 | 15.538 | 54.178 | 1.00 | 18.48 | |
| ATOM | 1209 | N | ALA | 368 | 10.868 | 13.552 | 53.147 | 1.00 | 20.47 | |
| ATOM | 1210 | CA | ALA | 368 | 9.447 | 13.787 | 52.893 | 1.00 | 19.05 | |
| ATOM | 1211 | CB | ALA | 368 | 8.854 | 12.668 | 51.988 | 1.00 | 18.93 | |
| ATOM | 1212 | C | ALA | 368 | 8.714 | 13.813 | 54.240 | 1.00 | 19.17 | |
| ATOM | 1213 | O | ALA | 368 | 7.863 | 14.682 | 54.487 | 1.00 | 18.81 | |
| ATOM | 1214 | N | LEU | 369 | 9.048 | 12.871 | 55.112 | 1.00 | 19.43 | |
| ATOM | 1215 | CA | LEU | 369 | 8.417 | 12.819 | 56.428 | 1.00 | 17.85 | |
| ATOM | 1216 | CB | LEU | 369 | 8.807 | 11.526 | 57.147 | 1.00 | 20.14 | |
| ATOM | 1217 | CG | LEU | 369 | 8.153 | 10.250 | 56.565 | 1.00 | 21.25 | |
| ATOM | 1218 | CD1 | LEU | 369 | 8.810 | 9.041 | 57.154 | 1.00 | 22.02 | |
| ATOM | 1219 | CD2 | LEU | 369 | 6.626 | 10.219 | 56.856 | 1.00 | 21.64 | |
| ATOM | 1220 | C | LEU | 369 | 8.772 | 14.076 | 57.235 | 1.00 | 19.07 | |
| ATOM | 1221 | O | LEU | 369 | 7.945 | 14.606 | 57.965 | 1.00 | 17.17 | |
| ATOM | 1222 | N | LYS | 370 | 10.007 | 14.567 | 57.120 | 1.00 | 18.01 | |
| ATOM | 1223 | CA | LYS | 370 | 10.363 | 15.799 | 57.826 | 1.00 | 19.26 | |
| ATOM | 1224 | CB | LYS | 370 | 11.819 | 16.199 | 57.522 | 1.00 | 18.97 | |
| ATOM | 1225 | CG | LYS | 370 | 12.888 | 15.349 | 58.203 | 1.00 | 21.31 | |
| ATOM | 1226 | CD | LYS | 370 | 14.304 | 15.860 | 57.845 | 1.00 | 25.49 | |
| ATOM | 1227 | CE | LYS | 370 | 14.445 | 17.367 | 58.009 | 1.00 | 25.79 | |
| ATOM | 1228 | NZ | LYS | 370 | 15.878 | 17.841 | 57.879 | 1.00 | 29.87 | |
| ATOM | 1229 | C | LYS | 370 | 9.440 | 16.949 | 57.388 | 1.00 | 17.68 | |
| ATOM | 1230 | O | LYS | 370 | 8.950 | 17.717 | 58.203 | 1.00 | 17.05 | |
| ATOM | 1231 | N | ALA | 371 | 9.209 | 17.075 | 56.081 | 1.00 | 17.98 | |
| ATOM | 1232 | CA | ALA | 371 | 8.368 | 18.154 | 55.596 | 1.00 | 17.26 | |
| ATOM | 1233 | CB | ALA | 371 | 8.344 | 18.154 | 54.065 | 1.00 | 19.00 | |
| ATOM | 1234 | C | ALA | 371 | 6.957 | 17.957 | 56.140 | 1.00 | 15.65 | |
| ATOM | 1235 | O | ALA | 371 | 6.299 | 18.909 | 56.533 | 1.00 | 17.13 | |
| ATOM | 1236 | N | ILE | 372 | 6.492 | 16.713 | 56.151 | 1.00 | 14.92 | |
| ATOM | 1237 | CA | ILE | 372 | 5.146 | 16.450 | 56.673 | 1.00 | 16.38 | |
| ATOM | 1238 | CB | ILE | 372 | 4.743 | 14.956 | 56.430 | 1.00 | 15.28 | |
| ATOM | 1239 | CG2 | ILE | 372 | 3.477 | 14.602 | 57.245 | 1.00 | 17.98 | |
| ATOM | 1240 | CG1 | ILE | 372 | 4.566 | 14.730 | 54.915 | 1.00 | 18.14 | |
| ATOM | 1241 | CD1 | ILE | 372 | 4.413 | 13.271 | 54.530 | 1.00 | 18.13 | |
| ATOM | 1242 | C | ILE | 372 | 5.038 | 16.795 | 58.163 | 1.00 | 16.98 | |
| ATOM | 1243 | O | ILE | 372 | 4.028 | 17.317 | 58.606 | 1.00 | 16.87 | |
| ATOM | 1244 | N | ILE | 373 | 6.089 | 16.522 | 58.942 | 1.00 | 14.47 | |
| ATOM | 1245 | CA | ILE | 373 | 6.080 | 16.829 | 60.369 | 1.00 | 15.13 | |
| ATOM | 1246 | CB | ILE | 373 | 7.392 | 16.251 | 61.042 | 1.00 | 13.81 | |
| ATOM | 1247 | CG2 | ILE | 373 | 7.530 | 16.783 | 62.463 | 1.00 | 15.08 | |
| ATOM | 1248 | CG1 | ILE | 373 | 7.340 | 14.717 | 61.052 | 1.00 | 14.66 | |
| ATOM | 1249 | CD1 | ILE | 373 | 8.709 | 14.031 | 61.219 | 1.00 | 17.60 | |
| ATOM | 1250 | C | ILE | 373 | 5.962 | 18.350 | 60.604 | 1.00 | 15.01 | |
| ATOM | 1251 | O | ILE | 373 | 5.212 | 18.817 | 61.477 | 1.00 | 15.67 | |
| ATOM | 1252 | N | LEU | 374 | 6.702 | 19.109 | 59.796 | 1.00 | 16.23 | |
| ATOM | 1253 | CA | LEU | 374 | 6.688 | 20.557 | 59.880 | 1.00 | 15.91 | |
| ATOM | 1254 | CB | LEU | 374 | 7.738 | 21.160 | 58.942 | 1.00 | 18.27 | |
| ATOM | 1255 | CG | LEU | 374 | 7.702 | 22.700 | 58.759 | 1.00 | 19.49 | |
| ATOM | 1256 | CD1 | LEU | 374 | 8.184 | 23.403 | 60.044 | 1.00 | 18.70 | |
| ATOM | 1257 | CD2 | LEU | 374 | 8.596 | 23.092 | 57.597 | 1.00 | 19.61 | |
| ATOM | 1258 | C | LEU | 374 | 5.316 | 21.128 | 59.490 | 1.00 | 17.14 | |
| ATOM | 1259 | O | LEU | 374 | 4.765 | 21.980 | 60.167 | 1.00 | 16.72 | |
| ATOM | 1260 | N | LEU | 375 | 4.803 | 20.662 | 58.367 | 1.00 | 17.78 | |
| ATOM | 1261 | CA | LEU | 375 | 3.561 | 21.202 | 57.831 | 1.00 | 17.01 | |
| ATOM | 1262 | CB | LEU | 375 | 3.573 | 21.071 | 56.301 | 1.00 | 19.06 | |
| ATOM | 1263 | CG | LEU | 375 | 4.788 | 21.715 | 55.581 | 1.00 | 18.84 | |
| ATOM | 1264 | CD1 | LEU | 375 | 4.903 | 21.197 | 54.151 | 1.00 | 18.22 | |
| ATOM | 1265 | CD2 | LEU | 375 | 4.644 | 23.234 | 55.610 | 1.00 | 19.41 | |
| ATOM | 1266 | C | LEU | 375 | 2.349 | 20.514 | 58.422 | 1.00 | 19.26 | |
| ATOM | 1267 | O | LEU | 375 | 1.603 | 19.823 | 57.705 | 1.00 | 19.50 | |
| ATOM | 1268 | N | ASN | 376 | 2.178 | 20.727 | 59.723 | 1.00 | 16.95 | |
| ATOM | 1269 | CA | ASN | 376 | 1.087 | 20.136 | 60.510 | 1.00 | 19.78 | |
| ATOM | 1270 | CB | ASN | 376 | 1.605 | 19.772 | 61.912 | 1.00 | 18.97 | |
| ATOM | 1271 | CG | ASN | 376 | 0.537 | 19.124 | 62.780 | 1.00 | 23.13 | |
| ATOM | 1272 | OD1 | ASN | 376 | −0.527 | 18.732 | 62.289 | 1.00 | 19.72 | |
| ATOM | 1273 | ND2 | ASN | 376 | 0.827 | 18.989 | 64.081 | 1.00 | 21.10 | |
| ATOM | 1274 | C | ASN | 376 | −0.088 | 21.080 | 60.641 | 1.00 | 17.80 | |
| ATOM | 1275 | O | ASN | 376 | −0.043 | 22.028 | 61.412 | 1.00 | 18.77 | |
| ATOM | 1276 | N | PRO | 377 | −1.188 | 20.820 | 59.909 | 1.00 | 20.03 | |
| ATOM | 1277 | CD | PRO | 377 | −1.463 | 19.684 | 59.010 | 1.00 | 19.34 | |
| ATOM | 1278 | CA | PRO | 377 | −2.342 | 21.728 | 60.011 | 1.00 | 20.40 | |
| ATOM | 1279 | CB | PRO | 377 | −3.239 | 21.293 | 58.858 | 1.00 | 18.78 | |
| ATOM | 1280 | CG | PRO | 377 | −2.979 | 19.796 | 58.774 | 1.00 | 19.22 | |
| ATOM | 1281 | C | PRO | 377 | −3.086 | 21.705 | 61.336 | 1.00 | 21.89 | |
| ATOM | 1282 | O | PRO | 377 | −3.922 | 22.583 | 61.594 | 1.00 | 22.57 | |
| ATOM | 1283 | N | ASP | 378 | −2.771 | 20.723 | 62.177 | 1.00 | 20.09 | |
| ATOM | 1284 | CA | ASP | 378 | −3.444 | 20.609 | 63.463 | 1.00 | 22.47 | |
| ATOM | 1285 | CB | ASP | 378 | −3.608 | 19.131 | 63.809 | 1.00 | 23.71 | |
| ATOM | 1286 | CG | ASP | 378 | −4.555 | 18.426 | 62.847 | 1.00 | 25.43 | |
| ATOM | 1287 | OD1 | ASP | 378 | −5.579 | 19.028 | 62.487 | 1.00 | 28.35 | |
| ATOM | 1288 | OD2 | ASP | 378 | −4.289 | 17.283 | 62.443 | 1.00 | 30.58 | |
| ATOM | 1289 | C | ASP | 378 | −2.823 | 21.403 | 64.603 | 1.00 | 22.30 | |
| ATOM | 1290 | O | ASP | 378 | −3.265 | 21.309 | 65.760 | 1.00 | 22.98 | |
| ATOM | 1291 | N | VAL | 379 | −1.793 | 22.181 | 64.279 | 1.00 | 22.51 | |
| ATOM | 1292 | CA | VAL | 379 | −1.167 | 23.053 | 65.266 | 1.00 | 21.52 | |
| ATOM | 1293 | CB | VAL | 379 | 0.044 | 23.807 | 64.662 | 1.00 | 20.60 | |
| ATOM | 1294 | CG1 | VAL | 379 | 0.424 | 25.004 | 65.532 | 1.00 | 22.27 | |
| ATOM | 1295 | CG2 | VAL | 379 | 1.226 | 22.848 | 64.516 | 1.00 | 22.31 | |
| ATOM | 1296 | C | VAL | 379 | −2.255 | 24.048 | 65.658 | 1.00 | 23.83 | |
| ATOM | 1297 | O | VAL | 379 | −2.918 | 24.655 | 64.793 | 1.00 | 25.06 | |
| ATOM | 1298 | N | LYS | 380 | −2.460 | 24.212 | 66.958 | 1.00 | 23.33 | |
| ATOM | 1299 | CA | LYS | 380 | −3.490 | 25.124 | 67.430 | 1.00 | 24.49 | |
| ATOM | 1300 | CB | LYS | 380 | −3.797 | 24.829 | 68.904 | 1.00 | 25.21 | |
| ATOM | 1301 | CG | LYS | 380 | −4.182 | 23.365 | 69.131 | 1.00 | 28.03 | |
| ATOM | 1302 | CD | LYS | 380 | −4.460 | 23.035 | 70.594 | 1.00 | 30.82 | |
| ATOM | 1303 | CE | LYS | 380 | −4.728 | 21.536 | 70.758 | 1.00 | 31.68 | |
| ATOM | 1304 | NZ | LYS | 380 | −4.904 | 21.079 | 72.177 | 1.00 | 33.50 | |
| ATOM | 1305 | C | LYS | 380 | −3.055 | 26.573 | 67.250 | 1.00 | 24.85 | |
| ATOM | 1306 | O | LYS | 380 | −1.874 | 26.891 | 67.408 | 1.00 | 25.55 | |
| ATOM | 1307 | N | GLY | 381 | −3.988 | 27.445 | 66.861 | 1.00 | 24.69 | |
| ATOM | 1308 | CA | GLY | 381 | −3.647 | 28.852 | 66.704 | 1.00 | 26.29 | |
| ATOM | 1309 | C | GLY | 381 | −3.384 | 29.378 | 65.295 | 1.00 | 25.30 | |
| ATOM | 1310 | O | GLY | 381 | −3.246 | 30.598 | 65.093 | 1.00 | 27.29 | |
| ATOM | 1311 | N | LEU | 382 | −3.313 | 28.490 | 64.311 | 1.00 | 26.35 | |
| ATOM | 1312 | CA | LEU | 382 | −3.054 | 28.914 | 62.941 | 1.00 | 26.95 | |
| ATOM | 1313 | CB | LEU | 382 | −2.997 | 27.706 | 61.995 | 1.00 | 25.01 | |
| ATOM | 1314 | CG | LEU | 382 | −1.865 | 26.700 | 62.265 | 1.00 | 24.87 | |
| ATOM | 1315 | CD1 | LEU | 382 | −2.011 | 25.507 | 61.314 | 1.00 | 24.53 | |
| ATOM | 1316 | CD2 | LEU | 382 | −0.518 | 27.384 | 62.064 | 1.00 | 24.40 | |
| ATOM | 1317 | C | LEU | 382 | −4.120 | 29.885 | 62.428 | 1.00 | 28.77 | |
| ATOM | 1318 | O | LEU | 382 | −5.310 | 29.719 | 62.695 | 1.00 | 28.39 | |
| ATOM | 1319 | N | LYS | 383 | −3.660 | 30.881 | 61.681 | 1.00 | 30.83 | |
| ATOM | 1320 | CA | LYS | 383 | −4.518 | 31.894 | 61.078 | 1.00 | 32.60 | |
| ATOM | 1321 | CB | LYS | 383 | −3.670 | 33.104 | 60.699 | 1.00 | 35.65 | |
| ATOM | 1322 | CG | LYS | 383 | −4.352 | 34.121 | 59.788 | 1.00 | 40.31 | |
| ATOM | 1323 | CD | LYS | 383 | −5.044 | 35.203 | 60.589 | 1.00 | 42.92 | |
| ATOM | 1324 | CE | LYS | 383 | −5.436 | 36.376 | 59.702 | 1.00 | 43.80 | |
| ATOM | 1325 | NZ | LYS | 383 | −6.040 | 37.476 | 60.505 | 1.00 | 44.52 | |
| ATOM | 1326 | C | LYS | 383 | −5.159 | 31.313 | 59.819 | 1.00 | 32.46 | |
| ATOM | 1327 | O | LYS | 383 | −6.374 | 31.411 | 59.620 | 1.00 | 33.42 | |
| ATOM | 1328 | N | ASN | 384 | −4.332 | 30.700 | 58.977 | 1.00 | 30.19 | |
| ATOM | 1329 | CA | ASN | 384 | −4.794 | 30.118 | 57.720 | 1.00 | 29.86 | |
| ATOM | 1330 | CB | ASN | 384 | −4.154 | 30.854 | 56.549 | 1.00 | 32.74 | |
| ATOM | 1331 | CG | ASN | 384 | −4.460 | 32.336 | 56.570 | 1.00 | 36.46 | |
| ATOM | 1332 | OD1 | ASN | 384 | −5.625 | 32.730 | 56.585 | 1.00 | 39.59 | |
| ATOM | 1333 | ND2 | ASN | 384 | −3.418 | 33.166 | 56.583 | 1.00 | 37.88 | |
| ATOM | 1334 | C | ASN | 384 | −4.466 | 28.636 | 57.636 | 1.00 | 26.29 | |
| ATOM | 1335 | O | ASN | 384 | −3.558 | 28.229 | 56.912 | 1.00 | 26.38 | |
| ATOM | 1336 | N | ARG | 385 | −5.218 | 27.845 | 58.387 | 1.00 | 24.62 | |
| ATOM | 1337 | CA | ARG | 385 | −5.005 | 26.406 | 58.431 | 1.00 | 24.85 | |
| ATOM | 1338 | CB | ARG | 385 | −5.994 | 25.779 | 59.413 | 1.00 | 24.62 | |
| ATOM | 1339 | CG | ARG | 385 | −5.910 | 24.275 | 59.561 | 1.00 | 27.77 | |
| ATOM | 1340 | CD | ARG | 385 | −6.705 | 23.839 | 60.785 | 1.00 | 30.37 | |
| ATOM | 1341 | NE | ARG | 385 | −5.976 | 24.188 | 62.004 | 1.00 | 31.78 | |
| ATOM | 1342 | CZ | ARG | 385 | −6.329 | 25.148 | 62.849 | 1.00 | 31.80 | |
| ATOM | 1343 | NH1 | ARG | 385 | −7.432 | 25.867 | 62.629 | 1.00 | 31.71 | |
| ATOM | 1344 | NH2 | ARG | 385 | −5.542 | 25.426 | 63.886 | 1.00 | 28.60 | |
| ATOM | 1345 | C | ARG | 385 | −5.079 | 25.727 | 57.070 | 1.00 | 24.38 | |
| ATOM | 1346 | O | ARG | 385 | −4.289 | 24.824 | 56.783 | 1.00 | 24.23 | |
| ATOM | 1347 | N | GLN | 386 | −6.003 | 26.165 | 56.214 | 1.00 | 24.39 | |
| ATOM | 1348 | CA | GLN | 386 | −6.128 | 25.542 | 54.906 | 1.00 | 25.99 | |
| ATOM | 1349 | CB | GLN | 386 | −7.320 | 26.124 | 54.128 | 1.00 | 29.68 | |
| ATOM | 1350 | CG | GLN | 386 | −7.256 | 27.619 | 53.863 | 1.00 | 34.93 | |
| ATOM | 1351 | CD | GLN | 386 | −7.246 | 28.442 | 55.136 | 1.00 | 37.35 | |
| ATOM | 1352 | OE1 | GLN | 386 | −8.016 | 28.183 | 56.072 | 1.00 | 39.44 | |
| ATOM | 1353 | NE2 | GLN | 386 | −6.373 | 29.443 | 55.183 | 1.00 | 39.95 | |
| ATOM | 1354 | C | GLN | 386 | −4.867 | 25.646 | 54.067 | 1.00 | 24.30 | |
| ATOM | 1355 | O | GLN | 386 | −4.561 | 24.727 | 53.318 | 1.00 | 25.10 | |
| ATOM | 1356 | N | GLU | 387 | −4.140 | 26.752 | 54.182 | 1.00 | 25.50 | |
| ATOM | 1357 | CA | GLU | 387 | −2.907 | 26.905 | 53.413 | 1.00 | 25.38 | |
| ATOM | 1358 | CB | GLU | 387 | −2.358 | 28.328 | 53.594 | 1.00 | 29.21 | |
| ATOM | 1359 | CG | GLU | 387 | −3.234 | 29.420 | 52.915 | 1.00 | 33.12 | |
| ATOM | 1360 | CD | GLU | 387 | −2.845 | 30.838 | 53.284 | 1.00 | 37.28 | |
| ATOM | 1361 | OE1 | GLU | 387 | −1.634 | 31.125 | 53.407 | 1.00 | 40.75 | |
| ATOM | 1362 | OE2 | GLU | 387 | −3.750 | 31.691 | 53.436 | 1.00 | 38.95 | |
| ATOM | 1363 | C | GLU | 387 | −1.877 | 25.841 | 53.852 | 1.00 | 25.54 | |
| ATOM | 1364 | O | GLU | 387 | −1.097 | 25.329 | 53.030 | 1.00 | 24.67 | |
| ATOM | 1365 | N | VAL | 388 | −1.897 | 25.476 | 55.132 | 1.00 | 24.28 | |
| ATOM | 1366 | CA | VAL | 388 | −0.960 | 24.447 | 55.617 | 1.00 | 21.40 | |
| ATOM | 1367 | CB | VAL | 388 | −0.845 | 24.439 | 57.168 | 1.00 | 19.68 | |
| ATOM | 1368 | CG1 | VAL | 388 | 0.182 | 23.410 | 57.597 | 1.00 | 22.22 | |
| ATOM | 1369 | CG2 | VAL | 388 | −0.477 | 25.824 | 57.691 | 1.00 | 20.28 | |
| ATOM | 1370 | C | VAL | 388 | −1.444 | 23.076 | 55.178 | 1.00 | 22.10 | |
| ATOM | 1371 | O | VAL | 388 | −0.666 | 22.211 | 54.782 | 1.00 | 21.10 | |
| ATOM | 1372 | N | GLU | 389 | −2.750 | 22.859 | 55.243 | 1.00 | 22.00 | |
| ATOM | 1373 | CA | GLU | 389 | −3.284 | 21.577 | 54.825 | 1.00 | 21.41 | |
| ATOM | 1374 | CB | GLU | 389 | −4.801 | 21.538 | 54.987 | 1.00 | 22.76 | |
| ATOM | 1375 | CG | GLU | 389 | −5.358 | 20.146 | 54.988 | 1.00 | 31.47 | |
| ATOM | 1376 | CD | GLU | 389 | −6.793 | 20.093 | 55.471 | 1.00 | 36.14 | |
| ATOM | 1377 | OE1 | GLU | 389 | −7.160 | 20.927 | 56.334 | 1.00 | 39.74 | |
| ATOM | 1378 | OE2 | GLU | 389 | −7.540 | 19.204 | 55.006 | 1.00 | 39.63 | |
| ATOM | 1379 | C | GLU | 389 | −2.936 | 21.294 | 53.382 | 1.00 | 19.32 | |
| ATOM | 1380 | O | GLU | 389 | −2.653 | 20.155 | 53.025 | 1.00 | 21.32 | |
| ATOM | 1381 | N | VAL | 390 | −3.001 | 22.313 | 52.534 | 1.00 | 19.72 | |
| ATOM | 1382 | CA | VAL | 390 | −2.685 | 22.148 | 51.125 | 1.00 | 19.82 | |
| ATOM | 1383 | CB | VAL | 390 | −2.950 | 23.447 | 50.316 | 1.00 | 19.77 | |
| ATOM | 1384 | CG1 | VAL | 390 | −2.307 | 23.344 | 48.919 | 1.00 | 22.22 | |
| ATOM | 1385 | CG2 | VAL | 390 | −4.442 | 23.652 | 50.169 | 1.00 | 23.95 | |
| ATOM | 1386 | C | VAL | 390 | −1.231 | 21.744 | 50.924 | 1.00 | 20.27 | |
| ATOM | 1387 | O | VAL | 390 | −0.924 | 20.894 | 50.093 | 1.00 | 20.79 | |
| ATOM | 1388 | N | LEU | 391 | −0.334 | 22.345 | 51.699 | 1.00 | 21.06 | |
| ATOM | 1389 | CA | LEU | 391 | 1.078 | 22.004 | 51.552 | 1.00 | 19.84 | |
| ATOM | 1390 | CB | LEU | 391 | 1.951 | 22.982 | 52.328 | 1.00 | 20.90 | |
| ATOM | 1391 | CG | LEU | 391 | 1.895 | 24.438 | 51.836 | 1.00 | 21.65 | |
| ATOM | 1392 | CD1 | LEU | 391 | 2.846 | 25.283 | 52.673 | 1.00 | 23.70 | |
| ATOM | 1393 | CD2 | LEU | 391 | 2.260 | 24.515 | 50.351 | 1.00 | 25.56 | |
| ATOM | 1394 | C | LEU | 391 | 1.327 | 20.583 | 52.031 | 1.00 | 19.59 | |
| ATOM | 1395 | O | LEU | 391 | 2.090 | 19.837 | 51.421 | 1.00 | 19.63 | |
| ATOM | 1396 | N | ARG | 392 | 0.680 | 20.192 | 53.130 | 0.50 | 19.30 | AC1 |
| ATOM | 1397 | CA | ARG | 392 | 0.862 | 18.823 | 53.628 | 0.50 | 19.65 | AC1 |
| ATOM | 1398 | CB | ARG | 392 | 0.212 | 18.620 | 55.016 | 0.50 | 19.22 | AC1 |
| ATOM | 1399 | CG | ARG | 392 | 0.060 | 17.123 | 55.382 | 0.50 | 18.74 | AC1 |
| ATOM | 1400 | CD | ARG | 392 | −0.380 | 16.857 | 56.840 | 0.50 | 19.04 | AC1 |
| ATOM | 1401 | NE | ARG | 392 | 0.710 | 17.001 | 57.811 | 0.50 | 18.30 | AC1 |
| ATOM | 1402 | CZ | ARG | 392 | 0.739 | 16.427 | 59.020 | 0.50 | 18.32 | AC1 |
| ATOM | 1403 | NH1 | ARG | 392 | −0.262 | 15.660 | 59.432 | 0.50 | 18.09 | AC1 |
| ATOM | 1404 | NH2 | ARG | 392 | 1.783 | 16.606 | 59.826 | 0.50 | 13.08 | AC1 |
| ATOM | 1405 | C | ARG | 392 | 0.259 | 17.833 | 52.624 | 0.50 | 19.79 | AC1 |
| ATOM | 1406 | O | ARG | 392 | 0.768 | 16.730 | 52.453 | 0.50 | 19.90 | AC1 |
| ATOM | 1407 | N | GLU | 393 | −0.839 | 18.220 | 51.976 | 1.00 | 19.51 | |
| ATOM | 1408 | CA | GLU | 393 | −1.484 | 17.355 | 50.979 | 1.00 | 21.32 | |
| ATOM | 1409 | CB | GLU | 393 | −2.858 | 17.922 | 50.575 | 1.00 | 23.83 | |
| ATOM | 1410 | CG | GLU | 393 | −3.631 | 17.066 | 49.588 | 1.00 | 29.44 | |
| ATOM | 1411 | CD | GLU | 393 | −4.209 | 15.767 | 50.173 | 1.00 | 34.23 | |
| ATOM | 1412 | OE1 | GLU | 393 | −3.940 | 15.434 | 51.348 | 1.00 | 34.71 | |
| ATOM | 1413 | OE2 | GLU | 393 | −4.952 | 15.071 | 49.433 | 1.00 | 37.12 | |
| ATOM | 1414 | C | GLU | 393 | −0.556 | 17.210 | 49.763 | 1.00 | 21.12 | |
| ATOM | 1415 | O | GLU | 393 | −0.486 | 16.147 | 49.157 | 1.00 | 20.35 | |
| ATOM | 1416 | N | LYS | 394 | 0.143 | 18.287 | 49.395 | 1.00 | 19.98 | |
| ATOM | 1417 | CA | LYS | 394 | 1.102 | 18.186 | 48.302 | 1.00 | 21.24 | |
| ATOM | 1418 | CB | LYS | 394 | 1.787 | 19.519 | 48.048 | 1.00 | 22.05 | |
| ATOM | 1419 | CG | LYS | 394 | 0.903 | 20.468 | 47.258 | 1.00 | 24.02 | |
| ATOM | 1420 | CD | LYS | 394 | 1.608 | 21.778 | 46.964 | 1.00 | 25.62 | |
| ATOM | 1421 | CE | LYS | 394 | 0.690 | 22.711 | 46.199 | 1.00 | 28.47 | |
| ATOM | 1422 | NZ | LYS | 394 | 1.369 | 24.012 | 45.959 | 1.00 | 30.41 | |
| ATOM | 1423 | C | LYS | 394 | 2.153 | 17.137 | 48.667 | 1.00 | 22.08 | |
| ATOM | 1424 | O | LYS | 394 | 2.555 | 16.341 | 47.825 | 1.00 | 20.82 | |
| ATOM | 1425 | N | MET | 395 | 2.590 | 17.136 | 49.932 | 1.00 | 21.43 | |
| ATOM | 1426 | CA | MET | 395 | 3.607 | 16.166 | 50.369 | 1.00 | 21.13 | |
| ATOM | 1427 | CB | MET | 395 | 4.177 | 16.520 | 51.746 | 1.00 | 24.05 | |
| ATOM | 1428 | CG | MET | 395 | 5.100 | 17.680 | 51.738 | 1.00 | 24.66 | |
| ATOM | 1429 | SD | MET | 395 | 6.457 | 17.503 | 50.571 | 1.00 | 27.36 | |
| ATOM | 1430 | CE | MET | 395 | 6.985 | 15.797 | 50.738 | 1.00 | 23.91 | |
| ATOM | 1431 | C | MET | 395 | 3.121 | 14.744 | 50.417 | 1.00 | 20.46 | |
| ATOM | 1432 | O | MET | 395 | 3.872 | 13.832 | 50.095 | 1.00 | 22.42 | |
| ATOM | 1433 | N | PHE | 396 | 1.862 | 14.542 | 50.827 | 1.00 | 20.87 | |
| ATOM | 1434 | CA | PHE | 396 | 1.285 | 13.202 | 50.858 | 1.00 | 21.65 | |
| ATOM | 1435 | CB | PHE | 396 | −0.176 | 13.203 | 51.374 | 1.00 | 20.90 | |
| ATOM | 1436 | CG | PHE | 396 | −0.320 | 13.286 | 52.875 | 1.00 | 22.60 | |
| ATOM | 1437 | CD1 | PHE | 396 | 0.733 | 12.996 | 53.727 | 1.00 | 22.79 | |
| ATOM | 1438 | CD2 | PHE | 396 | −1.544 | 13.640 | 53.436 | 1.00 | 23.58 | |
| ATOM | 1439 | CE1 | PHE | 396 | 0.570 | 13.063 | 55.116 | 1.00 | 20.29 | |
| ATOM | 1440 | CE2 | PHE | 396 | −1.712 | 13.702 | 54.826 | 1.00 | 23.72 | |
| ATOM | 1441 | CZ | PHE | 396 | −0.655 | 13.416 | 55.669 | 1.00 | 22.24 | |
| ATOM | 1442 | C | PHE | 396 | 1.281 | 12.652 | 49.429 | 1.00 | 22.43 | |
| ATOM | 1443 | O | PHE | 396 | 1.635 | 11.494 | 49.215 | 1.00 | 21.80 | |
| ATOM | 1444 | N | LEU | 397 | 0.844 | 13.462 | 48.458 | 1.00 | 22.96 | |
| ATOM | 1445 | CA | LEU | 397 | 0.813 | 13.022 | 47.061 | 1.00 | 21.97 | |
| ATOM | 1446 | CB | LEU | 397 | 0.139 | 14.099 | 46.176 | 1.00 | 22.88 | |
| ATOM | 1447 | CG | LEU | 397 | −1.385 | 14.161 | 46.354 | 1.00 | 23.93 | |
| ATOM | 1448 | CD1 | LEU | 397 | −1.981 | 15.078 | 45.315 | 1.00 | 25.27 | |
| ATOM | 1449 | CD2 | LEU | 397 | −1.972 | 12.759 | 46.190 | 1.00 | 25.99 | |
| ATOM | 1450 | C | LEU | 397 | 2.233 | 12.705 | 46.560 | 1.00 | 24.94 | |
| ATOM | 1451 | O | LEU | 397 | 2.450 | 11.692 | 45.873 | 1.00 | 25.61 | |
| ATOM | 1452 | N | CYS | 398 | 3.195 | 13.548 | 46.924 | 1.00 | 23.80 | |
| ATOM | 1453 | CA | CYS | 398 | 4.593 | 13.333 | 46.519 | 1.00 | 26.51 | |
| ATOM | 1454 | CB | CYS | 398 | 5.509 | 14.454 | 47.041 | 1.00 | 28.87 | |
| ATOM | 1455 | SG | CYS | 398 | 5.551 | 15.910 | 46.032 | 1.00 | 39.48 | |
| ATOM | 1456 | C | CYS | 398 | 5.111 | 12.027 | 47.090 | 1.00 | 25.35 | |
| ATOM | 1457 | O | CYS | 398 | 5.738 | 11.221 | 46.399 | 1.00 | 25.03 | |
| ATOM | 1458 | N | LEU | 399 | 4.861 | 11.821 | 48.371 | 1.00 | 24.81 | |
| ATOM | 1459 | CA | LEU | 399 | 5.366 | 10.620 | 49.009 | 1.00 | 24.04 | |
| ATOM | 1460 | CB | LEU | 399 | 5.232 | 10.739 | 50.530 | 1.00 | 24.26 | |
| ATOM | 1461 | CG | LEU | 399 | 5.806 | 9.580 | 51.344 | 1.00 | 24.01 | |
| ATOM | 1462 | CD1 | LEU | 399 | 7.281 | 9.343 | 50.985 | 1.00 | 22.08 | |
| ATOM | 1463 | CD2 | LEU | 399 | 5.672 | 9.931 | 52.821 | 1.00 | 22.85 | |
| ATOM | 1464 | C | LEU | 399 | 4.715 | 9.352 | 48.518 | 1.00 | 26.35 | |
| ATOM | 1465 | O | LEU | 399 | 5.398 | 8.363 | 48.267 | 1.00 | 25.02 | |
| ATOM | 1466 | N | ASP | 400 | 3.391 | 9.377 | 48.361 | 1.00 | 27.30 | |
| ATOM | 1467 | CA | ASP | 400 | 2.680 | 8.200 | 47.874 | 1.00 | 29.09 | |
| ATOM | 1468 | CB | ASP | 400 | 1.179 | 8.490 | 47.772 | 1.00 | 31.25 | |
| ATOM | 1469 | CG | ASP | 400 | 0.379 | 7.258 | 47.400 | 1.00 | 34.75 | |
| ATOM | 1470 | OD1 | ASP | 400 | 0.236 | 6.356 | 48.252 | 1.00 | 35.16 | |
| ATOM | 1471 | OD2 | ASP | 400 | −0.091 | 7.188 | 46.250 | 1.00 | 38.49 | |
| ATOM | 1472 | C | ASP | 400 | 3.217 | 7.816 | 46.497 | 1.00 | 28.91 | |
| ATOM | 1473 | O | ASP | 400 | 3.436 | 6.642 | 46.212 | 1.00 | 29.66 | |
| ATOM | 1474 | N | GLU | 401 | 3.420 | 8.813 | 45.644 | 1.00 | 28.16 | |
| ATOM | 1475 | CA | GLU | 401 | 3.936 | 8.577 | 44.299 | 1.00 | 29.87 | |
| ATOM | 1476 | CB | GLU | 401 | 4.052 | 9.895 | 43.533 | 1.00 | 32.50 | |
| ATOM | 1477 | CG | GLU | 401 | 4.735 | 9.751 | 42.180 | 1.00 | 38.83 | |
| ATOM | 1478 | CD | GLU | 401 | 4.784 | 11.048 | 41.406 | 1.00 | 42.93 | |
| ATOM | 1479 | OE1 | GLU | 401 | 5.449 | 12.003 | 41.865 | 1.00 | 45.46 | |
| ATOM | 1480 | OE2 | GLU | 401 | 4.147 | 11.114 | 40.334 | 1.00 | 45.87 | |
| ATOM | 1481 | C | GLU | 401 | 5.308 | 7.911 | 44.360 | 1.00 | 31.19 | |
| ATOM | 1482 | O | GLU | 401 | 5.574 | 6.926 | 43.650 | 1.00 | 31.37 | |
| ATOM | 1483 | N | TYR | 402 | 6.171 | 8.444 | 45.224 | 1.00 | 28.55 | |
| ATOM | 1484 | CA | TYR | 402 | 7.516 | 7.918 | 45.365 | 1.00 | 29.01 | |
| ATOM | 1485 | CB | TYR | 402 | 8.314 | 8.737 | 46.384 | 1.00 | 27.40 | |
| ATOM | 1486 | CG | TYR | 402 | 9.667 | 8.134 | 46.659 | 1.00 | 27.45 | |
| ATOM | 1487 | CD1 | TYR | 402 | 9.849 | 7.238 | 47.709 | 1.00 | 27.18 | |
| ATOM | 1488 | CE1 | TYR | 402 | 11.087 | 6.635 | 47.933 | 1.00 | 28.15 | |
| ATOM | 1489 | CD2 | TYR | 402 | 10.747 | 8.419 | 45.834 | 1.00 | 27.22 | |
| ATOM | 1490 | CE2 | TYR | 402 | 11.996 | 7.820 | 46.050 | 1.00 | 30.03 | |
| ATOM | 1491 | CZ | TYR | 402 | 12.147 | 6.936 | 47.097 | 1.00 | 30.78 | |
| ATOM | 1492 | OH | TYR | 402 | 13.374 | 6.358 | 47.316 | 1.00 | 31.66 | |
| ATOM | 1493 | C | TYR | 402 | 7.503 | 6.453 | 45.769 | 1.00 | 30.16 | |
| ATOM | 1494 | O | TYR | 402 | 8.206 | 5.641 | 45.187 | 1.00 | 29.67 | |
| ATOM | 1495 | N | CYS | 403 | 6.700 | 6.113 | 46.763 | 1.00 | 30.42 | |
| ATOM | 1496 | CA | CYS | 403 | 6.616 | 4.739 | 47.215 | 1.00 | 34.32 | |
| ATOM | 1497 | CB | CYS | 403 | 5.691 | 4.650 | 48.430 | 1.00 | 33.37 | |
| ATOM | 1498 | SG | CYS | 403 | 6.357 | 5.485 | 49.920 | 1.00 | 31.63 | |
| ATOM | 1499 | C | CYS | 403 | 6.116 | 3.820 | 46.104 | 1.00 | 36.81 | |
| ATOM | 1500 | O | CYS | 403 | 6.612 | 2.703 | 45.936 | 1.00 | 36.95 | |
| ATOM | 1501 | N | ARG | 404 | 5.140 | 4.289 | 45.335 | 1.00 | 39.50 | |
| ATOM | 1502 | CA | ARG | 404 | 4.597 | 3.453 | 44.271 | 1.00 | 42.67 | |
| ATOM | 1503 | CB | ARG | 404 | 3.280 | 4.045 | 43.746 | 1.00 | 43.44 | |
| ATOM | 1504 | CG | ARG | 404 | 2.135 | 3.802 | 44.718 | 1.00 | 46.08 | |
| ATOM | 1505 | CD | ARG | 404 | 0.746 | 4.164 | 44.188 | 1.00 | 46.89 | |
| ATOM | 1506 | NE | ARG | 404 | 0.490 | 5.602 | 44.159 | 1.00 | 46.69 | |
| ATOM | 1507 | CZ | ARG | 404 | 0.733 | 6.386 | 43.116 | 1.00 | 47.48 | |
| ATOM | 1508 | NH1 | ARG | 404 | 1.241 | 5.874 | 42.002 | 1.00 | 48.94 | |
| ATOM | 1509 | NH2 | ARG | 404 | 0.462 | 7.684 | 43.181 | 1.00 | 48.22 | |
| ATOM | 1510 | C | ARG | 404 | 5.578 | 3.206 | 43.137 | 1.00 | 43.67 | |
| ATOM | 1511 | O | ARG | 404 | 5.640 | 2.102 | 42.598 | 1.00 | 44.97 | |
| ATOM | 1512 | N | ARG | 405 | 6.356 | 4.220 | 42.785 | 1.00 | 45.45 | |
| ATOM | 1513 | CA | ARG | 405 | 7.330 | 4.098 | 41.710 | 1.00 | 47.72 | |
| ATOM | 1514 | CB | ARG | 405 | 7.700 | 5.481 | 41.167 | 1.00 | 49.80 | |
| ATOM | 1515 | CG | ARG | 405 | 6.535 | 6.298 | 40.624 | 1.00 | 53.09 | |
| ATOM | 1516 | CD | ARG | 405 | 7.005 | 7.695 | 40.233 | 1.00 | 56.62 | |
| ATOM | 1517 | NE | ARG | 405 | 5.931 | 8.506 | 39.661 | 1.00 | 59.35 | |
| ATOM | 1518 | CZ | ARG | 405 | 5.356 | 8.263 | 38.488 | 1.00 | 60.20 | |
| ATOM | 1519 | NH1 | ARG | 405 | 5.753 | 7.231 | 37.756 | 1.00 | 61.80 | |
| ATOM | 1520 | NH2 | ARG | 405 | 4.382 | 9.049 | 38.046 | 1.00 | 60.75 | |
| ATOM | 1521 | C | ARG | 405 | 8.609 | 3.410 | 42.166 | 1.00 | 48.23 | |
| ATOM | 1522 | O | ARG | 405 | 9.078 | 2.464 | 41.528 | 1.00 | 48.90 | |
| ATOM | 1523 | N | SER | 406 | 9.163 | 3.891 | 43.278 | 1.00 | 47.66 | |
| ATOM | 1524 | CA | SER | 406 | 10.421 | 3.382 | 43.819 | 1.00 | 48.66 | |
| ATOM | 1525 | CB | SER | 406 | 11.099 | 4.477 | 44.638 | 1.00 | 49.49 | |
| ATOM | 1526 | OG | SER | 406 | 11.118 | 5.698 | 43.919 | 1.00 | 51.64 | |
| ATOM | 1527 | C | SER | 406 | 10.336 | 2.112 | 44.656 | 1.00 | 48.77 | |
| ATOM | 1528 | O | SER | 406 | 11.355 | 1.507 | 44.968 | 1.00 | 48.91 | |
| ATOM | 1529 | N | ARG | 407 | 9.129 | 1.711 | 45.026 | 1.00 | 49.27 | |
| ATOM | 1530 | CA | ARG | 407 | 8.948 | 0.500 | 45.813 | 1.00 | 50.47 | |
| ATOM | 1531 | CB | ARG | 407 | 9.106 | 0.811 | 47.302 | 1.00 | 52.25 | |
| ATOM | 1532 | CG | ARG | 407 | 10.476 | 0.434 | 47.849 | 1.00 | 54.88 | |
| ATOM | 1533 | CD | ARG | 407 | 10.942 | 1.401 | 48.918 | 1.00 | 55.39 | |
| ATOM | 1534 | NE | ARG | 407 | 12.157 | 0.927 | 49.573 | 1.00 | 56.11 | |
| ATOM | 1535 | CZ | ARG | 407 | 12.885 | 1.653 | 50.416 | 1.00 | 56.40 | |
| ATOM | 1536 | NH1 | ARG | 407 | 12.524 | 2.895 | 50.707 | 1.00 | 56.21 | |
| ATOM | 1537 | NH2 | ARG | 407 | 13.973 | 1.133 | 50.972 | 1.00 | 55.90 | |
| ATOM | 1538 | C | ARG | 407 | 7.586 | −0.110 | 45.535 | 1.00 | 50.00 | |
| ATOM | 1539 | O | ARG | 407 | 6.847 | −0.468 | 46.451 | 1.00 | 50.06 | |
| ATOM | 1540 | N | SER | 408 | 7.279 | −0.233 | 44.248 | 1.00 | 49.76 | |
| ATOM | 1541 | CA | SER | 408 | 6.010 | −0.777 | 43.782 | 1.00 | 49.98 | |
| ATOM | 1542 | CB | SER | 408 | 6.060 | −0.957 | 42.262 | 1.00 | 50.23 | |
| ATOM | 1543 | OG | SER | 408 | 7.129 | −1.806 | 41.880 | 1.00 | 50.48 | |
| ATOM | 1544 | C | SER | 408 | 5.622 | −2.100 | 44.439 | 1.00 | 49.86 | |
| ATOM | 1545 | O | SER | 408 | 4.444 | −2.354 | 44.698 | 1.00 | 50.10 | |
| ATOM | 1546 | N | SER | 409 | 6.620 | −2.933 | 44.706 | 1.00 | 50.01 | |
| ATOM | 1547 | CA | SER | 409 | 6.412 | −4.245 | 45.307 | 1.00 | 49.71 | |
| ATOM | 1548 | CB | SER | 409 | 7.704 | −5.055 | 45.217 | 1.00 | 50.85 | |
| ATOM | 1549 | OG | SER | 409 | 8.799 | −4.306 | 45.730 | 1.00 | 52.55 | |
| ATOM | 1550 | C | SER | 409 | 5.954 | −4.195 | 46.758 | 1.00 | 49.15 | |
| ATOM | 1551 | O | SER | 409 | 5.476 | −5.195 | 47.300 | 1.00 | 48.48 | |
| ATOM | 1552 | N | GLU | 410 | 6.095 | −3.035 | 47.392 | 1.00 | 47.66 | |
| ATOM | 1553 | CA | GLU | 410 | 5.701 | −2.912 | 48.785 | 1.00 | 45.50 | |
| ATOM | 1554 | CB | GLU | 410 | 6.787 | −2.197 | 49.577 | 1.00 | 46.29 | |
| ATOM | 1555 | CG | GLU | 410 | 8.079 | −2.980 | 49.712 | 1.00 | 47.76 | |
| ATOM | 1556 | CD | GLU | 410 | 9.074 | −2.261 | 50.592 | 1.00 | 48.13 | |
| ATOM | 1557 | OE1 | GLU | 410 | 10.155 | −1.882 | 50.092 | 1.00 | 49.66 | |
| ATOM | 1558 | OE2 | GLU | 410 | 8.761 | −2.063 | 51.784 | 1.00 | 48.57 | |
| ATOM | 1559 | C | GLU | 410 | 4.373 | −2.197 | 48.973 | 1.00 | 44.10 | |
| ATOM | 1560 | O | GLU | 410 | 4.311 | −0.974 | 49.114 | 1.00 | 41.70 | |
| ATOM | 1561 | N | GLU | 411 | 3.306 | −2.985 | 48.986 | 1.00 | 42.76 | |
| ATOM | 1562 | CA | GLU | 411 | 1.973 | −2.451 | 49.154 | 1.00 | 41.57 | |
| ATOM | 1563 | CB | GLU | 411 | 0.945 | −3.529 | 48.814 | 1.00 | 44.42 | |
| ATOM | 1564 | CG | GLU | 411 | 1.166 | −4.132 | 47.439 | 1.00 | 48.49 | |
| ATOM | 1565 | CD | GLU | 411 | 0.151 | −5.191 | 47.100 | 1.00 | 49.60 | |
| ATOM | 1566 | OE1 | GLU | 411 | 0.111 | −6.226 | 47.806 | 1.00 | 49.80 | |
| ATOM | 1567 | OE2 | GLU | 411 | −0.605 | −4.979 | 46.129 | 1.00 | 50.58 | |
| ATOM | 1568 | C | GLU | 411 | 1.805 | −2.000 | 50.594 | 1.00 | 38.78 | |
| ATOM | 1569 | O | GLU | 411 | 2.254 | −2.682 | 51.526 | 1.00 | 38.53 | |
| ATOM | 1570 | N | GLY | 412 | 1.170 | −0.845 | 50.768 | 1.00 | 34.78 | |
| ATOM | 1571 | CA | GLY | 412 | 0.953 | −0.313 | 52.103 | 1.00 | 32.19 | |
| ATOM | 1572 | C | GLY | 412 | 2.163 | 0.402 | 52.695 | 1.00 | 29.28 | |
| ATOM | 1573 | O | GLY | 412 | 2.133 | 0.770 | 53.865 | 1.00 | 28.68 | |
| ATOM | 1574 | N | ARG | 413 | 3.207 | 0.625 | 51.901 | 1.00 | 28.70 | |
| ATOM | 1575 | CA | ARG | 413 | 4.413 | 1.286 | 52.407 | 1.00 | 27.65 | |
| ATOM | 1576 | CB | ARG | 413 | 5.513 | 1.319 | 51.348 | 1.00 | 29.50 | |
| ATOM | 1577 | CG | ARG | 413 | 6.850 | 1.839 | 51.905 | 1.00 | 29.56 | |
| ATOM | 1578 | CD | ARG | 413 | 8.004 | 1.721 | 50.903 | 1.00 | 32.68 | |
| ATOM | 1579 | NE | ARG | 413 | 9.242 | 2.244 | 51.484 | 1.00 | 34.48 | |
| ATOM | 1580 | CZ | ARG | 413 | 9.951 | 1.632 | 52.433 | 1.00 | 35.49 | |
| ATOM | 1581 | NH1 | ARG | 413 | 9.569 | 0.454 | 52.914 | 1.00 | 34.87 | |
| ATOM | 1582 | NH2 | ARG | 413 | 11.036 | 2.221 | 52.929 | 1.00 | 35.73 | |
| ATOM | 1583 | C | ARG | 413 | 4.153 | 2.715 | 52.874 | 1.00 | 27.61 | |
| ATOM | 1584 | O | ARG | 413 | 4.656 | 3.142 | 53.911 | 1.00 | 26.64 | |
| ATOM | 1585 | N | PHE | 414 | 3.377 | 3.447 | 52.091 | 1.00 | 27.23 | |
| ATOM | 1586 | CA | PHE | 414 | 3.025 | 4.829 | 52.406 | 1.00 | 25.72 | |
| ATOM | 1587 | CB | PHE | 414 | 2.109 | 5.368 | 51.290 | 1.00 | 27.69 | |
| ATOM | 1588 | CG | PHE | 414 | 1.553 | 6.756 | 51.552 | 1.00 | 26.95 | |
| ATOM | 1589 | CD1 | PHE | 414 | 2.359 | 7.882 | 51.454 | 1.00 | 28.43 | |
| ATOM | 1590 | CD2 | PHE | 414 | 0.217 | 6.921 | 51.891 | 1.00 | 29.19 | |
| ATOM | 1591 | CE1 | PHE | 414 | 1.842 | 9.165 | 51.692 | 1.00 | 28.49 | |
| ATOM | 1592 | CE2 | PHE | 414 | −0.315 | 8.199 | 52.134 | 1.00 | 29.46 | |
| ATOM | 1593 | CZ | PHE | 414 | 0.503 | 9.325 | 52.033 | 1.00 | 28.75 | |
| ATOM | 1594 | C | PHE | 414 | 2.336 | 4.891 | 53.777 | 1.00 | 26.40 | |
| ATOM | 1595 | O | PHE | 414 | 2.691 | 5.702 | 54.637 | 1.00 | 26.65 | |
| ATOM | 1596 | N | ALA | 415 | 1.355 | 4.027 | 53.997 | 1.00 | 25.07 | |
| ATOM | 1597 | CA | ALA | 415 | 0.652 | 4.002 | 55.271 | 1.00 | 24.63 | |
| ATOM | 1598 | CB | ALA | 415 | −0.531 | 3.028 | 55.193 | 1.00 | 23.72 | |
| ATOM | 1599 | C | ALA | 415 | 1.572 | 3.622 | 56.436 | 1.00 | 24.02 | |
| ATOM | 1600 | O | ALA | 415 | 1.433 | 4.142 | 57.547 | 1.00 | 23.15 | |
| ATOM | 1601 | N | ALA | 416 | 2.518 | 2.722 | 56.174 | 1.00 | 24.35 | |
| ATOM | 1602 | CA | ALA | 416 | 3.448 | 2.270 | 57.205 | 1.00 | 24.54 | |
| ATOM | 1603 | CB | ALA | 416 | 4.313 | 1.143 | 56.666 | 1.00 | 24.42 | |
| ATOM | 1604 | C | ALA | 416 | 4.319 | 3.436 | 57.639 | 1.00 | 23.97 | |
| ATOM | 1605 | O | ALA | 416 | 4.544 | 3.646 | 58.832 | 1.00 | 25.89 | |
| ATOM | 1606 | N | LEU | 417 | 4.799 | 4.192 | 56.664 | 1.00 | 23.99 | |
| ATOM | 1607 | CA | LEU | 417 | 5.638 | 5.348 | 56.952 | 1.00 | 23.65 | |
| ATOM | 1608 | CB | LEU | 417 | 6.056 | 6.013 | 55.651 | 1.00 | 23.88 | |
| ATOM | 1609 | CG | LEU | 417 | 6.940 | 5.132 | 54.768 | 1.00 | 22.81 | |
| ATOM | 1610 | CD1 | LEU | 417 | 7.104 | 5.745 | 53.381 | 1.00 | 25.97 | |
| ATOM | 1611 | CD2 | LEU | 417 | 8.313 | 4.978 | 55.464 | 1.00 | 24.86 | |
| ATOM | 1612 | C | LEU | 417 | 4.894 | 6.346 | 57.843 | 1.00 | 23.64 | |
| ATOM | 1613 | O | LEU | 417 | 5.434 | 6.825 | 58.851 | 1.00 | 24.26 | |
| ATOM | 1614 | N | LEU | 418 | 3.635 | 6.632 | 57.517 | 1.00 | 22.96 | |
| ATOM | 1615 | CA | LEU | 418 | 2.896 | 7.617 | 58.305 | 1.00 | 22.78 | |
| ATOM | 1616 | CB | LEU | 418 | 1.555 | 7.961 | 57.622 | 1.00 | 22.19 | |
| ATOM | 1617 | CG | LEU | 418 | 1.632 | 8.536 | 56.193 | 1.00 | 22.93 | |
| ATOM | 1618 | CD1 | LEU | 418 | 0.232 | 8.983 | 55.715 | 1.00 | 23.88 | |
| ATOM | 1619 | CD2 | LEU | 418 | 2.554 | 9.749 | 56.179 | 1.00 | 23.65 | |
| ATOM | 1620 | C | LEU | 418 | 2.646 | 7.201 | 59.762 | 1.00 | 24.12 | |
| ATOM | 1621 | O | LEU | 418 | 2.372 | 8.046 | 60.620 | 1.00 | 22.61 | |
| ATOM | 1622 | N | LEU | 419 | 2.706 | 5.901 | 60.049 | 1.00 | 25.06 | |
| ATOM | 1623 | CA | LEU | 419 | 2.496 | 5.440 | 61.404 | 1.00 | 27.92 | |
| ATOM | 1624 | CB | LEU | 419 | 2.406 | 3.909 | 61.451 | 1.00 | 31.65 | |
| ATOM | 1625 | CG | LEU | 419 | 1.114 | 3.343 | 60.884 | 1.00 | 34.11 | |
| ATOM | 1626 | CD1 | LEU | 419 | 1.026 | 1.848 | 61.218 | 1.00 | 36.06 | |
| ATOM | 1627 | CD2 | LEU | 419 | −0.068 | 4.081 | 61.480 | 1.00 | 34.83 | |
| ATOM | 1628 | C | LEU | 419 | 3.594 | 5.891 | 62.360 | 1.00 | 29.85 | |
| ATOM | 1629 | O | LEU | 419 | 3.400 | 5.885 | 63.574 | 1.00 | 31.55 | |
| ATOM | 1630 | N | ARG | 420 | 4.736 | 6.296 | 61.828 | 1.00 | 31.33 | |
| ATOM | 1631 | CA | ARG | 420 | 5.801 | 6.725 | 62.718 | 1.00 | 31.87 | |
| ATOM | 1632 | CB | ARG | 420 | 7.145 | 6.755 | 61.985 | 1.00 | 32.74 | |
| ATOM | 1633 | CG | ARG | 420 | 7.648 | 5.387 | 61.490 | 1.00 | 33.67 | |
| ATOM | 1634 | CD | ARG | 420 | 7.856 | 4.364 | 62.622 | 1.00 | 36.93 | |
| ATOM | 1635 | NE | ARG | 420 | 6.709 | 3.481 | 62.820 | 1.00 | 35.35 | |
| ATOM | 1636 | CZ | ARG | 420 | 6.179 | 3.194 | 64.003 | 1.00 | 37.82 | |
| ATOM | 1637 | NH1 | ARG | 420 | 6.692 | 3.714 | 65.116 | 1.00 | 39.26 | |
| ATOM | 1638 | NH2 | ARG | 420 | 5.112 | 2.408 | 64.079 | 1.00 | 39.04 | |
| ATOM | 1639 | C | ARG | 420 | 5.477 | 8.092 | 63.303 | 1.00 | 32.15 | |
| ATOM | 1640 | O | ARG | 420 | 5.995 | 8.456 | 64.362 | 1.00 | 31.60 | |
| ATOM | 1641 | N | LEU | 421 | 4.591 | 8.845 | 62.655 | 1.00 | 30.38 | |
| ATOM | 1642 | CA | LEU | 421 | 4.278 | 10.175 | 63.164 | 1.00 | 29.53 | |
| ATOM | 1643 | CB | LEU | 421 | 3.519 | 10.976 | 62.121 | 1.00 | 30.72 | |
| ATOM | 1644 | CG | LEU | 421 | 4.322 | 11.027 | 60.808 | 1.00 | 31.31 | |
| ATOM | 1645 | CD1 | LEU | 421 | 3.645 | 12.019 | 59.885 | 1.00 | 32.38 | |
| ATOM | 1646 | CD2 | LEU | 421 | 5.800 | 11.411 | 61.053 | 1.00 | 31.74 | |
| ATOM | 1647 | C | LEU | 421 | 3.582 | 10.256 | 64.521 | 1.00 | 28.39 | |
| ATOM | 1648 | O | LEU | 421 | 3.977 | 11.061 | 65.363 | 1.00 | 26.36 | |
| ATOM | 1649 | N | PRO | 422 | 2.533 | 9.446 | 64.762 | 1.00 | 27.69 | |
| ATOM | 1650 | CD | PRO | 422 | 1.678 | 8.672 | 63.839 | 1.00 | 27.47 | |
| ATOM | 1651 | CA | PRO | 422 | 1.915 | 9.559 | 66.090 | 1.00 | 26.04 | |
| ATOM | 1652 | CB | PRO | 422 | 0.717 | 8.603 | 66.005 | 1.00 | 28.29 | |
| ATOM | 1653 | CG | PRO | 422 | 0.350 | 8.664 | 64.557 | 1.00 | 28.91 | |
| ATOM | 1654 | C | PRO | 422 | 2.906 | 9.143 | 67.198 | 1.00 | 25.36 | |
| ATOM | 1655 | O | PRO | 422 | 2.832 | 9.637 | 68.322 | 1.00 | 24.06 | |
| ATOM | 1656 | N | ALA | 423 | 3.821 | 8.228 | 66.872 | 1.00 | 24.34 | |
| ATOM | 1657 | CA | ALA | 423 | 4.817 | 7.779 | 67.840 | 1.00 | 22.88 | |
| ATOM | 1658 | CB | ALA | 423 | 5.589 | 6.599 | 67.273 | 1.00 | 26.29 | |
| ATOM | 1659 | C | ALA | 423 | 5.765 | 8.942 | 68.158 | 1.00 | 24.20 | |
| ATOM | 1660 | O | ALA | 423 | 6.094 | 9.197 | 69.315 | 1.00 | 22.14 | |
| ATOM | 1661 | N | LEU | 424 | 6.212 | 9.640 | 67.119 | 1.00 | 22.92 | |
| ATOM | 1662 | CA | LEU | 424 | 7.103 | 10.773 | 67.309 | 1.00 | 22.78 | |
| ATOM | 1663 | CB | LEU | 424 | 7.512 | 11.306 | 65.936 | 1.00 | 23.40 | |
| ATOM | 1664 | CG | LEU | 424 | 8.405 | 12.531 | 65.875 | 1.00 | 24.54 | |
| ATOM | 1665 | CD1 | LEU | 424 | 9.777 | 12.226 | 66.517 | 1.00 | 22.52 | |
| ATOM | 1666 | CD2 | LEU | 424 | 8.589 | 12.892 | 64.405 | 1.00 | 22.78 | |
| ATOM | 1667 | C | LEU | 424 | 6.422 | 11.861 | 68.153 | 1.00 | 22.46 | |
| ATOM | 1668 | O | LEU | 424 | 7.036 | 12.468 | 69.038 | 1.00 | 21.68 | |
| ATOM | 1669 | N | ARG | 425 | 5.136 | 12.101 | 67.900 | 1.00 | 22.38 | |
| ATOM | 1670 | CA | ARG | 425 | 4.386 | 13.095 | 68.663 | 1.00 | 23.67 | |
| ATOM | 1671 | CB | ARG | 425 | 2.969 | 13.240 | 68.087 | 1.00 | 26.56 | |
| ATOM | 1672 | CG | ARG | 425 | 2.066 | 14.140 | 68.903 | 1.00 | 30.90 | |
| ATOM | 1673 | CD | ARG | 425 | 0.977 | 14.732 | 68.031 | 1.00 | 36.25 | |
| ATOM | 1674 | NE | ARG | 425 | 0.469 | 13.774 | 67.044 | 1.00 | 40.86 | |
| ATOM | 1675 | CZ | ARG | 425 | −0.070 | 12.592 | 67.339 | 1.00 | 42.76 | |
| ATOM | 1676 | NH1 | ARG | 425 | −0.183 | 12.196 | 68.604 | 1.00 | 46.63 | |
| ATOM | 1677 | NH2 | ARG | 425 | −0.498 | 11.800 | 66.367 | 1.00 | 44.97 | |
| ATOM | 1678 | C | ARG | 425 | 4.309 | 12.737 | 70.150 | 1.00 | 21.12 | |
| ATOM | 1679 | O | ARG | 425 | 4.418 | 13.604 | 71.021 | 1.00 | 21.30 | |
| ATOM | 1680 | N | SER | 426 | 4.124 | 11.452 | 70.436 | 1.00 | 19.60 | |
| ATOM | 1681 | CA | SER | 426 | 4.021 | 10.989 | 71.820 | 1.00 | 19.49 | |
| ATOM | 1682 | CB | SER | 426 | 3.534 | 9.539 | 71.855 | 1.00 | 20.63 | |
| ATOM | 1683 | OG | SER | 426 | 3.491 | 9.086 | 73.198 | 1.00 | 23.17 | |
| ATOM | 1684 | C | SER | 426 | 5.374 | 11.096 | 72.535 | 1.00 | 17.38 | |
| ATOM | 1685 | O | SER | 426 | 5.458 | 11.496 | 73.698 | 1.00 | 18.97 | |
| ATOM | 1686 | N | ILE | 427 | 6.419 | 10.742 | 71.812 | 1.00 | 18.11 | |
| ATOM | 1687 | CA | ILE | 427 | 7.770 | 10.812 | 72.368 | 1.00 | 15.88 | |
| ATOM | 1688 | CB | ILE | 427 | 8.763 | 10.095 | 71.449 | 1.00 | 15.75 | |
| ATOM | 1689 | CG2 | ILE | 427 | 10.213 | 10.351 | 71.913 | 1.00 | 17.78 | |
| ATOM | 1690 | CG1 | ILE | 427 | 8.479 | 8.591 | 71.526 | 1.00 | 18.23 | |
| ATOM | 1691 | CD1 | ILE | 427 | 9.082 | 7.789 | 70.440 | 1.00 | 20.33 | |
| ATOM | 1692 | C | ILE | 427 | 8.146 | 12.272 | 72.599 | 1.00 | 16.69 | |
| ATOM | 1693 | O | ILE | 427 | 8.779 | 12.589 | 73.606 | 1.00 | 16.41 | |
| ATOM | 1694 | N | SER | 428 | 7.767 | 13.159 | 71.685 | 1.00 | 17.02 | |
| ATOM | 1695 | CA | SER | 428 | 8.059 | 14.590 | 71.884 | 1.00 | 17.37 | |
| ATOM | 1696 | CB | SER | 428 | 7.636 | 15.393 | 70.633 | 1.00 | 18.11 | |
| ATOM | 1697 | OG | SER | 428 | 7.745 | 16.794 | 70.876 | 1.00 | 19.14 | |
| ATOM | 1698 | C | SER | 428 | 7.377 | 15.138 | 73.154 | 1.00 | 18.08 | |
| ATOM | 1699 | O | SER | 428 | 7.968 | 15.916 | 73.928 | 1.00 | 16.98 | |
| ATOM | 1700 | N | LEU | 429 | 6.134 | 14.741 | 73.425 | 1.00 | 18.84 | |
| ATOM | 1701 | CA | LEU | 429 | 5.518 | 15.249 | 74.633 | 1.00 | 19.23 | |
| ATOM | 1702 | CB | LEU | 429 | 4.054 | 14.807 | 74.723 | 1.00 | 22.17 | |
| ATOM | 1703 | CG | LEU | 429 | 3.187 | 15.432 | 73.625 | 1.00 | 25.96 | |
| ATOM | 1704 | CD1 | LEU | 429 | 1.800 | 14.809 | 73.624 | 1.00 | 28.92 | |
| ATOM | 1705 | CD2 | LEU | 429 | 3.087 | 16.943 | 73.855 | 1.00 | 29.57 | |
| ATOM | 1706 | C | LEU | 429 | 6.296 | 14.782 | 75.860 | 1.00 | 17.99 | |
| ATOM | 1707 | O | LEU | 429 | 6.465 | 15.527 | 76.816 | 1.00 | 18.52 | |
| ATOM | 1708 | N | LYS | 430 | 6.778 | 13.544 | 75.830 | 1.00 | 18.39 | |
| ATOM | 1709 | CA | LYS | 430 | 7.540 | 13.047 | 76.957 | 1.00 | 17.66 | |
| ATOM | 1710 | CB | LYS | 430 | 7.780 | 11.544 | 76.826 | 1.00 | 19.73 | |
| ATOM | 1711 | CG | LYS | 430 | 8.539 | 10.975 | 78.020 | 1.00 | 23.74 | |
| ATOM | 1712 | CD | LYS | 430 | 7.625 | 11.004 | 79.260 | 1.00 | 27.95 | |
| ATOM | 1713 | CE | LYS | 430 | 8.192 | 10.194 | 80.414 | 1.00 | 34.36 | |
| ATOM | 1714 | NZ | LYS | 430 | 7.167 | 10.122 | 81.490 | 1.00 | 36.64 | |
| ATOM | 1715 | C | LYS | 430 | 8.865 | 13.795 | 77.077 | 1.00 | 18.37 | |
| ATOM | 1716 | O | LYS | 430 | 9.346 | 14.034 | 78.191 | 1.00 | 18.47 | |
| ATOM | 1717 | N | SER | 431 | 9.435 | 14.174 | 75.936 | 0.50 | 17.09 | AC1 |
| ATOM | 1718 | CA | SER | 431 | 10.693 | 14.931 | 75.919 | 0.50 | 18.08 | AC1 |
| ATOM | 1719 | CB | SER | 431 | 11.102 | 15.238 | 74.471 | 0.50 | 17.50 | AC1 |
| ATOM | 1720 | OG | SER | 431 | 12.180 | 16.159 | 74.413 | 0.50 | 19.63 | AC1 |
| ATOM | 1721 | C | SER | 431 | 10.472 | 16.244 | 76.663 | 0.50 | 18.02 | AC1 |
| ATOM | 1722 | O | SER | 431 | 11.297 | 16.668 | 77.464 | 0.50 | 17.75 | AC1 |
| ATOM | 1723 | N | PHE | 432 | 9.326 | 16.875 | 76.415 | 1.00 | 17.68 | |
| ATOM | 1724 | CA | PHE | 432 | 9.020 | 18.162 | 77.057 | 1.00 | 17.81 | |
| ATOM | 1725 | CB | PHE | 432 | 7.778 | 18.817 | 76.425 | 1.00 | 18.74 | |
| ATOM | 1726 | CG | PHE | 432 | 8.099 | 19.713 | 75.249 | 1.00 | 17.54 | |
| ATOM | 1727 | CD1 | PHE | 432 | 8.649 | 20.976 | 75.448 | 1.00 | 18.72 | |
| ATOM | 1728 | CD2 | PHE | 432 | 7.851 | 19.290 | 73.958 | 1.00 | 17.78 | |
| ATOM | 1729 | CE1 | PHE | 432 | 8.941 | 21.800 | 74.351 | 1.00 | 18.20 | |
| ATOM | 1730 | CE2 | PHE | 432 | 8.135 | 20.093 | 72.863 | 1.00 | 18.04 | |
| ATOM | 1731 | CZ | PHE | 432 | 8.684 | 21.358 | 73.060 | 1.00 | 19.15 | |
| ATOM | 1732 | C | PHE | 432 | 8.817 | 17.989 | 78.550 | 1.00 | 18.32 | |
| ATOM | 1733 | O | PHE | 432 | 9.170 | 18.872 | 79.330 | 1.00 | 20.66 | |
| ATOM | 1734 | N | GLU | 433 | 8.249 | 16.852 | 78.964 | 1.00 | 17.15 | |
| ATOM | 1735 | CA | GLU | 433 | 8.073 | 16.592 | 80.382 | 1.00 | 20.50 | |
| ATOM | 1736 | CB | GLU | 433 | 7.520 | 15.192 | 80.613 | 1.00 | 19.84 | |
| ATOM | 1737 | CG | GLU | 433 | 6.045 | 15.053 | 80.320 | 1.00 | 27.71 | |
| ATOM | 1738 | CD | GLU | 433 | 5.533 | 13.688 | 80.744 | 1.00 | 31.07 | |
| ATOM | 1739 | OE1 | GLU | 433 | 5.964 | 13.224 | 81.823 | 1.00 | 34.98 | |
| ATOM | 1740 | OE2 | GLU | 433 | 4.714 | 13.094 | 80.006 | 1.00 | 34.76 | |
| ATOM | 1741 | C | GLU | 433 | 9.437 | 16.691 | 81.052 | 1.00 | 19.32 | |
| ATOM | 1742 | O | GLU | 433 | 9.574 | 17.322 | 82.106 | 1.00 | 20.27 | |
| ATOM | 1743 | N | HIS | 434 | 10.438 | 16.059 | 80.435 | 1.00 | 18.45 | |
| ATOM | 1744 | CA | HIS | 434 | 11.810 | 16.086 | 80.972 | 1.00 | 19.10 | |
| ATOM | 1745 | CB | HIS | 434 | 12.708 | 15.091 | 80.247 | 1.00 | 20.67 | |
| ATOM | 1746 | CG | HIS | 434 | 12.346 | 13.662 | 80.495 | 1.00 | 21.88 | |
| ATOM | 1747 | CD2 | HIS | 434 | 12.196 | 12.621 | 79.642 | 1.00 | 22.64 | |
| ATOM | 1748 | ND1 | HIS | 434 | 12.153 | 13.151 | 81.763 | 1.00 | 24.64 | |
| ATOM | 1749 | CE1 | HIS | 434 | 11.901 | 11.857 | 81.678 | 1.00 | 26.38 | |
| ATOM | 1750 | NE2 | HIS | 434 | 11.924 | 11.509 | 80.403 | 1.00 | 25.49 | |
| ATOM | 1751 | C | HIS | 434 | 12.448 | 17.471 | 80.900 | 1.00 | 19.47 | |
| ATOM | 1752 | O | HIS | 434 | 13.029 | 17.930 | 81.868 | 1.00 | 20.51 | |
| ATOM | 1753 | N | LEU | 435 | 12.360 | 18.122 | 79.749 | 1.00 | 17.30 | |
| ATOM | 1754 | CA | LEU | 435 | 12.926 | 19.464 | 79.614 | 1.00 | 17.70 | |
| ATOM | 1755 | CB | LEU | 435 | 12.660 | 20.017 | 78.219 | 1.00 | 17.54 | |
| ATOM | 1756 | CG | LEU | 435 | 13.350 | 19.239 | 77.100 | 1.00 | 17.06 | |
| ATOM | 1757 | CD1 | LEU | 435 | 12.933 | 19.793 | 75.764 | 1.00 | 19.34 | |
| ATOM | 1758 | CD2 | LEU | 435 | 14.874 | 19.343 | 77.242 | 1.00 | 19.09 | |
| ATOM | 1759 | C | LEU | 435 | 12.334 | 20.411 | 80.653 | 1.00 | 17.96 | |
| ATOM | 1760 | O | LEU | 435 | 13.034 | 21.260 | 81.193 | 1.00 | 18.73 | |
| ATOM | 1761 | N | PHE | 436 | 11.035 | 20.302 | 80.921 | 1.00 | 17.35 | |
| ATOM | 1762 | CA | PHE | 436 | 10.460 | 21.192 | 81.936 | 1.00 | 18.85 | |
| ATOM | 1763 | CB | PHE | 436 | 8.920 | 21.188 | 81.897 | 1.00 | 19.91 | |
| ATOM | 1764 | CG | PHE | 436 | 8.340 | 21.916 | 80.720 | 1.00 | 22.43 | |
| ATOM | 1765 | CD1 | PHE | 436 | 8.885 | 23.113 | 80.288 | 1.00 | 23.77 | |
| ATOM | 1766 | CD2 | PHE | 436 | 7.243 | 21.399 | 80.040 | 1.00 | 23.20 | |
| ATOM | 1767 | CE1 | PHE | 436 | 8.353 | 23.783 | 79.202 | 1.00 | 25.60 | |
| ATOM | 1768 | CE2 | PHE | 436 | 6.703 | 22.063 | 78.942 | 1.00 | 22.41 | |
| ATOM | 1769 | CZ | PHE | 436 | 7.250 | 23.249 | 78.521 | 1.00 | 26.06 | |
| ATOM | 1770 | C | PHE | 436 | 10.916 | 20.802 | 83.332 | 1.00 | 20.10 | |
| ATOM | 1771 | O | PHE | 436 | 11.195 | 21.670 | 84.161 | 1.00 | 22.89 | |
| ATOM | 1772 | N | PHE | 437 | 11.001 | 19.500 | 83.605 | 1.00 | 19.98 | |
| ATOM | 1773 | CA | PHE | 437 | 11.412 | 19.023 | 84.922 | 1.00 | 21.11 | |
| ATOM | 1774 | CB | PHE | 437 | 11.364 | 17.484 | 84.974 | 1.00 | 21.57 | |
| ATOM | 1775 | CG | PHE | 437 | 11.628 | 16.913 | 86.339 | 1.00 | 25.91 | |
| ATOM | 1776 | CD1 | PHE | 437 | 10.633 | 16.924 | 87.313 | 1.00 | 27.17 | |
| ATOM | 1777 | CD2 | PHE | 437 | 12.881 | 16.419 | 86.665 | 1.00 | 27.22 | |
| ATOM | 1778 | CE1 | PHE | 437 | 10.891 | 16.447 | 88.599 | 1.00 | 30.66 | |
| ATOM | 1779 | CE2 | PHE | 437 | 13.153 | 15.942 | 87.944 | 1.00 | 31.40 | |
| ATOM | 1780 | CZ | PHE | 437 | 12.158 | 15.957 | 88.910 | 1.00 | 29.78 | |
| ATOM | 1781 | C | PHE | 437 | 12.807 | 19.496 | 85.305 | 1.00 | 22.67 | |
| ATOM | 1782 | O | PHE | 437 | 13.046 | 19.895 | 86.464 | 1.00 | 24.17 | |
| ATOM | 1783 | N | PHE | 438 | 13.724 | 19.453 | 84.346 | 1.00 | 19.58 | |
| ATOM | 1784 | CA | PHE | 438 | 15.103 | 19.888 | 84.567 | 1.00 | 22.15 | |
| ATOM | 1785 | CB | PHE | 438 | 16.038 | 19.027 | 83.718 | 1.00 | 22.90 | |
| ATOM | 1786 | CG | PHE | 438 | 16.093 | 17.595 | 84.171 | 1.00 | 23.82 | |
| ATOM | 1787 | CD1 | PHE | 438 | 16.725 | 17.262 | 85.361 | 1.00 | 24.00 | |
| ATOM | 1788 | CD2 | PHE | 438 | 15.509 | 16.584 | 83.419 | 1.00 | 27.39 | |
| ATOM | 1789 | CE1 | PHE | 438 | 16.773 | 15.942 | 85.795 | 1.00 | 25.11 | |
| ATOM | 1790 | CE2 | PHE | 438 | 15.557 | 15.253 | 83.847 | 1.00 | 27.37 | |
| ATOM | 1791 | CZ | PHE | 438 | 16.188 | 14.935 | 85.033 | 1.00 | 27.90 | |
| ATOM | 1792 | C | PHE | 438 | 15.334 | 21.383 | 84.256 | 1.00 | 21.04 | |
| ATOM | 1793 | O | PHE | 438 | 16.454 | 21.875 | 84.352 | 1.00 | 22.98 | |
| ATOM | 1794 | N | HIS | 439 | 14.267 | 22.077 | 83.867 | 1.00 | 19.84 | |
| ATOM | 1795 | CA | HIS | 439 | 14.311 | 23.503 | 83.508 | 1.00 | 19.79 | |
| ATOM | 1796 | CB | HIS | 439 | 14.550 | 24.380 | 84.742 | 1.00 | 21.68 | |
| ATOM | 1797 | CG | HIS | 439 | 13.463 | 24.294 | 85.763 | 1.00 | 24.96 | |
| ATOM | 1798 | CD2 | HIS | 439 | 12.345 | 25.037 | 85.939 | 1.00 | 27.66 | |
| ATOM | 1799 | ND1 | HIS | 439 | 13.440 | 23.331 | 86.747 | 1.00 | 26.44 | |
| ATOM | 1800 | CE1 | HIS | 439 | 12.353 | 23.481 | 87.481 | 1.00 | 27.37 | |
| ATOM | 1801 | NE2 | HIS | 439 | 11.672 | 24.511 | 87.012 | 1.00 | 28.08 | |
| ATOM | 1802 | C | HIS | 439 | 15.375 | 23.803 | 82.469 | 1.00 | 20.93 | |
| ATOM | 1803 | O | HIS | 439 | 16.185 | 24.726 | 82.626 | 1.00 | 23.42 | |
| ATOM | 1804 | N | LEU | 440 | 15.318 | 23.068 | 81.366 | 1.00 | 16.51 | |
| ATOM | 1805 | CA | LEU | 440 | 16.289 | 23.200 | 80.301 | 1.00 | 18.97 | |
| ATOM | 1806 | CB | LEU | 440 | 16.820 | 21.813 | 79.917 | 1.00 | 19.31 | |
| ATOM | 1807 | CG | LEU | 440 | 17.585 | 21.101 | 81.017 | 1.00 | 21.82 | |
| ATOM | 1808 | CD1 | LEU | 440 | 17.920 | 19.672 | 80.567 | 1.00 | 24.43 | |
| ATOM | 1809 | CD2 | LEU | 440 | 18.839 | 21.874 | 81.341 | 1.00 | 21.97 | |
| ATOM | 1810 | C | LEU | 440 | 15.751 | 23.859 | 79.042 | 1.00 | 20.05 | |
| ATOM | 1811 | O | LEU | 440 | 16.497 | 24.030 | 78.099 | 1.00 | 21.82 | |
| ATOM | 1812 | N | VAL | 441 | 14.478 | 24.229 | 79.038 | 1.00 | 19.87 | |
| ATOM | 1813 | CA | VAL | 441 | 13.879 | 24.814 | 77.833 | 1.00 | 21.84 | |
| ATOM | 1814 | CB | VAL | 441 | 12.795 | 23.830 | 77.247 | 1.00 | 22.63 | |
| ATOM | 1815 | CG1 | VAL | 441 | 11.601 | 23.713 | 78.192 | 1.00 | 22.79 | |
| ATOM | 1816 | CG2 | VAL | 441 | 12.365 | 24.276 | 75.855 | 1.00 | 24.87 | |
| ATOM | 1817 | C | VAL | 441 | 13.306 | 26.224 | 78.045 | 1.00 | 22.01 | |
| ATOM | 1818 | O | VAL | 441 | 12.587 | 26.478 | 79.009 | 1.00 | 20.37 | |
| ATOM | 1819 | N | ALA | 442 | 13.646 | 27.122 | 77.120 | 1.00 | 23.96 | |
| ATOM | 1820 | CA | ALA | 442 | 13.233 | 28.529 | 77.172 | 1.00 | 27.08 | |
| ATOM | 1821 | CB | ALA | 442 | 14.246 | 29.393 | 76.392 | 1.00 | 28.21 | |
| ATOM | 1822 | C | ALA | 442 | 11.846 | 28.718 | 76.591 | 1.00 | 28.96 | |
| ATOM | 1823 | O | ALA | 442 | 11.655 | 29.494 | 75.658 | 1.00 | 30.12 | |
| ATOM | 1824 | N | ASP | 443 | 10.895 | 28.011 | 77.178 | 1.00 | 30.46 | |
| ATOM | 1825 | CA | ASP | 443 | 9.493 | 27.994 | 76.764 | 1.00 | 31.83 | |
| ATOM | 1826 | CB | ASP | 443 | 8.678 | 27.367 | 77.906 | 1.00 | 33.17 | |
| ATOM | 1827 | CG | ASP | 443 | 7.208 | 27.218 | 77.581 | 1.00 | 34.98 | |
| ATOM | 1828 | OD1 | ASP | 443 | 6.856 | 26.945 | 76.404 | 1.00 | 37.47 | |
| ATOM | 1829 | OD2 | ASP | 443 | 6.404 | 27.346 | 78.524 | 1.00 | 31.58 | |
| ATOM | 1830 | C | ASP | 443 | 8.873 | 29.320 | 76.318 | 1.00 | 31.97 | |
| ATOM | 1831 | O | ASP | 443 | 8.426 | 29.453 | 75.180 | 1.00 | 30.41 | |
| ATOM | 1832 | N | THR | 444 | 8.854 | 30.304 | 77.205 | 1.00 | 32.53 | |
| ATOM | 1833 | CA | THR | 444 | 8.236 | 31.586 | 76.891 | 1.00 | 32.98 | |
| ATOM | 1834 | CB | THR | 444 | 7.965 | 32.371 | 78.198 | 1.00 | 34.53 | |
| ATOM | 1835 | OG1 | THR | 444 | 9.196 | 32.581 | 78.900 | 1.00 | 37.04 | |
| ATOM | 1836 | CG2 | THR | 444 | 7.020 | 31.577 | 79.102 | 1.00 | 35.95 | |
| ATOM | 1837 | C | THR | 444 | 8.981 | 32.486 | 75.901 | 1.00 | 32.99 | |
| ATOM | 1838 | O | THR | 444 | 8.399 | 33.436 | 75.370 | 1.00 | 32.71 | |
| ATOM | 1839 | N | SER | 445 | 10.248 | 32.179 | 75.639 | 1.00 | 30.56 | |
| ATOM | 1840 | CA | SER | 445 | 11.071 | 32.977 | 74.727 | 1.00 | 29.90 | |
| ATOM | 1841 | CB | SER | 445 | 12.481 | 33.132 | 75.313 | 1.00 | 29.52 | |
| ATOM | 1842 | OG | SER | 445 | 12.418 | 33.766 | 76.576 | 1.00 | 31.76 | |
| ATOM | 1843 | C | SER | 445 | 11.199 | 32.429 | 73.308 | 1.00 | 29.39 | |
| ATOM | 1844 | O | SER | 445 | 11.580 | 33.153 | 72.387 | 1.00 | 28.30 | |
| ATOM | 1845 | N | ILE | 446 | 10.875 | 31.151 | 73.133 | 1.00 | 26.98 | |
| ATOM | 1846 | CA | ILE | 446 | 11.010 | 30.511 | 71.840 | 1.00 | 25.95 | |
| ATOM | 1847 | CB | ILE | 446 | 10.656 | 29.013 | 71.961 | 1.00 | 26.36 | |
| ATOM | 1848 | CG2 | ILE | 446 | 10.295 | 28.431 | 70.595 | 1.00 | 24.39 | |
| ATOM | 1849 | CG1 | ILE | 446 | 11.864 | 28.300 | 72.575 | 1.00 | 27.57 | |
| ATOM | 1850 | CD1 | ILE | 446 | 11.637 | 26.869 | 72.971 | 1.00 | 27.13 | |
| ATOM | 1851 | C | ILE | 446 | 10.312 | 31.144 | 70.640 | 1.00 | 25.28 | |
| ATOM | 1852 | O | ILE | 446 | 10.917 | 31.247 | 69.571 | 1.00 | 23.47 | |
| ATOM | 1853 | N | ALA | 447 | 9.058 | 31.549 | 70.809 | 1.00 | 26.37 | |
| ATOM | 1854 | CA | ALA | 447 | 8.316 | 32.183 | 69.725 | 1.00 | 28.80 | |
| ATOM | 1855 | CB | ALA | 447 | 6.932 | 32.606 | 70.219 | 1.00 | 30.52 | |
| ATOM | 1856 | C | ALA | 447 | 9.114 | 33.394 | 69.218 | 1.00 | 28.51 | |
| ATOM | 1857 | O | ALA | 447 | 9.229 | 33.608 | 68.005 | 1.00 | 29.41 | |
| ATOM | 1858 | N | GLY | 448 | 9.675 | 34.164 | 70.155 | 1.00 | 28.53 | |
| ATOM | 1859 | CA | GLY | 448 | 10.474 | 35.337 | 69.811 | 1.00 | 27.89 | |
| ATOM | 1860 | C | GLY | 448 | 11.762 | 34.993 | 69.095 | 1.00 | 28.77 | |
| ATOM | 1861 | O | GLY | 448 | 12.167 | 35.692 | 68.162 | 1.00 | 28.23 | |
| ATOM | 1862 | N | TYR | 449 | 12.435 | 33.927 | 69.536 | 1.00 | 27.26 | |
| ATOM | 1863 | CA | TYR | 449 | 13.666 | 33.502 | 68.872 | 1.00 | 28.14 | |
| ATOM | 1864 | CB | TYR | 449 | 14.262 | 32.267 | 69.553 | 1.00 | 26.16 | |
| ATOM | 1865 | CG | TYR | 449 | 14.683 | 32.492 | 70.990 | 1.00 | 28.82 | |
| ATOM | 1866 | CD1 | TYR | 449 | 14.913 | 33.782 | 71.482 | 1.00 | 29.84 | |
| ATOM | 1867 | CE1 | TYR | 449 | 15.336 | 33.988 | 72.802 | 1.00 | 32.66 | |
| ATOM | 1868 | CD2 | TYR | 449 | 14.881 | 31.412 | 71.853 | 1.00 | 29.90 | |
| ATOM | 1869 | CE2 | TYR | 449 | 15.306 | 31.604 | 73.173 | 1.00 | 30.71 | |
| ATOM | 1870 | CZ | TYR | 449 | 15.532 | 32.887 | 73.641 | 1.00 | 32.74 | |
| ATOM | 1871 | OH | TYR | 449 | 15.979 | 33.070 | 74.939 | 1.00 | 36.98 | |
| ATOM | 1872 | C | TYR | 449 | 13.361 | 33.150 | 67.420 | 1.00 | 27.51 | |
| ATOM | 1873 | O | TYR | 449 | 14.116 | 33.491 | 66.513 | 1.00 | 27.99 | |
| ATOM | 1874 | N | ILE | 450 | 12.254 | 32.442 | 67.207 | 1.00 | 27.41 | |
| ATOM | 1875 | CA | ILE | 450 | 11.876 | 32.053 | 65.861 | 1.00 | 27.70 | |
| ATOM | 1876 | CB | ILE | 450 | 10.662 | 31.102 | 65.863 | 1.00 | 26.64 | |
| ATOM | 1877 | CG2 | ILE | 450 | 10.292 | 30.744 | 64.413 | 1.00 | 26.88 | |
| ATOM | 1878 | CG1 | ILE | 450 | 11.003 | 29.846 | 66.690 | 1.00 | 27.46 | |
| ATOM | 1879 | CD1 | ILE | 450 | 9.811 | 28.956 | 67.032 | 1.00 | 24.45 | |
| ATOM | 1880 | C | ILE | 450 | 11.534 | 33.295 | 65.041 | 1.00 | 29.34 | |
| ATOM | 1881 | O | ILE | 450 | 11.994 | 33.440 | 63.911 | 1.00 | 30.32 | |
| ATOM | 1882 | N | ARG | 451 | 10.735 | 34.187 | 65.617 | 1.00 | 30.43 | |
| ATOM | 1883 | CA | ARG | 451 | 10.351 | 35.416 | 64.923 | 1.00 | 33.00 | |
| ATOM | 1884 | CB | ARG | 451 | 9.514 | 36.306 | 65.851 | 1.00 | 32.56 | |
| ATOM | 1885 | CG | ARG | 451 | 8.874 | 37.519 | 65.161 | 1.00 | 34.91 | |
| ATOM | 1886 | CD | ARG | 451 | 7.955 | 38.328 | 66.076 | 1.00 | 36.14 | |
| ATOM | 1887 | NE | ARG | 451 | 6.768 | 37.599 | 66.518 | 1.00 | 37.46 | |
| ATOM | 1888 | CZ | ARG | 451 | 6.669 | 36.943 | 67.672 | 1.00 | 40.02 | |
| ATOM | 1889 | NH1 | ARG | 451 | 7.690 | 36.921 | 68.521 | 1.00 | 41.16 | |
| ATOM | 1890 | NH2 | ARG | 451 | 5.547 | 36.299 | 67.976 | 1.00 | 40.59 | |
| ATOM | 1891 | C | ARG | 451 | 11.629 | 36.138 | 64.472 | 1.00 | 34.57 | |
| ATOM | 1892 | O | ARG | 451 | 11.761 | 36.516 | 63.298 | 1.00 | 34.59 | |
| ATOM | 1893 | N | ASP | 452 | 12.578 | 36.304 | 65.392 | 1.00 | 36.05 | |
| ATOM | 1894 | CA | ASP | 452 | 13.837 | 36.975 | 65.070 | 1.00 | 39.88 | |
| ATOM | 1895 | CB | ASP | 452 | 14.690 | 37.184 | 66.331 | 1.00 | 42.41 | |
| ATOM | 1896 | CG | ASP | 452 | 14.004 | 38.065 | 67.364 | 1.00 | 46.01 | |
| ATOM | 1897 | OD1 | ASP | 452 | 13.162 | 38.897 | 66.961 | 1.00 | 46.57 | |
| ATOM | 1898 | OD2 | ASP | 452 | 14.315 | 37.934 | 68.576 | 1.00 | 48.55 | |
| ATOM | 1899 | C | ASP | 452 | 14.657 | 36.212 | 64.039 | 1.00 | 40.80 | |
| ATOM | 1900 | O | ASP | 452 | 15.219 | 36.805 | 63.112 | 1.00 | 42.52 | |
| ATOM | 1901 | N | ALA | 453 | 14.730 | 34.896 | 64.197 | 1.00 | 41.16 | |
| ATOM | 1902 | CA | ALA | 453 | 15.493 | 34.071 | 63.272 | 1.00 | 42.41 | |
| ATOM | 1903 | CB | ALA | 453 | 15.585 | 32.643 | 63.796 | 1.00 | 41.71 | |
| ATOM | 1904 | C | ALA | 453 | 14.904 | 34.070 | 61.863 | 1.00 | 44.06 | |
| ATOM | 1905 | O | ALA | 453 | 15.635 | 33.910 | 60.887 | 1.00 | 44.96 | |
| ATOM | 1906 | N | LEU | 454 | 13.590 | 34.259 | 61.759 1.00 | 45.34 | | |
| ATOM | 1907 | CA | LEU | 454 | 12.921 | 34.261 | 60.460 1.00 | 47.12 | | |
| ATOM | 1908 | CB | LEU | 454 | 11.419 | 33.989 | 60.631 1.00 | 42.61 | | |
| ATOM | 1909 | CG | LEU | 454 | 11.078 | 32.514 | 60.884 1.00 | 38.89 | | |
| ATOM | 1910 | CD1 | LEU | 454 | 9.576 | 32.329 | 60.950 1.00 | 36.29 | | |
| ATOM | 1911 | CD2 | LEU | 454 | 11.660 | 31.657 | 59.764 1.00 | 36.23 | | |
| ATOM | 1912 | C | LEU | 454 | 13.149 | 35.544 | 59.669 1.00 | 50.37 | | |
| ATOM | 1913 | O | LEU | 454 | 13.255 | 35.506 | 58.443 1.00 | 51.61 | | |
| ATOM | 1914 | N | ARG | 455 | 13.218 | 36.679 | 60.360 1.00 | 54.48 | | |
| ATOM | 1915 | CA | ARG | 455 | 13.486 | 37.951 | 59.688 1.00 | 58.31 | | |
| ATOM | 1916 | CB | ARG | 455 | 13.128 | 39.147 | 60.582 1.00 | 59.22 | | |
| ATOM | 1917 | CG | ARG | 455 | 11.635 | 39.352 | 60.806 1.00 | 60.97 | | |
| ATOM | 1918 | CD | ARG | 455 | 11.318 | 40.787 | 61.228 1.00 | 62.50 | | |
| ATOM | 1919 | NE | ARG | 455 | 10.998 | 40.919 | 62.648 | 1.00 | 64.04 | |
| ATOM | 1920 | CZ | ARG | 455 | 11.873 | 40.784 | 63.640 | 1.00 | 64.71 | |
| ATOM | 1921 | NH1 | ARG | 455 | 13.146 | 40.511 | 63.378 | 1.00 | 64.92 | |
| ATOM | 1922 | NH2 | ARG | 455 | 11.470 | 40.923 | 64.897 | 1.00 | 64.48 | |
| ATOM | 1923 | C | ARG | 455 | 14.988 | 37.938 | 59.452 | 1.00 | 60.72 | |
| ATOM | 1924 | O | ARG | 455 | 15.597 | 38.964 | 59.147 | 1.00 | 61.55 | |
| ATOM | 1925 | N | ASN | 456 | 15.557 | 36.743 | 59.597 | 1.00 | 63.01 | |
| ATOM | 1926 | CA | ASN | 456 | 16.983 | 36.482 | 59.463 | 1.00 | 64.82 | |
| ATOM | 1927 | CB | ASN | 456 | 17.434 | 36.512 | 57.987 | 1.00 | 66.21 | |
| ATOM | 1928 | CG | ASN | 456 | 17.254 | 37.871 | 57.327 | 1.00 | 67.51 | |
| ATOM | 1929 | OD1 | ASN | 456 | 17.901 | 38.850 | 57.702 | 1.00 | 68.69 | |
| ATOM | 1930 | ND2 | ASN | 456 | 16.377 | 37.930 | 56.326 | 1.00 | 68.01 | |
| ATOM | 1931 | C | ASN | 456 | 17.795 | 37.442 | 60.317 | 1.00 | 65.37 | |
| ATOM | 1932 | O | ASN | 456 | 17.456 | 37.680 | 61.480 | 1.00 | 65.63 | |
| ATOM | 1933 | N | GLY | 457 | 18.858 | 37.997 | 59.749 | 1.00 | 65.99 | |
| ATOM | 1934 | CA | GLY | 457 | 19.704 | 38.896 | 60.510 | 1.00 | 66.46 | |
| ATOM | 1935 | C | GLY | 457 | 20.739 | 38.015 | 61.176 | 1.00 | 66.78 | |
| ATOM | 1936 | O | GLY | 457 | 21.568 | 38.471 | 61.968 | 1.00 | 67.21 | |
| ATOM | 1937 | N | GLY | 458 | 20.669 | 36.728 | 60.844 | 1.00 | 66.86 | |
| ATOM | 1938 | CA | GLY | 458 | 21.594 | 35.753 | 61.384 | 1.00 | 66.84 | |
| ATOM | 1939 | C | GLY | 458 | 22.018 | 34.761 | 60.315 | 1.00 | 66.86 | |
| ATOM | 1940 | O | GLY | 458 | 21.450 | 34.801 | 59.199 | 1.00 | 66.65 | |
| ATOM | 1941 | OXT | GLY | 458 | 22.922 | 33.943 | 60.593 | 1.00 | 65.49 | |
| ATOM | 1942 | OH2 | TIP | 1003 | 30.252 | 23.128 | 74.386 | 1.00 | 27.69 | |
| ATOM | 1943 | OH2 | TIP | 1005 | 14.203 | 25.558 | 89.644 | 1.00 | 25.22 | |
| ATOM | 1944 | OH2 | TIP | 1006 | 8.388 | 25.042 | 72.262 | 1.00 | 22.81 | |
| ATOM | 1945 | OH2 | TIP | 1008 | 8.367 | 21.538 | 69.460 | 1.00 | 19.23 | |
| ATOM | 1946 | OH2 | TIP | 1009 | −7.350 | 22.030 | 52.884 | 1.00 | 80.11 | |
| ATOM | 1947 | OH2 | TIP | 1010 | −4.017 | 19.644 | 67.897 | 1.00 | 33.26 | |
| ATOM | 1948 | OH2 | TIP | 1011 | 8.365 | 3.022 | 77.974 | 1.00 | 47.93 | |
| ATOM | 1949 | OH2 | TIP | 1012 | 30.690 | 8.779 | 67.839 | 1.00 | 26.30 | |
| ATOM | 1950 | OH2 | TIP | 1013 | 12.264 | 8.843 | 80.249 | 1.00 | 26.01 | |
| ATOM | 1951 | OH2 | TIP | 1014 | −1.764 | 16.382 | 62.652 | 1.00 | 44.82 | |
| ATOM | 1952 | OH2 | TIP | 1015 | 20.301 | 34.946 | 75.498 | 1.00 | 51.92 | |
| ATOM | 1953 | OH2 | TIP | 1016 | 14.443 | 15.693 | 61.296 | 1.00 | 22.04 | |
| ATOM | 1954 | OH2 | TIP | 1017 | 12.487 | 31.635 | 78.951 | 1.00 | 36.76 | |
| ATOM | 1955 | OH2 | TIP | 1018 | 16.579 | 6.557 | 83.739 | 1.00 | 27.86 | |
| ATOM | 1956 | OH2 | TIP | 1019 | −0.626 | 26.615 | 50.499 | 1.00 | 30.82 | |
| ATOM | 1957 | OH2 | TIP | 1021 | 3.543 | 20.127 | 64.859 | 1.00 | 23.80 | |
| ATOM | 1958 | OH2 | TIP | 1022 | 4.772 | 0.996 | 47.855 | 1.00 | 40.67 | |
| ATOM | 1959 | OH2 | TIP | 1023 | 9.799 | 29.451 | 51.621 | 1.00 | 30.93 | |
| ATOM | 1960 | OH2 | TIP | 1024 | 7.476 | 19.030 | 68.589 | 1.00 | 22.30 | |
| ATOM | 1961 | OH2 | TIP | 1025 | 20.355 | 7.131 | 58.551 | 1.00 | 52.44 | |
| ATOM | 1962 | OH2 | TIP | 1026 | −0.829 | 29.526 | 57.153 | 1.00 | 31.90 | |
| ATOM | 1963 | OH2 | TIP | 1027 | 11.560 | −6.342 | 53.442 | 1.00 | 52.29 | |
| ATOM | 1964 | OH2 | TIP | 1028 | 15.278 | 0.625 | 72.808 | 1.00 | 27.12 | |
| ATOM | 1965 | OH2 | TIP | 1029 | 22.593 | 26.832 | 76.012 | 1.00 | 35.56 | |
| ATOM | 1966 | OH2 | TIP | 1031 | 3.001 | 25.878 | 68.078 | 1.00 | 22.76 | |
| ATOM | 1967 | OH2 | TIP | 1032 | 13.489 | 25.800 | 47.958 | 1.00 | 47.50 | |
| ATOM | 1968 | OH2 | TIP | 1033 | −7.554 | 18.088 | 60.905 | 1.00 | 30.53 | |
| ATOM | 1969 | OH2 | TIP | 1034 | 24.742 | 18.595 | 64.446 | 1.00 | 44.88 | |
| ATOM | 1970 | OH2 | TIP | 1035 | 13.751 | 37.059 | 78.800 | 1.00 | 60.77 | |
| ATOM | 1971 | OH2 | TIP | 1036 | −0.515 | 10.167 | 75.163 | 1.00 | 36.51 | |
| ATOM | 1972 | OH2 | TIP | 1037 | 12.373 | 35.911 | 72.901 | 1.00 | 32.65 | |
| ATOM | 1973 | OH2 | TIP | 1039 | 23.543 | 26.270 | 78.523 | 1.00 | 24.40 | |
| ATOM | 1974 | OH2 | TIP | 1040 | 17.896 | 20.961 | 59.259 | 1.00 | 39.57 | |
| ATOM | 1975 | OH2 | TIP | 1041 | 8.248 | 15.187 | 89.930 | 1.00 | 59.85 | |
| ATOM | 1976 | OH2 | TIP | 1042 | 7.418 | 31.128 | 73.133 | 1.00 | 34.33 | |
| ATOM | 1977 | OH2 | TIP | 1043 | 21.123 | 8.890 | 53.894 | 1.00 | 67.39 | |
| ATOM | 1978 | OH2 | TIP | 1045 | 15.162 | 18.243 | 53.355 | 1.00 | 28.26 | |
| ATOM | 1979 | OH2 | TIP | 1050 | 4.216 | 23.224 | 44.827 | 1.00 | 46.56 | |
| ATOM | 1980 | OH2 | TIP | 1051 | 17.523 | 1.262 | 73.909 | 1.00 | 23.12 | |
| ATOM | 1981 | OH2 | TIP | 1052 | −0.169 | 20.149 | 67.166 | 1.00 | 67.45 | |
| ATOM | 1982 | OH2 | TIP | 1053 | 20.135 | 12.837 | 55.866 | 1.00 | 51.70 | |
| ATOM | 1983 | OH2 | TIP | 1054 | 10.612 | 35.387 | 77.215 | 1.00 | 57.20 | |
| ATOM | 1984 | OH2 | TIP | 1055 | 14.587 | 38.805 | 73.912 | 1.00 | 56.14 | |
| ATOM | 1985 | OH2 | TIP | 1056 | 22.658 | 15.094 | 55.769 | 1.00 | 63.46 | |
| ATOM | 1986 | OH2 | TIP | 1057 | 8.196 | 1.415 | 39.058 | 1.00 | 55.65 | |
| ATOM | 1987 | OH2 | TIP | 1058 | 10.807 | 2.725 | 77.173 | 1.00 | 22.15 | |
| ATOM | 1988 | OH2 | TIP | 1059 | 19.013 | 20.604 | 62.130 | 1.00 | 32.43 | |
| ATOM | 1989 | OH2 | TIP | 1061 | 2.388 | 16.861 | 45.084 | 1.00 | 25.70 | |
| ATOM | 1990 | OH2 | TIP | 1063 | 5.229 | 6.816 | 86.424 | 1.00 | 59.99 | |
| ATOM | 1991 | OH2 | TIP | 1501 | 18.919 | 15.965 | 66.146 | 1.00 | 24.36 | |
| ATOM | 1992 | OH2 | TIP | 1502 | 2.744 | 33.258 | 66.246 | 1.00 | 30.10 | |
| ATOM | 1993 | OH2 | TIP | 1503 | 4.527 | 17.244 | 77.877 | 1.00 | 23.98 | |
| ATOM | 1994 | OH2 | TIP | 1504 | −0.815 | 22.723 | 68.903 | 1.00 | 24.06 | |
| ATOM | 1995 | OH2 | TIP | 1506 | 22.697 | 1.204 | 69.760 | 1.00 | 28.71 | |
| ATOM | 1996 | OH2 | TIP | 1507 | 12.438 | 25.185 | 81.547 | 1.00 | 28.20 | |
| ATOM | 1997 | OH2 | TIP | 1508 | 17.107 | 31.275 | 76.636 | 1.00 | 33.34 | |
| ATOM | 1998 | OH2 | TIP | 1509 | 17.900 | 15.686 | 59.270 | 1.00 | 37.88 | |
| ATOM | 1999 | OH2 | TIP | 1510 | 7.197 | 12.183 | 44.002 | 1.00 | 29.62 | |
| ATOM | 2000 | OH2 | TIP | 1511 | −4.834 | 15.832 | 60.463 | 1.00 | 33.76 | |
| ATOM | 2001 | OH2 | TIP | 1512 | 11.093 | 1.186 | 74.736 | 1.00 | 29.08 | |
| ATOM | 2002 | OH2 | TIP | 1513 | −0.145 | 2.568 | 51.845 | 1.00 | 30.78 | |
| ATOM | 2003 | OH2 | TIP | 1514 | −6.100 | 23.488 | 73.541 | 1.00 | 27.96 | |
| ATOM | 2004 | OH2 | TIP | 1515 | 8.298 | 14.512 | 44.198 | 1.00 | 34.89 | |
| ATOM | 2005 | OH2 | TIP | 1516 | 0.418 | 26.098 | 68.989 | 1.00 | 28.71 | |
| ATOM | 2006 | OH2 | TIP | 1517 | −7.177 | 16.116 | 59.030 | 1.00 | 32.04 | |
| ATOM | 2007 | OH2 | TIP | 1519 | 18.000 | 18.387 | 62.314 | 1.00 | 32.49 | |
| ATOM | 2008 | OH2 | TIP | 1520 | 21.777 | 20.403 | 61.898 | 1.00 | 38.66 | |
| ATOM | 2009 | OH2 | TIP | 1521 | −1.379 | 32.714 | 63.883 | 1.00 | 40.86 | |
| ATOM | 2010 | OH2 | TIP | 1522 | 1.931 | 22.610 | 68.721 | 1.00 | 31.49 | |
| ATOM | 2011 | OH2 | TIP | 1523 | −3.158 | 9.157 | 64.790 | 1.00 | 46.08 | |
| ATOM | 2012 | OH2 | TIP | 1524 | 2.081 | 4.709 | 65.432 | 1.00 | 38.87 | |
| ATOM | 2013 | OH2 | TIP | 1525 | 3.829 | 11.325 | 75.940 | 1.00 | 34.36 | |
| ATOM | 2014 | OH2 | TIP | 1527 | 21.845 | 33.747 | 71.839 | 1.00 | 51.86 | |
| ATOM | 2015 | OH2 | TIP | 1528 | 12.196 | 0.941 | 78.760 | 1.00 | 46.53 | |
| ATOM | 2016 | OH2 | TIP | 1529 | 30.316 | 21.478 | 85.009 | 1.00 | 28.49 | |
| ATOM | 2017 | OH2 | TIP | 1530 | 9.786 | 2.798 | 91.182 | 1.00 | 58.36 | |
| ATOM | 2018 | OH2 | TIP | 1531 | 16.571 | 8.007 | 48.772 | 1.00 | 38.32 | |
| ATOM | 2019 | OH2 | TIP | 1532 | 3.764 | 24.595 | 70.409 | 1.00 | 31.40 | |
| ATOM | 2020 | OH2 | TIP | 1533 | −0.952 | 5.111 | 57.996 | 1.00 | 39.42 | |
| ATOM | 2021 | OH2 | TIP | 1534 | 8.395 | 29.793 | 48.733 | 1.00 | 45.92 | |
| ATOM | 2022 | OH2 | TIP | 1535 | 18.190 | −0.943 | 54.382 | 1.00 | 55.57 | |
| ATOM | 2023 | OH2 | TIP | 1536 | 4.583 | 13.859 | 64.203 | 1.00 | 30.44 | |
| ATOM | 2024 | OH2 | TIP | 1538 | 12.012 | 14.232 | 84.365 | 1.00 | 33.97 | |
| ATOM | 2025 | OH2 | TIP | 1539 | −1.284 | 36.017 | 69.736 | 1.00 | 57.91 | |
| ATOM | 2026 | OH2 | TIP | 1540 | 2.454 | 15.898 | 79.022 | 1.00 | 40.22 | |
| ATOM | 2027 | OH2 | TIP | 1544 | 2.719 | 2.670 | 49.088 | 1.00 | 32.00 | |
| ATOM | 2028 | OH2 | TIP | 1545 | 13.537 | 37.410 | 71.136 | 1.00 | 41.29 | |
| ATOM | 2029 | OH2 | TIP | 1546 | 22.697 | 0.071 | 79.248 | 1.00 | 32.01 | |
| ATOM | 2030 | OH2 | TIP | 1548 | −0.239 | 7.542 | 39.851 | 1.00 | 52.37 | |
| ATOM | 2031 | OH2 | TIP | 1549 | 0.076 | 10.603 | 44.453 | 1.00 | 41.67 | |
| ATOM | 2032 | OH2 | TIP | 1550 | 31.157 | 3.039 | 59.611 | 1.00 | 43.66 | |
| ATOM | 2033 | OH2 | TIP | 1551 | 4.226 | 34.045 | 72.549 | 1.00 | 53.58 | |
| ATOM | 2034 | OH2 | TIP | 1554 | 10.022 | 33.359 | 56.088 | 1.00 | 41.48 | |
| ATOM | 2035 | OH2 | TIP | 1555 | −1.058 | 37.708 | 61.917 | 1.00 | 54.81 | |
| ATOM | 2036 | OH2 | TIP | 1556 | −4.583 | 15.870 | 53.480 | 1.00 | 37.02 | |
| ATOM | 2037 | OH2 | TIP | 1557 | 23.851 | 8.517 | 92.595 | 1.00 | 36.91 | |
| ATOM | 2038 | OH2 | TIP | 1558 | −7.204 | 28.744 | 59.955 | 1.00 | 35.72 | |
| ATOM | 2039 | OH2 | TIP | 1560 | 19.483 | 6.334 | 88.331 | 1.00 | 34.61 | |
| ATOM | 2040 | OH2 | TIP | 1561 | 1.968 | 8.086 | 38.648 | 1.00 | 57.90 | |
| ATOM | 2041 | OH2 | TIP | 1562 | 32.430 | −4.459 | 71.625 | 1.00 | 63.22 | |
| ATOM | 2042 | OH2 | TIP | 1563 | 7.819 | 12.682 | 83.368 | 1.00 | 47.71 | |
| ATOM | 2043 | OH2 | TIP | 1564 | −5.435 | 18.376 | 72.810 | 1.00 | 41.90 | |
| ATOM | 2044 | OH2 | TIP | 1565 | 19.550 | 17.394 | 63.917 | 1.00 | 31.79 | |
| ATOM | 2045 | OH2 | TIP | 1566 | 24.069 | 28.502 | 85.703 | 1.00 | 50.24 | |
| ATOM | 2046 | OH2 | TIP | 1568 | 26.854 | 12.830 | 56.392 | 1.00 | 51.68 | |
| ATOM | 2047 | OH2 | TIP | 1570 | 3.595 | 32.325 | 68.760 | 1.00 | 45.07 | |
| ATOM | 2048 | OH2 | TIP | 1571 | 24.805 | 8.300 | 62.036 | 1.00 | 28.27 | |
| ATOM | 2049 | OH2 | TIP | 1572 | 4.194 | 17.554 | 63.640 | 1.00 | 26.21 | |
| ATOM | 2050 | OH2 | TIP | 1573 | 2.589 | 20.195 | 67.352 | 1.00 | 34.52 | |
| ATOM | 2051 | OH2 | TIP | 1574 | 15.713 | 17.937 | 61.017 | 1.00 | 52.03 | |
| ATOM | 2052 | OH2 | TIP | 1575 | −9.321 | 14.210 | 59.772 | 1.00 | 33.92 | |
| ATOM | 2053 | OH2 | TIP | 1576 | 13.215 | 7.332 | 82.542 | 1.00 | 31.45 | |
| ATOM | 2054 | OH2 | TIP | 1577 | 10.470 | 24.539 | 83.194 | 1.00 | 35.29 | |
| ATOM | 2055 | OH2 | TIP | 1578 | 25.712 | 17.999 | 53.496 | 1.00 | 41.46 | |
| ATOM | 2056 | OH2 | TIP | 1579 | 9.445 | −0.239 | 41.882 | 1.00 | 41.51 | |
| ATOM | 2057 | OH2 | TIP | 1580 | 6.603 | 16.005 | 42.611 | 1.00 | 32.35 | |
| ATOM | 2058 | OH2 | TIP | 1581 | −1.523 | 7.654 | 59.739 | 1.00 | 50.11 | |
| ATOM | 2059 | OH2 | TIP | 1582 | 8.397 | 34.515 | 72.891 | 1.00 | 33.81 | |
| ATOM | 2060 | OH2 | TIP | 1583 | 2.742 | 39.191 | 60.949 | 1.00 | 39.77 | |
| ATOM | 2061 | OH2 | TIP | 1584 | 18.933 | 6.009 | 52.002 | 1.00 | 45.27 | |
| ATOM | 2062 | OH2 | TIP | 1585 | −1.653 | 20.171 | 69.665 | 1.00 | 37.15 | |
| ATOM | 2063 | OH2 | TIP | 1586 | −2.633 | 4.655 | 52.475 | 1.00 | 49.05 | |
| ATOM | 2064 | OH2 | TIP | 1587 | 36.297 | 28.180 | 83.444 | 1.00 | 41.56 | |
| ATOM | 2065 | OH2 | TIP | 1588 | −0.851 | 31.806 | 55.808 | 1.00 | 34.41 | |
| ATOM | 2066 | OH2 | TIP | 1589 | 4.002 | 34.625 | 70.007 | 1.00 | 46.13 | |
| ATOM | 2067 | OH2 | TIP | 1590 | 32.711 | 20.152 | 84.581 | 1.00 | 53.56 | |
| ATOM | 2068 | OH2 | TIP | 1591 | 19.998 | 6.099 | 87.630 | 1.00 | 31.12 | |
| ATOM | 2069 | OH2 | TIP | 1593 | −0.189 | 3.637 | 35.682 | 1.00 | 54.58 | |
| ATOM | 2070 | OH2 | TIP | 1594 | 12.455 | 12.705 | 39.358 | 1.00 | 55.12 | |
| ATOM | 2071 | OH2 | TIP | 1596 | −2.554 | −6.074 | 47.925 | 1.00 | 55.01 | |
| ATOM | 2072 | OH2 | TIP | 1597 | 5.017 | 28.176 | 75.017 | 1.00 | 42.02 | |
| ATOM | 2073 | OH2 | TIP | 1598 | 28.617 | 32.433 | 80.891 | 1.00 | 65.40 | |
| ATOM | 2074 | OH2 | TIP | 1599 | 8.680 | 7.258 | 78.481 | 1.00 | 52.56 | |
| ATOM | 2075 | OH2 | TIP | 1600 | 18.188 | 12.950 | 87.437 | 1.00 | 47.03 | |
| ATOM | 2076 | OH2 | TIP | 1601 | −11.532 | 19.931 | 55.756 | 1.00 | 48.92 | |
| ATOM | 2077 | OH2 | TIP | 1602 | 22.073 | 14.215 | 52.571 | 1.00 | 49.32 | |
| ATOM | 2078 | OH2 | TIP | 1603 | −3.860 | 34.262 | 53.170 | 1.00 | 48.97 | |
| ATOM | 2079 | OH2 | TIP | 1604 | 1.118 | 10.847 | 82.180 | 1.00 | 44.11 | |
| ATOM | 2080 | OH2 | TIP | 1605 | 19.335 | 32.031 | 77.782 | 1.00 | 48.61 | |
| ATOM | 2081 | OH2 | TIP | 1606 | 19.174 | 9.955 | 48.654 | 1.00 | 40.42 | |
| ATOM | 2082 | OH2 | TIP | 1607 | 23.632 | −1.631 | 71.300 | 1.00 | 37.97 | |
| ATOM | 2083 | OH2 | TIP | 1608 | 26.622 | 26.695 | 85.361 | 1.00 | 44.14 | |
| ATOM | 2084 | OH2 | TIP | 1609 | 22.586 | −1.769 | 57.526 | 1.00 | 48.15 | |
| ATOM | 2085 | OH2 | TIP | 1610 | 21.977 | 5.567 | 60.712 | 1.00 | 37.76 | |
| ATOM | 2086 | OH2 | TIP | 1611 | 21.634 | 2.725 | 67.903 | 1.00 | 41.42 | |
| ATOM | 2087 | OH2 | TIP | 1612 | 4.046 | 4.187 | 75.513 | 1.00 | 55.86 | |
| ATOM | 2088 | OH2 | TIP | 1614 | 0.807 | 25.979 | 47.960 | 1.00 | 38.30 | |
| ATOM | 2089 | OH2 | TIP | 1615 | 17.333 | 37.351 | 72.160 | 1.00 | 55.70 | |
| ATOM | 2090 | OH2 | TIP | 1616 | 2.475 | 15.902 | 62.566 | 1.00 | 38.40 | |
| ATOM | 2091 | OH2 | TIP | 1618 | 0.658 | 14.983 | 64.592 | 1.00 | 60.57 | |
| ATOM | 2092 | OH2 | TIP | 1619 | −6.509 | 17.844 | 52.643 | 1.00 | 41.17 | |
| ATOM | 2093 | OH2 | TIP | 1621 | 27.000 | −1.287 | 80.946 | 1.00 | 51.49 | |
| ATOM | 2094 | OH2 | TIP | 1622 | 3.271 | 9.154 | 86.392 | 1.00 | 55.17 | |
| ATOM | 2095 | OH2 | TIP | 1627 | 3.433 | 19.409 | 44.225 | 1.00 | 50.54 | |
| ATOM | 2096 | OH2 | TIP | 1628 | 2.390 | 26.629 | 72.360 | 1.00 | 42.60 | |
| ATOM | 2097 | OH2 | TIP | 1629 | 9.893 | 39.104 | 69.833 | 1.00 | 54.40 | |
| ATOM | 2098 | OH2 | TIP | 1630 | 2.709 | 14.153 | 43.455 | 1.00 | 34.37 | |
| ATOM | 2099 | OH2 | TIP | 1631 | 11.049 | 12.448 | 88.232 | 1.00 | 45.81 | |
| ATOM | 2100 | OH2 | TIP | 1632 | 4.576 | 31.506 | 72.757 | 1.00 | 39.34 | |
| ATOM | 2101 | OH2 | TIP | 1634 | 6.784 | 36.285 | 71.148 | 1.00 | 51.53 | |
| ATOM | 2102 | OH2 | TIP | 1635 | 6.667 | 43.335 | 56.568 | 1.00 | 51.21 | |
| ATOM | 2103 | OH2 | TIP | 1636 | −5.771 | 9.260 | 60.442 | 1.00 | 44.79 | |
| ATOM | 2104 | OH2 | TIP | 1638 | 0.052 | 33.418 | 66.937 | 1.00 | 47.03 | |
| ATOM | 2105 | OH2 | TIP | 1641 | 0.354 | 1.055 | 46.133 | 1.00 | 54.03 | |
| ATOM | 2106 | OH2 | TIP | 1642 | 24.406 | 30.113 | 88.300 | 1.00 | 48.82 | |
| ATOM | 2107 | OH2 | TIP | 1643 | 26.619 | 20.182 | 66.495 | 1.00 | 38.01 | |
| ATOM | 2108 | OH2 | TIP | 1644 | 17.492 | 7.024 | 42.815 | 1.00 | 65.02 | |
| ATOM | 2109 | OH2 | TIP | 1645 | 25.942 | 26.481 | 82.676 | 1.00 | 49.52 | |
| ATOM | 2110 | OH2 | TIP | 1646 | 20.601 | 16.199 | 68.672 | 1.00 | 37.35 | |
| ATOM | 2111 | OH2 | TIP | 1649 | 27.616 | 9.156 | 63.460 | 1.00 | 37.92 | |
| ATOM | 2112 | OH2 | TIP | 1650 | 0.428 | −3.038 | 44.190 | 1.00 | 54.50 | |
| ATOM | 2113 | OH2 | TIP | 1652 | −7.028 | 20.462 | 59.299 | 1.00 | 33.58 | |
| ATOM | 2114 | OH2 | TIP | 1653 | −2.848 | 32.314 | 67.354 | 1.00 | 49.02 | |
| ATOM | 2115 | OH2 | TIP | 1654 | −0.686 | 17.762 | 66.362 | 1.00 | 46.03 | |
| ATOM | 2116 | OH2 | TIP | 1655 | 19.583 | 17.275 | 60.162 | 1.00 | 41.00 | |
| ATOM | 2117 | OH2 | TIP | 1656 | 13.719 | 36.618 | 75.139 | 1.00 | 51.89 | |
| ATOM | 2118 | OH2 | TIP | 1657 | 9.386 | −0.422 | 71.399 | 1.00 | 43.15 | |
| ATOM | 2119 | OH2 | TIP | 1659 | 23.690 | 28.880 | 79.578 | 1.00 | 42.62 | |
| ATOM | 2120 | OH2 | TIP | 1660 | 22.069 | 3.800 | 58.682 | 1.00 | 46.06 | |
| ATOM | 2121 | OH2 | TIP | 1661 | 20.671 | 13.353 | 58.841 | 1.00 | 57.05 | |
| ATOM | 2122 | OH2 | TIP | 1662 | 27.473 | 10.135 | 82.332 | 1.00 | 47.43 | |
| ATOM | 2123 | OH2 | TIP | 1664 | 9.564 | 26.542 | 84.601 | 1.00 | 44.55 | |
| ATOM | 2124 | OH2 | TIP | 1666 | 29.122 | 9.606 | 65.764 | 1.00 | 45.20 | |
| ATOM | 2125 | OH2 | TIP | 1668 | 13.135 | 20.507 | 41.865 | 1.00 | 59.09 | |
| ATOM | 2126 | OH2 | TIP | 1669 | 22.639 | 11.672 | 58.999 | 1.00 | 54.98 | |
| ATOM | 2127 | OH2 | TIP | 1670 | −1.845 | 6.027 | 76.197 | 1.00 | 48.89 | |
| ATOM | 2128 | OH2 | TIP | 1672 | 4.883 | 25.252 | 42.734 | 1.00 | 51.13 | |
| ATOM | 2129 | OH2 | TIP | 1675 | 1.329 | 39.322 | 66.763 | 1.00 | 68.30 | |
| ATOM | 2130 | OH2 | TIP | 1676 | 12.783 | 29.313 | 87.079 | 1.00 | 54.62 | |
| ATOM | 2131 | OH2 | TIP | 1679 | 25.035 | 18.339 | 57.364 | 1.00 | 54.53 | |
| ATOM | 2132 | OH2 | TIP | 1682 | 29.392 | −1.856 | 57.721 | 1.00 | 37.30 | |
| ATOM | 2133 | OH2 | TIP | 1683 | 28.780 | 9.970 | 58.622 | 1.00 | 54.22 | |
| ATOM | 2134 | OH2 | TIP | 1685 | 4.741 | 39.274 | 62.499 | 1.00 | 46.58 | |
| ATOM | 2135 | OH2 | TIP | 1686 | −3.084 | 6.977 | 49.478 | 1.00 | 57.17 | |
| ATOM | 2136 | OH2 | TIP | 1687 | 26.519 | 30.868 | 83.197 | 1.00 | 64.53 | |
| ATOM | 2137 | OH2 | TIP | 1688 | −2.784 | 37.278 | 67.289 | 1.00 | 59.53 | |
| ATOM | 2138 | OH2 | TIP | 1689 | 18.691 | 10.604 | 88.296 | 1.00 | 52.44 | |
| ATOM | 2139 | OH2 | TIP | 1690 | 27.919 | 6.703 | 82.226 | 1.00 | 44.84 | |
| ATOM | 2140 | OH2 | TIP | 1691 | −4.338 | 11.103 | 48.033 | 1.00 | 55.91 | |
| ATOM | 2141 | OH2 | TIP | 1692 | −7.853 | 9.429 | 46.864 | 1.00 | 63.74 | |
| ATOM | 2142 | OH2 | TIP | 1693 | 10.901 | −1.686 | 67.477 | 1.00 | 41.21 | |
| ATOM | 2143 | OH2 | TIP | 1694 | −2.114 | 6.315 | 55.259 | 1.00 | 56.21 | |
| ATOM | 2144 | OH2 | TIP | 1695 | 17.482 | 15.932 | 44.391 | 1.00 | 41.18 | |
| ATOM | 2145 | OH2 | TIP | 1696 | −12.326 | 38.088 | 61.786 | 1.00 | 53.30 | |
| ATOM | 2146 | OH2 | TIP | 1697 | −2.176 | 40.471 | 68.230 | 1.00 | 68.88 | |
| ATOM | 2147 | OH2 | TIP | 1700 | 6.514 | −1.974 | 53.366 | 1.00 | 51.67 | |
| ATOM | 2148 | OH2 | TIP | 1701 | 21.800 | 10.610 | 55.773 | 1.00 | 60.93 | |
| ATOM | 2149 | OH2 | TIP | 1702 | 3.975 | 27.046 | 41.446 | 1.00 | 44.88 | |
| ATOM | 2150 | OH2 | TIP | 1703 | 26.678 | −3.660 | 64.081 | 1.00 | 62.42 | |
| ATOM | 2151 | OH2 | TIP | 1704 | 2.958 | 12.027 | 86.133 | 1.00 | 53.52 | |
| ATOM | 2152 | OH2 | TIP | 1705 | 4.264 | 22.050 | 63.018 | 1.00 | 16.96 | |
| ATOM | 2153 | OH2 | TIP | 1706 | 22.999 | 26.329 | 63.006 | 1.00 | 32.17 | |
| ATOM | 2154 | OH2 | TIP | 1707 | 5.614 | 2.688 | 68.201 | 1.00 | 42.08 | |
| ATOM | 2155 | OH2 | TIP | 1708 | −2.967 | 17.730 | 54.394 | 1.00 | 38.89 | |
| ATOM | 2156 | OH2 | TIP | 1709 | 25.853 | 10.594 | 62.118 | 1.00 | 43.50 | |
| ATOM | 2157 | OH2 | TIP | 1711 | 13.060 | 12.966 | 86.563 | 1.00 | 41.22 | |
| ATOM | 2158 | OH2 | TIP | 1712 | 19.784 | 15.472 | 45.489 | 1.00 | 58.17 | |
| ATOM | 2159 | OH2 | TIP | 1713 | 10.567 | 14.806 | 42.991 | 1.00 | 43.48 | |
| ATOM | 2160 | OH2 | TIP | 1714 | 24.079 | 30.190 | 83.477 | 1.00 | 47.61 | |
| ATOM | 2161 | OH2 | TIP | 1715 | 23.927 | 21.975 | 63.464 | 1.00 | 44.77 | |
| ATOM | 2162 | OH2 | TIP | 1716 | 15.801 | 20.193 | 58.769 | 1.00 | 34.32 | |
| ATOM | 2163 | OH2 | TIP | 1717 | 23.867 | 27.717 | 72.712 | 1.00 | 40.47 | |
| ATOM | 2164 | OH2 | TIP | 1718 | 24.567 | 27.201 | 69.884 | 1.00 | 45.97 | |
| ATOM | 2165 | OH2 | TIP | 1719 | 32.141 | −1.375 | 73.278 | 1.00 | 62.31 | |
| ATOM | 2166 | OH2 | TIP | 1720 | 19.799 | 24.122 | 57.454 | 1.00 | 35.07 | |
| ATOM | 2167 | OH2 | TIP | 1721 | 18.297 | 23.286 | 53.598 | 1.00 | 43.88 | |
| ATOM | 2168 | OH2 | TIP | 1722 | 8.617 | 1.105 | 73.470 | 1.00 | 48.55 | |
| ATOM | 2169 | OH2 | TIP | 1723 | 28.598 | 25.728 | 64.296 | 1.00 | 46.24 | |
| ATOM | 2170 | OH2 | TIP | 1725 | 19.225 | 33.547 | 73.276 | 1.00 | 44.07 | |
| ATOM | 2171 | OH2 | TIP | 1726 | 1.762 | 4.546 | 47.584 | 1.00 | 50.27 | |
| ATOM | 2172 | OH2 | TIP | 1727 | 10.895 | 28.774 | 83.657 | 1.00 | 54.87 | |
| ATOM | 2173 | OH2 | TIP | 1728 | 9.989 | 36.628 | 73.713 | 1.00 | 46.56 | |
| ATOM | 2174 | OH2 | TIP | 1729 | −1.331 | 8.332 | 70.133 | 1.00 | 46.76 | |
| ATOM | 2175 | OH2 | TIP | 1730 | 24.262 | 12.802 | 55.386 | 1.00 | 59.24 | |
| ATOM | 2176 | OH2 | TIP | 1731 | 28.623 | 25.788 | 86.798 | 1.00 | 51.87 | |
| ATOM | 2177 | OH2 | TIP | 1732 | −0.501 | 4.843 | 68.521 | 1.00 | 47.96 | |
| ATOM | 2178 | OH2 | TIP | 1736 | 18.422 | 4.635 | 54.793 | 1.00 | 51.00 | |
| ATOM | 2179 | OH2 | TIP | 1737 | −5.388 | 27.319 | 50.727 | 1.00 | 46.53 | |
| ATOM | 2180 | OH2 | TIP | 1738 | −2.286 | 20.842 | 72.915 | 1.00 | 45.95 | |
| ATOM | 2181 | OH2 | TIP | 1739 | 0.996 | 4.268 | 39.511 | 1.00 | 52.67 | |
| ATOM | 2182 | OH2 | TIP | 1740 | −10.886 | 28.616 | 64.116 | 1.00 | 45.22 | |
| ATOM | 2183 | OH2 | TIP | 1741 | 20.353 | −4.883 | 70.512 | 1.00 | 61.31 | |
| ATOM | 2184 | OH2 | TIP | 1742 | 22.491 | 16.164 | 60.365 | 1.00 | 58.19 | |
| ATOM | 2185 | OH2 | TIP | 3001 | 15.272 | 21.419 | 87.789 | 1.00 | 27.44 | |
| ATOM | 2186 | OH2 | TIP | 3002 | 13.055 | 32.876 | 52.925 | 1.00 | 53.35 | |
| ATOM | 2187 | OH2 | TIP | 3006 | 16.014 | 18.841 | 64.083 | 1.00 | 56.45 | |
| ATOM | 2188 | OH2 | TIP | 3008 | 16.802 | 30.100 | 54.388 | 1.00 | 48.87 | |
| ATOM | 2189 | OH2 | TIP | 3009 | 13.673 | 27.099 | 82.740 | 1.00 | 32.07 | |
| ATOM | 2190 | OH2 | TIP | 3010 | 30.041 | 24.325 | 84.969 | 1.00 | 41.40 | |
| ATOM | 2191 | OH2 | TIP | 3007 | −2.102 | 35.612 | 60.958 | 1.00 | 52.05 | |
| ATOM | 2192 | OH2 | TIP | 3011 | 7.242 | 14.501 | 40.017 | 1.00 | 51.46 | |
| ATOM | 2193 | OH2 | TIP | 3012 | 1.031 | 36.834 | 60.593 | 1.00 | 49.05 | |
| ATOM | 2194 | OH2 | TIP | 3013 | 0.026 | 24.244 | 72.355 | 1.00 | 42.35 | |
| ATOM | 2195 | OH2 | TIP | 3015 | −7.871 | 31.986 | 57.037 | 1.00 | 46.09 | |
| ATOM | 2196 | OH2 | TIP | 3019 | 8.655 | −3.490 | 62.423 | 1.00 | 44.56 | |
| ATOM | 2197 | OH2 | TIP | 3020 | −0.191 | 30.553 | 51.677 | 1.00 | 54.31 | |
| ATOM | 2198 | OH2 | TIP | 3023 | 3.107 | 37.905 | 57.599 | 1.00 | 48.07 | |
| ATOM | 2199 | OH2 | TIP | 3024 | 26.217 | 6.182 | 84.277 | 1.00 | 47.75 | |
| ATOM | 2200 | OH2 | TIP | 3025 | 2.594 | 16.520 | 65.838 | 1.00 | 40.67 | |
| ATOM | 2201 | C1 | EPH | 4000 | 23.874 | 12.843 | 85.264 | 1.00 | 42.79 | |
| ATOM | 2202 | C2 | EPH | 4000 | 23.099 | 13.772 | 86.129 | 1.00 | 44.39 | |
| ATOM | 2203 | C4 | EPH | 4000 | 24.923 | 13.000 | 83.062 | 1.00 | 45.55 | |
| ATOM | 2204 | O2 | EPH | 4000 | 24.221 | 13.543 | 84.059 | 1.00 | 44.96 | |
| ATOM | 2205 | O4 | EPH | 4000 | 25.350 | 11.876 | 83.028 | 1.00 | 47.53 | |
| ATOM | 2206 | C18 | EPH | 4000 | 25.126 | 14.045 | 81.931 | 1.00 | 47.05 | |
| ATOM | 2207 | C19 | EPH | 4000 | 24.674 | 13.569 | 80.547 | 1.00 | 46.98 | |
| ATOM | 2208 | C20 | EPH | 4000 | 23.168 | 13.412 | 80.473 | 1.00 | 49.00 | |
| ATOM | 2209 | C21 | EPH | 4000 | 22.772 | 12.939 | 79.111 | 1.00 | 51.06 | |
| ATOM | 2210 | C22 | EPH | 4000 | 21.365 | 12.327 | 79.073 | 1.00 | 52.88 | |
| ATOM | 2211 | C23 | EPH | 4000 | 20.291 | 13.230 | 78.423 | 1.00 | 54.24 | |
| ATOM | 2212 | C24 | EPH | 4000 | 20.651 | 13.777 | 77.022 | 1.00 | 55.08 | |
| ATOM | 2213 | C25 | EPH | 4000 | 19.987 | 15.129 | 76.704 | 1.00 | 56.13 | |
| ATOM | 2214 | C26 | EPH | 4000 | 20.807 | 16.375 | 77.147 | 1.00 | 56.19 | |
| ATOM | 2215 | C27 | EPH | 4000 | 19.981 | 17.687 | 77.287 | 1.00 | 56.74 | |
| ATOM | 2216 | C28 | EPH | 4000 | 19.188 | 18.077 | 76.015 | 1.00 | 56.93 | |
| ATOM | 2217 | C29 | EPH | 4000 | 20.055 | 18.393 | 74.792 | 1.00 | 57.07 | |
| ATOM | 2218 | C30 | EPH | 4000 | 19.294 | 18.387 | 73.442 | 1.00 | 58.24 | |
| ATOM | 2219 | C31 | EPH | 4000 | 19.391 | 17.124 | 72.550 | 1.00 | 58.70 | |
| ATOM | 2220 | C32 | EPH | 4000 | 18.019 | 16.479 | 72.198 | 1.00 | 59.07 | |
| ATOM | 2221 | C33 | EPH | 4000 | 16.762 | 17.158 | 72.768 | 1.00 | 59.61 | |
| ATOM | 2222 | C34 | EPH | 4000 | 15.463 | 16.541 | 72.231 | 1.00 | 60.17 | |
| ATOM | 2223 | C37 | EPH | 4000 | 22.780 | 13.059 | 87.421 | 1.00 | 47.03 | |
| ATOM | 2224 | O5 | EPH | 4000 | 22.047 | 13.939 | 88.273 | 1.00 | 53.23 | |
| ATOM | 2225 | P1 | EPH | 4000 | 21.699 | 13.222 | 89.578 | 1.00 | 56.71 | |
| ATOM | 2226 | O6 | EPH | 4000 | 20.350 | 13.536 | 89.939 | 1.00 | 58.07 | |
| ATOM | 2227 | O7 | EPH | 4000 | 22.579 | 12.360 | 90.311 | 1.00 | 56.72 | |
| ATOM | 2228 | O8 | EPH | 4000 | 22.167 | 14.340 | 90.336 | 1.00 | 55.93 | |
| ATOM | 2229 | C3 | EPH | 4000 | 21.561 | 15.394 | 85.253 | 1.00 | 42.20 | |
| ATOM | 2230 | O1 | EPH | 4000 | 21.886 | 14.117 | 85.454 | 1.00 | 40.69 | |
| ATOM | 2231 | O3 | EPH | 4000 | 22.221 | 16.347 | 85.571 | 1.00 | 40.97 | |
| ATOM | 2232 | C5 | EPH | 4000 | 20.215 | 15.530 | 84.546 | 1.00 | 40.77 | |
| ATOM | 2233 | C6 | EPH | 4000 | 20.313 | 15.776 | 83.050 | 1.00 | 42.19 | |
| ATOM | 2234 | C7 | EPH | 4000 | 18.924 | 15.916 | 82.453 | 1.00 | 43.05 | |
| ATOM | 2235 | C8 | EPH | 4000 | 18.900 | 15.944 | 80.947 | 1.00 | 44.57 | |
| ATOM | 2236 | C9 | EPH | 4000 | 17.477 | 16.101 | 80.445 | 1.00 | 45.49 | |
| ATOM | 2237 | C10 | EPH | 4000 | 17.167 | 15.209 | 79.273 | 1.00 | 47.18 | |
| ATOM | 2238 | C11 | EPH | 4000 | 16.561 | 15.987 | 78.117 | 1.00 | 47.85 | |
| ATOM | 2239 | C12 | EPH | 4000 | 15.158 | 15.514 | 77.781 | 1.00 | 49.75 | |
| ATOM | 2240 | C13 | EPH | 4000 | 15.158 | 14.254 | 76.932 | 1.00 | 49.27 | |
| ATOM | 2241 | C14 | EPH | 4000 | 14.899 | 14.563 | 75.454 | 1.00 | 51.41 | |
| ATOM | 2242 | C15 | EPH | 4000 | 14.958 | 13.341 | 74.490 | 1.00 | 51.93 | |
| ATOM | 2243 | C16 | EPH | 4000 | 16.376 | 12.870 | 74.074 | 1.00 | 52.39 | |
| ATOM | 2244 | C17 | EPH | 4000 | 16.681 | 11.465 | 74.554 | 1.00 | 52.37 | |
| ATOM | 2245 | C35 | EPH | 4000 | 17.830 | 11.518 | 75.525 | 1.00 | 52.96 | |
| ATOM | 2246 | C36 | EPH | 4000 | 17.968 | 10.168 | 76.193 | 1.00 | 53.00 | |
| ATOM | 2247 | C38 | EPH | 4000 | 22.197 | 10.885 | 90.057 | 1.00 | 56.75 | |
| ATOM | 2248 | C39 | EPH | 4000 | 23.458 | 10.026 | 89.911 | 1.00 | 56.42 | |
| ATOM | 2249 | N1 | EPH | 4000 | 24.546 | 10.825 | 89.334 | 1.00 | 54.45 | |
| ATOM | 2250 | N | SER | 236 | 17.914 | 25.370 | 86.674 | 0.50 | 20.48 | AC2 |
| ATOM | 2251 | CA | SER | 236 | 18.176 | 23.976 | 86.323 | 0.50 | 19.91 | AC2 |
| ATOM | 2252 | CB | SER | 236 | 19.157 | 23.889 | 85.166 | 0.50 | 18.72 | AC2 |
| ATOM | 2253 | OG | SER | 236 | 19.325 | 22.538 | 84.787 | 0.50 | 17.70 | AC2 |
| ATOM | 2254 | C | SER | 236 | 18.741 | 23.171 | 87.483 | 0.50 | 21.62 | AC2 |
| ATOM | 2255 | O | SER | 236 | 19.744 | 23.549 | 88.075 | 0.50 | 20.94 | AC2 |
| ATOM | 2256 | N | SER | 247 | 25.235 | 21.608 | 79.357 | 0.50 | 19.68 | AC2 |
| ATOM | 2257 | CA | SER | 247 | 25.203 | 22.865 | 78.619 | 0.50 | 20.48 | AC2 |
| ATOM | 2258 | CB | SER | 247 | 26.051 | 23.917 | 79.337 | 0.50 | 19.95 | AC2 |
| ATOM | 2259 | OG | SER | 247 | 26.032 | 25.152 | 78.637 | 0.50 | 20.60 | AC2 |
| ATOM | 2260 | C | SER | 247 | 25.725 | 22.638 | 77.203 | 0.50 | 20.84 | AC2 |
| ATOM | 2261 | O | SER | 247 | 25.203 | 23.202 | 76.238 | 0.50 | 21.05 | AC2 |
| ATOM | 2262 | N | SER | 271 | 7.551 | 30.448 | 53.176 | 0.50 | 29.97 | AC2 |
| ATOM | 2263 | CA | SER | 271 | 7.680 | 31.880 | 53.442 | 0.50 | 31.61 | AC2 |
| ATOM | 2264 | CB | SER | 271 | 8.888 | 32.443 | 52.695 | 0.50 | 32.22 | AC2 |
| ATOM | 2265 | OG | SER | 271 | 8.666 | 32.395 | 51.295 | 0.50 | 32.71 | AC2 |
| ATOM | 2266 | C | SER | 271 | 6.432 | 32.648 | 53.010 | 0.50 | 32.99 | AC2 |
| ATOM | 2267 | O | SER | 271 | 6.229 | 33.796 | 53.408 | 0.50 | 34.05 | AC2 |
| ATOM | 2268 | N | PRO | 319 | 18.143 | −4.099 | 74.681 | 0.50 | 41.20 | AC2 |
| ATOM | 2269 | CD | PRO | 319 | 18.070 | −4.311 | 76.139 | 0.50 | 40.63 | AC2 |
| ATOM | 2270 | CA | PRO | 319 | 18.053 | −2.673 | 74.356 | 0.50 | 38.50 | AC2 |
| ATOM | 2271 | CB | PRO | 319 | 17.702 | −2.038 | 75.699 | 0.50 | 39.26 | AC2 |
| ATOM | 2272 | CG | PRO | 319 | 18.406 | −2.938 | 76.680 | 0.50 | 39.94 | AC2 |
| ATOM | 2273 | C | PRO | 319 | 19.321 | −2.077 | 73.756 | 0.50 | 35.96 | AC2 |
| ATOM | 2274 | O | PRO | 319 | 20.410 | −2.230 | 74.313 | 0.50 | 35.87 | AC2 |
| ATOM | 2275 | N | GLN | 343 | 13.913 | 5.584 | 80.085 | 0.50 | 18.73 | AC2 |
| ATOM | 2276 | CA | GLN | 343 | 12.714 | 5.137 | 79.387 | 0.50 | 19.83 | AC2 |
| ATOM | 2277 | CB | GLN | 343 | 11.463 | 5.365 | 80.243 | 0.50 | 21.34 | AC2 |
| ATOM | 2278 | CG | GLN | 343 | 10.903 | 4.078 | 80.837 | 0.50 | 26.09 | AC2 |
| ATOM | 2279 | CD | GLN | 343 | 9.539 | 4.244 | 81.488 | 0.50 | 27.02 | AC2 |
| ATOM | 2280 | OE1 | GLN | 343 | 9.412 | 4.823 | 82.562 | 0.50 | 29.15 | AC2 |
| ATOM | 2281 | NE2 | GLN | 343 | 8.508 | 3.730 | 80.829 | 0.50 | 29.67 | AC2 |
| ATOM | 2282 | C | GLN | 343 | 12.545 | 5.813 | 78.025 | 0.50 | 19.53 | AC2 |
| ATOM | 2283 | O | GLN | 343 | 12.317 | 5.141 | 77.022 | 0.50 | 19.10 | AC2 |
| ATOM | 2284 | N | SER | 353 | 14.027 | 4.461 | 65.783 | 0.50 | 19.97 | AC2 |
| ATOM | 2285 | CA | SER | 353 | 15.191 | 3.950 | 65.107 | 0.50 | 20.43 | AC2 |
| ATOM | 2286 | CB | SER | 353 | 16.391 | 4.058 | 66.033 | 0.50 | 20.57 | AC2 |
| ATOM | 2287 | OG | SER | 353 | 17.540 | 4.234 | 65.262 | 0.50 | 19.64 | AC2 |
| ATOM | 2288 | C | SER | 353 | 15.054 | 2.524 | 64.574 | 0.50 | 21.08 | AC2 |
| ATOM | 2289 | O | SER | 353 | 15.234 | 2.291 | 63.378 | 0.50 | 21.51 | AC2 |
| ATOM | 2290 | N | ARG | 392 | 0.696 | 20.186 | 53.122 | 0.50 | 19.50 | AC2 |
| ATOM | 2291 | CA | ARG | 392 | 0.935 | 18.822 | 53.549 | 0.50 | 19.96 | AC2 |
| ATOM | 2292 | CB | ARG | 392 | 0.325 | 18.551 | 54.925 | 0.50 | 19.99 | AC2 |
| ATOM | 2293 | CG | ARG | 392 | 0.603 | 17.129 | 55.384 | 0.50 | 19.08 | AC2 |
| ATOM | 2294 | CD | ARG | 392 | 0.140 | 16.884 | 56.805 | 0.50 | 19.85 | AC2 |
| ATOM | 2295 | NE | ARG | 392 | −1.315 | 16.885 | 56.925 | 0.50 | 19.23 | AC2 |
| ATOM | 2296 | CZ | ARG | 392 | −1.962 | 16.577 | 58.046 | 0.50 | 21.63 | AC2 |
| ATOM | 2297 | NH1 | ARG | 392 | −1.283 | 16.242 | 59.138 | 0.50 | 19.92 | AC2 |
| ATOM | 2298 | NH2 | ARG | 392 | −3.289 | 16.611 | 58.086 | 0.50 | 22.19 | AC2 |
| ATOM | 2299 | C | ARG | 392 | 0.338 | 17.878 | 52.501 | 0.50 | 19.95 | AC2 |
| ATOM | 2300 | O | ARG | 392 | 0.940 | 16.867 | 52.149 | 0.50 | 20.64 | AC2 |
| ATOM | 2301 | N | SER | 431 | 9.466 | 14.172 | 75.955 | 0.50 | 16.96 | AC2 |
| ATOM | 2302 | CA | SER | 431 | 10.735 | 14.900 | 76.047 | 0.50 | 17.25 | AC2 |
| ATOM | 2303 | CB | SER | 431 | 11.346 | 15.109 | 74.659 | 0.50 | 17.09 | AC2 |
| ATOM | 2304 | OG | SER | 431 | 10.765 | 16.211 | 73.998 | 0.50 | 16.93 | AC2 |
| ATOM | 2305 | C | SER | 431 | 10.466 | 16.249 | 76.719 | 0.50 | 17.71 | AC2 |
| ATOM | 2306 | O | SER | 431 | 11.267 | 16.716 | 77.521 | 0.50 | 17.14 | AC2 |

6. A LBD according to claim 5, comprising a ligand-binding pocket which is defined by the amino acids LEU230, VAL238, PRO239, PHE242, LEU249, LEU291, ILE294, MET323, LEU331, GLN338, ALA339, VAL341, PHE345, SER431, HIS434, LEU435, PHE438 and LEU440 as shown in SEQ ID NO: 2 and Table 1.

7. A LBD according to claim 5, comprising a ligand-binding pocket which is defined by the amino acids LEU230, VAL238, PRO239, PHE242, PRO245, VAL246, LEU249, CYS250, GLY253, ASN287, LEU290, LEU291, ILE294, MET323, LEU325, LEU331, SER335, ALA336, GLN338, ALA339, VAL341, ILE344, PHE345, VAL348, SER431, HIS434, LEU435, PHE438, LEU440 as shown in SEQ ID NO: 2 and Table 1.

8. A computer-readable data-storage medium comprising a data-storage material in which the structure coordinates of an LBD according to claim 1 are stored.

9. A computer-readable data-storage medium comprising a data-storage material in which the structure coordinates of an LBD according to claim 1 are stored in a form which makes it possible to generate a three-dimensional image of an LBD according to claim 1 on a computer screen.

10. A method for generating protein models of USP-LBDs, comprising the step of preparing by computer-aided generation a three-dimensional image of an LBD according to claim 1.

11. A method for generating protein models of USP-LBDs in an agonistic conformation comprising the step of preparing by computer-aided generation a three-dimensional image of an LBD according to claim 1 in an agonistic conformation.

12. A method for generating protein models of nuclear receptors with homologies to USP-LBDs, comprising the step of preparing by computer-aided generation a three-dimensional image of an LBD according to claim 1 with a mutated amino acid sequence.

13. A method for generating protein models of nuclear receptors with homologies to USP-LBDs, in an agonistic conformation, comprising the step of preparing by computer-aided generation a three-dimensional image of an LBD according to claim 1 with a mutated amino acid sequence in an agonistic conformation.

14. A method of finding USP ligands, comprising the steps of:
(a) preparing by computer-aided generation a three-dimensional image of an LBD according to claim 1, and
(b) performing computer-aided (virtual) screening of databases which contain structural data of chemical compounds for those structures which are capable of undergoing specific interactions with an LBD according to claim 1.

15. A method of finding USP ligands, comprising the steps of:
(a) preparing by computer-aided generation a three-dimensional image of an LBD according to claim 1, and
(b) performing computer-aided modelling of chemical compounds with structures which are capable of undergoing specific interactions with an LBD according to claim 1.

16. A method of finding USP-LBD ligands in an agonistic conformation, comprising the steps of:
(a) preparing by computer-aided generation a three-dimensional image of an LBD according to claim 1 in an agonistic conformation, and
(b) performing computer-aided (virtual) screening of databases which contain structural data of chemical compounds for those structures which are capable of undergoing specific interactions with an LBD in an agonistic conformation.

17. A method of finding USP-LBD ligands in an agonistic conformation, comprising the steps of:
(a) preparing by computer-aided generation a three-dimensional image of an LBD according to claim 1 in an agonistic conformation, and
(b) performing computer-aided modelling of chemical compounds with structures which are capable of undergoing specific interactions with an LBD in an agonistic conformation.

18. A method of finding active compounds for crop protection, in particular chemical compounds which, owing to binding to an LBD according to claim 1, bring about the activation or inhibition of USP, comprising the steps of:
(a) preparing by computer-aided generation a three-dimensional image of an LBD according to claim 1,
(b) performing computer-aided (virtual) screening of databases which contain structural data of chemical compounds for those structures which are capable of undergoing specific interactions with an LBD according to claim 1,
(c) synthesizing the compound(s) identified as ligands, and
(d) detecting the bioactivity of the compound synthesized in step (c) by transactivation assays, displacement assays or bioassays.

19. A method of finding active compounds for crop protection, in particular chemical compounds which, owing to binding to an LBD according to claim 1 in an agonistic conformation, bring about the activation or inhibition of USP, comprising the steps of:
(a) preparing by computer-aided generation a three-dimensional image of an LBD according to claim 1 in an agonistic conformation,
(b) performing computer-aided (virtual) screening of databases which contain structural data of chemical compounds for those structures which are capable of undergoing specific interactions with an LBD in an agonistic conformation,
(c) synthesizing the compound(s) identified as ligands, and
(d) detecting the bioactivity of the compound synthesized in step (c) by transactivation assays, displacement assays or bioassays.

20. A method of finding effectors for systems for the inducible expression of target genes by means of USP, comprising the steps of:
(a) preparing by computer-aided generation a three-dimensional image of an LBD according to claim 1,
(b) performing computer-aided (virtual) screening of databases which contain structural data of chemical compounds for those structures which are capable of undergoing specific interactions with an LBD according to claim 1,
(c) synthesizing the compound(s) identified as ligands,
(d) applying a compound synthesized in step (c) to host cells or host organisms comprising a USP-based expression system, and
(e) detecting an induction or inhibition of the expression system.

21. A method of finding active compounds for crop protection, in particular chemical compounds which, owing to binding to an LBD according to claim 1, bring about the activation or inhibition of USP, comprising the steps of:
(a) preparing by computer-aided generation a three-dimensional image of an LBD according to claim 1,
(b) performing computer-aided modelling of chemical compounds with structures which are capable of undergoing specific interactions with an LBD according to claim 1,
(c) synthesizing the compound(s) identified as ligands, and
(d) detecting the bioactivity of the compound synthesized in step (c) by transactivation assays, displacement assays or bioassays.

22. A method of finding active compounds for crop protection, in particular chemical compounds which, owing to binding to an LBD according to claim 1 in an agonistic conformation, bring about the activation or inhibition of USP, comprising the steps of:
(a) preparing by computer-aided generation a three-dimensional image of an LBD according to claim 1 in an agonistic conformation, and
(b) performing computer-aided modelling of chemical compounds with structures which are capable of undergoing specific interactions with an LBD in an agonistic conformation.
(c) synthesizing the compound(s) identified as ligands, and
(d) detecting the bioactivity of the compound synthesized in step (c) by transactivation assays, displacement assays or bioassays.

23. A method of finding effectors for systems for the inducible expression of target genes by means of USP, comprising the steps of:
(a) preparing by computer-aided generation a three-dimensional image of an LBD according to claim 1,
(b) performing computer-aided modelling of chemical compounds with structures which are capable of undergoing specific interactions with an LBD according to claim 1,
(c) synthesizing the compound(s) identified as ligands,
(d) applying a compound synthesized in step (c) to host cells or host organisms comprising a USP-based expression system, and
(e) detecting an induction or inhibition of the expression system.
